(19) United States
(12) Patent Application Publication
(10) Pub. No.: US 2024/0400547 A1
(43) Pub. Date: Dec. 5, 2024

(54) 2,6-PIPERIDINEDIONE COMPOUND AND APPLICATION THEREOF

(71) Applicant: MEDSHINE DISCOVERY INC., Nanjing, Jiangsu (CN)

(72) Inventors: Maoyi LEI, Shanghai (CN); Yu XU, Shanghai (CN); Shaohui WANG, Shanghai (CN); Yunfu LUO, Shanghai (CN); Shuhui CHEN, Shanghai (CN)

(21) Appl. No.: 18/695,174

(22) PCT Filed: Sep. 21, 2022
(86) PCT No.: PCT/CN2022/120267

§ 371 Date: Mar. 25, 2024

(30) Foreign Application Priority Data
Sep. 26, 2021 (CN) 202111131041.0
Sep. 9, 2022 (CN) 2022111104698.2

(51) Int. Cl. C07D 413/14; A61K 31/4545; A61K 31/496; A61P 35/00; C07D 417/14

(52) U.S. Cl. CPC C07D 413/14; A61K 31/4545; A61K 31/496; A61P 35/00; C07D 417/14

(57) ABSTRACT

Provided are a 2,6-piperidinedione compound and an application thereof. Particularly provided are a compound represented by formula (II), a stereoisomer thereof, and a pharmaceutically acceptable salt thereof.

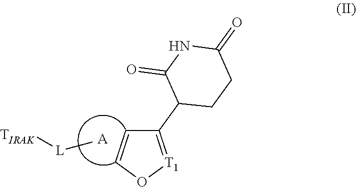

The present application claims right of the following priorities:

This application claims 1) the priority and rights of Chinese Patent Application No. 2021111310410 submitted to CNIPA on Sep. 26, 2021, and 2) the priority and rights of Chinese Patent Application No. 2022111046982 submitted to CNIPA on Sep. 9, 2022, the contents of which are incorporated herein by reference in their entireties.

### TECHNICAL FIELD

The invention relates to a class of 2,6-piperidinedione compounds and an application thereof. Specifically, the invention relates to a compound of formula (II), a stereoisomer thereof, and a pharmaceutically acceptable salt thereof.

### BACKGROUND

Interleukin-1 Receptor-Associated Kinase 4 (IRAK4) plays a connecting role in the signal transduction pathway between Toll-like receptor family (TLRs) and interleukin-1 receptor family (IL-1R), and receives upstream signals to activate its downstream JNK and NF-κB signal pathways, which is closely related to the development and progression of human inflammatory immune diseases and tumors.

The Toll-like receptor (TLR) signal transduction protein myeloid differentiation factor (MyD88) often mutates in various lymphomas, such as Waldenstrom macroglobulinemia, lymphoplasmacytic lymphoma, anti-immune large B-cell lymphoma and marginal zone lymphoma, with mutation rates of 95-97%, 79%, 50-80%, 15-29% and 6-10% respectively. IRAK4 is involved in almost all biological functions of MyD88, making it an extremely attractive drug target with unlimited potentials, especially for the treatment of MyD88-driven lymphomas.

Researches have found that IRAK4 can not only phosphorylate protein, but also form a complex with MyD88 to exert its biological functions. The activation of the JNK signaling pathway by IRAK4 requires its phosphorylation function, yet activation of the NF-κB signaling pathway does not require its phosphorylation function, which indicates that IRAK4 possesses both protein kinase and scaffold protein functions, and plays roles in signaling pathways. Therefore, traditional small-molecule kinase inhibitors targeting IRAK4 cannot completely block all biological functions of IRAK4.

Proteolysis targeting chimera (PROTAC) is a technique that applies the ubiquitin-proteasome system to target specific proteins and induce their intracellular degradation. The ubiquitin-proteasome system is the primary pathway for intracellular protein degradation, mainly responsible for the clearance of denatured, mutated, or harmful proteins as its normal physiological functions. Over 80% of intracellular protein degradation is dependent on the ubiquitin-proteasome system. PROTAC utilizes the cell's own protein destruction mechanism to clear specific targeted proteins within the cell. To date, the PROTAC technology has become increasingly mature, and can be used to target a variety of proteins, including scaffold proteins, transcription factors, enzymes, and regulatory proteins. In addition, domide-class drugs, known as immunomodulatory drugs (IMiDs), activate the E3 ubiquitin ligase complex formed with Cereblon (CRBN) to ubiquitinate the transcription factors IKZF1 and IKZF3, which are then recognized and degraded by the proteasome, thereby producing a toxic effect on tumors. CRBN, as an important target for antitumor and immunomodulatory drugs, has been proven to have definitive therapeutic effects in a variety of hematological malignancies, skin diseases such as erythema nodosum leprosum, and autoimmune diseases like systemic lupus erythematosus.

Therefore, the development of PROTAC and IMiD bifunctional molecules targeting IRAK4 can more thoroughly block all functions of IRAK4 by degrading and removing IRAK4, and thus fundamentally inhibits the IRAK4 signaling pathway comprehensively, while also possessing good CRBN modulating effects, leading to synergistic therapeutic effects. Consequently, the strategy can better exert anti-tumor effects and improve clinical treatment outcomes.

### CONTENT OF THE PRESENT INVENTION

The present disclosure provides a compound of formula (II), a stereoisomer thereof, or a pharmaceutically acceptable salt thereof,

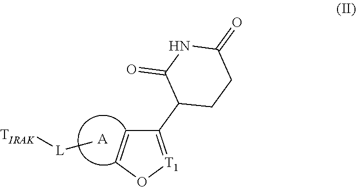

wherein
T_{IRAK} is selected from

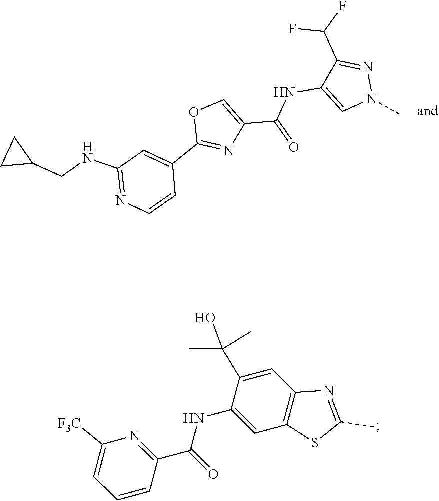

L is selected from C₂₋₁₀ alkylene, any 2, 3 or 4 CH₂ on the C₂₋₁₀ alkylene are each independently substituted by Ra, and each C₂₋₁₀ alkylene is independently and optionally substituted by 1, 2, 3, 4, 5, or 6 halogens;
Each Ra is independently selected from —N(R)—, —O—, —C(O)NH—, —C₃₋₆ cycloalkyl- and -4- to 8-membered heterocycloalkyl-;
R is selected from H and C₁₋₄ alkyl;
T₁ is selected from CH and N;
ring A is selected from C₆₋₁₀ aryl and 5- to 10-membered heteroaryl.

In some embodiments of the present disclosure, the Ra is independently selected from —NH—, —N(CH₃)—, —O—, —C(O)NH—, -cyclopropyl-, -cyclobutyl-, -cyclopentyl-, -cyclohexyl-, -piperidyl-, -piperazinyl-, -azaspiro[3.3]heptyl-, -diazspiro[3.3]heptyl-, and -azabicyclic[3.1.0]hexyl-, and other variables are as defined in the present disclosure.

In some embodiments of the present disclosure, each Ra is independently selected from —NH—, —N(CH₃)—, —O—, —C(O)NH—,

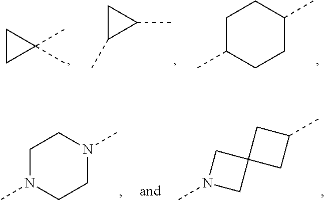

and other variables are as defined in the present disclosure.

In some embodiments of the present disclosure, the L is selected from C₄₋₁₀ alkylene, any 3 CH₂ on the C₄₋₁₀ alkylene are each independently substituted by Ra, each C₄₋₁₀ alkylene is independently and optionally substituted by 1, 2, 3, 4, 5 or 6 halogens, and other variables are as defined in the present disclosure.

In some embodiments of the present disclosure, the Lis selected from —C₃₋₆ cycloalkyl-CH₂—Ra—C₁₋₃ alkylene-Ra—C₀₋₃ alkylene-, —C₃₋₆ cycloalkyl-C₁₋₃ alkylene-4- to 8-membered heterocycloalkyl-C₁₋₃ alkylene-Ra—, and —C₃₋₆ cycloalkyl-C₁₋₃ alkylene-4- to 8-membered heterocycloalkyl-C₁₋₃ alkylene-, each C₁₋₃ alkylene is independently and optionally substituted by 1 or 2 halogens, and other variables are as defined in the present disclosure.

In some embodiments of the present disclosure, the L is selected from -cyclohexyl-CH₂—N(R)—C₁₋₃ alkylene-O—C₁₋₃ alkylene-, -cyclohexyl-CH₂-4- to 8-membered heterocycloalkyl-C₁₋₃ alkylene-N (R)—, -cyclohexyl-CH₂-piperidyl-C₁₋₃ alkylene-C(O)NH—, -cyclohexyl-CH₂ piperidyl-CF₂—C(O)NH—, -cyclohexyl-CH₂-piperazinyl-C₁₋₃ alkylene-C(O)NH—, and -cyclohexyl-CH₂-piperazinyl-C₁₋₃ alkylene-, and other variables are as defined in the present disclosure.

In some embodiments of the present disclosure, the L is selected from

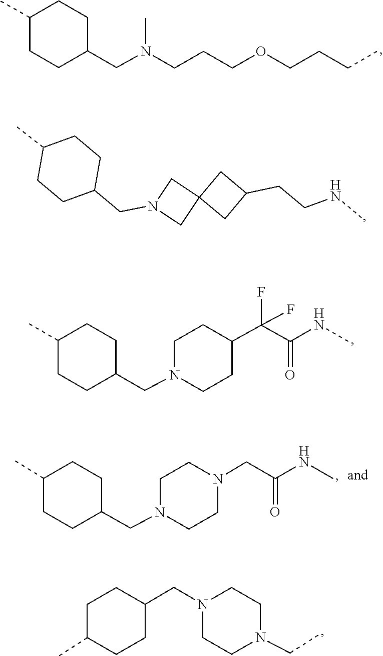

and other variables are as defined in the present disclosure.

In some embodiments of the present disclosure, the ring A is selected from phenyl and naphthyl, and other variables are as defined in the present disclosure.

In some embodiments of the present disclosure, the structural moiety

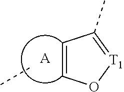

is selected from

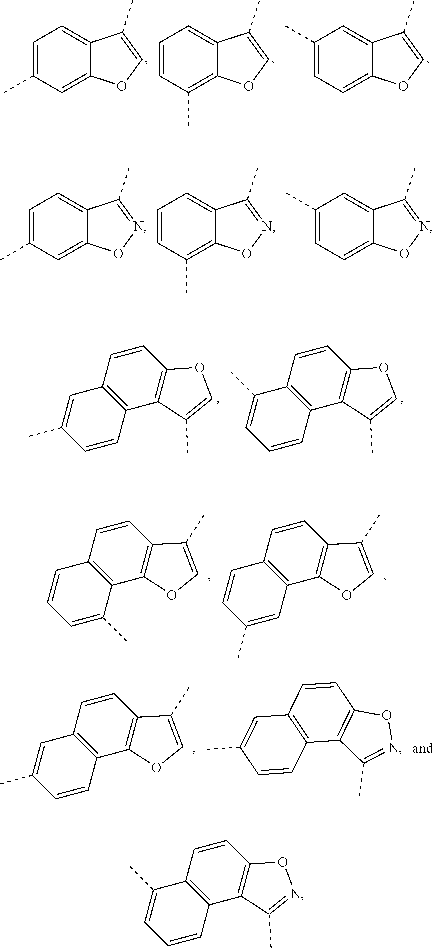

and other variables are as defined in the present disclosure.

In some embodiments of the present disclosure, the structural moiety

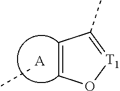

is selected from

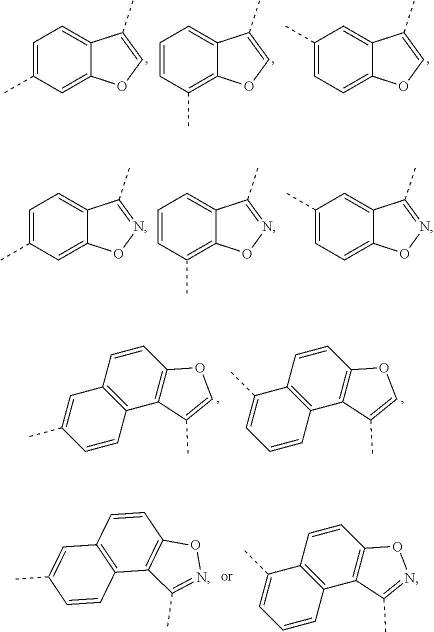

and other variables are as defined in the present disclosure.

The present disclosure also provides compounds of formulas (II-1), (II-2), (II-3), and (II-4), a stereoisomer thereof, or a pharmaceutically acceptable salt thereof,

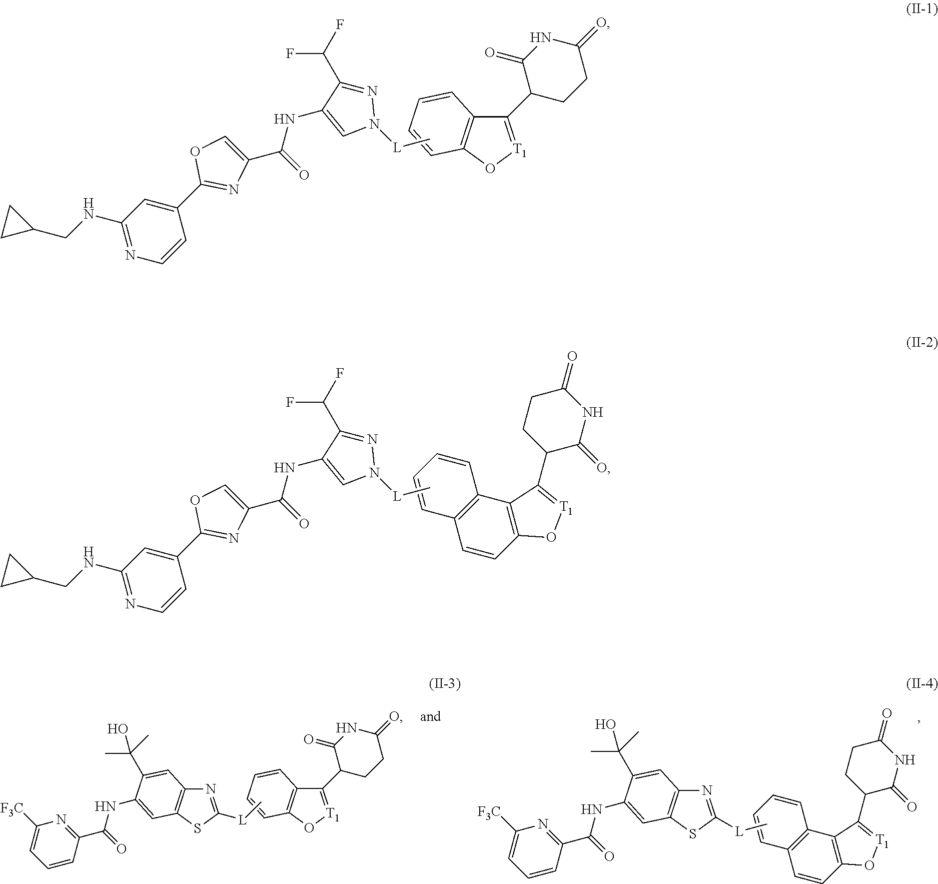

wherein L and T₁ are as defined in formula (II).

The present disclosure also provides compounds of formulas (II-1a), (II-2a), (II-3a), and (II-4a), a stereoisomer thereof, or a pharmaceutically acceptable salt thereof,

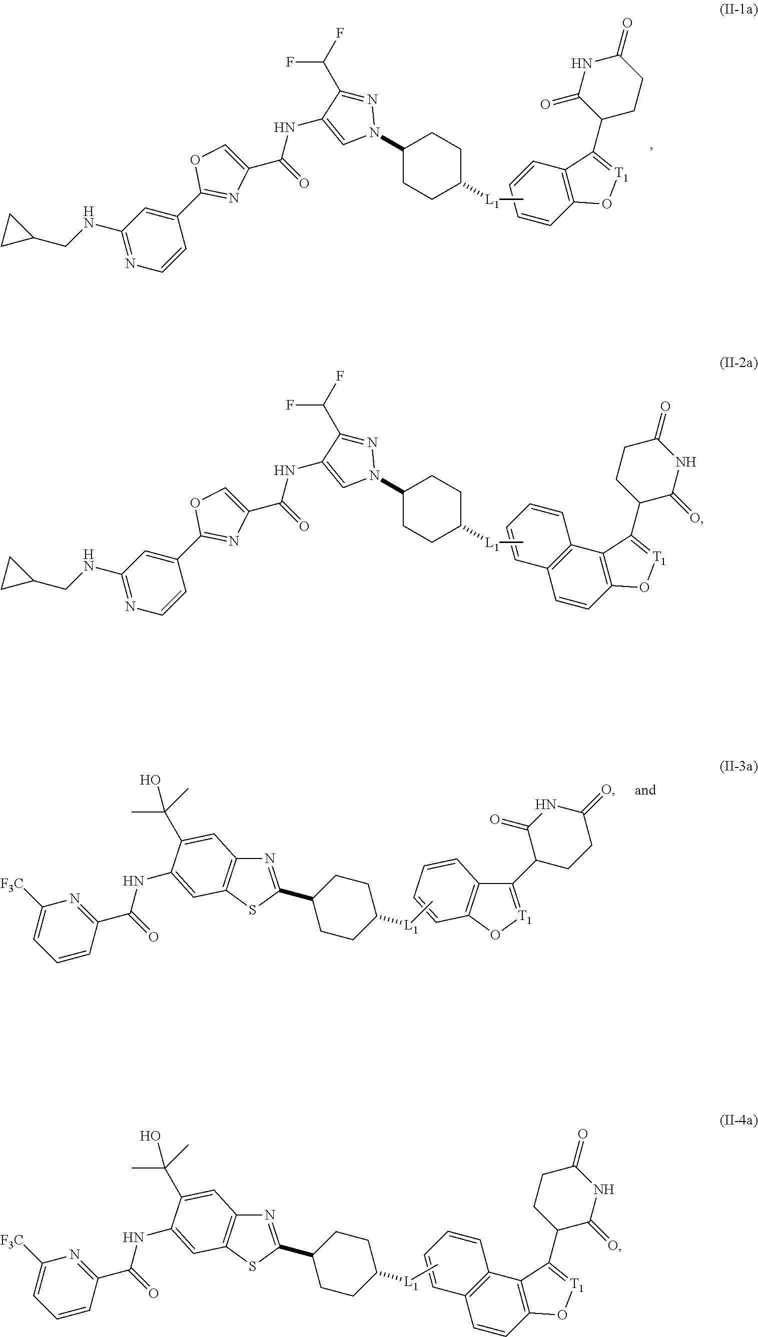

wherein
L₁ is selected from —C₁₋₃ alkylene-Ra—C₁₋₃ alkylene-Ra—C₀₋₃ alkylene-, —C₁₋₃ alkylene-4- to 8-membered heterocycloalkyl-C₁₋₃ alkylene-Ra—, and —C₁₋₃ alkylene-4- to 8-membered heterocycloalkyl-C₁₋₃ alkylene-, and each C₁₋₃ alkylene is independently and optionally substituted by 1 or 2 halogen atoms;
T₁ and Ra are as defined in formula (II).

The present disclosure also provides compounds of formula (I), a stereoisomer thereof, or a pharmaceutically acceptable salt thereof,

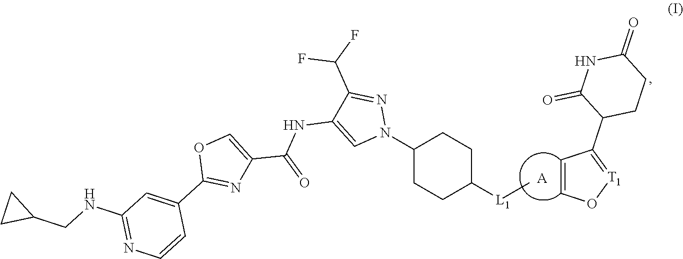

wherein
L₁ is selected from C₁₋₁₀ alkylene, wherein 1, 2, or 3 CH₂ are optionally substituted by Ra, and the C₁₋₁₀ alkylene is optionally substituted by 1 or 2 halogen atoms;
Ra is selected from —N(R)—, —O—, —C(O)NH—, —C₃₋₆ cycloalkyl-, or -4- to 8-membered heterocycloalkyl-;
R is selected from H or C₁₋₄ alkyl;
T₁ is selected from CH or N;
ring A is selected from C₆₋₁₀ aryl or 5- to 10-membered heteroaryl;
the “4- to 8-membered heterocycloalkyl and 5- to 10-membered heteroaryl” each contains 1, 2 or 3 heteroatoms independently selected from N, O, S, or Se.

In some embodiments of the present disclosure, the Ra in formula (I) is selected from —N(CH₃)—, —O—, —C(O)NH—, -cyclopropyl- or -piperazinyl-, and other variables are as defined in the present disclosure.

In some embodiments of the present disclosure, the Ra in formula (I) is selected from —N(CH₃)—, —O—, —C(O)NH—, or

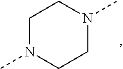

and other variables are as defined in the present disclosure.

In some embodiments of the present disclosure, the L₁ in formula (I) is selected from

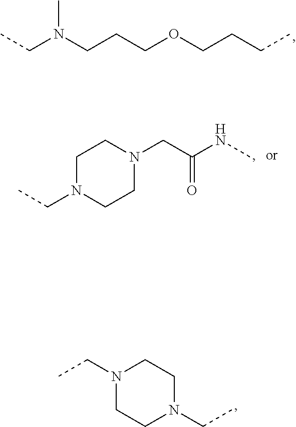

and other variables are as defined in the present disclosure.

In some embodiments of the present disclosure, the ring A in formula (I) is selected from phenyl or naphthyl, and other variables are as defined in the present disclosure.

In some embodiments of the present disclosure, the structural moiety

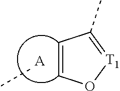

in formula (I) is selected from

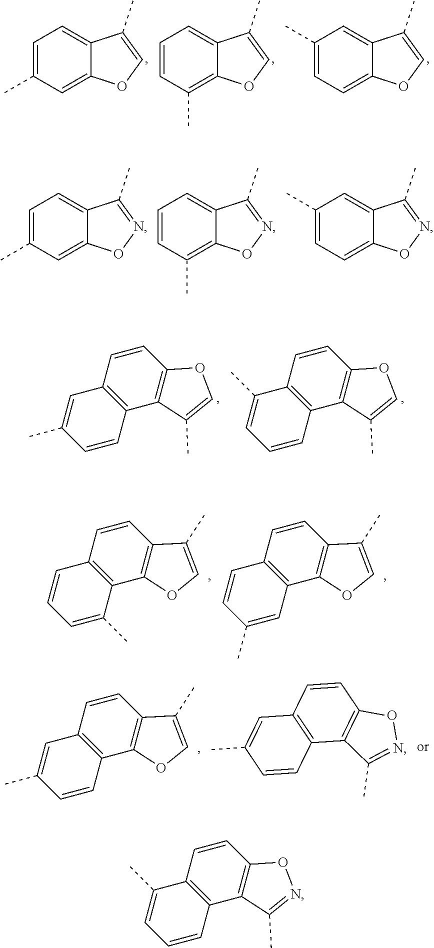

or and other variables are as defined in the present disclosure.

The present disclosure also provides compounds of formulas (I-1), (I-2), and (I-3), a stereoisomer thereof, or a pharmaceutically acceptable salt thereof,

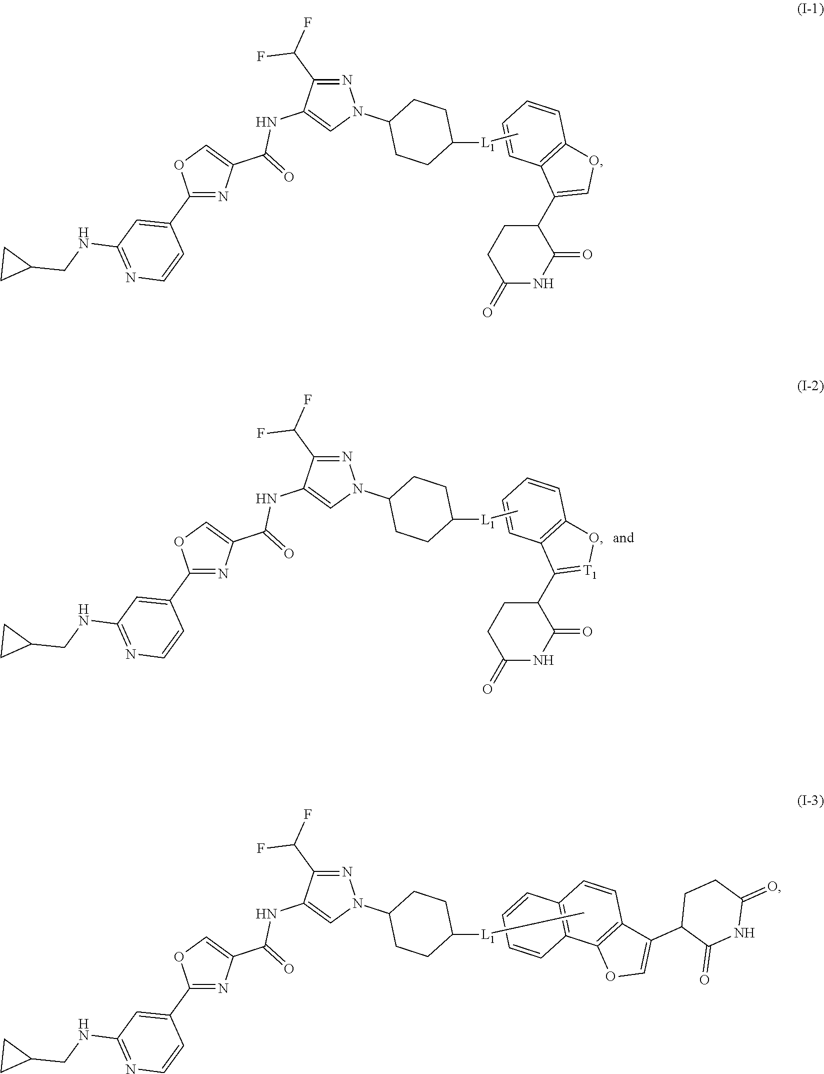

wherein L₁ and T₁ are as defined in the present disclosure.

The present disclosure also provides a compound, a stereoisomer thereof, or a pharmaceutically acceptable salt thereof as shown below,

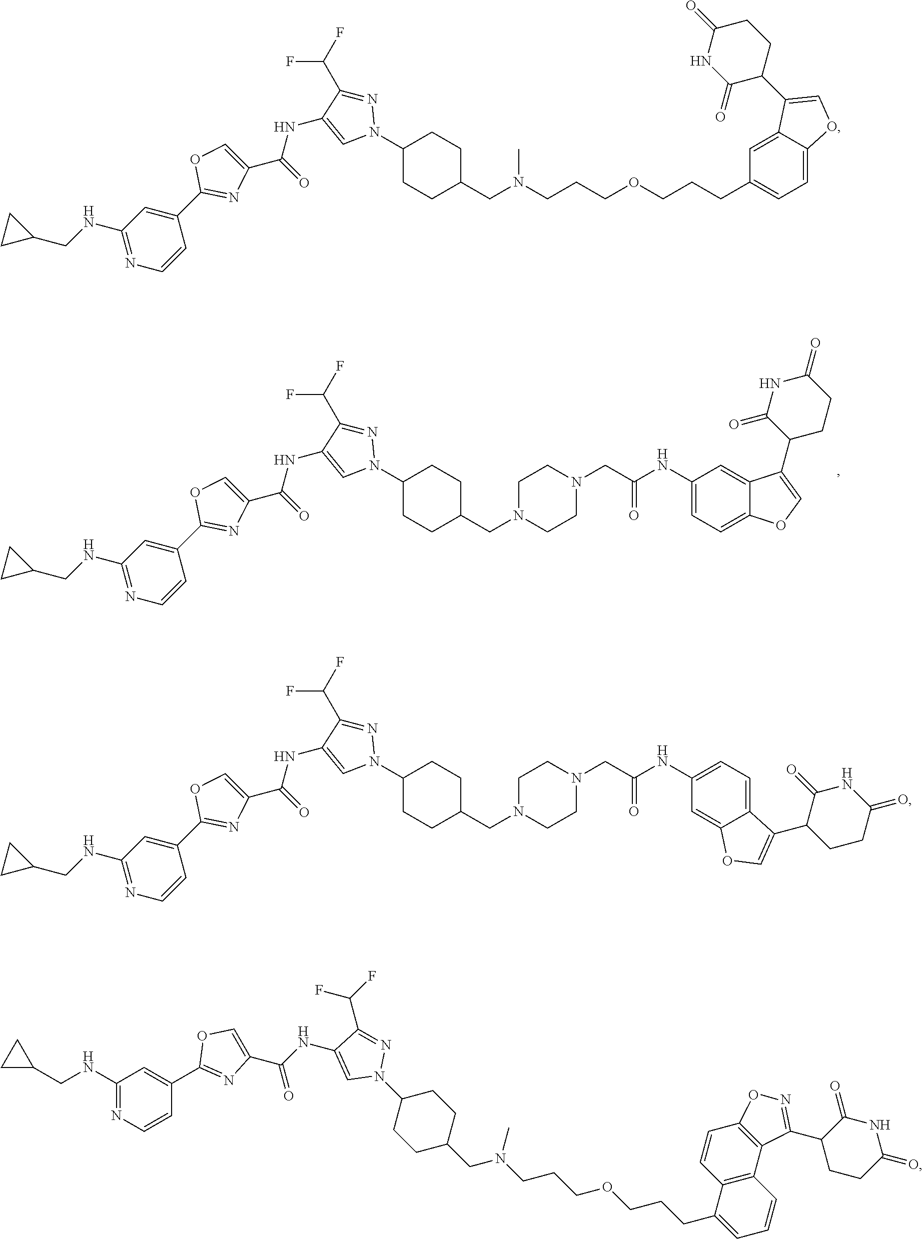 [SMILES: CN(CCCOCCCc1ccc2occ(C3CCC(=O)NC3=O)c2c1)CC1CCC(n2cc(NC(=O)c3coc(-c4ccnc(NCC5CC5)c4)n3)c(C(F)F)n2)CC1 ; O=C1CCC(c2coc3ccc(NC(=O)CN4CCN(CC5CCC(n6cc(NC(=O)c7coc(-c8ccnc(NCC9CC9)c8)n7)c(C(F)F)n6)CC5)CC4)cc23)C(=O)N1 ; O=C1CCC(c2coc3cc(NC(=O)CN4CCN(CC5CCC(n6cc(NC(=O)c7coc(-c8ccnc(NCC9CC9)c8)n7)c(C(F)F)n6)CC5)CC4)ccc23)C(=O)N1 ; CN(CCCOCCCc1cccc2c1ccc1onc(C3CCC(=O)NC3=O)c12)CC1CCC(n2cc(NC(=O)c3coc(-c4ccnc(NCC5CC5)c4)n3)c(C(F)F)n2)CC1]

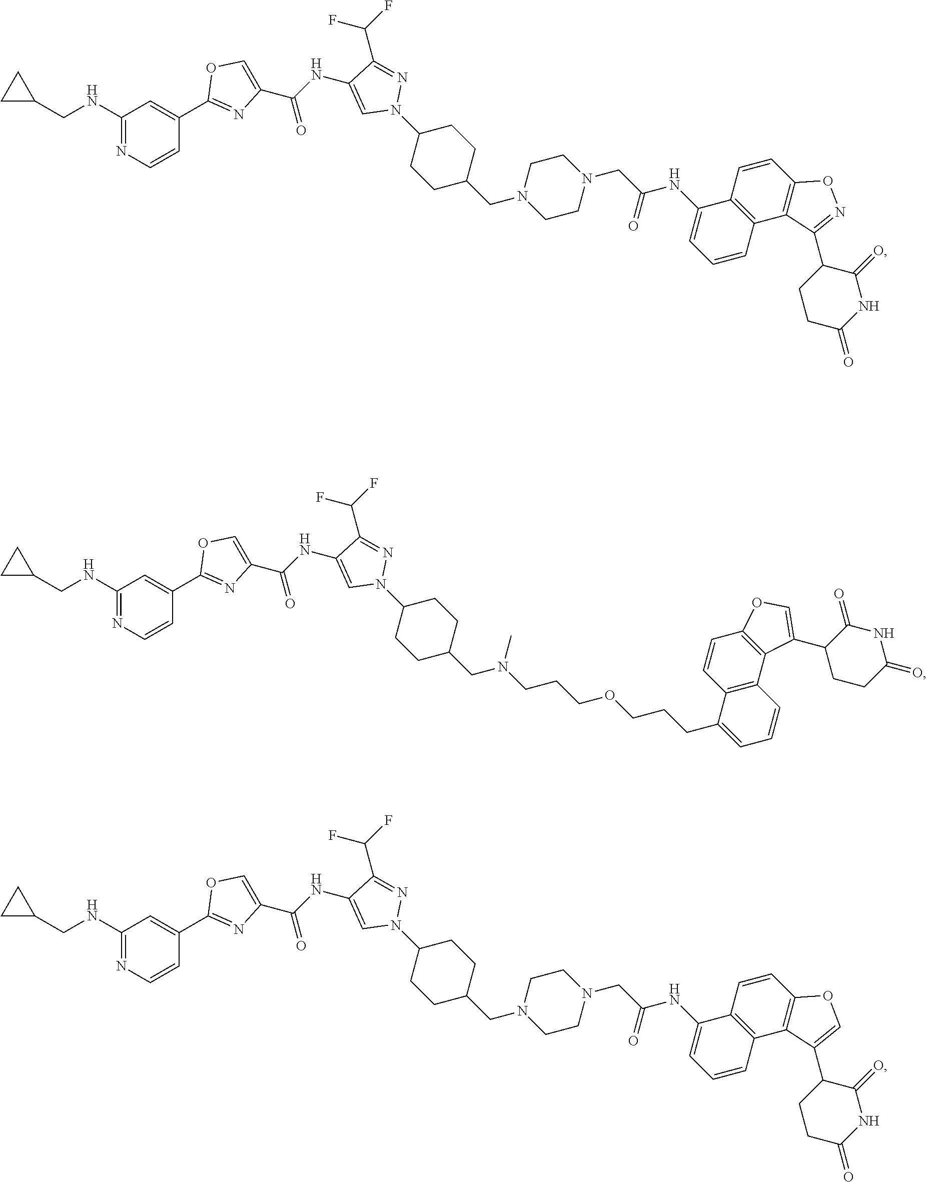 [SMILES: O=C1CCC(c2noc3ccc4c(NC(=O)CN5CCN(CC6CCC(n7cc(NC(=O)c8coc(-c9ccnc(NCC%10CC%10)c9)n8)c(C(F)F)n7)CC6)CC5)cccc4c23)C(=O)N1 ; CN(CCCOCCCc1cccc2c1ccc1occ(C3CCC(=O)NC3=O)c12)CC1CCC(n2cc(NC(=O)c3coc(-c4ccnc(NCC5CC5)c4)n3)c(C(F)F)n2)CC1 ; O=C1CCC(c2coc3ccc4c(NC(=O)CN5CCN(CC6CCC(n7cc(NC(=O)c8coc(-c9ccnc(NCC%10CC%10)c9)n8)c(C(F)F)n7)CC6)CC5)cccc4c23)C(=O)N1]

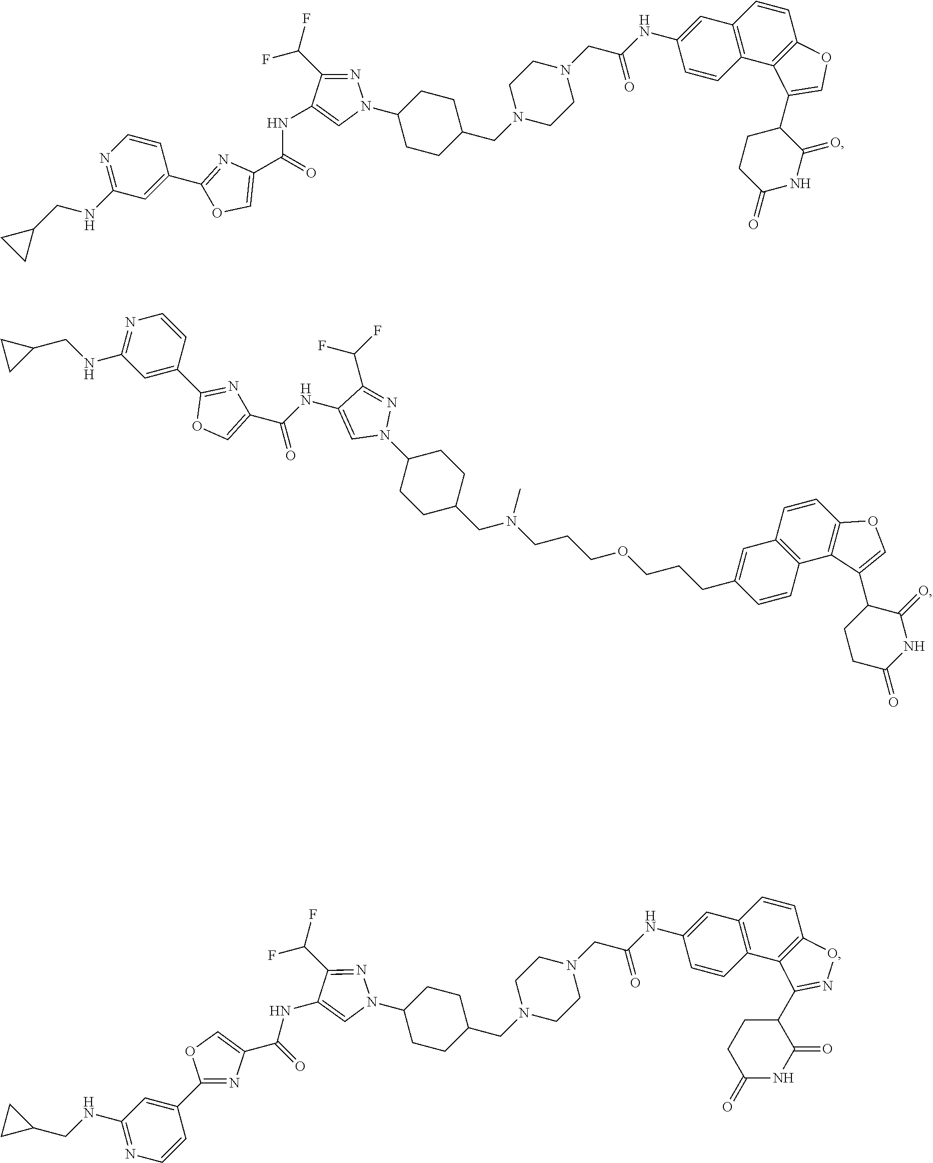 [SMILES: O=C1CCC(c2coc3ccc4cc(NC(=O)CN5CCN(CC6CCC(n7cc(NC(=O)c8coc(-c9ccnc(NCC%10CC%10)c9)n8)c(C(F)F)n7)CC6)CC5)ccc4c23)C(=O)N1 ; CN(CCCOCCCc1ccc2c(ccc3occ(C4CCC(=O)NC4=O)c32)c1)CC1CCC(n2cc(NC(=O)c3coc(-c4ccnc(NCC5CC5)c4)n3)c(C(F)F)n2)CC1 ; O=C1CCC(c2noc3ccc4cc(NC(=O)CN5CCN(CC6CCC(n7cc(NC(=O)c8coc(-c9ccnc(NCC%10CC%10)c9)n8)c(C(F)F)n7)CC6)CC5)ccc4c23)C(=O)N1]

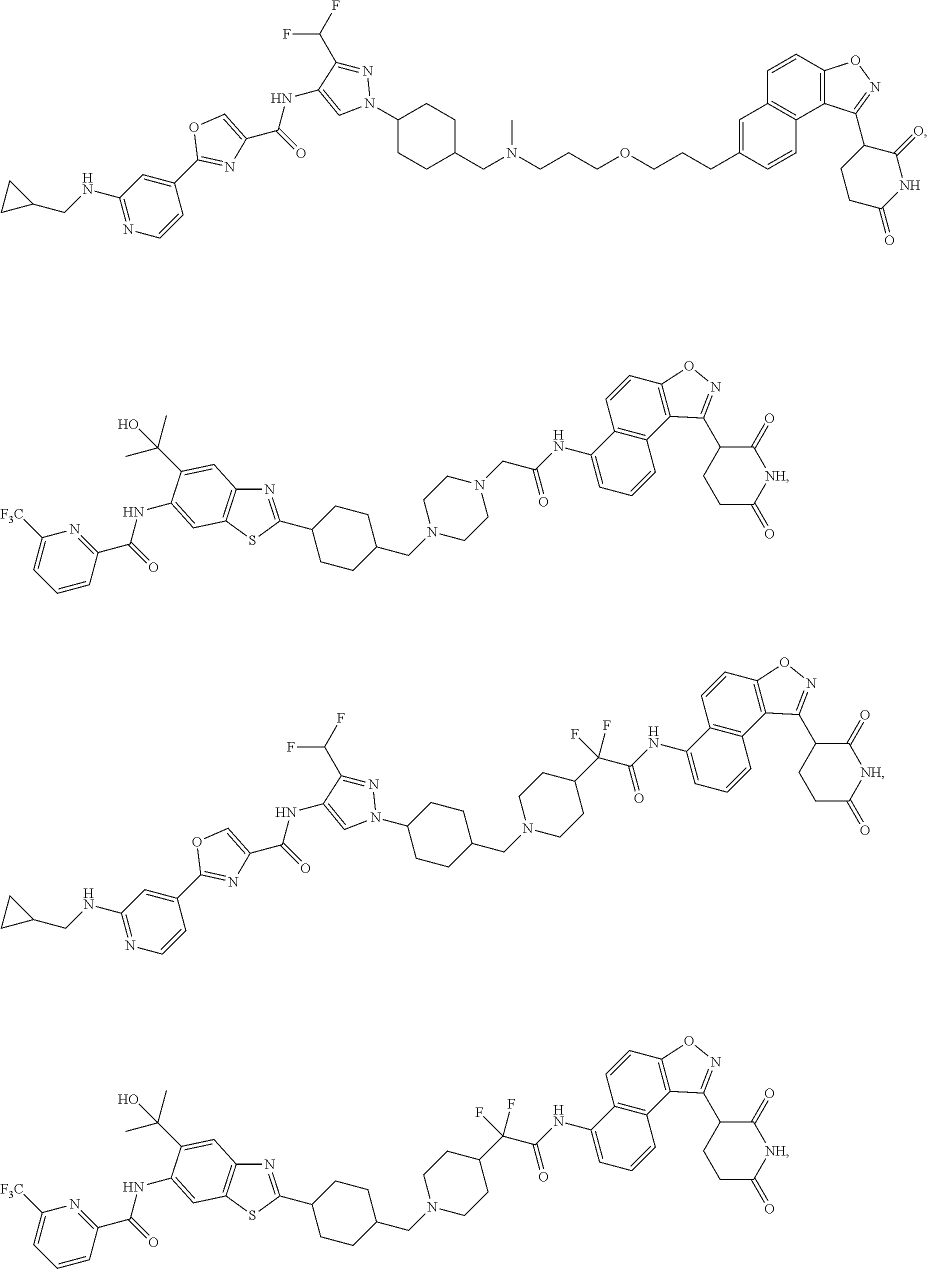 [SMILES: CN(CCCOCCCc1ccc2c(ccc3onc(C4CCC(=O)NC4=O)c32)c1)CC1CCC(n2cc(NC(=O)c3coc(-c4ccnc(NCC5CC5)c4)n3)c(C(F)F)n2)CC1 ; CC(C)(O)c1cc2nc(C3CCC(CN4CCN(CC(=O)Nc5cccc6c5ccc5onc(C7CCC(=O)NC7=O)c56)CC4)CC3)sc2cc1NC(=O)c1cccc(C(F)(F)F)n1 ; O=C1CCC(c2noc3ccc4c(NC(=O)C(F)(F)C5CCN(CC6CCC(n7cc(NC(=O)c8coc(-c9ccnc(NCC%10CC%10)c9)n8)c(C(F)F)n7)CC6)CC5)cccc4c23)C(=O)N1 ; CC(C)(O)c1cc2nc(C3CCC(CN4CCC(C(F)(F)C(=O)Nc5cccc6c5ccc5onc(C7CCC(=O)NC7=O)c56)CC4)CC3)sc2cc1NC(=O)c1cccc(C(F)(F)F)n1]

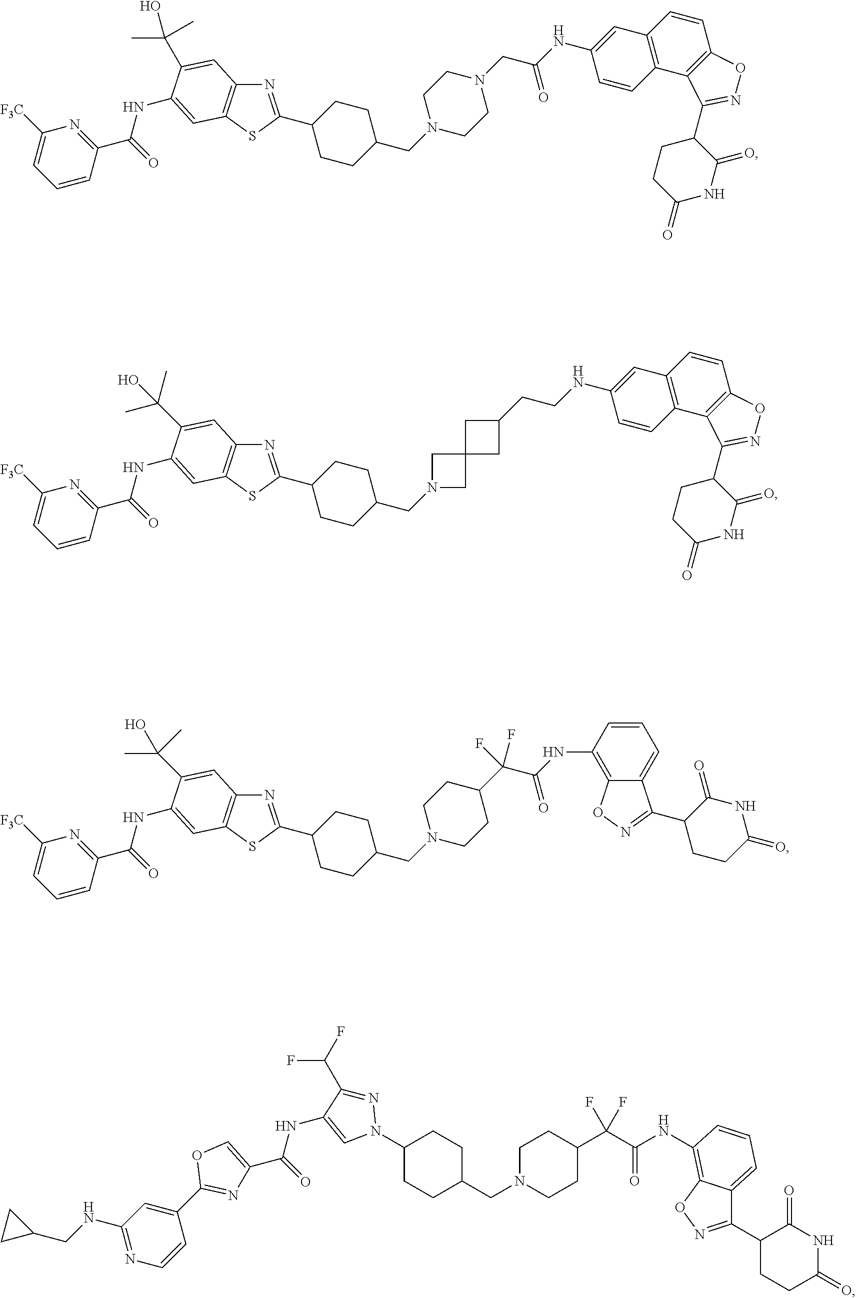 [SMILES: CC(C)(O)c1cc2nc(C3CCC(CN4CCN(CC(=O)Nc5ccc6c(ccc7onc(C8CCC(=O)NC8=O)c76)c5)CC4)CC3)sc2cc1NC(=O)c1cccc(C(F)(F)F)n1 ; CC(C)(O)c1cc2nc(C3CCC(CN4CC5(CC(CCNc6ccc7c(ccc8onc(C9CCC(=O)NC9=O)c87)c6)C5)C4)CC3)sc2cc1NC(=O)c1cccc(C(F)(F)F)n1 ; CC(C)(O)c1cc2nc(C3CCC(CN4CCC(C(F)(F)C(=O)Nc5cccc6c(C7CCC(=O)NC7=O)noc56)CC4)CC3)sc2cc1NC(=O)c1cccc(C(F)(F)F)n1 ; O=C1CCC(c2noc3c(NC(=O)C(F)(F)C4CCN(CC5CCC(n6cc(NC(=O)c7coc(-c8ccnc(NCC9CC9)c8)n7)c(C(F)F)n6)CC5)CC4)cccc23)C(=O)N1]

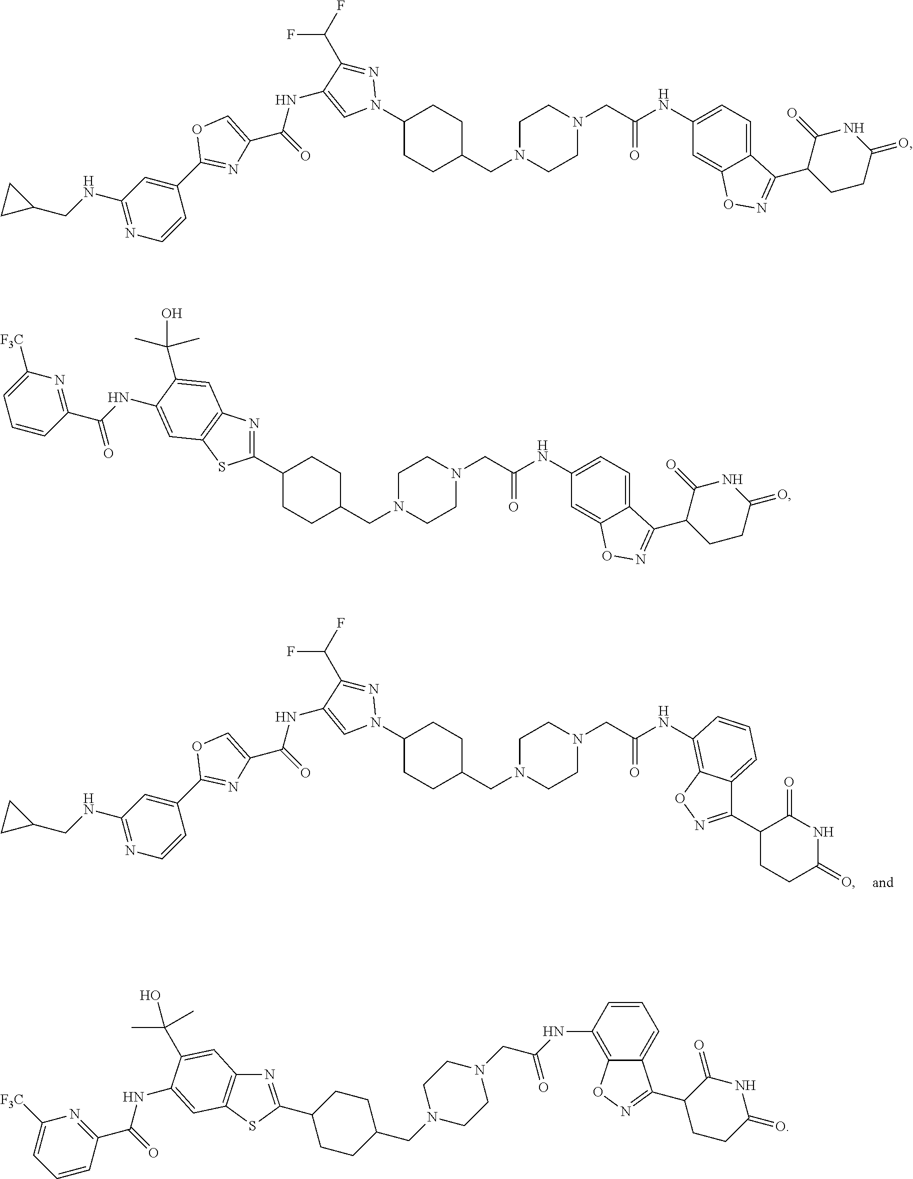 [SMILES: O=C1CCC(c2noc3cc(NC(=O)CN4CCN(CC5CCC(n6cc(NC(=O)c7coc(-c8ccnc(NCC9CC9)c8)n7)c(C(F)F)n6)CC5)CC4)ccc23)C(=O)N1 ; CC(C)(O)c1cc2nc(C3CCC(CN4CCN(CC(=O)Nc5ccc6c(C7CCC(=O)NC7=O)noc6c5)CC4)CC3)sc2cc1NC(=O)c1cccc(C(F)(F)F)n1 ; O=C1CCC(c2noc3c(NC(=O)CN4CCN(CC5CCC(n6cc(NC(=O)c7coc(-c8ccnc(NCC9CC9)c8)n7)c(C(F)F)n6)CC5)CC4)cccc23)C(=O)N1 ; CC(C)(O)c1cc2nc(C3CCC(CN4CCN(CC(=O)Nc5cccc6c(C7CCC(=O)NC7=O)noc56)CC4)CC3)sc2cc1NC(=O)c1cccc(C(F)(F)F)n1]

The present disclosure also provides a compound, a stereoisomer thereof, or a pharmaceutically acceptable salt thereof as shown below,

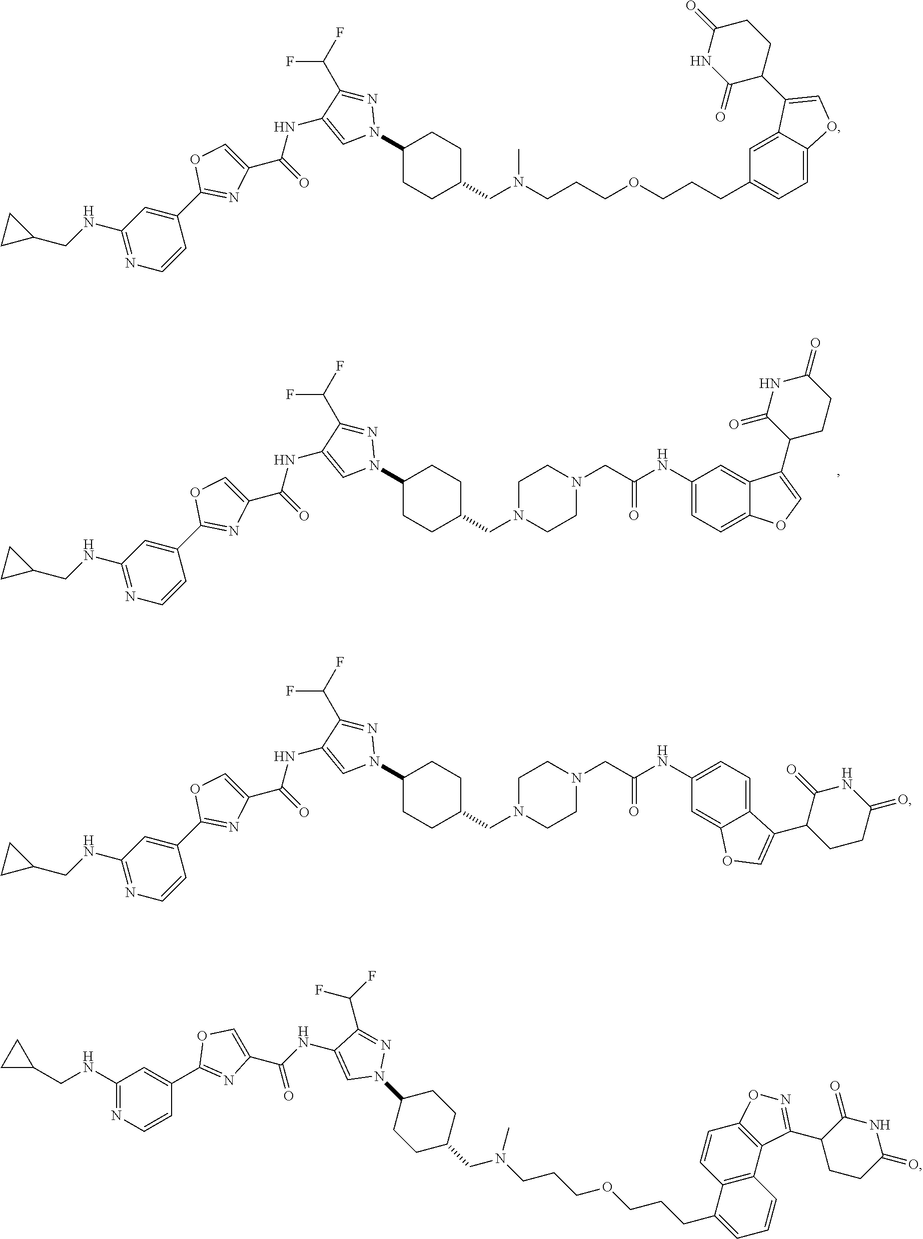

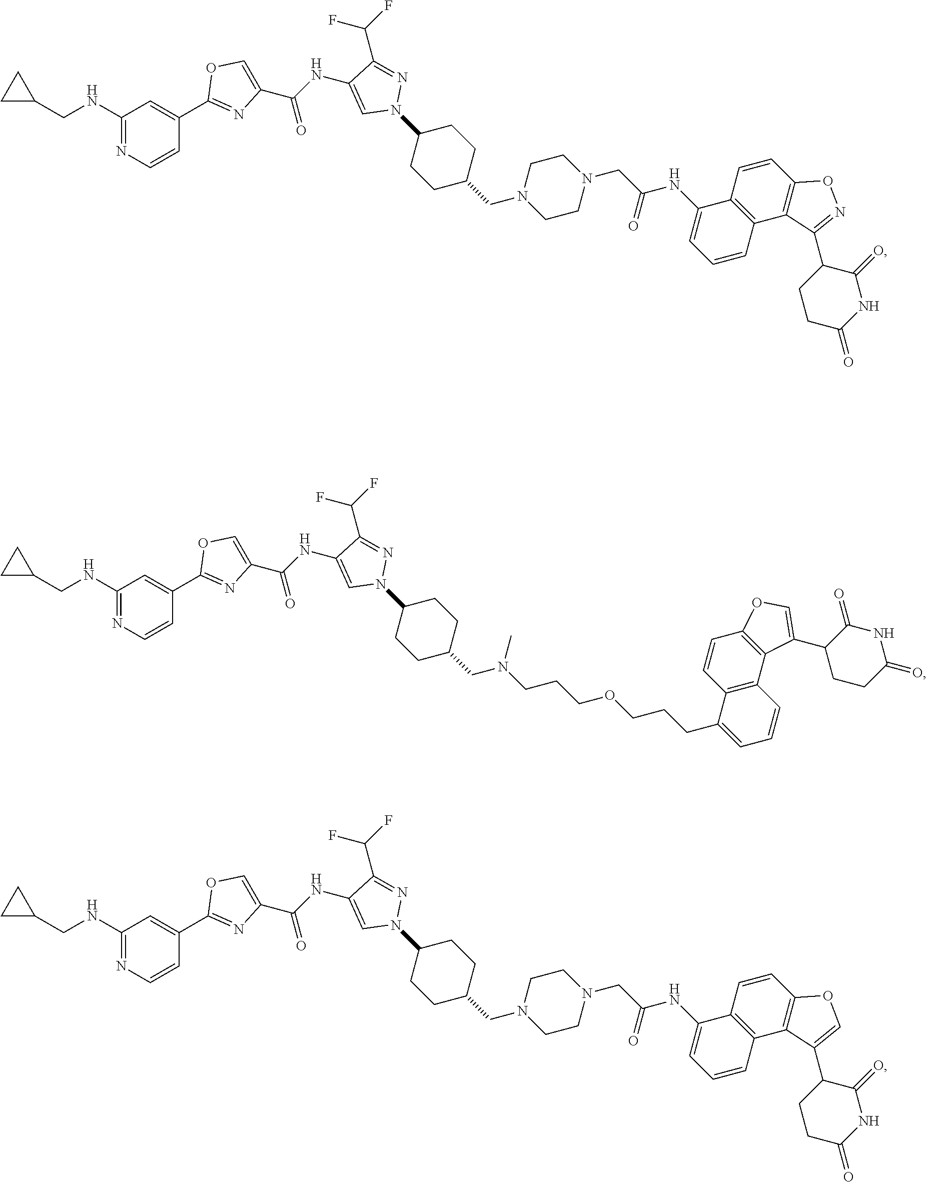

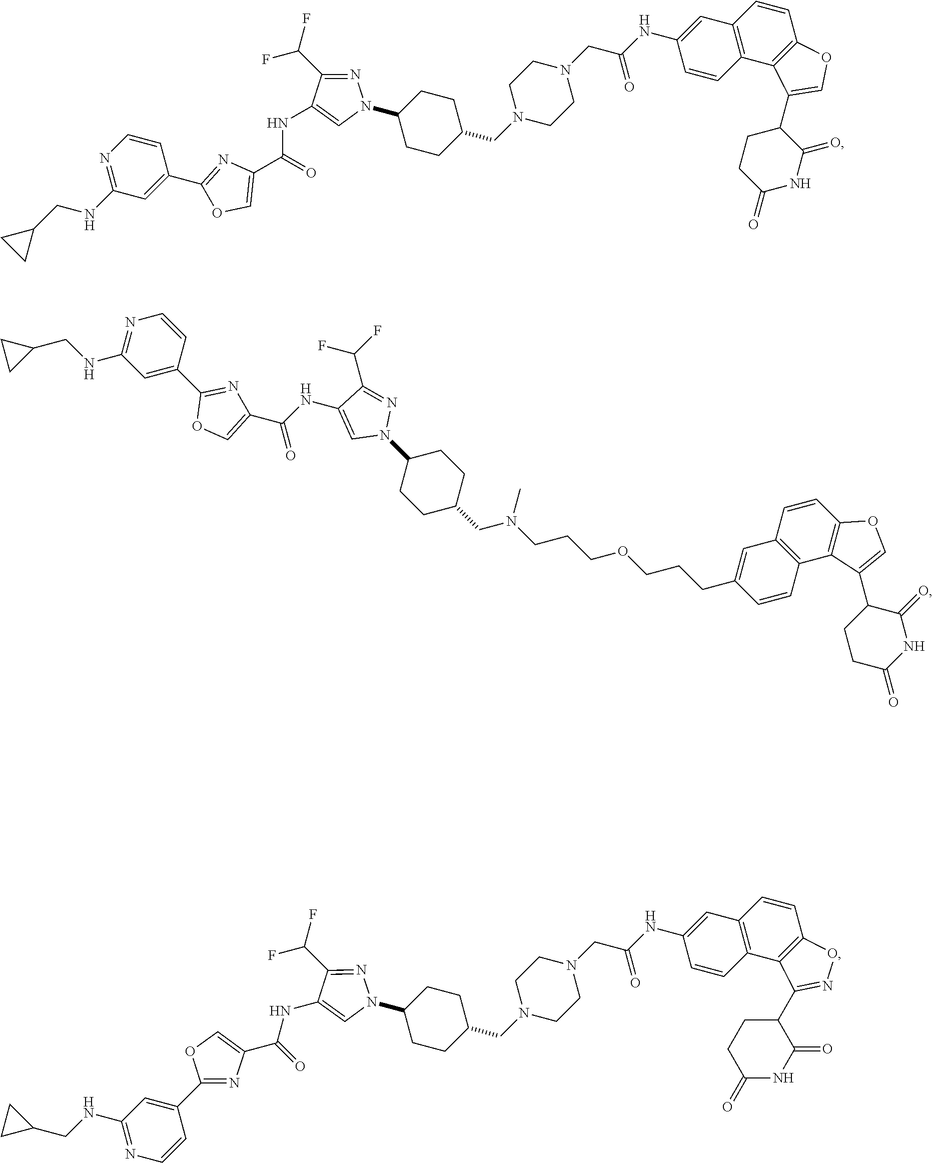

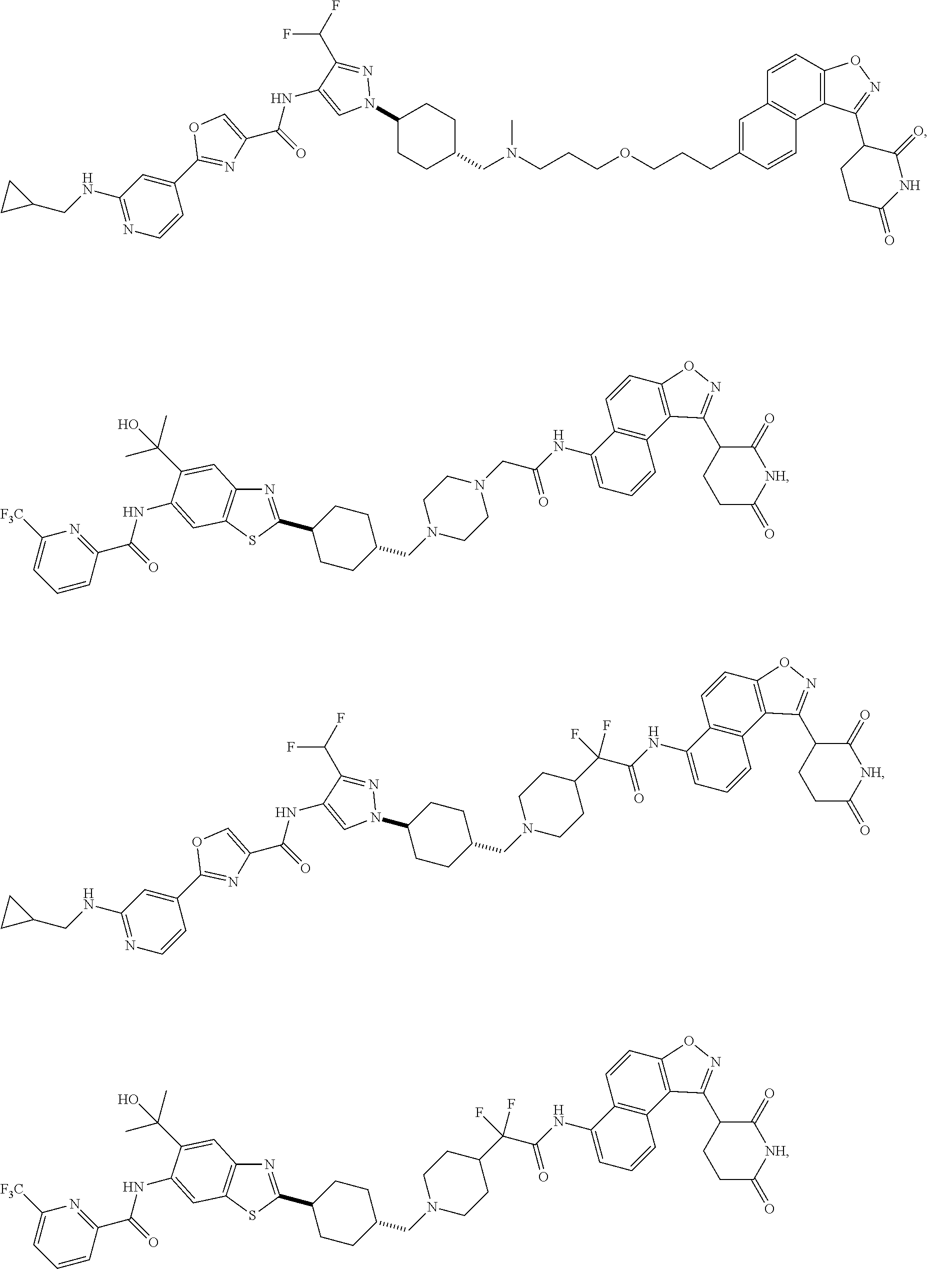

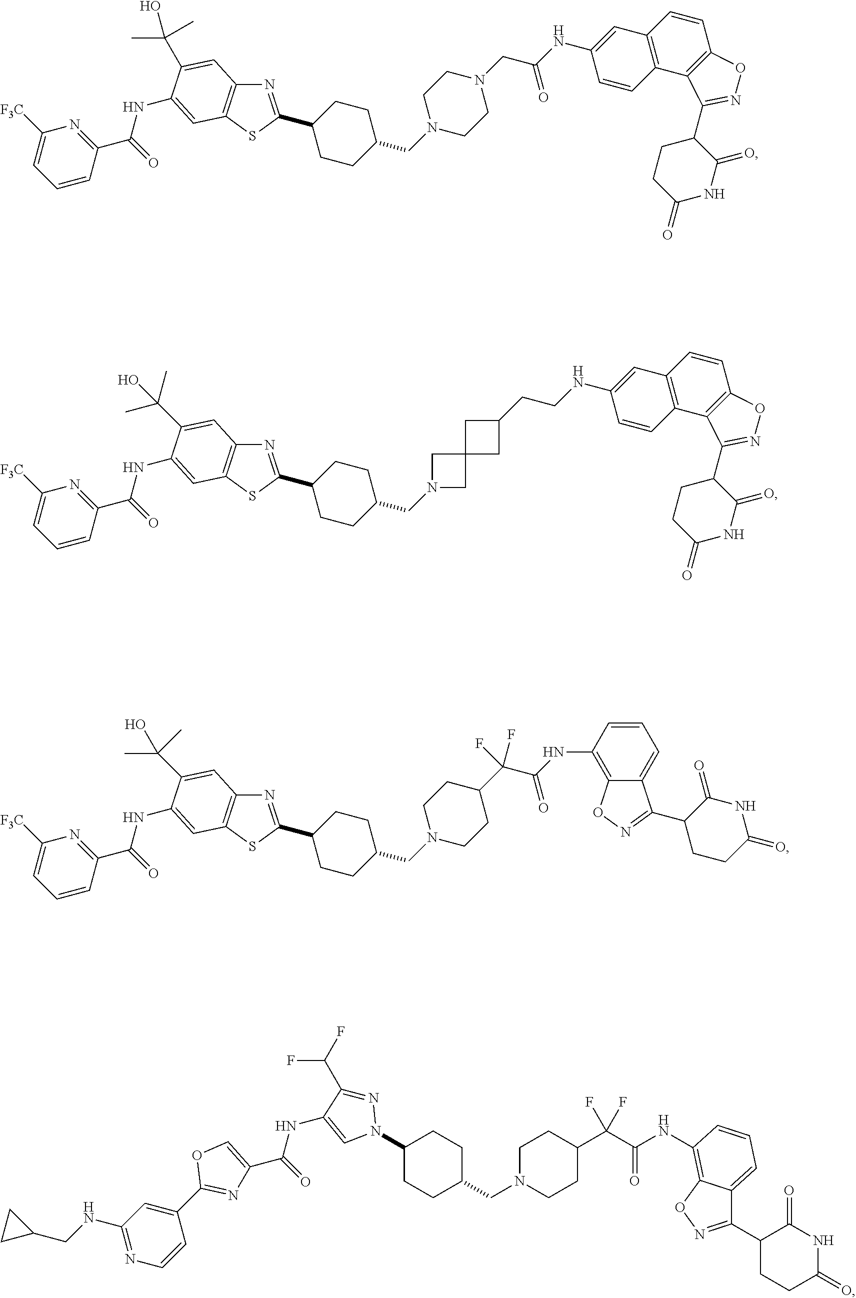

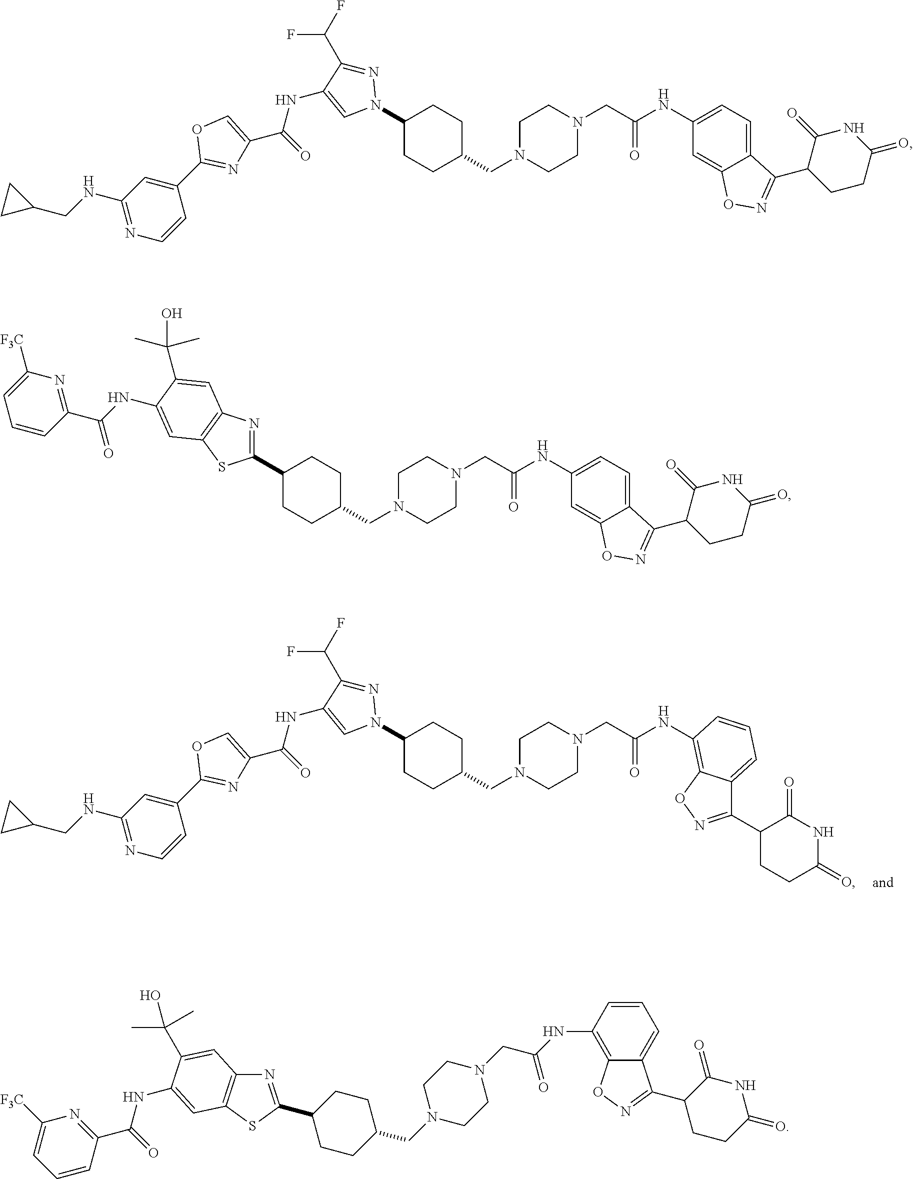

In some embodiments of the present disclosure, the compound is selected from:

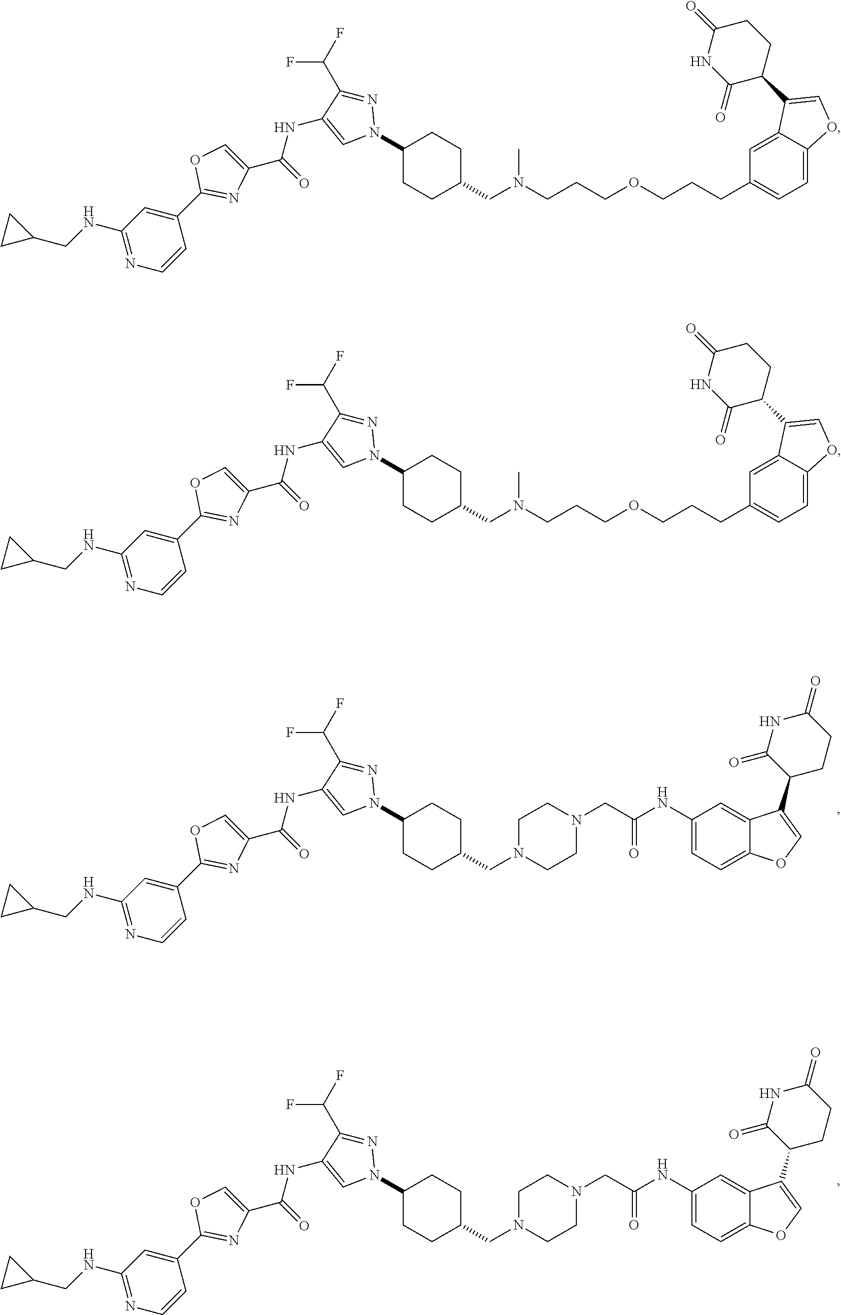

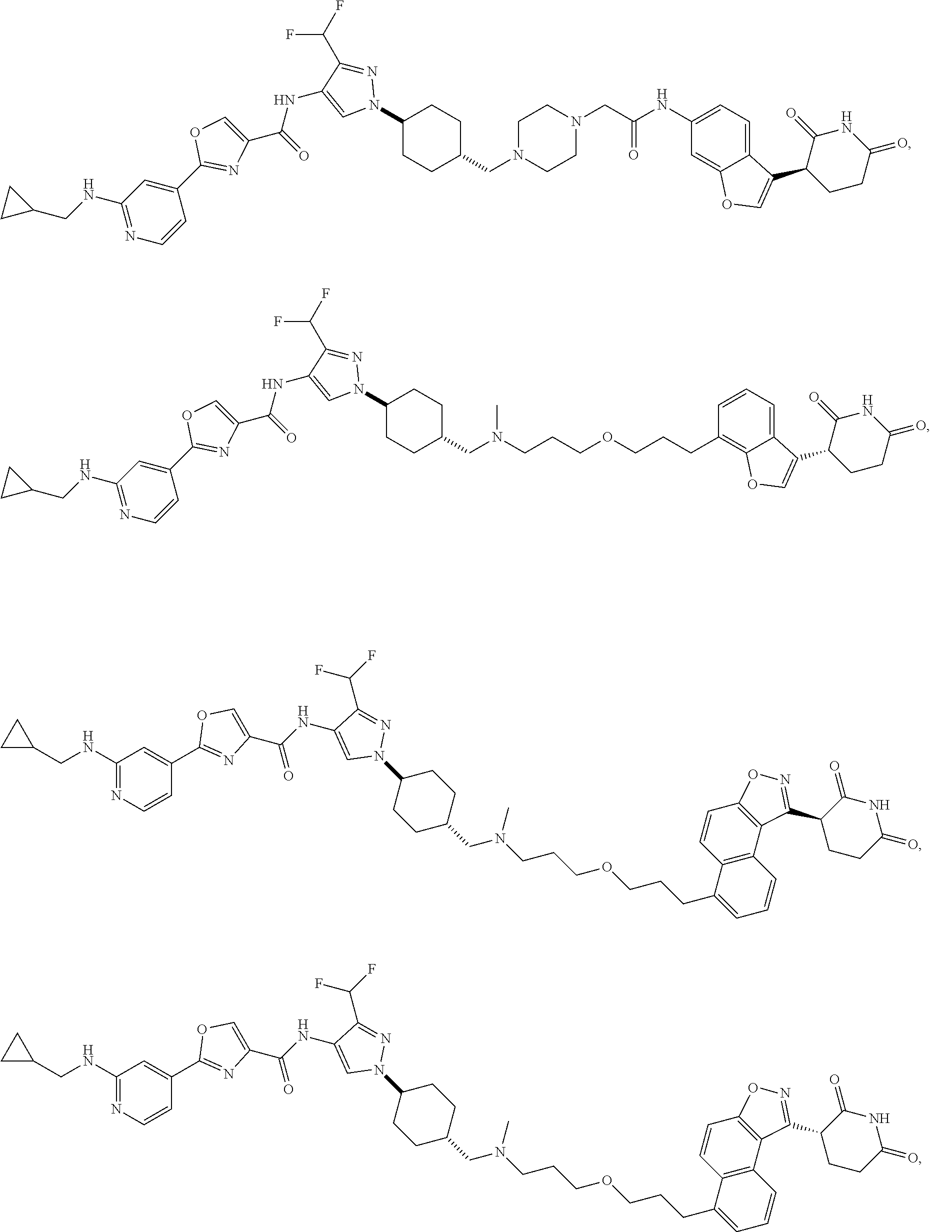

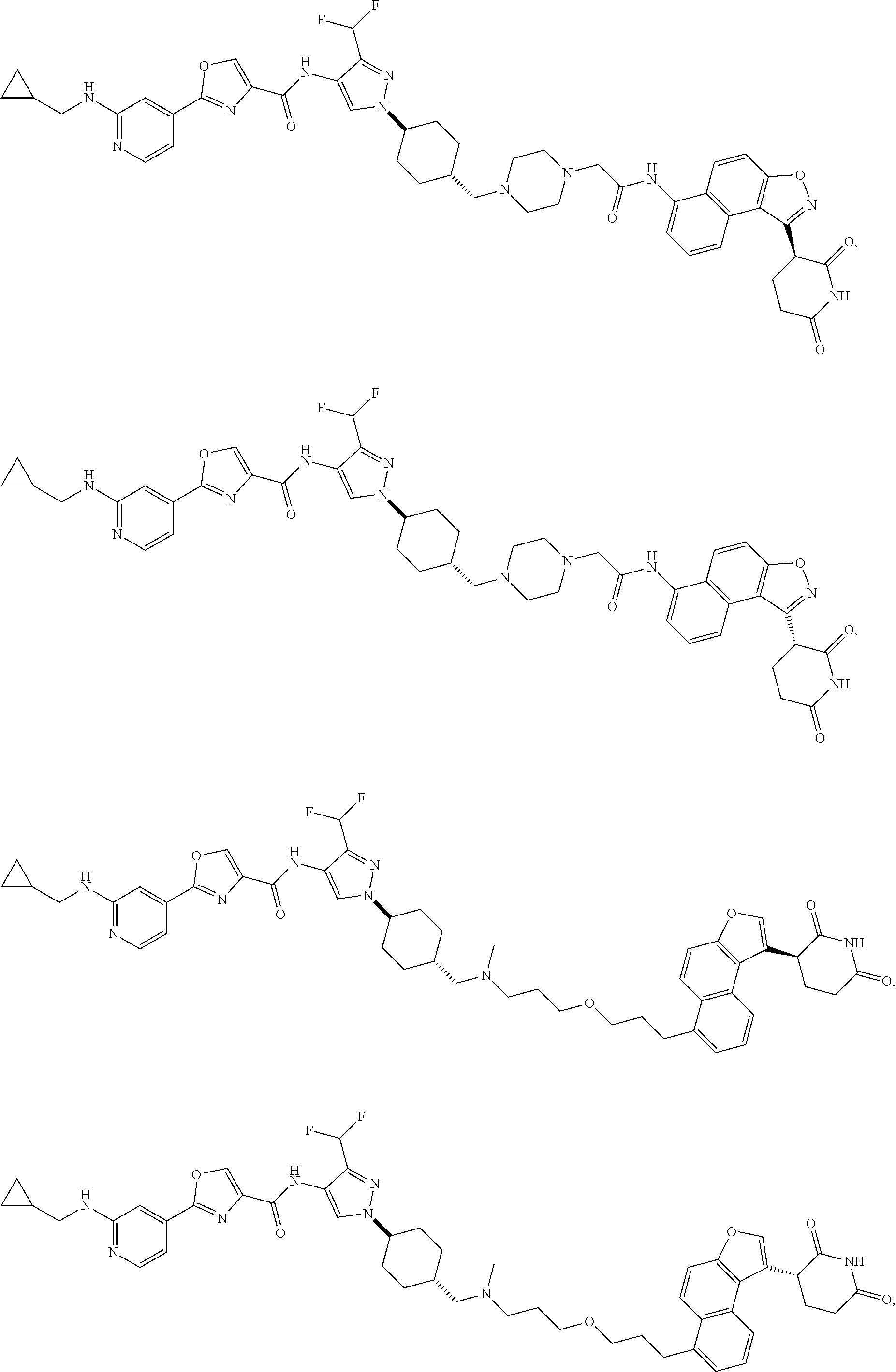

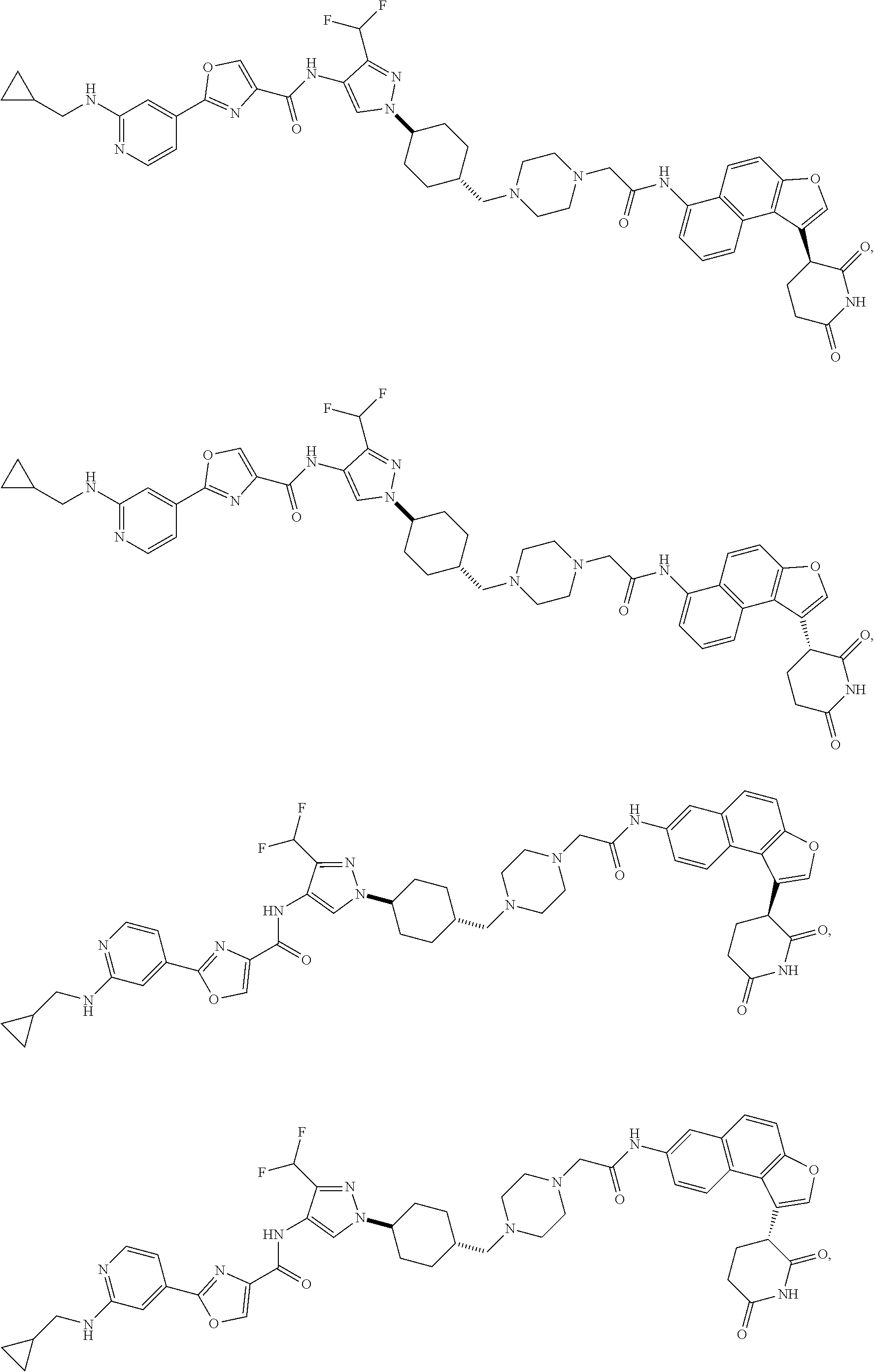

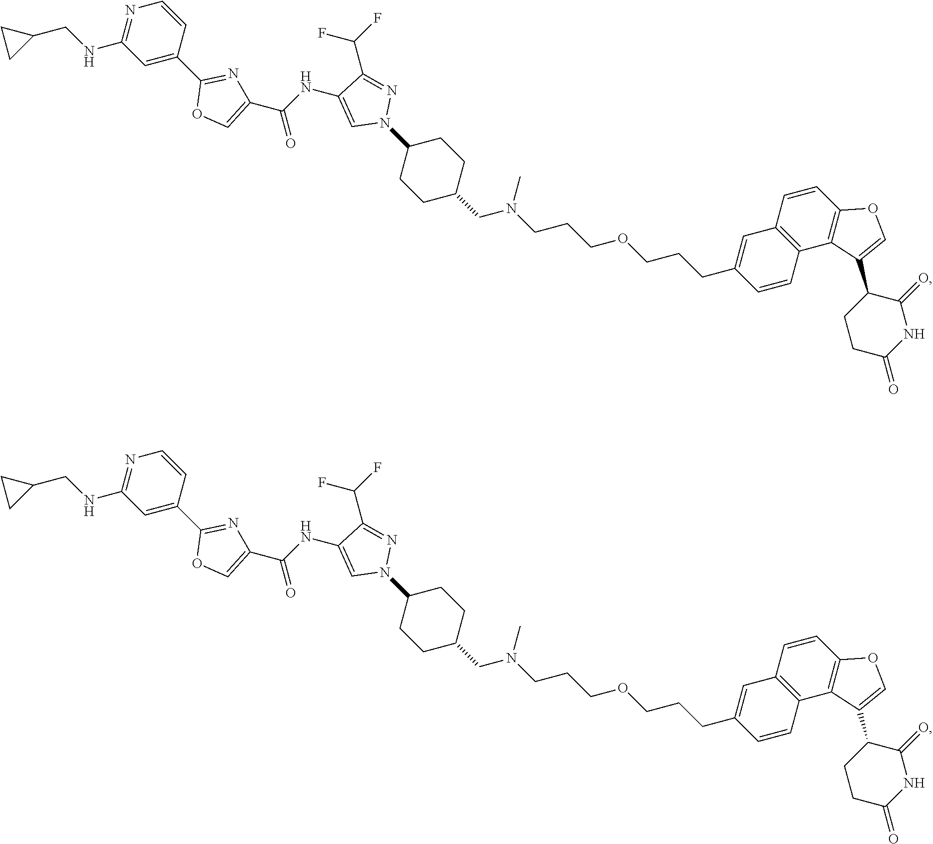

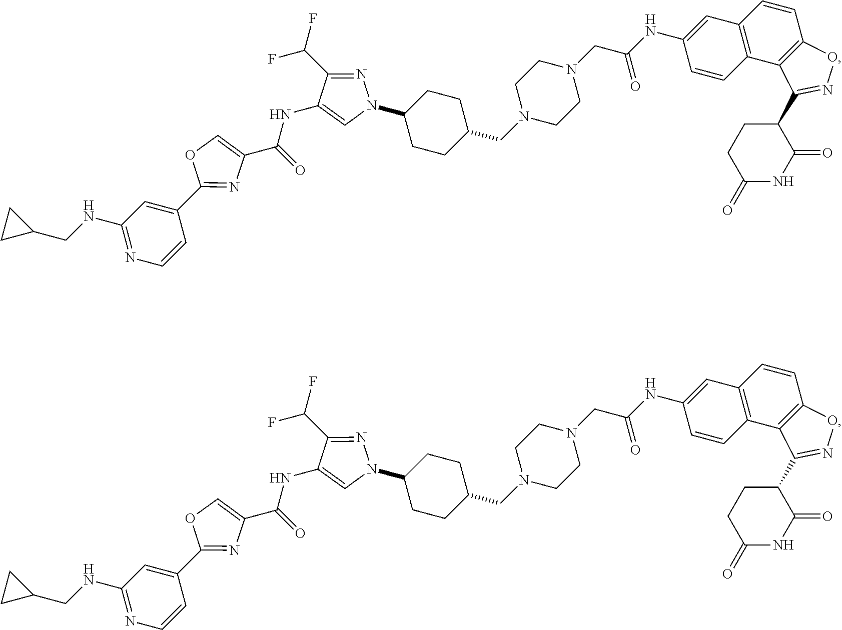

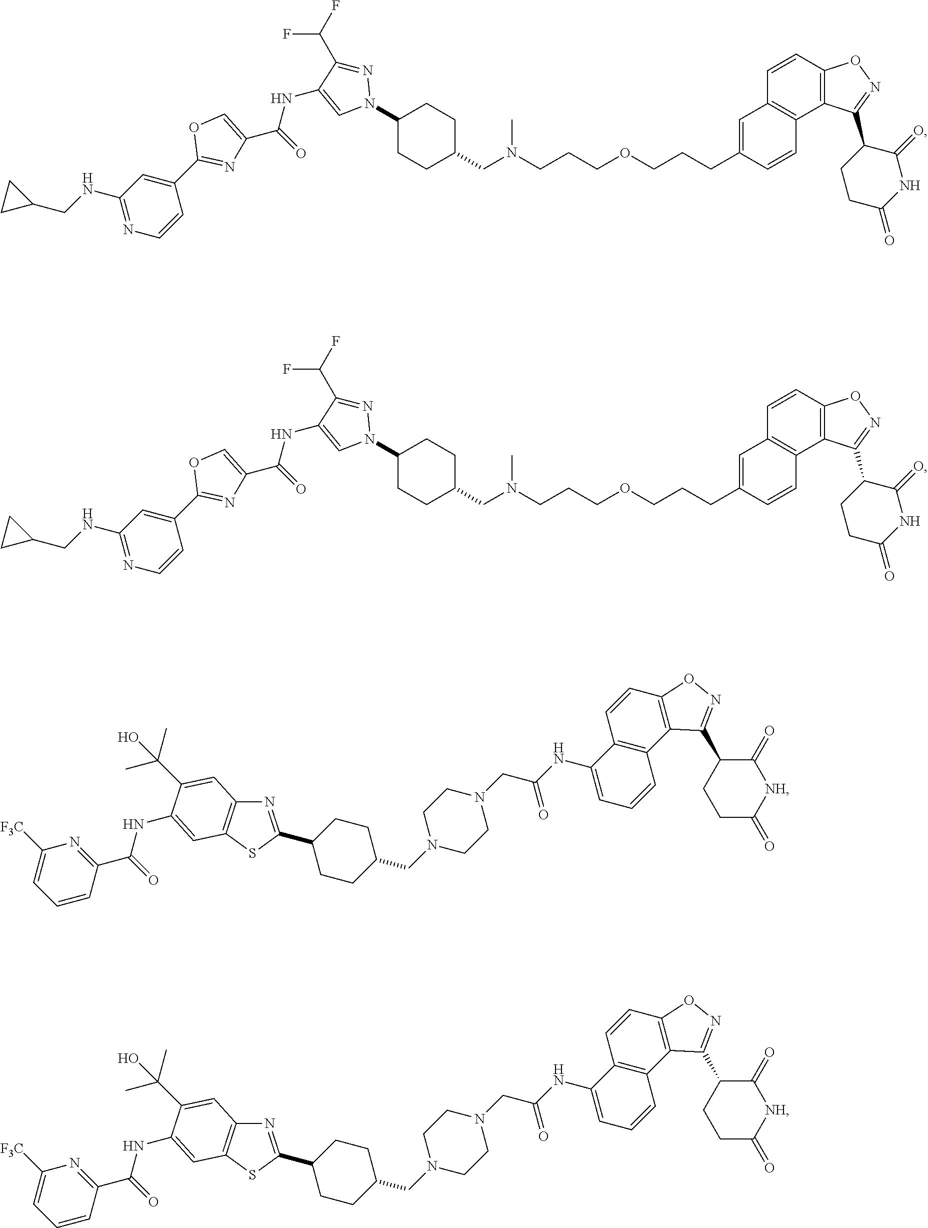

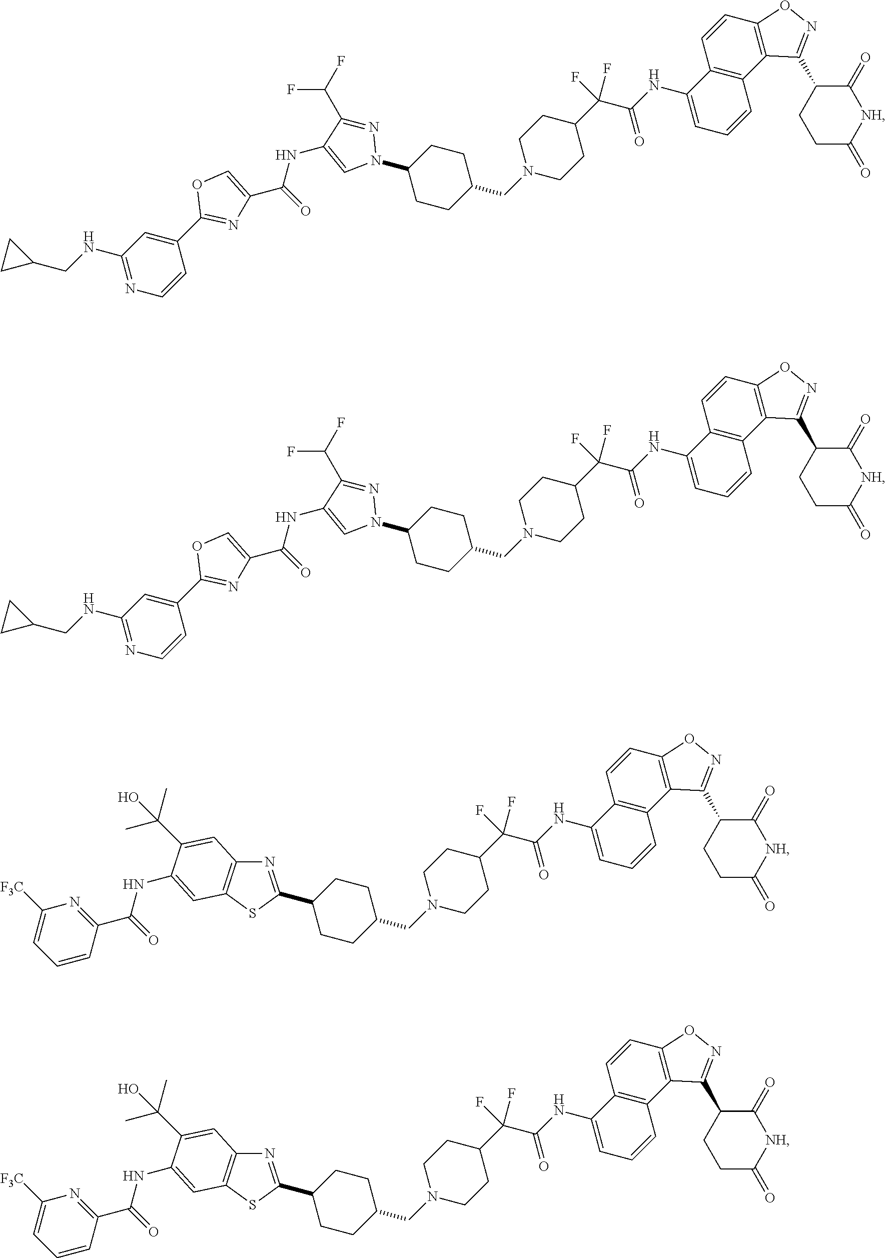

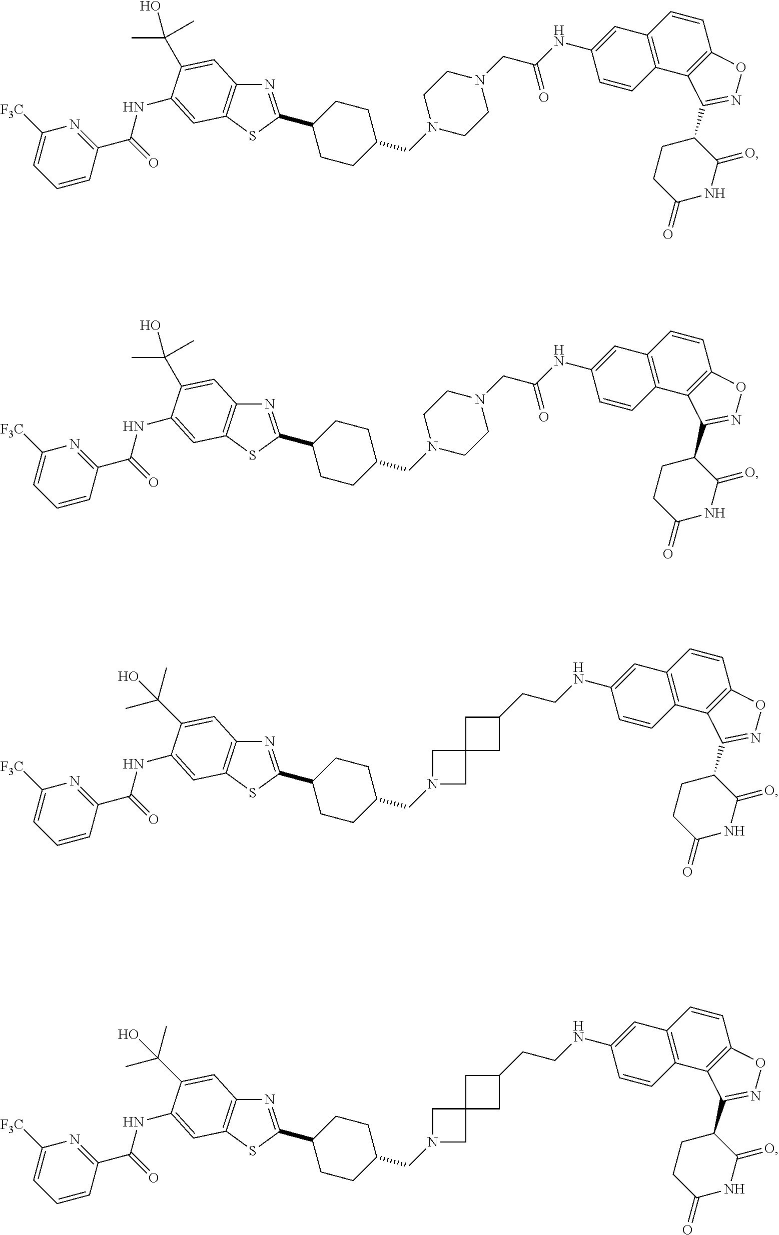

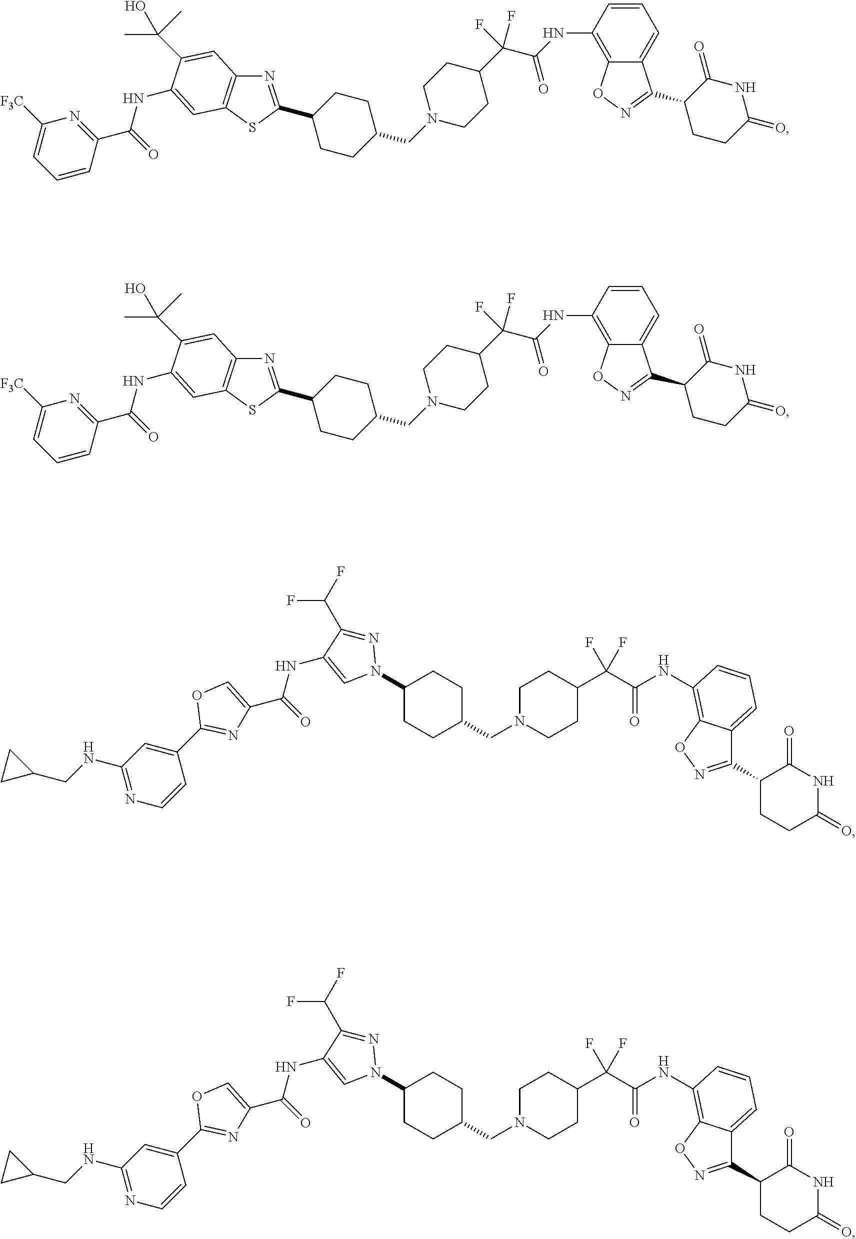

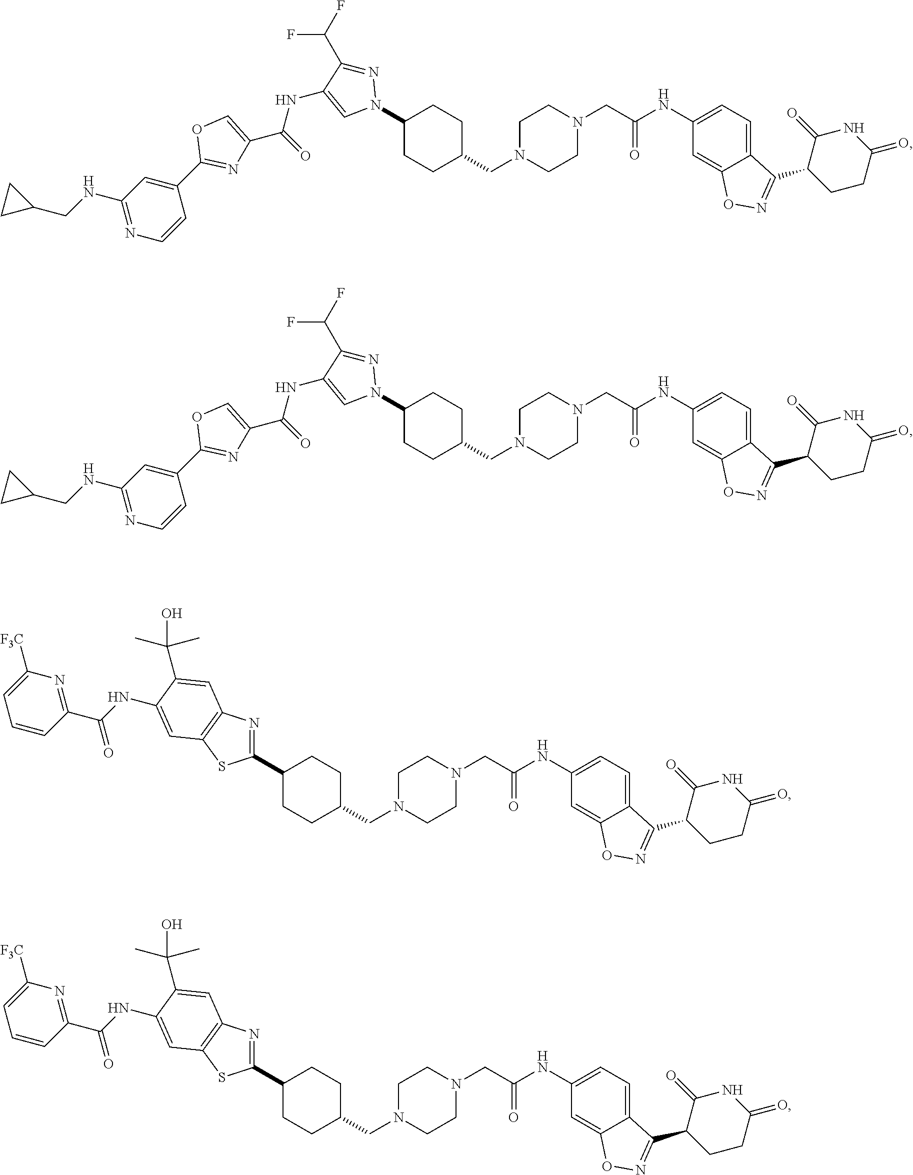

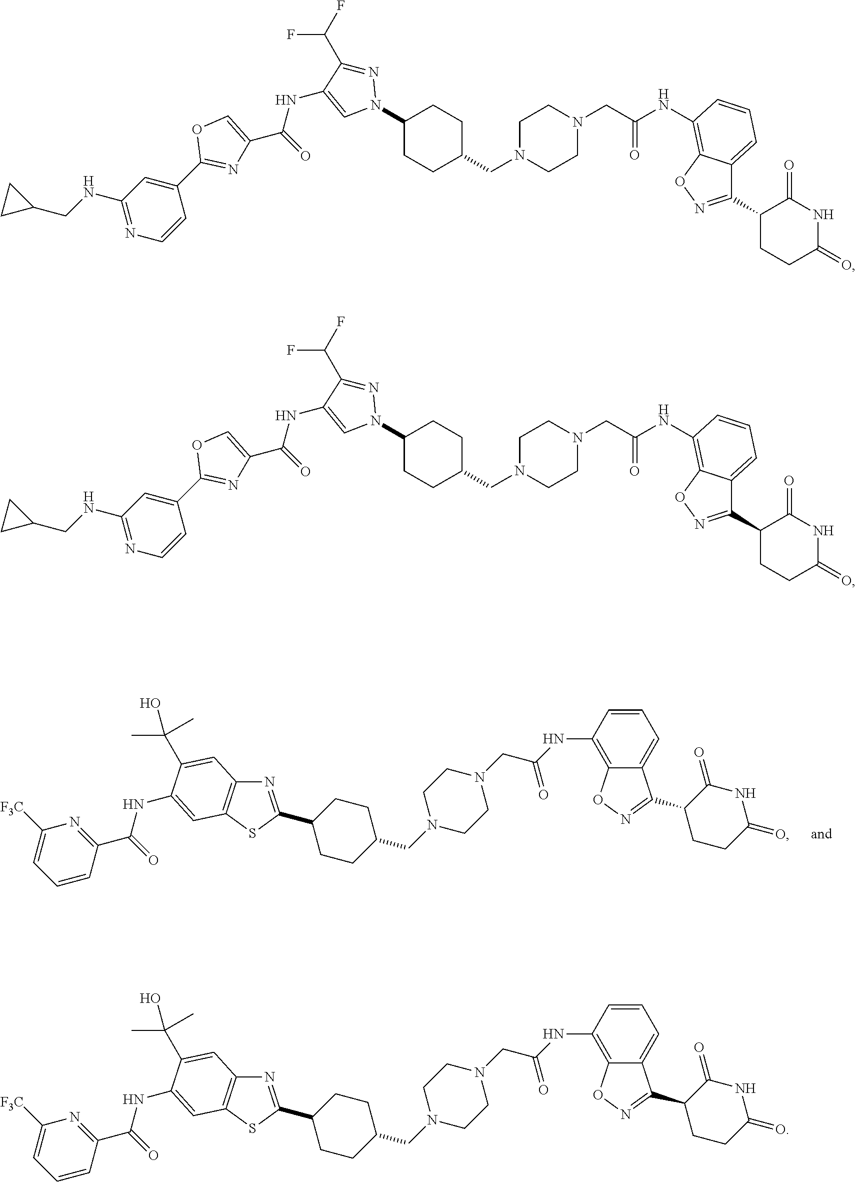

The present disclosure also provides a pharmaceutical composition comprising a therapeutically effective amount of the compound of the present disclosure, the stereoisomer thereof, or the pharmaceutically acceptable salt thereof.

The present disclosure also provides a use of the compound, the stereoisomer thereof, the pharmaceutically acceptable salt thereof, and the pharmaceutical composition in the manufacture of a medicament for treating tumors associated with proteolysis targeting chimeras of Interleukin-1 Receptor-Associated Kinase 4.

In some embodiments of the present disclosure, the tumor associated with proteolysis targeting chimeras of Interleukin-1 Receptor-Associated Kinase 4 is B-cell lymphoma.

### Technical Effect

The compounds of the present disclosure exhibit excellent degradation effect on target proteins IRAK4, IKZF1, and IKZF3. The compounds of the present disclosure exhibit excellent inhibitory effect on cell proliferation in lymphoma cell lines OCI-LY10, TMD-8, and SU-DHL-2. The compounds of the present disclosure exhibit high plasma exposure when administered orally. The compounds of the present disclosure exhibit superior pharmacokinetic properties in rodents like mice and rats, and non-rodents like beagles. The compounds of the present disclosure have remarkable tumor-suppressing effects and is dose-dependent in a SCID mouse xenograft tumor model using human B-cell lymphoma OCI-LY10 cells. The compounds of the present disclosure have remarkable tumor-suppressing effects in a CB17 SCID mouse model with subcutaneous xenograft tumors of human lymphoma SU-DHL-2 cells.

### Definition and Description

Unless otherwise specified, the following terms and phrases when used herein have the following meanings. A specific term or phrase should not be considered indefinite or unclear in the absence of a particular definition, but should be understood in the ordinary sense. When a trading name appears herein, it is intended to refer to its corresponding commodity or active ingredient thereof.

The term “pharmaceutically acceptable” is used herein in terms of those compounds, materials, compositions, and/or dosage forms, which are suitable for use in contact with human and animal tissues within the scope of reliable medical judgment, with no excessive toxicity, irritation, an allergic reaction, or other problems or complications, commensurate with a reasonable benefit/risk ratio.

“Pharmaceutical composition” refers to a formulation containing one or more of the compounds of the present disclosure, the stereoisomer thereof, or the pharmaceutically acceptable salts thereof, as well as other components such as physiologically/pharmaceutically acceptable carriers and excipients. The purpose of the pharmaceutical compositions is to facilitate administration to the organism and enhance the absorption of the active ingredients, and thereby exert biological activity.

The term “targeting chimera” refers to a bifunctional molecule containing two small-molecule ligands, one with high affinity for a target protein and the second for recruiting an E3 ligase. The E3 ligase ubiquitinates the protein and target it for proteolysis via the 26S proteasome.

The term “therapeutically effective amount” or “effective amount” refer to the quantity of a compound of the present disclosure that achieves the following effects: (i) treating or preventing a specific disease, condition, or disorder; (ii) reducing, improving, or eliminating one or more symptoms of a specific disease, condition, or disorder, or (iii) preventing or delaying the onset of one or more symptoms of a specific disease, condition, or disorder described herein. In the case of cancer, a therapeutically effective amount of a drug can reduce the number of cancer cells; decrease tumor size; inhibit (i.e., slow down to some extent and preferably stop) the infiltration of cancer cells into surrounding organs; inhibit (i.e., slow down to some extent and preferably stop) tumor metastasis; inhibit tumor growth to some extent; and/or alleviate one or more symptoms associated with cancer to some extent. In terms of the extent to which a drug can prevent the growth of existing cancer cells and/or kill existing cancer cells, it may have antiproliferative and/or cytotoxic properties.

The term “pharmaceutically acceptable salt” refers to a salt of the compound of the present disclosure that is prepared by reacting the compound having a specific substituent of the present disclosure with a relatively non-toxic acid or base. When the compound of the present disclosure contains a relatively acidic functional group, a base addition salt can be obtained by bringing the neutral form of the compound into contact with a sufficient amount of base in a pure solution or a suitable inert solvent. The pharmaceutically acceptable base addition salt includes a salt of sodium, potassium, calcium, ammonium, organic amine, magnesium, or similar salts. When the compound of the present disclosure contains a relatively basic functional group, an acid addition salt can be obtained by bringing the neutral form of the compound into contact with a sufficient amount of acid in a pure solution or a suitable inert solvent. Certain specific compounds of the present disclosure contain both basic and acidic functional groups, thus can be converted to any base or acid addition salt.

The pharmaceutically acceptable salt of the present disclosure can be prepared from the parent compound that contains an acidic or basic moiety by conventional chemical method. Generally, such salt can be prepared by reacting the free acid or base form of the compound with a stoichiometric amount of an appropriate base or acid in water or an organic solvent or a mixture thereof.

The term “treatment” includes the suppression, slowing, halting, or reversal of the progression or severity of existing symptoms or conditions.

Unless otherwise specified, the term “isomer” is intended to include a geometric isomer, a cis-trans isomer, a stereoisomer, an enantiomer, an optical isomer, a diastereoisomer, and a tautomeric isomer.

The compounds of the present disclosure may exist in specific geometric or stereoisomeric forms. The present disclosure contemplates all such compounds, including cis and trans isomers, (−)- and (+)-enantiomers, (R)- and (S)-enantiomers, diastereoisomers, (D)-isomers, (L)-isomers, racemic, and other mixtures thereof, such as enantiomer or diastereomer enriched mixtures, all of which are within the scope of the present disclosure. Additional asymmetric carbon atoms may be present in substituents such as alkyl. All these isomers and their mixtures are included within the scope of the present disclosure.

Unless otherwise specified, the term “enantiomer” or “optical isomer” refers to stereoisomers that are mirror images of each other.

Unless otherwise specified, the term “cis-trans isomer” or “geometric isomer” is caused by the inability to rotate freely of double bonds or single bonds of ring-forming carbon atoms.

Unless otherwise specified, the term “diastereomer” refers to a stereoisomer in which a molecule has two or more chiral centers and the relationship between the molecules is not mirror images.

Unless otherwise specified, “(+)” refers to dextrorotation, “(−)” refers to levorotation, and “(±)” refers to racemic.

Unless otherwise specified, the absolute configuration of a stereogenic center is represented by a wedged solid bond ( [symbol] ) and a wedged dashed bond ( [symbol] ); the relative configuration is represented by a straight solid bond ( [symbol] ) and a straight dashed bond ( [symbol] ), for example, trans-1, 4-dimethylcyclohexane is represented by or

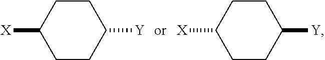

and cis-1,4-disubstituted cyclohexane is represented by

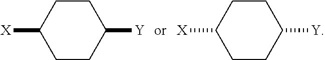

Unless otherwise specified, the terms “enriched in one isomer”, “enriched in isomers”, “enriched in one enantiomer”, or “enriched in enantiomers” refer to the content of one of the isomers or enantiomers is less than 100%, and the content of the isomer or enantiomer is greater than or equal to 60%, or greater than or equal to 70%, or greater than or equal to 80%, or greater than or equal to 90%, or greater than or equal to 95%, or greater than or equal to 96%, or greater than or equal to 97%, or greater than or equal to 98%, or greater than or equal to 99%, or greater than or equal to 99.50%, or greater than or equal to 99.600, or greater than or equal to 99.70%, or greater than or equal to 99.8%, or greater than or equal to 99.90%.

Unless otherwise specified, the term “isomer excess” or “enantiomeric excess” refers to the difference between the relative percentages of two isomers or two enantiomers. For example, if the content of one isomer or enantiomer is 90%, and the content of the other isomer or enantiomer is 10%, the isomer or enantiomer excess (ee value) is 80%.

Optically active (R)- and (S)-isomers, or D and L isomers can be prepared using chiral synthesis, chiral reagents, or other conventional techniques. If one kind of enantiomer of certain compound of the present disclosure is to be obtained, it can be obtained by asymmetric synthesis or derivative action of chiral auxiliary, wherein the resulting diastereomeric mixture is separated and the auxiliary group is cleaved to provide the pure desired enantiomer. Alternatively, when the molecule contains a basic functional group (such as amino) or an acidic functional group (such as carboxyl), a salt of a diastereoisomer is formed with an appropriate optically active acid or base, and then diastereomeric resolution is performed by conventional methods known in the art, and then the pure enantiomer is recovered. In addition, the enantiomer and the diastereoisomer are generally separated through chromatography which uses a chiral stationary phase and optionally combines with a chemical derivative method (such as carbamate generated from amine).

The compound of the present disclosure may contain an unnatural proportion of atomic isotope at one or more atoms that constitute the compound. For example, the compound can be radiolabeled with a radioactive isotope, such as tritium (³H), iodine-125 (¹²⁵ or C-14 (¹⁴C). For another example, deuterated drugs can be formed by replacing hydrogen with deuterium, the bond formed by deuterium and carbon is stronger than that of ordinary hydrogen and carbon, compared with non-deuterated drugs, deuterated drugs have the advantages of reduced toxic and side effects, increased drug stability, enhanced efficacy, extended biological half-life of drugs, etc. All isotopic variations of the compound of the present disclosure, whether radioactive or not, are encompassed within the scope of the present disclosure.

When the enumerative linking group does not indicate the direction for linking, the direction for linking is arbitrary, for example, the linking group L contained in

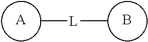

is -M-W—, then -M-W— can link ring A and ring B to form

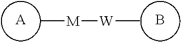

in the direction same as left-to-right reading order, and form

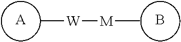

in the direction contrary to left-to-right reading order. A combination of the linking groups, substituents and/or variables thereof is allowed only when such combination can result in a stable compound.

Unless otherwise specified, when a group has one or more linkable sites, any one or more sites of the group can be linked to other groups through chemical bonds. When the linking site of the chemical bond is not positioned, and there is an H atom at the linkable site, then the number of H atoms at the site will decrease correspondingly with the number of the chemical bonds linking thereto so as to meet the corresponding valence. The chemical bond between the site and other groups can be represented by a straight solid bond ( [symbol] ), a straight dashed bond ( [symbol] ), or a wavy line ( [symbol] ). For example, the straight solid bond in —OCH₃ means that it is linked to other groups through the oxygen atom in the group; the straight dashed bond in

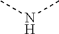

means that it is linked to other groups through the two ends of nitrogen atom in the group; the wave line in

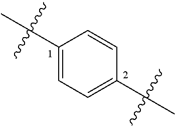 [SMILES: *c1ccc(*)cc1]

means that the phenyl group is linked to other groups through carbon atoms at position 1 and position 2; another example

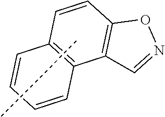

means that it can be linked to other groups through any linkable sites on the naphthalene ring by one chemical bond, including at least six types of linkage, including

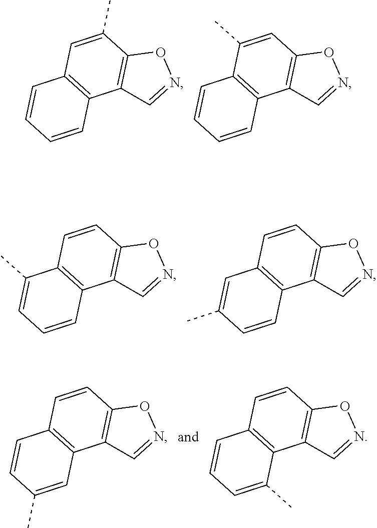

“Optional” or “optionally” means that the subsequent event or condition may occur but not requisite, that the term includes the instance in which the event or condition occurs and the instance in which the event or condition does not occur.

The term “substituted” means one or more than one hydrogen atom on a specific atom is substituted by the substituent, including deuterium and hydrogen variables, as long as the valence of the specific atom is normal and the substituted compound is stable. When the substituent is an oxygen (i.e., ═O), it means two hydrogen atoms are substituted. Positions on an aromatic ring cannot be substituted with a ketone. The term “optionally substituted” means an atom can be substituted with a substituent or not, unless otherwise specified, the type and number of the substituent may be arbitrary as long as being chemically achievable.

When any variable (such as R) occurs in the constitution or structure of the compound more than once, the definition of the variable at each occurrence is independent. Thus, for example, if a group is substituted by 0 to 2 R, the group can be optionally substituted with up to two R, wherein the definition of R at each occurrence is independent. Moreover, a combination of the substituent and/or the variant thereof is allowed only when the combination results in a stable compound.

The term “replaced” means that a specified atom or group can be replaced by another atom or group as specified. For example, CH₂ in CH₃CH₂CH₃ can be replaced by O, S and NH to obtain CH₃OCH₃, CH₃SCH₃ and CH₃NHCH₃.

Unless otherwise specified, Cₙ₋ₙ₊ₘ or Cₙ-Cₙ₊ₘ includes any specific case of n to n+m carbons, for example, C₁₋₁₂ includes C₁, C₂, C₃, C₄, C₅, C₆, C₇, C₈, C₉, C₁₀, C₁₁, and C₁₂, and any range from n to n+m is also included, for example C₁₋₁₂ includes C₁₋₃, C₁₋₆, C₁₋₉, C₃₋₆, C₃₋₉, C₃₋₁₂, C₆₋₉, C₆₋₁₂, and C₉₋₁₂, etc.; similarly, n-membered to n+m-membered means that the number of atoms on the ring is from n to n+m, for example, 3- to 12-membered ring includes 3-membered ring, 4-membered ring, 5-membered ring, 6-membered ring, 7-membered ring, 8-membered ring, 9-membered ring, 10-membered ring, 11-membered ring, and 12-membered ring, and any range from n to n+m is also included, for example, 3- to 12-membered ring includes 3- to 6-membered ring, 3- to 9-membered ring, 5- to 6-membered ring, 5- to 7-membered ring, 6- to 7-membered ring, 6- to 8-membered ring, and 6- to 10-membered ring, etc.

Unless otherwise specified, the term “C₁₋₁₀” refers to a linear or branched saturated hydrocarbon group consisting of 1 to 10 carbon atoms. The C₁₋₁₀ alkyl includes C₁₋₉, C₁₋₈, C₁₋₇, C₁₋₆, C₁₋₅, C₁₋₄, C₁₋₃, C₁₋₂, C₁₈, C₁₇, C₁₆, C₁₅, C₁₄, C₁₃, C₁₂, C₁₁, C₁₀, C₁₋₉, C₈, C₇, C₆, and C₅ alkyl, etc.; it can be monovalent (e.g., methyl), divalent (e.g., methylene), or multivalent (e.g., methine). Examples of C₁₋₈ alkyl include, but are not limited to, methyl (Me), ethyl (Et), propyl (including n-propyl and isopropyl), butyl (including n-butyl, isobutyl, s-butyl, and t-butyl), pentyl (including n-pentyl, isopentyl, and neopentyl), hexyl, heptyl, and octyl, etc. The term “C₁₋₁₀ alkylene” refers to a divalent C₁₋₁₀ alkyl.

When alkyl serves as a linking group, then “alkyl” refer to the linked alkylene group. The term “alkylene” refers to a saturated divalent hydrocarbon group obtained by removing two hydrogen atoms from a saturated linear or branched hydrocarbon. Examples of alkyl denoting a linked alkylene group include, but are not limited to, —CH₂—, —CH₂CH₂—, —CH(CH₃)CH₂—, —CH(CH₂CH₃)—, —CH₂CH(CH₃)—, —CH₂CH₂CH₂—, —CH₂CH₂CH₂CH₂—, etc.

Unless otherwise specified, the term “C₁₋₄ alkyl” refers to a linear or branched saturated hydrocarbon group consisting of 1 to 4 carbon atoms. The C₁₋₄ alkyl includes C₁₋₃ alkyl, C₁₋₂ alkyl, C₂ alkyl, C₃ alkyl, and methyl, etc.; it can be monovalent (such as methyl), divalent (such as methylene), or multivalent (such as methine). Examples of C₁₋₆ alkyl include, but are not limited to, methyl (Me), ethyl (Et), propyl (including n-propyl and isopropyl), butyl (including n-butyl, isobutyl, s-butyl, and t-butyl), etc.

Unless otherwise specified, “C₃₋₆ cycloalkyl” refers to a saturated cyclic hydrocarbon group consisting of 3 to 6 carbon atoms, which can be monocyclic and bicyclic, and the C₃₋₆ cycloalkyl includes C₃₋₅, C₄₋₅, and C₅₋₆ cycloalkyl, etc.; it can be monovalent, divalent, or multivalent. Examples of C₃₋₆ cycloalkyl include, but are not limited to, cyclopropyl, cyclobutyl, cyclopentyl, cyclohexyl, etc.

Unless otherwise specified, the term “4- to 8-membered heterocycloalkyl” by itself or in combination with other terms refers to a saturated cyclic group consisting of 4 to 8 ring atoms, wherein 1, 2, 3, or 4 ring atoms are heteroatoms independently selected from O, S, N, and Se, and the rest are carbon atoms, wherein nitrogen atoms are optionally quaternized, and nitrogen and sulfur heteroatoms can be optionally oxidized (i.e., NO and S(O)ₚ, p is 1 or 2). It includes monocyclic and bicyclic systems, wherein the bicyclic system includes a spiro ring, a fused ring, and a bridged ring. In addition, with regard to the “4- to 8-membered heterocycloalkyl”, a heteroatom may occupy the connection position of the heterocycloalkyl with the rest of the molecule. The 4- to 8-membered heterocycloalkyl includes 4- to 7-membered, 4- to 6-membered, 4- to 5-membered, 4-membered, 5-membered, 6-membered heterocycloalkyl, etc. Examples of 4- to 8-membered heterocycloalkyl include, but are not limited to, azetidinyl, oxetanyl, thietanyl, pyrrolidinyl, pyrazolidinyl, imidazolidinyl, tetrahydrothienyl (including tetrahydrothiophen-2-yl, tetrahydrothiophen-3-yl, etc.), tetrahydrofuranyl (including tetrahydrofuran-2-yl, etc.), tetrahydropyranyl, piperidinyl (including 1-piperidinyl, 2-piperidinyl, 3-piperidinyl, etc.), piperazinyl (including 1-piperazinyl, 2-piperazinyl, etc.), morpholinyl (including 3-morpholinyl, 4-morpholinyl, etc.), dioxinyl, dithianyl, isoxazolidinyl, isothiazolidinyl, 1,2-oxazinyl, 1,2-thiazinyl, hexahydropyridazinyl, homopiperazinyl, homopiperidinyl, dioxacycloheptyl, etc.

Unless otherwise specified, the terms “C₆₋₁₀ aromatic ring” and “C₆₋₁₀ aryl” in the present disclosure can be used interchangeably, and the term “C₆₋₁₀ aromatic ring” or “C₆₋₁₀ aryl” refers to a cyclic hydrocarbon group consisting of 6 to 10 carbon atoms with a conjugated π-electron system, which can be a monocyclic, condensed bicyclic, or condensed tricyclic system, wherein each ring is aromatic. It can be monovalent, divalent, or multivalent, and the C₆₋₁₀ aryl includes C₆₋₉, C₉, C₁₀, and C₆ aryl, etc. Examples of C₆₋₁₀ aryl include, but are not limited to, phenyl, naphthyl (including 1-naphthyl, 2-naphthyl, etc.).

Unless otherwise specified, the terms “5- to 10-membered heteroaryl ring” and “5- to 10-membered heteroaryl” in the present disclosure can be used interchangeably, and the term “5- to 10-membered heteroaryl” refers to a cyclic group consisting of 5 to 10 ring atoms with a conjugated π-electron system, wherein 1, 2, 3, or 4 ring atoms are heteroatoms independently selected from O, S, N, and Se, and the rest are carbon atoms. It can be a monocyclic, condensed bicyclic, or condensed tricyclic system, wherein each ring is aromatic. Here, the nitrogen atom is optionally quaternized, and nitrogen and sulfur heteroatoms can be optionally oxidized (i.e., NO and S(O)ₚ, wherein p is 1 or 2). The 5- to 10-membered heteroaryl can be linked to the rest of the molecule through a heteroatom or a carbon atom. The 5- to 10-membered heteroaryl includes 5- to 8-membered, 5- to 7-membered, 5- to 6-membered, 5-membered, and 6-membered heteroaryl, etc. Examples of the 5- to 10-membered heteroaryl include, but are not limited to, pyrrolyl (including N-pyrrolyl, 2-pyrrolyl, 3-pyrrolyl, etc.), pyrazolyl (including 2-pyrazolyl, 3-pyrrolyl, etc.), imidazolyl (including N-imidazolyl, 2-imidazolyl, 4-imidazolyl, 5-imidazolyl, etc.), oxazolyl (including 2-oxazolyl, 4-oxazolyl, 5-oxazolyl, etc.), triazolyl (1H-1,2,3-triazolyl, 2H-1,2,3-triazolyl, 1H-1,2,4-triazolyl, 4H-1,2,4-triazolyl, etc.), tetrazolyl, isoxazolyl (3-isoxazolyl, 4-isoxazolyl, 5-isoxazolyl, etc.), thiazolyl (including 2-thiazolyl, 4-thiazolyl, 5-thiazolyl, etc.), furyl (including 2-furyl, 3-furyl, etc.), thienyl (including 2-thienyl, 3-thienyl, etc.), pyridyl (including 2-pyridyl, 3-pyridyl, 4-pyridyl, etc.), pyrazinyl, pyrimidyl (including 2-pyrimidyl, 4-pyrimidyl, etc.), benzothiazolyl (including 5-benzothiazolyl, etc.), purinyl, benzimidazolyl (including 2-benzimidazolyl, etc.), benzoxazolyl, indolyl (including 5-indolyl, etc.), isoquinolyl (including 1-isoquinolyl, 5-isoquinolinyl, etc.), quinoxalinyl (including 2-quinoxalinyl, 5-quinoxalinyl, etc.), or quinolinyl (including 3-quinolinyl, 6-quinolinyl, etc.).

Unless otherwise specified, the term “halogen element” or “halogen” by itself or as part of another substituent refers to fluorine, chlorine, bromine, or iodine atom.

The structure of the compounds of the present disclosure can be confirmed by conventional methods known to those skilled in the art, and if the present disclosure involves an absolute configuration of a compound, then the absolute configuration can be confirmed by means of conventional techniques in the art. For example, in the case of single crystal X-ray diffraction (SXRD), diffraction intensity data are collected from the cultured single crystal using a Bruker D8 venture diffractometer with CuKα radiation as the light source and scanning mode: φ/ω scan, and after collecting the relevant data, the crystal structure is further analyzed by direct method (Shelxs97), so that the absolute configuration can be confirmed.

The compounds of the present disclosure can be prepared by a variety of synthetic methods known to those skilled in the art, including the specific embodiments listed below, the embodiments formed by their combination with other chemical synthesis methods, and equivalent alternatives known to those skilled in the art, preferred embodiments include but are not limited to the examples of the present disclosure.

The compounds of the present disclosure are named according to the conventional naming principles in the art or by ChemDraw® software, and the commercially available compounds use the supplier catalog names.

The solvent used in the present disclosure is commercially available.

The present adopts the following abbreviations: Ph stands for phenyl; Me stands for methyl; Et stands for ethyl; M means moles per liter; Boc stands for tert-butoxycarbonyl, which is an amino protecting group.

### DETAILED DESCRIPTION OF THE PREFERRED EMBODIMENT

The present disclosure is described in detail by the examples below, but it does not mean that there are any adverse restrictions on the present disclosure. The present disclosure has been described in detail herein, and its specific examples have also been disclosed; for one skilled in the art, it is obvious to make various modifications and improvements to the examples of the present disclosure without departing from the spirit and scope of the present disclosure.

### Reference Example 1: Fragment BB-1

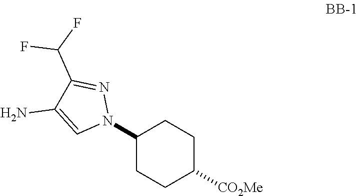 [SMILES: COC(=O)[C@H]1CC[C@H](n2cc(N)c(C(F)F)n2)CC1]

### Synthetic Route:

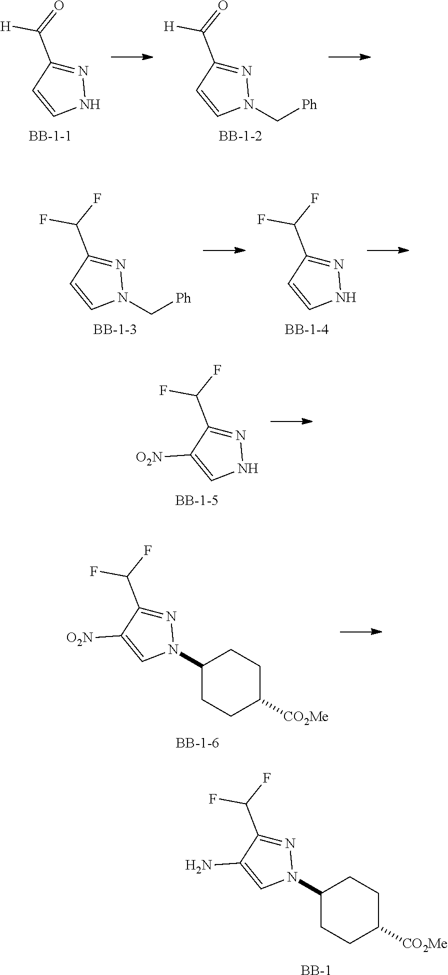

### Step 1: Synthesis of Compound BB-1-2

Compound BB-1-1 (40 g, 416.29 mmol) was dissolved in N, N-dimethylformamide (400 mL) at room temperature under nitrogen atmosphere, then benzyl bromide (74.76 g, 437.10 mmol, 51.92 mL) and cesium carbonate (339.09 g, 1.04 mol) were added thereto. The reaction mixture was stirred and reacted at room temperature under nitrogen atmosphere for 2 hours. After the reaction was completed, the reaction mixture was added with water (1500 mL), and extracted with ethyl acetate (5×1000 mL). The organic phases were combined, washed with 10% brine (3×1000 mL), dried over anhydrous sodium sulfate, and filtered, and the filtrate was concentrated under reduced pressure to remove the solvent. The resulting residue was separated by column chromatography (eluent: petroleum ether/ethyl acetate=1/0 to 20/1, v/v) to obtain compound BB-1-2. MS-ESI m/z: 187.2 [M+H]⁺.

### Step 2: Synthesis of Compound BB-1-3

Compound BB-1-2 (58 g, 311.48 mmol) was dissolved in dichloromethane (500 mL) at room temperature under nitrogen atmosphere, and the mixture was cooled to 0° C., and then diethylaminosulfur trifluoride (150.62 g, 934.43 mmol, 123.46 mL) was added thereto. The reaction mixture was stirred and reacted at room temperature for 16 hours. After the reaction was completed, the mixture was cooled to 0° C. The reaction mixture was slowly dropwise added with methanol (200 mL), and quenched, The reaction mixture was concentrated under reduced pressure to remove the solvent, added with water (1000 mL), and extracted with ethyl acetate (3×1000 mL). The organic phases were combined, washed sequentially with saturated sodium bicarbonate solution (1000 mL) and saturated brine (1000 mL), dried over anhydrous sodium sulfate, and filtered. The filtrate was concentrated under reduced pressure to remove the solvent. The resulting residue was separated by column chromatography (eluent: petroleum ether/ethyl acetate=1/0 to 20/1, v/v). The crude product was added with petroleum ether (20 mL), stirred at room temperature for 2 hours, and filtered. The filter cake was rinsed with petroleum ether (10 mL), collected, and dried under vacuum to obtain compound BB-1-3. MS-ESI m/z: 209.1 [M+H]⁺. ¹H NMR (400 MHz, CDCl₃) δ: 7.42-7.31 (m, 4H), 7.26-7.21 (m, 2H), 6.73 (t, J=55.2 Hz, 1H), 6.51 (t, J=1.0 Hz, 1H), 5.33 (s, 2H).

### Step 3: Synthesis of Compound BB-1-4

Compound BB-1-3 (26.5 g, 127.28 mmol) was dissolved in methanol (300 mL) at room temperature, then palladium hydroxide/carbon (5 g, purity of 20%) and hydrochloric acid (2 M, 25 mL) were added thereto. The reaction mixture was heated to 60° C., and stirred and reacted under hydrogen atmosphere (50 psi) for 30 hours. After the reaction was completed, the reaction mixture was filtered through diatomite. The filter cake was rinsed with methanol (800 mL). The filtrate was collected and concentrated under reduced pressure to remove the solvent to obtain compound BB-1-4. ¹H NMR (400 MHz, DMSO_d₆) δ: 11.41 (s, 1H), 7.84 (d, J=2.0 Hz, 1H), 7.00 (t, J=54.8 Hz, 1H), 6.51 (t, J=1.2 Hz, 1H).

### Step 4: Synthesis of Compound BB-1-5

Compound BB-1-4 (30 g, 254.06 mmol) was dissolved in concentrated sulfuric acid (300 mL, purity of 98%) at 0° C., then nitric acid (68.950 g, 711.24 mmol, 49.25 mL, purity of 65 to 68%) was dropwise added thereto. The reaction mixture was stirred at 0° C. for 10 minutes, then heated to 115° C., and stirred and reacted for 16 hours. After the reaction was completed, the reaction mixture was slowly poured into ice water (1000 mL), and extracted with ethyl acetate (3×500 mL). The organic phases were combined, washed sequentially with saturated sodium bicarbonate aqueous solution (500 mL) and saturated brine (500 mL). The organic phase was dried over anhydrous sodium sulfate, and filtered. The filtrate was concentrated under reduced pressure to remove the solvent to obtain compound BB-1-5. MS-ESI m/z: 164.1 [M+H]⁺. ¹H NMR (400 MHz, DMSO_d₆) δ: 14.39 (s, 1H), 9.00 (s, 1H), 7.31 (t, J=53.0 Hz, 1H).

### Step 5: Synthesis of Compound BB-1-6

Cis-4-hydroxycyclohexanecarboxylic acid methyl ester (35 g, 221.25 mmol) was dissolved in dichloromethane (350 mL) at room temperature under nitrogen atmosphere, then triethylamine (22.39 g, 221.25 mmol, 30.79 mL) was added thereto. The mixture was cooled to 0° C., then methanesulfonyl chloride (31.99 g, 279.26 mmol, 21.61 mL) was dropwise added thereto, and the reaction mixture was stirred and reacted at 0° C. for 0.5 hours. After the reaction was completed, the reaction mixture was slowly added with water (300 mL), and extracted with dichloromethane (300 mL). The organic phases were combined, washed with saturated brine (300 mL), dried over anhydrous sodium sulfate, and filtered. The filtrate was concentrated under reduced pressure to obtain a crude product, which was used in the next step directly.

Compound BB-1-5 (13.5 g, 82.78 mmol) and the crude product obtained above were dissolved in N,N-dimethylformamide (300 mL) at room temperature, then potassium carbonate (24 g, 173.65 mmol) was added thereto. The reaction mixture was heated to 80° C., and stirred and reacted for 16 hours. After the reaction was completed, the reaction mixture was cooled to room temperature, added with water (100 mL), and extracted with ethyl acetate (40 mL×2). The organic phases were combined, dried over anhydrous sodium sulfate, and filtered. The filtrate was concentrated under reduced pressure to remove the solvent. The resulting residue was separated by column chromatography (eluent: ethyl acetate/petroleum ether=1/3, v/v) to obtain compound BB-1-6. ¹H NMR (400 MHz, CDCl₃) δ: 8.22 (s, 1H), 7.12 (t, J=53.4 Hz, 1H), 4.25-4.16 (m, 1H), 3.72 (s, 3H), 2.46-2.37 (m, 1H), 2.36-2.28 (m, 2H), 2.27-2.20 (m, 2H), 1.89-1.77 (m, 2H), 1.72-1.60 (m, 2H).

### Step 6: Synthesis of Compound BB-1

Wet palladium on carbon (1.5 g, purity of 10%) was added to tetrahydrofuran (200 mL) at room temperature under argon atmosphere, then compound BB-1-6 (7.5 g, 24.73 mmol) was added thereto. The reaction mixture was stirred and reacted at room temperature under hydrogen (15 psi) atmosphere for 15 hours. After the reaction was completed, the reaction mixture was filtered. The filter cake was washed with dichloromethane (50 mL×2), and the filtrate was concentrated under reduced pressure to remove the solvent. The resulting residue was separated by column chromatography (eluent: ethyl acetate/petroleum ether=1/2, v/v) to obtain compound BB-1. MS-ESI m/z: 274.1 [M+H]⁺.

### Reference Example 2: Fragment BB-2

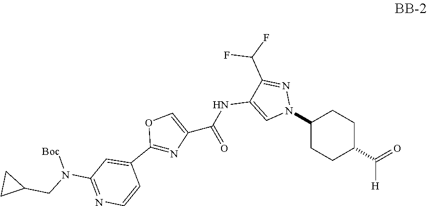 [SMILES: CC(C)(C)OC(=O)N(CC1CC1)c1cc(-c2nc(C(=O)Nc3cn([C@H]4CC[C@H](C=O)CC4)nc3C(F)F)co2)ccn1]

### Synthetic Route:

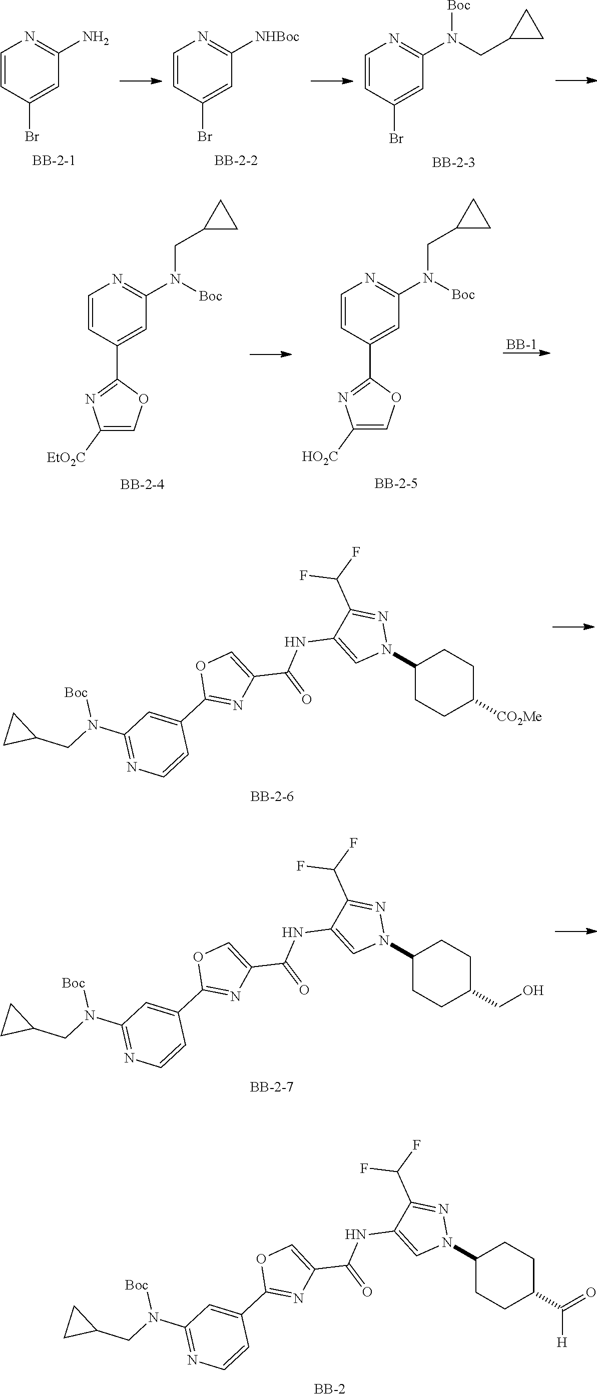

### Step 1: Synthesis of Compound BB-2-2

Compound BB-2-1 (50 g, 289.00 mmol) was dissolved in tetrahydrofuran (750 mL) at room temperature under nitrogen atmosphere, and the mixture was cooled to −5° C., and then a solution of lithium bis(trimethylsilyl)amide in n-hexane (1 M, 578.00 mL) was added thereto. The reaction mixture was stirred at −5° C. for 10 minutes, and di-tert-butyl dicarbonate (63.07 g, 289.00 mmol, 66.39 mL) was added thereto. The reaction mixture was slowly warmed to room temperature, and stirred and reacted for 1 hour. After the reaction was completed, the reaction mixture was slowly added to a saturated solution of ammonium chloride (1000 mL), and extracted with ethyl acetate (1000 mL×3). The organic phases were combined, washed with saturated brine (2000 mL×2). The organic phase was dried over anhydrous sodium sulfate, and filtered. The filtrate was concentrated under reduced pressure to remove the solvent to obtain compound BB-2-2. MS-ESI m/z: 273.1 [M+H]⁺, 275.1 [M+H+2]⁺. ¹H NMR (400 MHz, DMSO_d₆) δ: 10.10 (s, 1H), 8.14 (d, J=5.2 Hz, 1H), 8.04 (d, J=1.6 Hz, 1H), 7.27 (t, J=1.8, 5.4 Hz, 1H), 1.47 (s, 9H).

### Step 2: Synthesis of Compound BB-2-3

Compound BB-2-2 (39 g, 142.79 mmol) was dissolved in N, N-dimethylformamide (400 mL) at room temperature under nitrogen atmosphere, and the mixture was cooled to 0° C., and then sodium hydride (8.57 g, 214.19 mmol, purity of 60%) was added thereto. The reaction mixture was reacted at 0 to 5° C. for 30 minutes, then bromomethyl cyclopropane (23.13 g, 171.35 mmol, 16.41 mL) was added thereto. The reaction mixture was slowly warmed to room temperature, and stirred and reacted for 15 hours. After the reaction was completed, the reaction mixture was slowly added to water (1.5 mL), and extracted with ethyl acetate (1 L×3). The organic phases were combined, washed with saturated brine (3 L×3). The organic phase was dried over anhydrous sodium sulfate, and filtered. The filtrate was concentrated under reduced pressure to remove the solvent. The resulting residue was separated by column chromatography (eluent: petroleum ether/ethyl acetate=50/1 to 20/1, v/v) to obtain compound BB-2-3. ¹H NMR (400 MHz, CDCl₃) δ: 8.17 (d, J=5.2 Hz, 1H), 7.96 (d, J=1.6 Hz, 1H), 7.15 (dd, J=1.6, 5.2 Hz, 1H), 3.87 (d, J=7.2 Hz, 2H), 1.54 (s, 9H), 1.21-1.14 (m, 1H), 0.44-0.39 (m, 2H), 0.27-0.23 (m, 2H).

### Step 3: Synthesis of Compound BB-2-4

Compound BB-2-3 (47.53 g, 145.26 mmol) and ethyl oxazole-4-carboxylate (20.5 g, 145.26 mmol) were dissolved in N, N-dimethylformamide (500 mL) at room temperature under nitrogen atmosphere, then tri(o-tolyl)phosphine (8.84 g, 29.05 mmol), palladium acetate (3.26 g, 14.53 mmol), and cesium carbonate (94.66 g, 290.52 mmol) were added thereto. The reaction mixture was heated to 80° C., and stirred for 14 hours. After the reaction was completed, the reaction mixture was added to water (1 L), and extracted with ethyl acetate (1 L×3). The organic phases were combined, washed with saturated brine (3 L×3). The organic phases were dried over anhydrous sodium sulfate, and filtered. The filtrate was concentrated under reduced pressure to remove the solvent. The resulting residue was separated by column chromatography (eluent: petroleum ether/ethyl acetate=5/1 to 2/1, v/v) to obtain compound BB-2-4. MS-ESI m/z: 388.2 [M+H]⁺. ¹H NMR (400 MHz, CDCl₃) δ: 8.49 (d, J=5.2 Hz, 1H), 8.37 (s, 1H), 8.33 (s, 1H), 7.70 (dd, J=1.2, 5.2 Hz, 1H), 4.45 (q, J=7.0 Hz, 2H), 3.92 (d, J=6.8 Hz, 2H), 1.55 (s, 9H), 1.42 (t, J=7.2 Hz, 3H), 1.22-1.13 (m, 1H), 0.44-0.39 (m, 2H), 0.27-0.22 (m, 2H).

### Step 4: Synthesis of Compound BB-2-5

Compound BB-2-4 (18 g, 46.46 mmol) was dissolved in water (80 mL) and tetrahydrofuran (400 mL) at room temperature under nitrogen atmosphere, then lithium hydroxide monohydrate (5.85 g, 139.38 mmol) was added thereto. The reaction mixture was stirred and reacted at room temperature for 4 hours. After the reaction was completed. The reaction mixture was concentrated under reduced pressure, added with 2 M hydrochloric acid to adjust the pH to 2 to 3, and extracted with ethyl acetate (100 mL×5). The organic phases were combined, dried over anhydrous sodium sulfate, and filtered. The filtrate was concentrated under reduced pressure to remove the solvent to obtain compound BB-2-5. ¹H NMR (400 MHz, CDCl₃) δ: 8.57 (d, J=5.2 Hz, 1H), 8.44 (s, 1H), 8.38 (s, 1H), 7.77 (dd, J=1.2, 5.2 Hz, 1H), 3.94 (d, J=6.8 Hz, 2H), 1.55 (s, 9H), 1.20-1.11 (m, 1H), 0.45-0.39 (m, 2H), 0.29-0.20 (m, 2H).

### Step 5: Synthesis of Compound BB-2-6

Compound BB-2-5 (5 g, 13.91 mmol) were dissolved in N, N-dimethylformamide (50 mL) at room temperature, then N, N-diisopropylethylamine (5.39 g, 41.74 mmol, 7.27 mL) and 2-(7-azabenzotriazol)-N, N, N′, N′-tetramethyluronium hexafluorophosphate (6.35 g, 16.70 mmol) were added thereto. The reaction mixture was stirred and reacted for 0.5 hours, then compound BB-1 (4.18 g, 15.30 mmol) was added thereto. The reaction continued for 1.5 hours. After the reaction was completed, the reaction mixture was poured into water (500 mL), and extracted with ethyl acetate (150 mL×2). The organic phases were combined, washed with 10% brine (150 mL×2), dried over anhydrous sodium sulfate, and filtered. The filtrate was concentrated under reduced pressure to remove the solvent. The resulting residue was separated by column chromatography (eluent: petroleum ether/ethyl acetate=2/1, v/v) to obtain compound BB-2-6. MS-ESI m/z: 615.4 [M+H]⁺. ¹H NMR (400 MHz, DMSO_d₆) δ: 9.77 (s, 1H), 9.00 (s, 1H), 8.59 (d, J=5.2 Hz, 1H), 8.29 (s, 1H), 8.19 (s, 1H), 7.68 (dd, J=1.2, 5.2 Hz, 1H), 7.16 (t, J=54.2 Hz, 1H), 4.32-4.22 (m, 1H), 3.86 (d, J=6.8 Hz, 2H), 3.62 (s, 3H), 2.45-2.38 (m, 1H), 2.11-1.99 (m, 4H), 1.88-1.75 (m, 2H), 1.61-1.54 (m, 1H), 1.51 (s, 9H), 1.22-1.12 (m, 1H), 0.44-0.37 (m, 2H), 0.27-0.19 (m, 2H).

### Step 6: Synthesis of Compound BB-2-7

Compound BB-2-6 (5 g, 8.13 mmol) was dissolved in tetrahydrofuran (50 mL) at room temperature under nitrogen atmosphere, and the mixture was cooled to −40° C., and then a solution of diisobutylaluminium hydride in toluene (1 M, 48.81 mL) was slowly added thereto. After the dropwise addition was completed, the reaction mixture was heated to 0° C., and stirred and reacted for 30 minutes. After the reaction was completed, the reaction mixture was added with 1 M sodium hydroxide solution (10 mL), then added with water (50 mL), and extracted with ethyl acetate (4×50 mL). The organic phases were combined, washed with saturated brine (100 mL), dried over anhydrous sodium sulfate, and filtered. The filtrate was concentrated under reduced pressure to remove the solvent. The resulting residue was purified by column chromatography (eluent: petroleum ether/ethyl acetate=1/0 to 1/3, v/v) to obtain compound BB-2-7. MS-ESI m/z: 587.4 [M+H]⁺. ¹H NMR (400 MHz, CDCl₃) δ: 9.07 (s, 1H), 8.52 (d, J=5.2 Hz, 1H), 8.38 (s, 1H), 8.33 (s, 2H), 7.64-7.62 (m, 1H), 7.01-6.65 (m, 1H), 4.14-4.10 (m, 1H), 3.94 (d, J=7.2 Hz, 2H), 3.54 (d, J=6.4 Hz, 2H), 2.29-2.23 (m, 2H), 2.05-1.99 (m, 2H), 1.88-1.78 (m, 2H), 1.57 (s, 9H), 1.23-1.14 (m, 4H), 0.45-0.41 (m, 2H), 0.29-0.25 (m, 2H).

### Step 7: Synthesis of Compound BB-2

Compound BB-2-7 (1 g, 1.70 mmol) was dissolved in dichloromethane (10 mL) at room temperature under nitrogen atmosphere, then Dess-Martin periodinane (1.45 g, 3.41 mmol) was added thereto. The reaction mixture was stirred and reacted at room temperature for 1 hour. After the reaction was completed, the reaction mixture was added with a saturated solution of sodium sulfite (20 mL), and extracted with dichloromethane (3×20 mL). The organic phases were washed with saturated brine (30 mL), dried over anhydrous sodium sulfate, and filtered. The filtrate was concentrated under reduced pressure to remove the solvent. The resulting residue was separated by column chromatography (eluent: petroleum ether/ethyl acetate=4/1 to 1/1, v/v) to obtain compound BB-2. MS-ESI m/z: 585.4 [M+H]⁺. ¹H NMR (400 MHz, CDCl₃) δ: 9.70 (s, 1H), 9.08 (s, 1H), 8.52 (d, J=5.6 Hz, 1H), 8.38 (s, 1H), 8.34 (s, 1H), 8.33 (s, 1H), 7.63 (dd, J=1.6, 5.2 Hz, 1H), 6.84 (t, J=54.6 Hz, 1H), 4.16-4.06 (m, 1H), 3.94 (d, J=6.8 Hz, 2H), 2.41-2.33 (m, 1H), 2.34-2.28 (m, 2H), 2.25-2.19 (m, 2H), 1.95-1.82 (m, 2H), 1.57 (s, 9H), 1.53-1.42 (m, 2H), 1.24-1.17 (m, 1H), 0.47-0.39 (m, 2H), 0.30-0.24 (m, 2H).

### Reference Example 3: Fragment BB-3

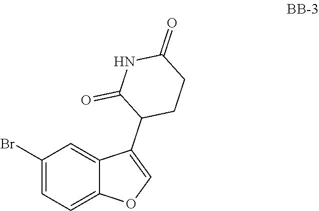 [SMILES: O=C1CCC(c2coc3ccc(Br)cc23)C(=O)N1]

### Synthetic Route:

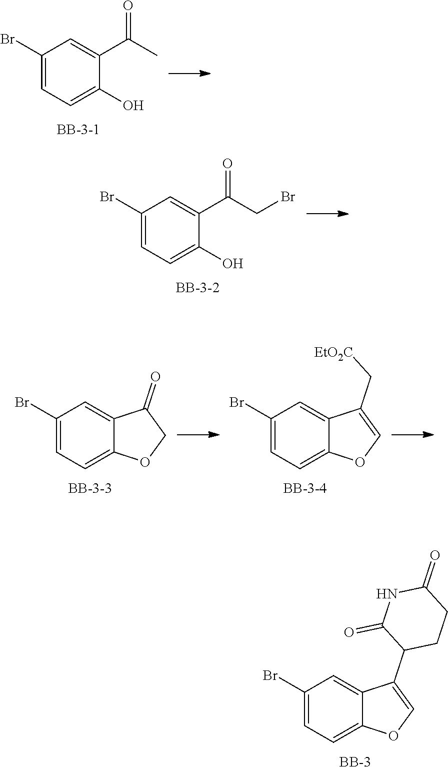

### Step 1: Synthesis of Compound BB-3-2

Compound BB-3-1 (200 g, 930.04 mmol) was dissolved in chloroform (1000 mL) and ethyl acetate (1000 mL) at room temperature under nitrogen atmosphere, then copper bromide (415.45 g, 1.86 mol) was added thereto. The reaction mixture was heated to 90° C., and stirred and reacted for 16 hours. After the reaction was completed, the mixture was cooled to room temperature, and filtered. The filter cake was rinsed with dichloromethane (1.5 L) to obtain a solution of compound BB-3-2, which was used in the next step directly.

### Step 2: Synthesis of Compound BB-3-3

The solution of compound BB-3-2 (3.5 L) obtained above was cooled to 0° C., and then triethylamine (141.17 g, 1.40 mol, 194.18 mL) was slowly added dropwise thereto. After the dropwise addition was completed, the reaction mixture was slowly warmed to room temperature, and stirred and reacted for 2 hours. After the reaction was completed, the mixture was added with water (2 L) and the phases were separated. The aqueous phase was extracted with dichloromethane (500 mL). The organic phases were combined, washed with saturated brine (1 L×2), dried over anhydrous sodium sulfate, and filtered. The filtrate was concentrated under reduced pressure to remove the solvent to obtain compound BB-3-3.

### Step 3: Synthesis of Compound BB-3-4

Compound BB-3-3 (198.1 g, 929.93 mmol) was dissolved in toluene (1.5 L) at room temperature under nitrogen atmosphere, and then (ethoxycarbonylmethylene)triphenylphosphorane (388.76 g, 1.12 mol) was added thereto. The reaction mixture was heated to 130° C., and stirred and reacted for 36 hours. After the reaction was completed, the mixture was cooled to room temperature, concentrated under reduced pressure to remove the solvent. The resulting residue was added with methyl tert-butyl ether (700 mL×3), stirred at room temperature for 20 minutes, and filtered. The filter cake was rinsed with methyl tert-butyl ether (100 mL), and the filtrate was collected. The filtrate was concentrated under reduced pressure to remove the solvent. The resulting residue was separated by column chromatography (eluent: petroleum ether/ethyl acetate=1/0 to 70/1, v/v) to obtain compound BB-3-4. ¹H NMR (400 MHz, CDCl₃) δ: 7.72 (d, J=2.0 Hz, 1H), 7.64 (s, 1H), 7.41 (dd, J=2.0 Hz, 8.8 Hz, 1H), 7.36 (d, J=8.8 Hz, 1H), 4.21 (q, J=7.0 Hz, 2H), 3.66 (d, J=0.8 Hz, 2H), 1.30 (t, J=7.2 Hz, 3H).

### Step 4: Synthesis of Compound BB-3

Compound BB-3-4 (3 g, 10.60 mmol) was dissolved in N, N-dimethylformamide (20 mL) at room temperature under nitrogen atmosphere, then acrylamide (903.79 mg, 12.72 mmol) and potassium tert-butoxide (1.78 g, 15.89 mmol) were sequentially added thereto. The reaction mixture was stirred and reacted at room temperature for 1 hour. After the reaction was completed, the reaction mixture was poured into 1 N hydrochloric acid (15 mL), added with water (25 mL), and extracted with ethyl acetate (35 mL×3). The organic phases were combined, washed with saturated brine (30 mL×2), dried over anhydrous sodium sulfate, and filtered. The filtrate was concentrated under reduced pressure to remove the solvent. The resulting residue was separated by column chromatography (eluent: petroleum ether/ethyl acetate=5/1 to 1/1, v/v) to obtain compound BB-3. MS-ESI m/z: 308.0 [M+H]⁺, 310.0 [M+H+2]⁺. ¹H NMR (400 MHz, DMSO_d₆) δ: 10.87 (s, 1H), 7.97 (s, 1H), 7.85 (d, J=2.0 Hz, 1H), 7.57 (d, J=8.4 Hz, 1H), 7.45 (dd, J=2.0, 8.8 Hz, 1H), 4.15 (dd, J=4.8, 12.4 Hz, 1H), 2.78-2.67 (m, 1H), 2.62-2.54 (m, 1H), 2.43-2.29 (m, 1H), 2.14-2.04 (m, 1H).

### Reference Example 4: Fragment BB-4

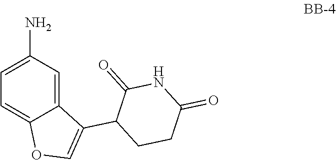 [SMILES: Nc1ccc2occ(C3CCC(=O)NC3=O)c2c1]

### Synthetic Route:

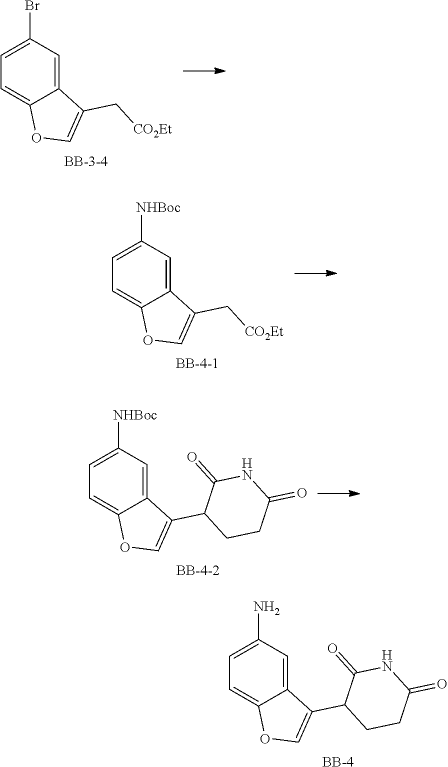

### Step 1: Synthesis of Compound BB-4-1

Compound BB-3-4 (5 g, 17.66 mmol), tert-butyl carbamate (2.48 g, 21.19 mmol), potassium phosphate (11.25 g, 52.98 mmol), tris(dibenzylideneacetone)dipalladium (323.44 mg, 353.21 μmol) and 2-di-tert-butylphosphine-2′,4′,6′-triisopropyl biphenyl (299.98 mg, 706.42 μmol) were sequentially added into a mixed solvent of toluene (100 mL) and water (20 mL) at room temperature under nitrogen atmosphere. The reaction mixture was heated to 110° C., and stirred and reacted for 16 hours. After the reaction was completed, the reaction mixture was cooled to room temperature, added with water (100 mL), and filtered. The filter cake was rinsed with ethyl acetate (100 mL), and the filtrate was combined and allowed to stand for phase separation. The organic phase was collected. The organic phase was dried over anhydrous sodium sulfate, and filtered. The filtrate was concentrated under reduced pressure to remove the solvent. The resulting residue was separated by column chromatography (eluent: ethyl acetate/petroleum ether=1/5, v/v) to obtain compound BB-4-1. ¹H NMR (400 MHz, CDCl₃) δ: 7.71 (br s, 1H), 7.62 (s, 1H), 7.37 (d, J=8.8 Hz, 1H), 7.15 (dd, J=2.0 Hz, 8.8 Hz, 1H), 6.58 (s, 1H), 4.20 (q, J=7.2 Hz, 2H), 3.67 (q, J=1.2 Hz, 2H), 1.53 (s, 9H), 1.29 (t, J=7.2 Hz, 3H).

### Step 2: Synthesis of Compound BB-4-2

Compound BB-4-1 (5.6 g, 17.54 mmol) and acrylamide (1.50 g, 21.05 mmol) were dissolved in N, N-dimethylformamide (100 mL) at room temperature, and then potassium tert-butoxide (1.8 g, 16.04 mmol) was added thereto. The reaction mixture was stirred and reacted at room temperature for 2 hours, and an additional amount of potassium tert-butoxide (0.6 g, 5.35 mmol) was added thereto. The reaction mixture was further stirred and reacted at room temperature for 0.5 hours, After the reaction was completed, the reaction mixture was poured into 1 M dilute hydrochloric acid (300 mL), stirred for 10 minutes, and filtered. The filter cake was rinsed with water (100 mL), and collected. The resulting filter cake was separated by column chromatography (eluent: ethyl acetate/petroleum ether=1/1, v/v) to obtain compound BB-4-2. ¹H NMR (400 MHz, CDCl₃) δ: 8.32 (br s, 1H), 7.72 (br s, 1H), 7.53 (s, 1H), 7.39 (d, J=8.8 Hz, 1H), 7.12 (dd, J=2.0 Hz, 8.8 Hz, 1H), 6.71 (br s, 1H), 3.97 (t, J=7.6 Hz, 1H), 2.82-2.63 (m, 2H), 2.39-2.29 (m, 2H), 1.53 (s, 9H).

Step 3: Synthesis of hydrochloride of compound BB-4

Compound BB-4-2 (2.3 g, 6.68 mmol) was dissolved in ethyl acetate (20 mL) at room temperature, then a solution of hydrochloric acid in ethyl acetate (4 M, 40 mL) was added thereto. The reaction mixture was stirred and reacted at room temperature for 16 hours. After the reaction was completed, the reaction mixture was filtered. The filter cake was rinsed with ethyl acetate (20 mL), collected, and dried under vacuum to obtain the hydrochloride of compound BB-4. ¹H NMR (400 MHz, D₂O) δ: 7.85 (s, 1H), 7.67 (d, J=8.8 Hz, 1H), 7.56 (d, J=1.6 Hz, 1H), 7.34 (dd, J=2.0 Hz, 8.8 Hz, 1H), 4.24 (dd, J=5.2 Hz, 12.4 Hz, 1H), 2.91-2.74 (m, 2H), 2.50-2.37 (m, 1H), 2.35-2.25 (m, 1H).

### Reference Example 5: Fragment BB-5

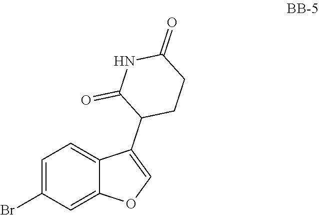 [SMILES: O=C1CCC(c2coc3cc(Br)ccc23)C(=O)N1]

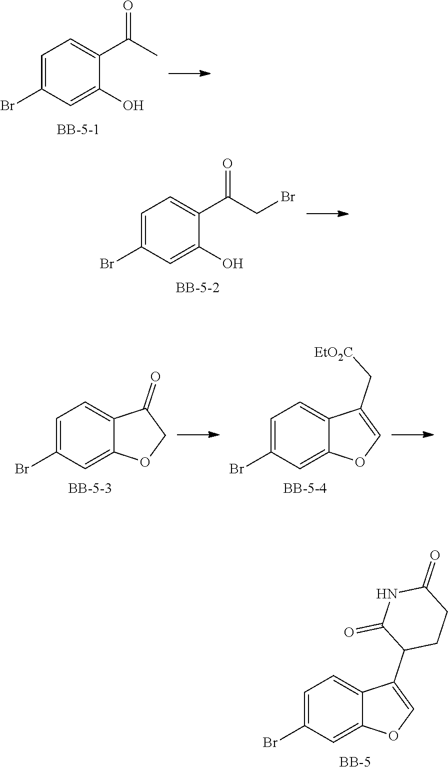

### Step 1: Synthesis of Compound BB-5-2

Compound BB-5-1 (200 g, 930.04 mmol) was dissolved in chloroform (1 mL) and ethyl acetate (1 L) at room temperature under nitrogen atmosphere, then copper bromide (415.45 g, 1.86 mol) was added thereto. The reaction mixture was heated to 90° C., and stirred and reacted for 12 hours. After the reaction was completed, the mixture was cooled to room temperature, and filtered. The filter cake was rinsed with dichloromethane (300 mL×2). The filtrate was collected to obtain a solution of compound BB-5-2, which was used in the next step directly.

### Step 2: Synthesis of Compound BB-5-3

The solution of compound BB-5-2 (273 g, 928.76 mmol) obtained above was cooled to 0° C., and then triethylamine (140.97 g, 1.39 mol, 193.91 mL) was slowly added dropwise thereto. After the dropwise addition was completed, the reaction mixture was slowly warmed to room temperature, and stirred and reacted for 1 hour. After the reaction was completed, the mixture was added with water (600 mL) and extracted. The phases were separated. The organic phase was washed with saturated brine (1 L), dried over anhydrous sodium sulfate, and filtered. The filtrate was concentrated under reduced pressure to remove half of the solvent, and added with toluene (500 mL). The mixture was continued to be concentrated under reduced pressure to remove the residual low-boiling solvent to obtain a solution of compound BB-5-3 in toluene.

### Step 3: Synthesis of Compound BB-5-4

Toluene (2 L) was added to half of the solution of the compound BB-5-3 in toluene at room temperature under nitrogen atmosphere, and then (ethoxycarbonylmethylene)triphenylphosphorane (161.90 g, 464.73 mmol) was added thereto. The reaction mixture was heated to 130° C., and stirred and reacted for 20 hours. After the reaction was completed, the mixture was cooled to room temperature, and the two batches were combined for processing. The mixture was concentrated under reduced pressure to remove the solvent. The resulting residue was added with methyl tert-butyl ether (800 mL), stirred at room temperature for 30 minutes, and filtered. The filter cake was rinsed with methyl tert-butyl ether (100 mL×2). The filtrate was concentrated under reduced pressure to remove the solvent. The resulting residue was separated by column chromatography (eluent: petroleum ether/ethyl acetate=100/1 to 10/1, v/v) to obtain compound BB-5-4. ¹H NMR (400 MHz, CDCl₃) δ: 7.94 (s, 1H), 7.88 (s, 1H), 7.56 (d, J=8.4 Hz, 1H), 7.44 (d, J=8.4 Hz, 1H), 4.11 (q, J=7.2 Hz, 2H), 3.79 (s, 2H), 1.19 (t, J=7.2 Hz, 3H).

### Step 4: Synthesis of Compound BB-5

Compound BB-5-4 (5.00 g, 17.66 mmol) was dissolved in N, N-dimethylformamide (50 mL) at room temperature under nitrogen atmosphere, then acrylamide (1.51 g, 21.19 mmol) and potassium tert-butoxide (2.97 g, 26.49 mmol) were sequentially added thereto. The reaction mixture was stirred and reacted at room temperature for 2 hours. After the reaction was completed, the mixture was poured into 1 M dilute hydrochloric acid (100 mL), and extracted with ethyl acetate (30 mL×2). The organic phases were combined, washed with 10% brine (100 mL×2), dried over anhydrous sodium sulfate, and filtered. The filtrate was concentrated under reduced pressure to remove the solvent. The resulting residue was separated by column chromatography (eluent: ethyl acetate/petroleum ether=1/1, v/v) to obtain compound BB-5. MS-ESI m/z: 308.0 [M+H]⁺, 310.0 [M+H+2]⁺. ¹H NMR (400 MHz, DMSO_d₆) δ: 10.91 (s, 1H), 7.94 (s, 1H), 7.89 (d, J=1.6 Hz, 1H), 7.56 (d, J=8.4 Hz, 1H), 7.42 (dd, J=1.6 Hz, 8.0 Hz, 1H), 4.15 (dd, J=4.8 Hz, 12.0 Hz, 1H), 2.80-2.68 (m, 1H), 2.58 (dt, J=4.0 Hz, 17.2 Hz, 1H), 2.38-2.25 (m, 1H), 2.15-2.05 (m, 1H).

### Reference Example 6: Fragment BB-6

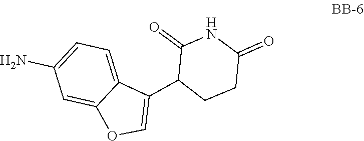 [SMILES: Nc1ccc2c(C3CCC(=O)NC3=O)coc2c1]

### Synthetic Route:

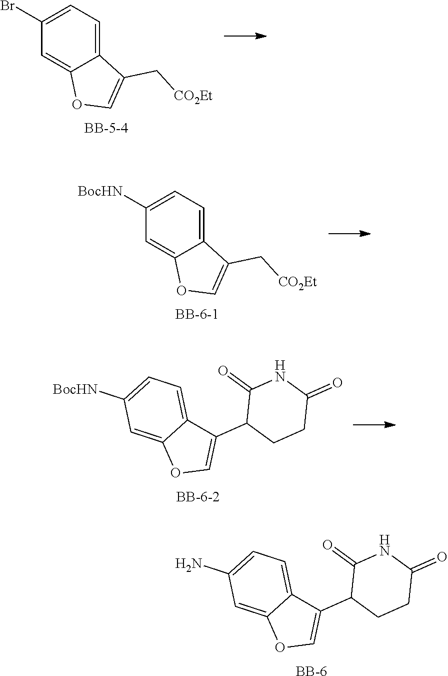

### Step 1: Synthesis of Compound BB-6-1

Compound BB-5-4 (10 g, 35.32 mmol), tert-butyl carbamate (4.97 g, 42.39 mmol), potassium phosphate (22.49 g, 105.96 mmol), tris(dibenzylideneacetone)dipalladium (646.88 mg, 706.42 μmol) and 2-di-tert-butylphosphine-2′,4′,6′-triisopropyl biphenyl (599.95 mg, 1.41 mmol) were dissolved in a mixed solvent of toluene (100 mL) and water (20 mL) at room temperature under nitrogen atmosphere. The reaction mixture was heated to 110° C., and stirred and reacted for 12 hours. After the reaction was completed, the mixture was cooled to room temperature, added with water (100 mL), and extracted with ethyl acetate (70 mL×3). The organic phases were combined, washed with saturated brine (70 mL×2), dried over anhydrous sodium sulfate, and filtered. The filtrate was concentrated under reduced pressure to remove the solvent. The residue was added with n-heptane (70 mL), stirred at room temperature for 10 minutes, and filtered. The filter cake was collected. The filter cake was again added with n-heptane (50 mL), stirred at room temperature for 10 minutes, and filtered. The filter cake was rinsed with n-heptane (10 mL×3), collected, and dried under vacuum to obtain compound BB-6-1. MS-ESI m/z: 264.2 [M−55]⁺. ¹H NMR (400 MHz, CDCl₃) δ: 7.77 (s, 1H), 7.56 (s, 1H), 7.44 (d, J=8.4 Hz, 1H), 7.06 (dd, J=1.6 Hz, 8.4 Hz, 1H), 6.58 (s, 1H), 4.19 (q, J=7.0 Hz, 2H), 3.66 (s, 2H), 1.54 (s, 9H), 1.27 (t, J=7.2 Hz, 3H).

### Step 2: Synthesis of Compound BB-6-2

Compound BB-6-1 (9.8 g, 30.69 mmol) and acrylamide (2.62 g, 36.83 mmol) were dissolved in N, N-dimethylformamide (100 mL) at room temperature, and then potassium tert-butoxide (6.20 g, 55.24 mmol) was added thereto. The reaction mixture was stirred and reacted at room temperature for 1 hour. After the reaction was completed, the reaction mixture was poured into 0.5 M hydrochloric acid (120 mL), then added with water (300 mL), and extracted with ethyl acetate (150 mL×3). The organic phases were combined, washed with saturated brine (150 mL), dried over anhydrous sodium sulfate, and filtered. The filtrate was concentrated under reduced pressure to remove the solvent. The resulting residue was added with methyl tert-butyl ether (70 mL), stirred at room temperature for 0.5 hours, and filtered. The filter cake was rinsed with methyl tert-butyl ether (10 mL×3), collected, and dried under vacuum to obtain compound BB-6-2. ¹H NMR (400 MHz, DMSO_d₆) δ: 10.88 (s, 1H), 9.47 (s, 1H), 7.78 (s, 2H), 7.43 (d, J=8.8 Hz, 1H), 7.23 (dd, J=1.4 Hz, 8.6 Hz, 1H), 4.07 (dd, J=4.8 Hz, 12.0 Hz, 1H), 2.78-2.65 (m, 1H), 2.62-2.53 (m, 1H), 2.35-2.23 (m, 1H), 2.15-2.06 (m, 1H), 1.49 (s, 9H).

Step 3: Synthesis of hydrochloride of compound BB-6

Compound BB-6-2 (6.6 g, 19.17 mmol) was dissolved in dichloromethane (20 mL) at room temperature, then a solution of hydrochloric acid in ethyl acetate (4 M, 150 mL) was added thereto. The reaction mixture was stirred and reacted at room temperature for 4 hours. After the reaction was completed, the mixture was concentrated under reduced pressure to remove the solvent to obtain the hydrochloride of compound BB-6. ¹H NMR (400 MHz, DMSO_d₆) δ: 10.92 (s, 1H), 10.06 (s, 2H), 7.98 (s, 1H), 7.66 (d, J=8.4 Hz, 1H), 7.57 (d, J=1.6 Hz, 1H), 7.21 (dd, J=1.8 Hz, 8.2 Hz, 1H), 4.16 (dd, J=4.8 Hz, 12.0 Hz, 1H), 2.81-2.69 (m, 1H), 2.63-2.54 (m, 1H), 2.39-2.26 (m, 1H), 2.16-2.07 (m, 1H).

### Reference Example 7: Fragment BB-7

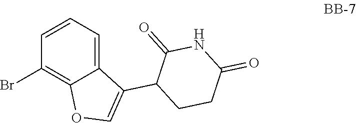 [SMILES: O=C1CCC(c2coc3c(Br)cccc23)C(=O)N1]

### Synthetic Route:

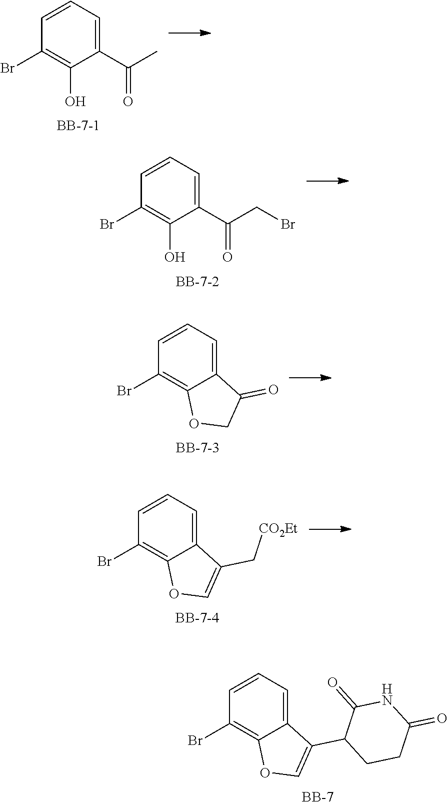

### Step 1: Synthesis of Compound BB-7-2

Compound BB-7-1 (11.39 g, 52.97 mmol) was dissolved in chloroform (100 mL) and ethyl acetate (100 mL) at room temperature under nitrogen atmosphere, then copper bromide (23.66 g, 105.93 mmol) was added thereto. The reaction mixture was heated to 110° C., and stirred and reacted for 16 hours. After the reaction was completed, the mixture was cooled to room temperature, and filtered. The filtrate was concentrated under reduced pressure to remove the solvent, then the residue was added with water (20 mL), and extracted with ethyl acetate (20 mL×3). The organic phases were combined, dried over anhydrous sodium sulfate, and filtered. The filtrate was concentrated under reduced pressure to remove the solvent. The resulting residue was separated by column chromatography (eluent: petroleum ether/ethyl acetate=1/0 to 100/1, v/v) to obtain compound BB-7-2. ¹H NMR (400 MHz, CDCl₃) δ: 12.39 (s, 1H), 7.81 (dd, J=1.2 Hz, 8.0 Hz, 1H), 7.76 (dd, J=1.2 Hz, 8.0 Hz, 1H), 6.88 (t, J=8.0 Hz, 1H), 4.47 (s, 2H).

### Step 2: Synthesis of Compound BB-7-3

Compound BB-7-2 (6.86 g, 18.44 mmol) was dissolved in dichloromethane (80 mL) at 0° C., and then triethylamine (1.87 g, 18.44 mmol) was slowly added dropwise thereto. The reaction mixture was slowly warmed to room temperature, and stirred and reacted for 1 hour. After the reaction was completed, the reaction mixture was directly concentrated under reduced pressure to remove the solvent to obtain a crude product of compound BB-7-3, which was used in the next step directly.

### Step 3: Synthesis of Compound BB-7-4

The crude product of compound BB-7-3 (3.93 g, 18.45 mmol) was dissolved in toluene (80 mL) at room temperature under nitrogen atmosphere, and then (ethoxycarbonylmethylene)triphenylphosphorane (9.64 g, 27.67 mmol) was added thereto. The reaction mixture was heated to 130° C., and stirred and reacted for 36 hours. After the reaction was completed, the reaction mixture was cooled to room temperature, concentrated under reduced pressure to remove the solvent. Then the residue was added with water (100 mL), and extracted with ethyl acetate (50 mL×3). The organic phases were combined, washed with saturated brine (50 mL), dried over anhydrous sodium sulfate, and filtered. The filtrate was concentrated under reduced pressure to remove the solvent. The resulting residue was separated by column chromatography (eluent: petroleum ether/ethyl acetate=1/0 to 100/1, v/v) to obtain compound BB-7-4. ¹H NMR (400 MHz, CDCl₃) δ: 7.72 (s, 1H), 7.52 (dd, J=0.8 Hz, 7.6 Hz, 1H), 7.48 (dd, J=0.8 Hz, 7.6 Hz, 1H), 7.15 (t, J=7.6 Hz, 1H), 4.20 (q, J=7.2 Hz, 2H), 3.70 (d, J=0.8 Hz, 2H), 1.28 (t, J=7.2 Hz, 3H).

### Step 4: Synthesis of Compound BB-7

Compound BB-7-4 (4.77 g, 16.85 mmol) was dissolved in N, N-dimethylformamide (30 mL) at 0° C. under nitrogen atmosphere, then acrylamide (1.20 g, 16.85 mmol) and potassium tert-butoxide (1.89 g, 16.85 mmol) were sequentially added thereto. The reaction mixture was stirred and reacted at 0° C. for 1 hour. After the reaction was completed, the mixture was poured into saturated ammonium chloride solution (50 mL), producing a large amount of white solid. The mixture was filtered and the solid was collected. The solid was slurried with methanol (20 mL×2) at room temperature and filtered. The filter cake was collected, and dried under vacuum to obtain compound BB-7. ¹H NMR (400 MHz, DMSO_d₆) δ: 10.92 (s, 1H), 8.04 (s, 1H), 7.62 (dd, J=0.8 Hz, 7.6 Hz, 1H), 7.56 (d, J=7.6 Hz, 1H), 7.21 (t, J=7.6 Hz, 1H), 4.17 (dd, J=4.8 Hz, 12.0 Hz, 1H), 2.79-2.70 (m, 1H), 2.63-2.55 (m, 1H), 2.40-2.27 (m, 1H), 2.16-2.08 (m, 1H).

### Reference Example 8: Fragment BB-8

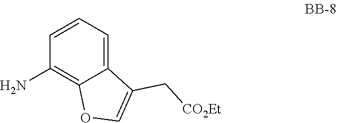 [SMILES: CCOC(=O)Cc1coc2c(N)cccc12]

### Synthetic Route:

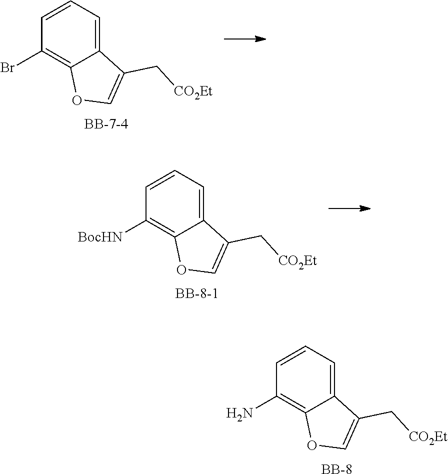

### Step 1: Synthesis of Compound BB-8-1

Compound BB-7-4 (2 g, 7.06 mmol) was dissolved in toluene (50 mL) and water (5 mL) at room temperature under nitrogen atmosphere, and then tris(dibenzylideneacetone)dipalladium (647 mg, 706.42 μmol), 2-di-tert-butylphosphino-2′,4′,6′-triisopropyl biphenyl (450 mg, 1.06 mmol), potassium phosphate (6 g, 28.26 mmol), and tert-butyl carbamate (1.66 g, 14.13 mmol) were sequentially added thereto. The reaction mixture was heated to 100° C., and stirred and reacted for 16 hours. After the reaction was completed, the mixture was cooled to room temperature. The reaction mixture was poured into water (20 mL) and extracted with ethyl acetate (10 mL×3). The organic phases were combined, washed with saturated brine (10 mL), dried over anhydrous sodium sulfate, and filtered. The filtrate was concentrated under reduced pressure to remove the solvent, and the resulting residue was separated by column chromatography (eluent: petroleum ether/ethyl acetate=10/0 to 10/1, v/v) to obtain compound BB-8-1. ¹H NMR (400 MHz, CDCl₃) δ: 7.93 (br s, 1H), 7.62 (s, 1H), 7.24-7.20 (m, 2H), 6.97 (s, 1H), 4.20 (q, J=6.8 Hz, 2H), 3.69 (d, J=0.8 Hz, 2H), 1.56 (s, 9H), 1.28 (t, J=7.2 Hz, 3H).

Step 2: Synthesis of hydrochloride of compound BB-8

Compound BB-8-1 (1.32 g, 4.13 mmol) was dissolved in ethyl acetate (3 mL) at room temperature, then a solution of hydrochloric acid in ethyl acetate (4 M, 15 mL) was added thereto. The reaction mixture was stirred and reacted at room temperature for 2 hours. After the reaction was completed, the reaction mixture was concentrated under reduced pressure to remove the solvent directly to obtain the hydrochloride of compound BB-8.

### Reference Example 9: Fragment BB-9

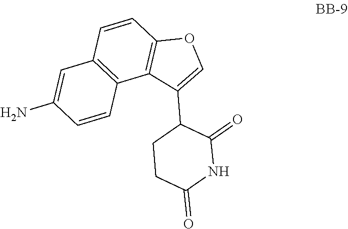 [SMILES: Nc1ccc2c(ccc3occ(C4CCC(=O)NC4=O)c32)c1]

### Synthetic Route:

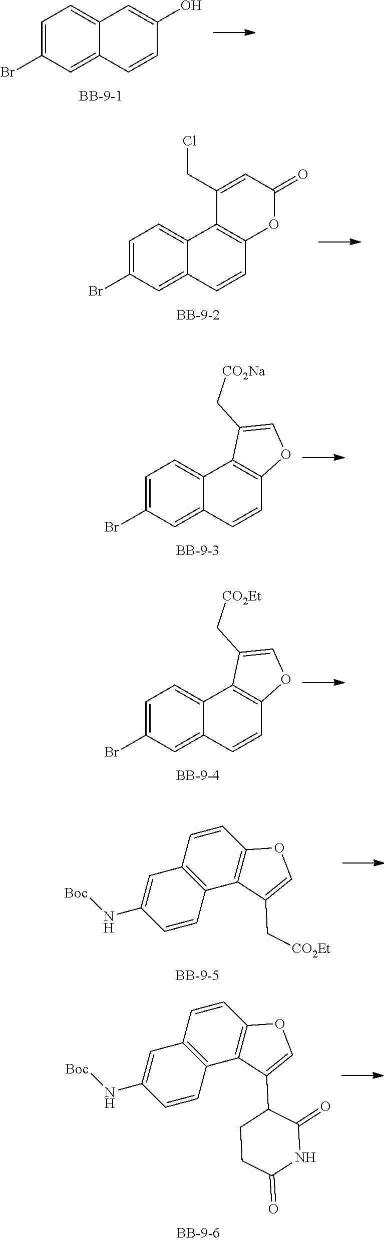

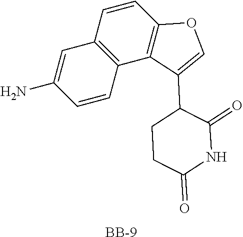 [SMILES: Nc1ccc2c(ccc3occ(C4CCC(=O)NC4=O)c32)c1 ; "BB-9"]

### Step 1: Synthesis of Compound BB-9-2

Concentrated sulfuric acid (103.08 g, 1.03 mol, 56.02 mL, purity of 98%) was slowly added dropwise to a mixture of compound BB-9-1 (25 g, 112.07 mmol) and ethyl 4-chloroacetoacetate (18.45 g, 112.07 mmol) at 0° C., with internal temperature controlled at 0 to 5° C. After the dropwise addition was completed, the reaction mixture was warmed to room temperature, and stirred and reacted for 12 hours. The four batches were combined for processing. After the reaction was completed, the reaction mixture was slowly poured into ice water (3 L) with stirring, stirred at room temperature for 10 minutes, and filtered. The filter cake was collected and dried under vacuum to obtain compound BB-9-2. MS-ESI m/z: 323.0 [M+H]⁺, 325.0 [M+H+2]⁺.

### Step 2: Synthesis of Compound BB-9-3

Sodium hydroxide (17.65 g, 441.36 mmol) was dissolved in water (700 mL) at room temperature, then compound BB-9-2 (54.40 g, 110.34 mmol, purity of 65.63%) was added thereto. The reaction mixture was heated to 80° C., and stirred and reacted for 12 hours. The three batches were combined for processing. After the reaction was completed, the mixture was cooled to room temperature, and filtered. The filter cake was washed with water (500 mL), collected, and dried under vacuum to obtain compound BB-9-3. ¹H NMR (400 MHz, DMSO_d₆) δ: 8.50 (d, J=9.2 Hz, 1H), 8.26 (d, J=2.4 Hz, 1H), 7.84 (s, 1H), 7.76 (d, J=1.6 Hz, 2H), 7.63 (dd, J=2.0 Hz, 8.8 Hz, 1H), 3.50 (s, 2H).

### Step 3: Synthesis of Compound BB-9-4

Compound BB-9-3 (33.16 g, 101.37 mmol) was dissolved in ethanol (300 mL) at room temperature, then concentrated sulfuric acid (27.19 g, 271.68 mmol, 14.78 mL, purity of 98%) was slowly added dropwise thereto. The reaction mixture was heated to 80° C., and stirred and reacted for 12 hours. The two batches were combined for processing. After the reaction was completed, the mixture was cooled to room temperature and concentrated under reduced pressure to remove the solvent. The resulting residue was added with water (600 mL) and extracted with ethyl acetate (200 mL×2). The organic phases were combined, sequentially washed with an aqueous solution of sodium hydroxide (2 M, 300 mL) and saturated brine (500 mL), dried over anhydrous sodium sulfate, and filtered. The filtrate was concentrated under reduced pressure to remove the solvent to obtain compound BB-9-4. ¹H NMR (400 MHz, CDCl₃) δ: 8.13-8.06 (m, 2H), 7.78 (s, 1H), 7.69-7.60 (m, 3H), 4.23 (q, J=7.2 Hz, 2H), 4.03 (s, 2H), 1.27 (t, J=7.2 Hz, 3H).

### Step 4: Synthesis of Compound BB-9-5

Compound BB-9-4 (1 g, 3.00 mmol), tris(dibenzylideneacetone)dipalladium (275 mg, 300.14 μmol), 2-di-tert-butylphosphino-2′,4′,6′-triisopropyl biphenyl (191.18 mg, 450.21 mmol), potassium phosphate (2.55 g, 12.01 mmol), and tert-butyl carbamate (527.41 mg, 4.50 mmol) were dissolved in a mixed solvent of toluene (50 mL) and water (5 mL) at room temperature under nitrogen atmosphere. The reaction mixture was heated to 100° C., and stirred and reacted for 12 hours. After the reaction was completed, the mixture was cooled to room temperature. The reaction mixture was poured into water (20 mL) and extracted with ethyl acetate (10 mL×3). The organic phases were combined, washed with saturated brine (10 mL), dried over anhydrous sodium sulfate, and filtered. The filtrate was concentrated under reduced pressure to remove the solvent. The resulting residue was separated by column chromatography (eluent: petroleum ether/ethyl acetate=10/0 to 10/1, v/v) to obtain intermediate BB-9-5. MS-ESI m/z: 392.2 [M+Na]⁺. ¹H NMR (400 MHz, CDCl₃) δ: 8.14 (d, J=9.2 Hz, 1H), 8.12 (br s, 1H), 7.74 (s, 1H), 7.67 (d, J=9.2 Hz, 1H), 7.62 (d, J=8.8 Hz, 1H), 7.45 (dd, J=2.2 Hz, 9.0 Hz, 1H), 6.64 (br s, 1H), 4.22 (q, J=7.0 Hz, 2H), 4.04 (s, 2H), 1.57 (s, 9H), 1.27 (t, J=7.2 Hz, 3H).

### Step 5: Synthesis of Compound BB-9-6

Compound BB-9-5 (1.13 g, 2.74 mmol, purity of 89.57%) and acrylamide (234 mg, 3.29 mmol) were dissolved in N, N-dimethylformamide (10 mL) at 0° C. under nitrogen atmosphere, and then potassium tert-butoxide (368.93 mg, 3.29 mmol) was added thereto. The reaction mixture was stirred and reacted at 0° C. for 1 hour. After the reaction was completed, the reaction mixture was slowly poured into an aqueous solution of saturated ammonium chloride (30 mL), and extracted with ethyl acetate (15 mL×3). The organic phases were combined, washed with saturated brine (10 mL), dried over anhydrous sodium sulfate, and filtered. The filtrate was concentrated under reduced pressure to remove the solvent. The resulting residue was added with methanol (10 mL), stirred at room temperature for 0.5 hours, and filtered. The filter cake was collected and dried under vacuum to obtain compound BB-9-6. MS-ESI m/z: 417.0 [M+Na]⁺. ¹H NMR (400 MHz, CDCl₃) δ: 8.13 (br s, 1H), 8.08 (br s, 1H), 7.90 (d, J=8.8 Hz, 1H), 7.71 (d, J=9.2 Hz, 1H), 7.67-7.62 (m, 2H), 7.48 (dd, J=2.4 Hz, 8.8 Hz, 1H), 6.67 (br s, 1H), 4.47 (dd, J=5.4 Hz, 8.6 Hz, 1H), 2.85-2.71 (m, 2H), 2.55-2.40 (m, 2H), 1.57 (s, 9H).

### Step 6: Synthesis of Hydrochloride of Compound BB-9

Compound BB-9-6 (421 mg, 1.07 mmol) was dissolved in hydrochloric acid/dioxane solution (4 M, 20 mL) at room temperature. The reaction mixture was stirred and reacted at room temperature for 12 hours. After the reaction was completed, the reaction mixture was cooled, then poured into a saturated solution of ammonium chloride (100 mL), and extracted with ethyl acetate (10 mL×3). The organic phases were combined, washed with saturated brine (10 mL), dried over anhydrous sodium sulfate, filtered, and evaporated to dryness by rotary evaporation. The resulting residue was separated by column chromatography (eluent: petroleum ether/ethyl acetate=1/1, v/v) to obtain the hydrochloride of compound BB-9. MS-ESI m/z: 294.9 [M+H]⁺. ¹H NMR (400 MHz, DMSO_d₆) δ: 10.95 (br s, 1H), 8.23 (d, J=9.2 Hz, 1H), 8.04 (s, 1H), 7.88-7.81 (m, 3H), 7.48-7.42 (m, 1H), 4.66 (dd, J=4.4 Hz, 12.4 Hz, 1H), 2.92-2.81 (m, 1H), 2.69-2.60 (m, 1H), 2.46-2.38 (m, 1H), 2.31-2.23 (m, 1H).

### Reference Example 10: Fragment BB-10

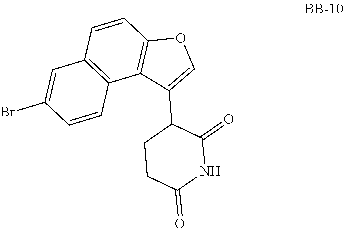 [SMILES: O=C1CCC(c2coc3ccc4cc(Br)ccc4c23)C(=O)N1]

### Synthetic Route:

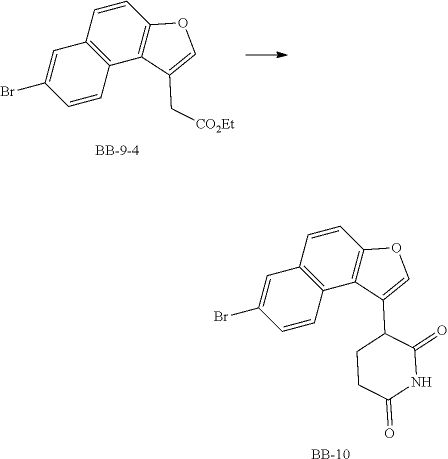 [SMILES: CCOC(=O)Cc1coc2ccc3cc(Br)ccc3c12 ; O=C1CCC(c2coc3ccc4cc(Br)ccc4c23)C(=O)N1 ; "BB-10"]

### Synthesis of Compound BB-10

Compound BB-9-4 (1 g, 3.00 mmol) was dissolved in N, N-dimethylformamide (10 mL) at room temperature under nitrogen atmosphere, then acrylamide (256 mg, 3.60 mmol) and potassium tert-butoxide (404.15 mg, 3.60 mmol) were sequentially added thereto. The reaction mixture was stirred and reacted at room temperature for 1 hour. After the reaction was completed, the reaction mixture was slowly poured into a saturated aqueous solution of ammonium chloride (100 mL), stirred at room temperature for 1 hour, and filtered. The filter cake was collected. The resulting filter cake was added with methanol (20 mL), stirred at room temperature for 0.5 hours, and filtered. The filter cake was collected, and dried under vacuum to obtain compound BB-10. MS-ESI m/z: 357.7 [M+H]⁺, 359.7 [M+H+2]⁺. ¹H NMR (400 MHz, CDCl₃) δ: 8.15 (d, J=2.0 Hz, 1H), 8.06 (br s, 1H), 7.87 (d, J=8.8 Hz, 1H), 7.72-7.65 (m, 4H), 4.47 (dd, J=5.2 Hz, 9.6 Hz, 1H), 2.89-2.74 (m, 2H), 2.58-2.41 (m, 2H).

### Reference Example 11: Fragment BB-11

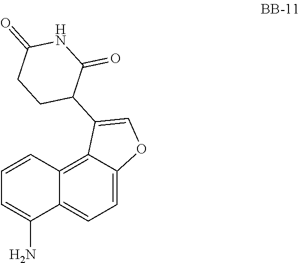 [SMILES: Nc1cccc2c1ccc1occ(C3CCC(=O)NC3=O)c12]

### Synthetic Route:

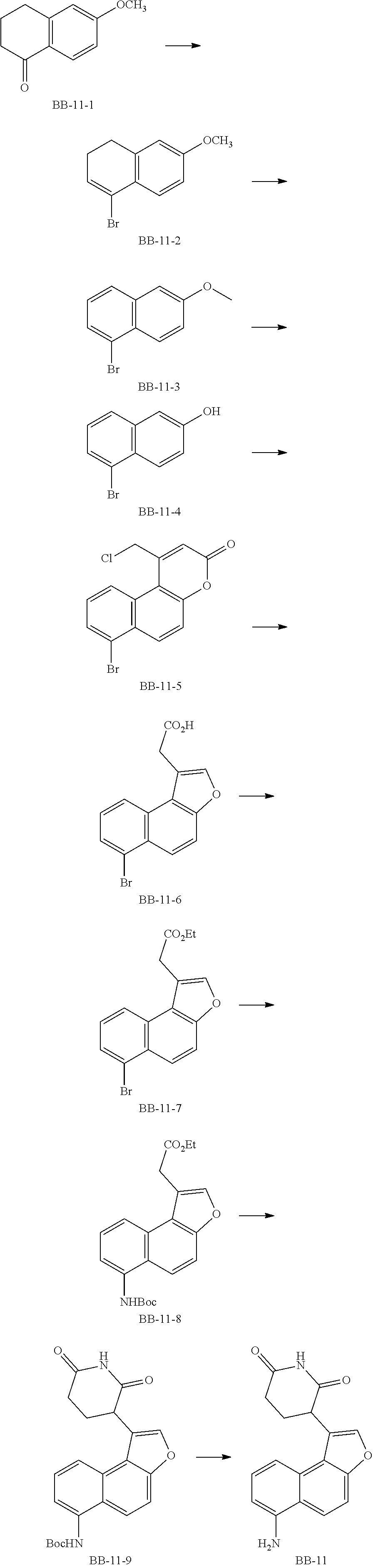

### Step 1: Synthesis of Compound BB-11-2

Triphenyl phosphite (149.14 g, 480.67 mmol) was dissolved in dichloromethane (1.3 L) at room temperature under nitrogen atmosphere. The mixture was cooled to −70° C., and liquid bromine (83.80 g, 524.37 mmol, 27.03 mL) was dropwise added thereto. After the dropwise addition was completed, a solution of triethylamine (57.48 g, 568.07 mmol, 79.07 mL) and a solution of compound BB-11-1 (77 g, 436.98 mmol) in dichloromethane (200 mL) were sequentially added dropwise to the mixture. After the dropwise addition was completed, the reaction mixture was slowly warmed to room temperature, and stirred and reacted for 15 hours. After the reaction was completed, the reaction mixture was slowly poured into a saturated aqueous solution of sodium sulfite (1.5 L), stirred for 10 minutes, and extracted with dichloromethane (1 L). The organic phases were washed with saturated brine (1 L), dried over anhydrous sodium sulfate, and filtered. The filtrate was concentrated under reduced pressure to remove the solvent. The resulting residue was separated by column chromatography (eluent: petroleum ether) to obtain compound BB-11-2.

### Step 2: Synthesis of Compound BB-11-3

Compound BB-11-2 (87 g, 363.85 mmol) was dissolved in toluene (1 L) at room temperature under nitrogen atmosphere. The mixture was cooled to 0° C., and 2,3-dichloro-5,6-dicyano-p-benzoquinone (90.86 g, 400.24 mmol) was slowly added thereto in batches. The reaction mixture was slowly warmed to room temperature, and stirred and reacted for 15 hours. After the reaction was completed, a saturated aqueous solution of sodium sulfite (2 L) was added dropwise. The mixture was stirred for 10 minutes, added with 1 N sodium hydroxide aqueous solution (1 L), and extracted with ethyl acetate (500 mL×3). The organic phases were combined, washed with saturated brine (1 L), dried over anhydrous sodium sulfate, and filtered. The filtrate was concentrated under reduced pressure to remove the solvent. The resulting residue was added with petroleum ether (500 mL), stirred for 10 minutes, and filtered. The filter cake was rinsed with petroleum ether (50 mL×2). The filtrate was collected, and concentrated under reduced pressure to remove the solvent. The resulting residue was separated by column chromatography (eluent: petroleum ether) to obtain compound BB-11-3. ¹H NMR (400 MHz, CDCl₃) δ: 8.12 (d, J=9.2 Hz, 1H), 7.68 (d, J=8.4 Hz, 1H), 7.60 (d, J=7.6 Hz, 1H), 7.26-7.20 (m, 2H), 7.11 (d, J=2.4 Hz, 1H), 3.92 (s, 3H).

### Step 3: Synthesis of Compound BB-11-4

Compound BB-11-3 (21.4 g, 90.26 mmol) was dissolved in dichloromethane (250 mL) at room temperature under nitrogen atmosphere. The mixture was cooled to 0° C., and boron tribromide (27.13 g, 108.31 mmol, 10.44 mL) was slowly added dropwise thereto. After the dropwise addition was completed, the reaction mixture was slowly warmed to room temperature, and stirred and reacted for 3 hours. After the reaction was completed, the reaction mixture was poured into ice water (500 mL) and extracted with dichloromethane (200 mL). The organic phase was washed with saturated brine (500 mL), dried over anhydrous sodium sulfate, and filtered. The filtrate was concentrated under reduced pressure to remove the solvent to obtain compound BB-11-4. ¹H NMR (400 MHz, CDCl₃) δ: 8.15 (d, J=8.8 Hz, 1H), 7.65-7.59 (m, 2H), 7.25 (t, J=7.8 Hz, 1H), 7.19 (dd, J=2.4 Hz, 9.2 Hz, 1H), 7.14 (d, J=2.4 Hz, 1H), 5.03 (s, 1H).

### Step 4: Synthesis of Compound BB-11-5

Compound BB-11-4 (20 g, 89.66 mmol) was dissolved in methanesulfonic acid (200 mL) at room temperature, and ethyl 4-chloroacetoacetate (22.14 g, 134.49 mmol) was added dropwise thereto. The reaction mixture was stirred and reacted at room temperature for 15 hours. After the reaction was completed, the reaction mixture was poured into ice water (1 L), stirred at room temperature for 10 minutes, and filtered. The filter cake was rinsed with water (200 mL×3), collected, and dried under vacuum to obtain compound BB-11-5. ¹H NMR (400 MHz, DMSO_d₆) δ: 8.58 (d, J=9.2 Hz, 1H), 8.49 (d, J=9.2 Hz, 1H), 8.02 (d, J=7.2 Hz, 1H), 7.74 (d, J=9.2 Hz, 1H), 7.65 (dd, J=7.6 Hz, 8.4 Hz, 1H), 6.93 (s, 1H), 5.41 (s, 2H).

### Step 5: Synthesis of Compound BB-11-6

Compound BB-11-5 (29 g, 89.63 mmol) was added to an aqueous solution of sodium hydroxide (2 M, 300 mL) at room temperature. The reaction mixture was heated to 80° C., and stirred and reacted for 3 hours. After the reaction was completed, the mixture was cooled to room temperature, added with water (200 mL), added with 6 N dilute hydrochloric acid to adjust the pH to 4, and extracted with ethyl acetate (300 mL×3). The organic phases were combined, washed with saturated brine (200 mL), dried over anhydrous sodium sulfate, and filtered. The filtrate was concentrated under reduced pressure to remove the solvent. The resulting residue was added with methyl tert-butyl ether (50 mL), stirred at room temperature for 10 minutes, and filtered. The filter cake was rinsed with methyl tert-butyl ether (10 mL×2), collected, and dried under vacuum to obtain compound BB-11-6. MS-ESI m/z: 305.0 [M+H]⁺, 306.9 [M+H+2]⁺. ¹H NMR (400 MHz, DMSO_d₆) δ: 12.68 (br s, 1H), 8.24 (d, J=8.4 Hz, 1H), 8.12 (t, J=4.6 Hz, 2H), 7.96 (d, J=9.2 Hz, 1H), 7.90 (d, J=7.6 Hz, 1H), 7.54 (t, J=7.8 Hz, 1H), 4.09 (s, 2H).

### Step 6: Synthesis of Compound BB-11-7

Compound BB-11-6 (18 g, 58.99 mmol) was dissolved in ethanol (180 mL) at room temperature, then concentrated sulfuric acid (5.31 g, 53.09 mmol, 2.89 mL, purity of 98%) was added thereto. The reaction mixture was heated to 80° C., and stirred and reacted for 15 hours. After the reaction was completed, the mixture was cooled to room temperature, and concentrated under reduced pressure to remove the solvent. The residue was added with ethyl acetate (300 mL) and a saturated aqueous solution of sodium bicarbonate (500 mL), and extracted. The phases were separated. The organic phase was washed with saturated brine (300 mL), dried over anhydrous sodium sulfate, and filtered. The filtrate was concentrated under reduced pressure to remove the solvent. The resulting residue was added with petroleum ether (50 mL), stirred at room temperature for 10 minutes, and filtered. The filter cake was rinsed with petroleum ether (20 mL×2), collected, and dried under vacuum to obtain compound BB-11-7. ¹H NMR (400 MHz, CDCl₃) δ: 8.22 (t, J=8.4 Hz, 2H), 7.84-7.78 (m, 2H), 7.75 (d, J=9.2 Hz, 1H), 7.41 (dd, J=7.6 Hz, 8.4 Hz, 1H), 4.23 (q, J=7.0 Hz, 2H), 4.06 (d, J=0.8 Hz, 2H), 1.26 (t, J=7.2 Hz, 3H).

### Step 7: Synthesis of Compound BB-11-8

Compound BB-11-7 (5 g, 15.01 mmol), tris(dibenzylideneacetone)dipalladium (961.96 mg, 1.05 mmol), 2-di-tert-butylphosphino-2′,4′,6′-triisopropyl biphenyl (892.16 mg, 2.10 mmol), potassium phosphate (12.74 g, 60.03 mmol), and tert-butyl carbamate (2.64 g, 22.51 mmol) were dissolved in a mixed solvent of toluene (50 mL) and water (10 mL) at room temperature under nitrogen atmosphere. The reaction mixture was heated to 100° C., and stirred and reacted for 15 hours. After the reaction was completed, the mixture was cooled to room temperature, and filtered. The filter cake was rinsed with ethyl acetate (30 mL×3). The filtrate was washed with saturated brine (100 mL), dried over anhydrous sodium sulfate, and filtered. The filtrate was concentrated under reduced pressure to remove the solvent. The resulting residue was added with methyl tert-butyl ether (50 mL), stirred at room temperature for 10 minutes, and filtered. The filter cake was rinsed with methyl tert-butyl ether (10 mL×2), collected, and dried under vacuum to obtain compound BB-11-8.

### Step 8: Synthesis of Compound BB-11-9

Compound BB-11-8 (4.2 g, 11.37 mmol) and acrylamide (888.93 mg, 12.51 mmol) were dissolved in N, N-dimethylformamide (40 mL) at room temperature under nitrogen atmosphere. The mixture was cooled to 0° C. Potassium tert-butoxide (2.55 g, 22.74 mmol) dissolved in N, N-dimethylformamide (10 mL) was added dropwise thereto. The reaction mixture was warmed to room temperature, and stirred and reacted for 2 hours. After the reaction was completed, the reaction mixture was poured into 0.2 N hydrochloric acid (200 mL), and extracted with ethyl acetate (100 mL×3). The organic phases were combined, washed with saturated brine (100 mL), dried over anhydrous sodium sulfate, and filtered. The filtrate was concentrated under reduced pressure to remove the solvent. The resulting residue was added with dichloromethane (20 mL), stirred at room temperature for 10 minutes, and filtered. The filter cake was rinsed with dichloromethane (10 mL), collected, and dried under vacuum to obtain compound BB-11-9. ¹H NMR (400 MHz, DMSO_d₆) δ: 10.94 (s, 1H), 9.28 (s, 1H), 8.05-7.92 (m, 3H), 7.79 (d, J=9.6 Hz, 1H), 7.58-7.47 (m, 2H), 4.68 (dd, J=4.4 Hz, 12.0 Hz, 1H), 2.95-2.81 (m, 1H), 2.70-2.56 (m, 1H), 2.47-2.34 (m, 1H), 2.33-2.22 (m, 1H), 1.49 (s, 9H).

Step 9: Synthesis of hydrochloride of compound BB-11

Compound BB-11-9 (1.3 g, 3.30 mmol) was dissolved in ethyl acetate (5 mL) at room temperature, then a solution of hydrochloric acid in ethyl acetate (4 M, 50 mL) was added thereto. The reaction mixture was stirred and reacted at room temperature for 3 hours. After the reaction was completed, the reaction mixture was concentrated under reduced pressure to remove the solvent to obtain the hydrochloride of compound BB-11.

### Reference Example 12: Fragment BB-12

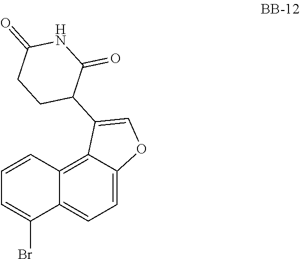 [SMILES: O=C1CCC(c2coc3ccc4c(Br)cccc4c23)C(=O)N1]

### Synthetic Route:

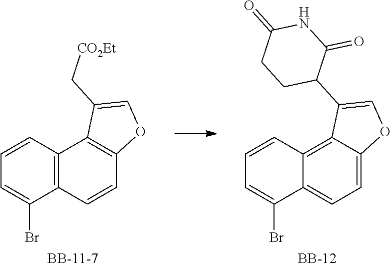

### Synthesis of Compound BB-12

Acrylamide (234.67 mg, 3.30 mmol) and compound BB-11-7 (1 g, 3.00 mmol) were dissolved in N, N-dimethylformamide (15 mL) at room temperature under nitrogen atmosphere, then potassium tert-butoxide (370.47 mg, 3.30 mmol) was added thereto. The reaction mixture was stirred and reacted at room temperature for 0.5 hours. After the reaction was completed, the reaction mixture was poured into 0.1 N hydrochloric acid (10 mL) and extracted with dichloromethane (5 mL×2). The organic phases were combined, washed with saturated brine (5 mL), dried over anhydrous sodium sulfate, and filtered. The filtrate was concentrated under reduced pressure to remove the solvent. The resulting residue was added with dichloromethane (5 mL), stirred at room temperature for 1 hour, and filtered. The filter cake was rinsed with dichloromethane (1 mL), collected, and dried under vacuum to obtain compound BB-12. MS-ESI m/z: 358.1 [M+H]⁺, 360.1 [M+H+2]⁺.

### Reference Example 14: Fragment BB-14

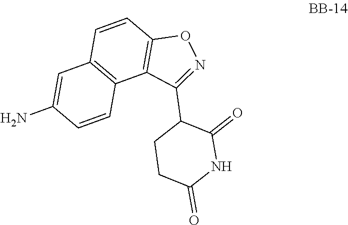 [SMILES: Nc1ccc2c(ccc3onc(C4CCC(=O)NC4=O)c32)c1]

### Synthetic Route:

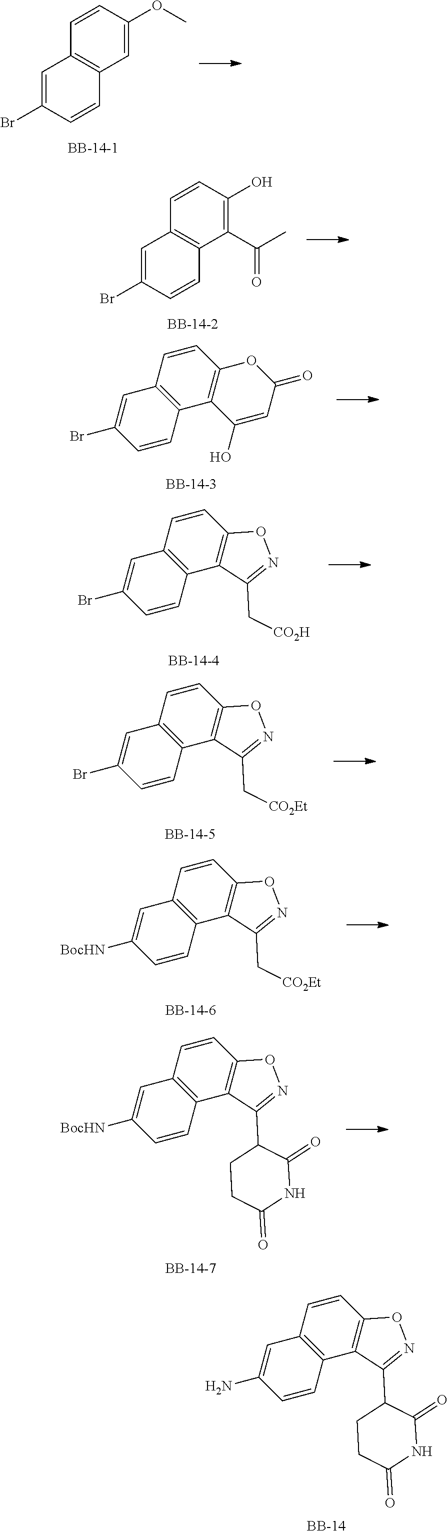

### Step 1: Synthesis of Compound BB-14-2

Compound BB-14-1 (150 g, 632.67 mmol) was dissolved in dichloromethane (3 L) at room temperature under nitrogen atmosphere, then acetyl chloride (49.66 g, 632.67 mmol, 45.15 mL) was added thereto. The reaction mixture was cooled to 5 to 15° C., and aluminum trichloride (177.16 g, 1.33 mol) was then added thereto in batches. The reaction mixture was warmed to room temperature, and stirred and reacted for 4 hours. Additional aluminum trichloride (29.53 g, 221.43 mmol) was added, and the reaction mixture was further stirred and reacted at room temperature for 12 hours. After the reaction was completed, the reaction mixture was slowly poured into ice water (3 L) and extracted. The phases were separated. The aqueous phase was again extracted with dichloromethane (2 L×2). The organic phases were combined, washed with saturated brine (6 L×2), dried over anhydrous sodium sulfate, and filtered. The filtrate was concentrated under reduced pressure to remove the solvent to obtain compound BB-14-2. MS-ESI m/z: 265.1 [M+H]⁺, 267.1 [M+H+2]⁺.

### Step 2: Synthesis of Compound BB-14-3

Compound BB-14-2 (200 g, 754.43 mmol) and dimethyl carbonate (271.83 g, 3.02 mol, 254.04 mL) was dissolved in tetrahydrofuran (2 L) at room temperature under nitrogen atmosphere. The mixture was cooled to 0° C., and potassium tert-butoxide (507.93 g, 4.53 mol) was slowly added thereto. The reaction mixture was heated to 70° C., and stirred and reacted for 12 hours. After the reaction was completed, the mixture was cooled to room temperature, concentrated under reduced pressure to remove the solvent. The resulting residue was added with ice water (4 L), and added with 6 N hydrochloric acid to adjust the pH to 2 to 3, resulting in the precipitation of a large amount of solid. The mixture was filtered. The filter cake was sequentially rinsed with water (1 L) and methyl tert-butyl ether (1 L), collected, and dried under vacuum to obtain compound BB-14-3. MS-ESI m/z: 291.0 [M+H]⁺, 293.0 [M+H+2]⁺. ¹H NMR (400 MHz, DMSO_d₆) δ: 9.18 (d, J=9.2 Hz, 1H), 8.30 (d, J=1.2 Hz, 1H), 8.16 (d, J=8.8 Hz, 1H), 7.79 (d, J=7.6 Hz, 1H), 7.56 (d, J=8.8 Hz, 1H), 5.83 (s, 1H).

### Step 3: Synthesis of Compound BB-14-4

Compound BB-14-3 (200 g, 687.06 mmol), sodium acetate (309.99 g, 3.78 mol), and hydroxylamine hydrochloride (262.59 g, 3.78 mol) were dissolved in ethanol (2 L) at room temperature. The reaction mixture was heated to 80° C., and stirred and reacted for 12 hours. After the reaction was completed. The mixture was cooled to room temperature, poured into water (2 L), and concentrated under reduced pressure to remove the solvent. The mixture was added with 2 N hydrochloric acid to adjust the pH to 2 to 3, and extracted with ethyl acetate/tetrahydrofuran=1/1 (2 L×3, v/v). The organic phases were combined, dried over anhydrous sodium sulfate, and filtered. The filtrate was concentrated under reduced pressure to remove the solvent. The resulting residue was added with methyl tert-butyl ether (600 mL), stirred at room temperature for 1 hour, and filtered. The filter cake was collected, and dried under vacuum to obtain compound BB-14-4. MS-ESI m/z: 306.0 [M+H]⁺, 308.0 [M+H+2]⁺.

### Step 4: Synthesis of Compound BB-14-5

Concentrated sulfuric acid (33.38 g, 333.49 mmol, 18.14 mL, purity of 98%) was dissolved in ethanol (1.3 L) at room temperature, then compound BB-14-4 (135 g, 373.32 mmol, purity of 84.65%) was added thereto. The reaction mixture was heated to 75° C., and stirred and reacted for 16 hours. After the reaction was completed, the mixture was cooled to room temperature, and concentrated under reduced pressure to remove the solvent. The resulting residue was added with ethanol (600 mL), stirred at room temperature for 5 minutes, and filtered. The filter cake was rinsed with ethanol (100 mL×2), collected, and dried under vacuum to obtain compound BB-14-5. MS-ESI m/z: 334.1 [M+H]⁺, 336.0 [M+H+2]⁺. ¹H NMR (400 MHz, DMSO_d₆) δ: 8.47 (d, J=1.6 Hz, 1H), 8.19 (d, J=9.2 Hz, 1H), 8.08 (d, J=8.8 Hz, 1H), 8.00 (d, J=9.2 Hz, 1H), 7.89 (dd, J=2.0 Hz, 8.8 Hz, 1H), 4.51 (s, 2H), 4.14 (q, J=7.2 Hz, 2H), 1.16 (t, J=7.2 Hz, 3H).

### Step 5: Synthesis of Compound BB-14-6

Compound BB-14-5 (15 g, 44.89 mmol), tris(dibenzylideneacetone)dipalladium (1.44 g, 1.57 mmol), 2-di-tert-butylphosphino-2′,4′,6′-triisopropyl biphenyl (1.33 g, 3.14 mmol), potassium phosphate (38.11 g, 179.55 mmol), and tert-butyl carbamate (7.89 g, 67.33 mmol) were sequentially added to a mixed solvent of toluene (150 mL) and water (30 mL) at room temperature under nitrogen atmosphere. The reaction mixture was heated to 100° C., and stirred and reacted for 12 hours. After the reaction was completed, the mixture was cooled to room temperature, added with water (100 mL) and ethyl acetate (150 mL), and extracted. The phases were separated. The aqueous phase was again extracted with ethyl acetate (200 mL×3). The organic phases were combined, washed with saturated brine (200 mL×3), dried over anhydrous sodium sulfate, and filtered. The filtrate was concentrated under reduced pressure to remove the solvent. The resulting residue was separated by column chromatography (eluent: petroleum ether/ethyl acetate=20/1 to 1/1, v/v) to obtain compound BB-14-6. MS-ESI m/z: 371.2 [M+H]⁺. ¹H NMR (400 MHz, CDCl₃) δ: 8.20 (br s, 1H), 8.03 (d, J=8.8 Hz, 1H), 7.90 (d, J=9.2 Hz, 1H), 7.67 (d, J=9.2 Hz, 1H), 7.53 (dd, J=2.0 Hz, 8.8 Hz, 1H), 6.75 (s, 1H), 4.31 (s, 2H), 4.21 (q, J=7.2 Hz, 2H), 1.57 (s, 9H), 1.20 (t, J=7.2 Hz, 3H).

### Step 6: Synthesis of Compound BB-14-7

Compound BB-14-6 (7.3 g, 19.71 mmol) and acrylamide (1.54 g, 21.68 mmol) was dissolved in tetrahydrofuran (70 mL) at room temperature under nitrogen atmosphere, The mixture was cooled to 0° C., and potassium tert-butoxide (2.43 g, 21.68 mmol) was added thereto. The reaction mixture was warmed to room temperature, and stirred and reacted for 1 hour. After the reaction was completed, the reaction mixture was poured into 0.1 N hydrochloric acid (30 mL), and extracted with ethyl acetate (80 mL×3). The organic phases were combined, washed with saturated brine (50 mL×2), dried over anhydrous sodium sulfate, and filtered. The filtrate was concentrated under reduced pressure to remove the solvent. The resulting residue was added with dichloromethane (5 mL), stirred at room temperature for 5 minutes, and filtered. The filter cake was rinsed with dichloromethane (3 mL), collected, and dried under vacuum to obtain compound BB-14-7. MS-ESI m/z: 396.2 [M+H]⁺. ¹H NMR (400 MHz, DMSO_d₆) δ:11.12 (s, 1H), 9.66 (s, 1H), 8.36 (s, 1H), 8.13 (d, J=8.8 Hz, 1H), 8.07 (d, J=8.8 Hz, 1H), 7.85 (d, J=9.2 Hz, 1H), 7.68 (dd, J=2.0 Hz, 8.8 Hz, 1H), 5.00 (dd, J=4.8 Hz, 11.6 Hz, 1H), 2.89-2.76 (m, 1H), 2.68-2.60 (m, 1H), 2.59-2.53 (m, 1H), 2.42-2.30 (m, 1H), 1.52 (s, 9H).

Step 7: Synthesis of hydrochloride of compound BB-14

Compound BB-14-7 (1 g, 2.53 mmol) was dissolved in ethyl acetate (5 mL) at room temperature, then a solution of hydrochloric acid in ethyl acetate (4 M, 20 mL) was added thereto. The reaction mixture was stirred and reacted at room temperature for 1 hour. After the reaction was completed, the reaction mixture was filtered. The filter cake was rinsed with ethyl acetate (3 mL×3), collected, and dried under vacuum to obtain the hydrochloride of compound BB-14. MS-ESI m/z: 295.9 [M+H]⁺.

### Reference Example 15: Fragment BB-15

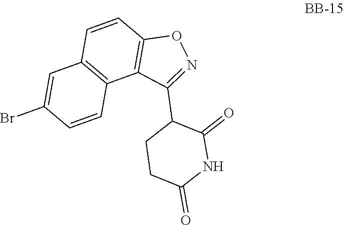 [SMILES: O=C1CCC(c2noc3ccc4cc(Br)ccc4c23)C(=O)N1]

### Synthetic Route:

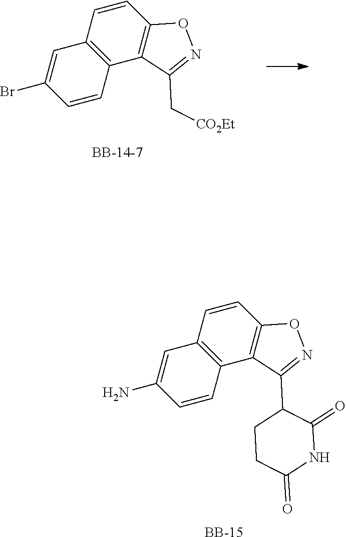 [SMILES: CCOC(=O)Cc1noc2ccc3cc(Br)ccc3c12 ; Nc1ccc2c(ccc3onc(C4CCC(=O)NC4=O)c32)c1 ; "BB-15"]

### Synthesis of Compound BB-15

Compound BB-14-5 (3 g, 8.98 mmol) and acrylamide (701.92 mg, 9.88 mmol) were dissolved in tetrahydrofuran (70 mL) at room temperature under nitrogen atmosphere. The mixture was cooled to 0° C., and potassium tert-butoxide (1.21 g, 10.77 mmol) was added thereto in batches. The reaction mixture was warmed to room temperature, and stirred and reacted for 1 hour. After the reaction was completed, the reaction mixture was poured into 1 N hydrochloric acid (20 mL), and extracted with ethyl acetate (40 mL×3). The organic phases were combined, washed with saturated brine (50 mL), dried over anhydrous sodium sulfate, and filtered. The filtrate was concentrated under reduced pressure to remove the solvent. The resulting residue was added with methyl tert-butyl ether (20 mL), stirred at room temperature for 10 minutes, and filtered. The filter cake was rinsed with methyl tert-butyl ether (5 mL×2), collected, and dried under vacuum to obtain compound BB-15. MS-ESI m/z: 359.0 [M+H]⁺, 361.0 [M+H+2]⁺. ¹H NMR (400 MHz, DMSO_d₆) δ: 11.14 (s, 1H), 8.47 (d, J=2.0 Hz, 1H), 8.24-8.17 (m, 2H), 8.01 (d, J=9.2 Hz, 1H), 7.85 (dd, J=2.0 Hz, 8.8 Hz, 1H), 5.06 (dd, J=4.8 Hz, 11.6 Hz, 1H), 2.89-2.78 (m, 1H), 2.70-2.56 (m, 2H), 2.44-2.34 (m, 1H).

### Reference Example 16: Fragment BB-16

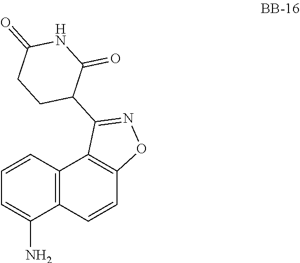 [SMILES: Nc1cccc2c1ccc1onc(C3CCC(=O)NC3=O)c12]

### Synthetic Route:

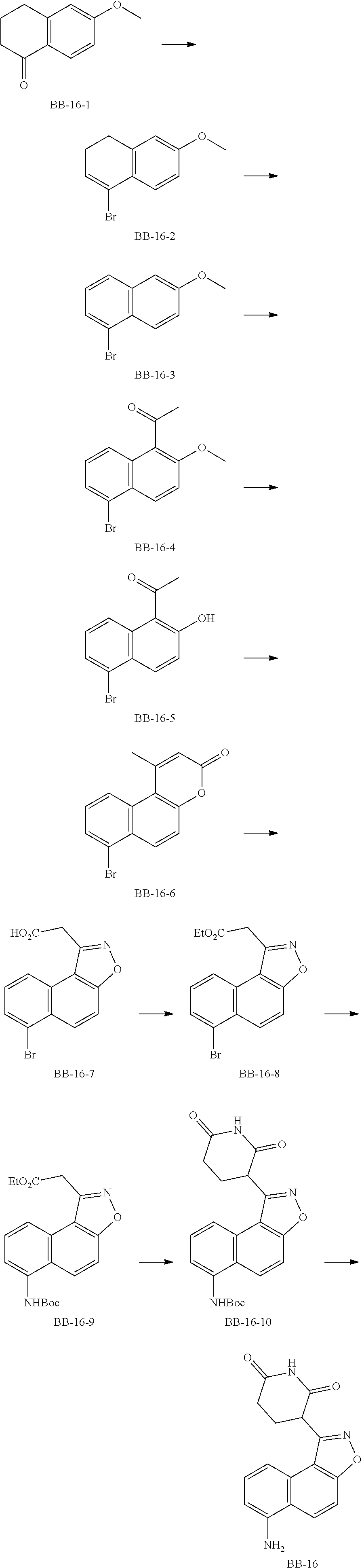

### Step 1: Synthesis of Compound BB-16-2

Triphenyl phosphite (193.69 g, 624.25 mmol) was dissolved in dichloromethane (1 L) at room temperature under nitrogen atmosphere. The mixture was cooled to −70° C., and liquid bromine (108.83 g, 681.00 mmol, 35.11 mL) was added dropwise thereto. After the dropwise addition was completed, triethylamine (74.65 g, 737.75 mmol, 102.69 mL) and compound BB-16-1 (100, 567.50 mmol) dissolved in dichloromethane (500 mL) were sequentially added dropwise thereto. After the dropwise addition was completed, the reaction mixture was slowly warmed to room temperature, and stirred and reacted for 15 hours. After the reaction was completed, the reaction mixture was poured into a saturated aqueous solution of sodium sulfite (700 mL), stirred for 10 minutes, and extracted with dichloromethane (800 mL). The organic phases were washed with saturated brine (800 mL), dried over anhydrous sodium sulfate, and filtered. The filtrate was concentrated under reduced pressure to remove the solvent. The resulting residue was separated by column chromatography (eluent: petroleum ether/ethyl acetate=20/1 to 7/1, v/v) to obtain compound BB-16-2. ¹H NMR (400 MHz, DMSO_d₆) δ: 7.33 (d, J=8.0 Hz, 1H), 6.84-6.75 (m, 2H), 6.33 (t, J=4.8 Hz, 1H), 3.75 (s, 3H), 2.76 (t, J=8.0 Hz, 2H), 2.32-2.25 (m, 2H).

### Step 2: Synthesis of Compound BB-16-3

Compound BB-16-2 (39.5 g, 165.20 mmol) was dissolved in toluene (500 mL) at room temperature under nitrogen atmosphere. The mixture was cooled to 0° C., and 2,3-dichloro-5,6-dicyano-p-benzoquinone (41.25 g, 181.72 mmol) was slowly added thereto in batches. The reaction mixture was slowly warmed to room temperature, and stirred and reacted for 12 hours. After the reaction was completed, the mixture was cooled to 0 to 10° C. A saturated aqueous solution of sodium sulfite (1 L) and an aqueous solution of 1 N sodium hydroxide (1 L) were added dropwise thereto. The mixture was filtered. The filter cake was rinsed with ethyl acetate (300 mL×3), and discarded. The filtrate was extracted with ethyl acetate (500 mL×3). The organic phases were combined, washed with saturated brine (500 mL×2), dried over anhydrous sodium sulfate, and filtered. The filtrate was concentrated under reduced pressure to remove the solvent. The resulting residue was separated by column chromatography (eluent: petroleum ether) to obtain compound BB-16-3. MS-ESI m/z: 237.1 [M+H]⁺, 239.1 [M+H+2]⁺. ¹H NMR (400 MHz, CDCl₃) δ: 8.05 (d, J=9.2 Hz, 1H), 7.60 (d, J=8.0 Hz, 1H), 7.53 (d, J=7.2 Hz, 1H), 7.22-7.13 (m, 2H), 7.04 (s, 1H), 3.85 (s, 3H).

### Step 3: Synthesis of Compound BB-16-4

Acetic anhydride (21.53 g, 210.89 mmol, 19.75 mL) was dissolved in dichloromethane (400 mL) at room temperature under nitrogen atmosphere. The mixture was cooled to −60° C., and a solution of boron trifluoride diethyl etherate (63.68 g, 210.89 mmol, 55.38 mL, purity of 47%) was added dropwise thereto. The mixture was stirred at −60° C. for 10 minutes. Compound BB-16-3 (25, 105.44 mmol) dissolved in dichloromethane (250 mL) was added dropwise thereto. The reaction mixture was slowly warmed to room temperature, and stirred and reacted for 12 hours. After the reaction was completed, the reaction mixture was added with ice water (200 mL), and extracted with dichloromethane (100 mL×3). The organic phases were combined, washed with saturated brine (100 mL), dried over anhydrous sodium sulfate, and filtered. The filtrate was concentrated under reduced pressure to remove the solvent. The resulting residue was separated by column chromatography (eluent: petroleum ether/ethyl acetate=100/1 to 20/1, v/v) to obtain compound BB-16-4. MS-ESI m/z: 279.0 [M+H]⁺, 281.0 [M+H+2]⁺. ¹H NMR (400 MHz, CDCl₃) δ: 8.34 (d, J=9.2 Hz, 1H), 7.72 (d, J=8.4 Hz, 1H), 7.67 (dd, J=0.8 Hz, 7.2 Hz, 1H), 7.38 (d, J=9.2 Hz, 1H), 7.31 (dd, J=7.6 Hz, 8.8 Hz, 1H), 4.01 (s, 3H), 2.65 (s, 3H).

### Step 4: Synthesis of Compound BB-16-5

Compound BB-16-4 (18.8 g, 67.35 mmol) was dissolved in dichloromethane (200 mL) at room temperature under nitrogen atmosphere. The mixture was cooled to 0° C., and boron tribromide (20.25 g, 80.82 mmol, 7.79 mL) was added dropwise thereto. The mixture was stirred and reacted at 0° C. for 1 hour. After the reaction was completed, the reaction mixture was poured into ice water (300 mL), and extracted with dichloromethane (100 mL×3). The organic phases were combined, washed with saturated brine (100 mL×3), dried over anhydrous sodium sulfate, and filtered. The filtrate was concentrated under reduced pressure to remove the solvent to obtain compound BB-16-5. ¹H NMR (400 MHz, CDCl₃) δ: 13.05 (s, 1H), 8.41 (d, J=9.2 Hz, 1H), 8.04 (d, J=8.8 Hz, 1H), 7.70 (dd, J=0.8 Hz, 7.6 Hz, 1H), 7.40 (dd, J=7.6 Hz, 8.4 Hz, 1H), 7.25 (d, J=9.6 Hz, 1H), 2.85 (s, 3H).

### Step 5: Synthesis of Compound BB-16-6

Compound BB-16-5 (13 g, 49.04 mmol) and dimethyl carbonate (17.67 g, 196.15 mmol, 16.51 mL) were dissolved in tetrahydrofuran (130 mL) at room temperature under nitrogen atmosphere. The mixture was cooled to 0 to 10° C., and potassium tert-butoxide (33.02 g, 294.23 mmol) was added thereto in batches. The reaction mixture was heated to 70° C., and stirred and reacted for 12 hours. After the reaction was completed, the mixture was cooled to room temperature, and concentrated under reduced pressure to remove the solvent. The residue was added with ice water (200 mL), and extracted with methyl tert-butyl ether (70 mL×2). The pH of the aqueous phase was adjusted to 2 with 6 N hydrochloric acid, resulting in the precipitation of a large amount of solid. The mixture was filtered. The filter cake was washed with water (30 mL×2), collected, and dried under vacuum to obtain compound BB-16-6. MS-ESI m/z: 291.0 [M+H]⁺, 293.0 [M+H+2]⁺. ¹H NMR (400 MHz, DMSO_d₆) δ: 13.28 (br s, 1H), 9.38 (t, J=7.2 Hz, 1H), 8.43 (dd, J=6.8 Hz, 9.2 Hz, 1H), 7.96 (t, J=7.2 Hz, 1H), 7.71-7.65 (m, 1H), 7.63-7.56 (m, 1H), 5.92-5.87 (m, 1H).

### Step 6: Synthesis of Compound BB-16-7

Compound BB-16-6 (22 g, 75.58 mmol), sodium acetate (43.40 g, 529.03 mmol), and hydroxylamine hydrochloride (36.76 g, 529.03 mmol) were dissolved in ethanol (400 mL) at room temperature. The reaction mixture was heated to 80° C., and stirred and reacted for 12 hours. After the reaction was completed, the mixture was cooled to room temperature, and concentrated under reduced pressure to remove the solvent. The resulting residue was added with water (100 mL) and methyl tert-butyl ether (50 mL), stirred at room temperature for 0.5 hours, and filtered. The filter cake was rinsed with methyl tert-butyl ether (20 mL×3), collected, and dried under vacuum to obtain compound BB-16-7. MS-ESI m/z: 306.0 [M+H]⁺, 308.0 [M+H+2]⁺. ¹H NMR (400 MHz, DMSO_d₆) δ: 8.40 (d, J=9.2 Hz, 1H), 8.27 (d, J=8.4 Hz, 1H), 8.05 (d, J=9.2 Hz, 1H), 7.96 (d, J=7.6 Hz, 1H), 7.62 (t, J=8.0 Hz, 1H), 4.25 (s, 2H).

### Step 7: Synthesis of Compound BB-16-8

Compound BB-16-7 (15 g, 49.00 mmol) was dissolved in ethanol (300 mL) at room temperature, then concentrated sulfuric acid (1.29 g, 12.87 mmol, 0.7 mL, purity of 98%) was added thereto. The reaction mixture was heated to 80° C., and stirred and reacted for 12 hours. The mixture was cooled to room temperature. Additional concentrated sulfuric acid (4 mL, purity of 98%) was added thereto. The reaction mixture was heated to 80° C., and stirred and reacted for 3 hours. After the reaction was completed, the mixture was cooled to room temperature, and concentrated under reduced pressure to remove the solvent. The resulting residue was added with a saturated solution of sodium bicarbonate at 0 to 10° C. (100 mL), and extracted with ethyl acetate (70 mL×3). The organic phases were combined, washed with saturated brine (70 mL×2), dried over anhydrous sodium sulfate, and filtered. The filtrate was concentrated under reduced pressure to remove the solvent to obtain compound BB-16-8. MS-ESI m/z: 334.0 [M+H]⁺, 336.0 [M+H+2]⁺. ¹H NMR (400 MHz, CDCl₃) δ: 8.50 (d, J=9.6 Hz, 1H), 8.10 (d, J=8.4 Hz, 1H), 7.88 (dd, J=0.8 Hz, 7.6 Hz, 1H), 7.81 (d, J=9.2 Hz, 1H), 7.52 (t, J=8.0 Hz, 1H), 4.34 (s, 2H), 4.22 (q, J=7.2 Hz, 2H), 1.21 (t, J=7.2 Hz, 3H).

### Step 8: Synthesis of Compound BB-16-9

Compound BB-16-8 (9.5 g, 28.43 mmol), tris(dibenzylideneacetone)dipalladium (1.30 g, 1.42 mmol), 2-di-tert-butylphosphino-2′,4′,6′-triisopropyl biphenyl (1.21 g, 2.84 mmol), potassium phosphate (24.14 g, 113.72 mmol), and tert-butyl carbamate (3.66 g, 31.27 mmol) were sequentially added to a mixed solvent of toluene (90 mL) and water (30 mL) at room temperature under nitrogen atmosphere. The reaction mixture was heated to 100° C., and stirred and reacted for 12 hours. After the reaction was completed, the mixture was cooled to room temperature, added with water (200 mL), and extracted with ethyl acetate (150 mL×3). The organic phases were combined, washed with saturated brine (200 mL×2), dried over anhydrous sodium sulfate, and filtered. The filtrate was concentrated under reduced pressure to remove the solvent. The resulting residue was separated by column chromatography (eluent: petroleum ether/ethyl acetate=6/1 to 2/1, v/v) to obtain compound BB-16-9. MS-ESI m/z: 371.2 [M+H]⁺. ¹H NMR (400 MHz, DMSO_d₆) δ: 8.48 (s, 1H), 8.28 (d, J=9.2 Hz, 1H), 7.95-7.87 (m, 2H), 7.75-7.60 (m, 2H), 4.50 (s, 2H), 4.14 (q, J=7.2 Hz, 2H), 1.50 (s, 9H), 1.16 (t, J=7.2 Hz, 3H).

### Step 9: Synthesis of Compound BB-16-10

Compound BB-16-9 (3.0 g, 8.10 mmol) was dissolved in tetrahydrofuran (25 mL) at room temperature under nitrogen atmosphere, then acrylamide (690.82 mg, 9.72 mmol) and potassium tert-butoxide (1.36 g, 12.15 mmol) were added thereto. The reaction mixture was stirred and reacted at room temperature for 1 hour. After the reaction was completed, the reaction mixture was poured into 1 N hydrochloric acid (30 mL), and extracted with ethyl acetate (45 mL×3). The organic phases were combined, washed with saturated brine (30 mL×2), dried over anhydrous sodium sulfate, and filtered. The filtrate was concentrated under reduced pressure to remove the solvent. The resulting residue was separated by column chromatography (eluent: petroleum ether/ethyl acetate=5/1 to 1/1, v/v) to obtain compound BB-16-10. MS-ESI m/z: 396.2 [M+H]⁺. ¹H NMR (400 MHz, DMSO_d₆) δ: 11.14 (s, 1H), 9.47 (s, 1H), 8.28 (d, J=9.6 Hz, 1H), 8.04 (br d, J=6.8 Hz, 1H), 7.93 (d, J=9.2 Hz, 1H), 7.75-7.59 (m, 2H), 5.07 (dd, J=4.6 Hz, 11.4 Hz, 1H), 2.93-2.77 (m, 1H), 2.71-2.56 (m, 2H), 2.43-2.31 (m, 1H), 1.50 (s, 9H).

Step 10: Synthesis of hydrochloride of compound BB-16

Compound BB-16-10 (1.3 g, 3.29 mmol) was dissolved in a solution of hydrochloric acid in ethyl acetate (4 M, 20 mL) at room temperature. The reaction mixture was stirred and reacted at room temperature for 12 hours. After the reaction was completed, the mixture was filtered. The filter cake was rinsed with ethyl acetate (30 mL), collected, and dried under vacuum to obtain the hydrochloride of compound BB-16. MS-ESI m/z: 296.1 [M+H]⁺. ¹H NMR (400 MHz, DMSO_d₆) δ: 11.14 (s, 1H), 8.34 (d, J=9.6 Hz, 1H), 7.96 (d, J=9.2 Hz, 1H), 7.85 (t, J=8.0 Hz, 1H), 7.61 (t, J=7.8 Hz, 1H), 7.33 (d, J=7.6 Hz, 1H), 5.03 (dd, J=4.6 Hz, 11.4 Hz, 1H), 2.90-2.76 (m, 1H), 2.70-2.55 (m, 2H), 2.42-2.30 (m, 1H).

### Reference Example 17: Fragment BB-17

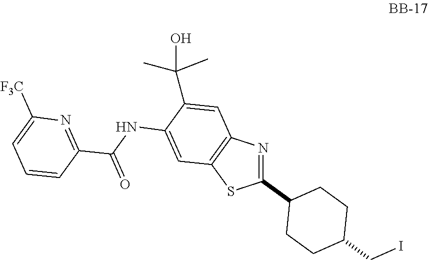

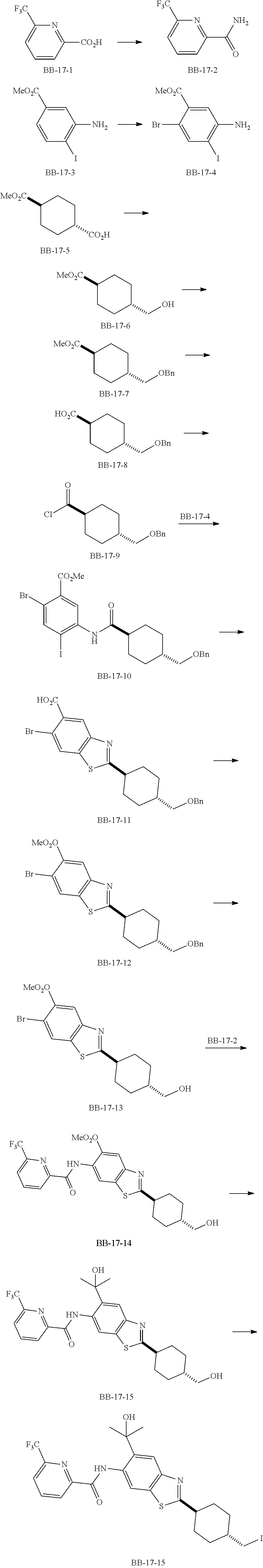

### Step 1: Synthesis of Compound BB-17-2

N, N-dimethylformamide (422.74 μL) was added to a solution of compound BB-17-1 (21 g 109.89 mmol) in dichloromethane (300 mL) at room temperature under nitrogen atmosphere. The reaction mixture was cooled to 0° C., and oxalyl chloride (27.89 g, 219.77 mmol, 19.24 mL) was added dropwise thereto. After the dropwise addition was completed, the mixture was slowly warmed to room temperature, and stirred and reacted for 1 hour. After the reaction was completed, the reaction mixture was directly concentrated under reduced pressure to obtain a residue, which was used in the next step directly. The crude product was dissolved in tetrahydrofuran (100 mL). The mixture was cooled to 0° C., ammonia water (169.08 mL, purity of 25%) was added thereto. The reaction mixture was stirred and reacted at room temperature for 1 hour. After the reaction was completed, the reaction mixture was concentrated under reduced pressure to remove tetrahydrofuran. The resulting residue was filtered. The filter cake was collected, and dried under vacuum to obtain compound BB-17-2. MS-ESI m/z: 191.0 [M+H]⁺. ¹H NMR (400 MHz, DMSO_d₆) δ: 8.33-8.25 (m, 2H), 8.09 (dd, J=2.4, 6.4 Hz, 1H), 8.04 (s, 1H), 7.87 (s, 1H).

### Step 2: Synthesis of Compound BB-17-4

Compound BB-17-3 (25 g, 90.23 mmol) was dissolved in N, N-dimethylformamide (125 mL) at room temperature under nitrogen atmosphere. The reaction system was cooled to 0° C., then N-bromosuccinimide (17.67 g, 99.26 mmol) was added thereto in batches. The reaction mixture was stirred and reacted at 0° C. for 2 hours. After the reaction was completed, the reaction mixture was poured into water (200 L), resulting in the precipitation of solid. The mixture was filtered. The filter cake was rinsed with water (100 mL×3), collected, and dried under vacuum to obtain compound BB-17-4. MS-ESI m/z: 355.8 [M+H]⁺, 357.8 [M+H+2]⁺. ¹H NMR (400 MHz, DMSO_d₆) δ: 7.84 (s, 1H), 7.13 (s, 1H), 5.66 (s, 2H), 3.81 (s, 3H).

### Step 3: Synthesis of Compound BB-17-6

Compound BB-17-5 (10 g, 53.70 mmol) was dissolved in dichloromethane (50 mL) at room temperature under nitrogen atmosphere. The mixture was cooled to −10° C., and a solution of borane in tetrahydrofuran (1 M, 59.07 mL) was added dropwise thereto. The reaction mixture was stirred and reacted at 0° C. for 1 hour. After the reaction was completed, the mixture was added with methanol (10 mL) and stirred at room temperature for 30 minutes, then 2 M hydrochloric acid (200 mL) was added thereto. The mixture was stirred at room temperature for 30 minutes, extracted with ethyl acetate (200 mL×2). The organic phases were combined, washed with saturated brine (100 mL×2), dried over anhydrous sodium sulfate, and filtered. The filtrate was concentrated under reduced pressure to obtain a residue. The resulting residue was separated by column chromatography (eluent: petroleum ether/ethyl acetate=1/0 to 7/3, v/v) to obtain compound BB-17-6. ¹H NMR (400 MHz, CDCl₃) δ: 3.67 (s, 3H), 3.47 (d, J=6.4 Hz, 2H), 2.32-2.20 (m, 1H), 2.05-1.98 (m, 2H), 1.92-1.83 (m, 2H), 1.56-1.38 (m, 3H), 1.07-0.93 (m, 2H).

### Step 4: Synthesis of Compound BB-17-7

Compound BB-17-6 (8.2 g, 47.61 mmol) was dissolved in tetrahydrofuran (80 mL) at room temperature under nitrogen atmosphere, then potassium hydroxide (4.01 g, 71.42 mmol), benzyl bromide (10.59 g, 61.90 mmol, 7.35 mL), tetrabutylamine iodide (3.52 g, 9.52 mmol), and potassium iodide (1.58 g, 9.52 mmol) were added thereto. The reaction mixture was stirred and reacted at room temperature for 12 hours. After the reaction was completed, the mixture was filtered. The filter cake was rinsed with tetrahydrofuran (50 mL×3). The filtrate was collected and concentrated under reduced pressure to obtain a residue. The resulting residue was separated by column chromatography (eluent: petroleum ether/ethyl acetate=1/0 to 19/1, v/v) to obtain compound BB-17-7. ¹H NMR (400 MHz, CDCl₃) δ: 7.39-7.27 (m, 5H), 4.50 (s, 2H), 3.29 (d, J=6.0 Hz, 2H), 2.32-2.19 (m, 1H), 2.08-1.97 (m, 2H), 1.96-1.86 (m, 2H), 1.72-1.58 (m, 1H), 1.53-1.38 (m, 2H), 1.09-0.95 (m, 2H).

### Step 5: Synthesis of Compound BB-17-8

Compound BB-17-7 (5.9, 22.49 mmol) was dissolved in tetrahydrofuran (50 mL) and methanol (10 mL) at room temperature, then an aqueous solution (10 mL) of lithium hydroxide monohydrate (4.72 g, 112.45 mmol) was added thereto. The reaction mixture was stirred and reacted at room temperature for 12 hours. After the reaction was completed, the reaction mixture was concentrated under reduced pressure to remove the solvent, added with water (200 mL), and extracted with petroleum ether (200 mL). The organic phase was discarded. The aqueous phase was collected, added with 6 N hydrochloric acid to adjust the pH 5 to 6, and extracted with dichloromethane (100 mL×3). The organic phases were combined, washed with saturated brine (100 mL), dried over anhydrous sodium sulfate, and filtered. The filtrate was concentrated under reduced pressure to obtain compound BB-17-8. MS-ESI m/z: 266.2 [M+18]⁺. ¹H NMR (400 MHz, CDCl₃) δ: 11.27 (s, 1H), 7.40-7.27 (m, 5H), 4.51 (s, 2H), 3.30 (d, J=6.4 Hz, 2H), 2.35-2.23 (m, 1H), 2.11-2.00 (m, 2H), 1.98-1.87 (m, 2H), 1.75-1.56 (m, 1H), 1.53-1.39 (m, 2H), 1.11-0.91 (m, 2H).

### Step 6: Synthesis of Compound BB-17-9

Compound BB-17-8 (5 g, 20.14 mmol) was dissolved in dichloromethane (50 mL) at room temperature under nitrogen atmosphere. The mixture was cooled to 0° C., and N, N-dimethylformamide (147.18 mg, 2.01 mmol, 154.92 μL) and oxalyl chloride (3.83 g, 30.20 mmol, 2.64 mL) were sequentially added thereto. The reaction mixture was stirred and reacted at 0° C. for 1 hour. After the reaction was completed, the reaction mixture was directly concentrated under reduced pressure to remove the solvent to obtain compound BB-17-9, which was used in the next step directly.

### Step 7: Synthesis of Compound BB-17-10

Compound BB-17-4 (6.5 g, 18.26 mmol) was dissolved in dichloromethane (100 mL) at room temperature under nitrogen atmosphere. The mixture was cooled to 0° C., and then triethylamine (5.54 g, 54.78 mmol, 7.62 mL) and a solution of compound BB-17-9 (5.36 g, 20.09 mmol) in dichloromethane (100 mL) were added thereto. The reaction mixture was returned to room temperature, and stirred and reacted for 12 hours. After the reaction was completed, the mixture was added with water (100 mL), and extracted with dichloromethane (100 mL×2). The organic phases were combined, dried over anhydrous sodium sulfate, and filtered. The filtrate was concentrated under reduced pressure to obtain a residue. The residue was added with ethyl acetate (50 mL), stirred at room temperature for 30 minutes, and filtered. The solid was collected, and dried under vacuum to obtain compound BB-17-10. MS-ESI m/z: 585.8 [M+H]⁺, 587.8 [M+H+2]⁺. ¹H NMR (400 MHz, CDCl₃) δ: 8.76 (s, 1H), 8.08 (s, 1H), 7.52 (s, 1H), 7.40-7.27 (m, 5H), 4.52 (s, 2H), 3.92 (s, 3H), 3.33 (d, J=6.4 Hz, 2H), 2.35-2.24 (m, 1H), 2.16-2.06 (m, 2H), 2.05-1.93 (m, 2H), 1.78-1.68 (m, 1H), 1.65-1.51 (m, 2H), 1.20-1.03 (m, 2H).

### Step 8: Synthesis of Compound BB-17-11

Compound BB-17-10 (7.5 g, 12.79 mmol) was dissolved in N, N-dimethylformamide (80 mL) at room temperature under nitrogen atmosphere, then cuprous iodide (487.29 mg, 2.56 mmol) and sodium sulfide nonahydrate (18.44 g, 76.76 mmol) were added thereto. The reaction mixture was heated to 80° C., and stirred and reacted for 6 hours. After the LCMS tracked the complete consumption of raw materials, the reaction mixture was cooled to room temperature, and trifluoroacetic acid (20.42 g, 179.10 mmol, 13.26 mL) was added thereto. The reaction mixture was stirred and reacted at room temperature for 16 hours. Additional trifluoroacetic acid (7 mL) was added thereto, and the reaction mixture was further stirred and reacted for 2 hours. After the reaction was completed, the reaction mixture was poured into water (300 mL), diluted with ethyl acetate (200 mL), and filtered. The filter cake was rinsed with ethyl acetate (100 mL×2). The filtrate was collected and the phases were separated. The aqueous phase was extracted with ethyl acetate (200 mL×2). The organic phases were combined, washed with 10% brine (300 mL×2), dried over anhydrous sodium sulfate, and filtered. The filtrate was concentrated under reduced pressure to obtain compound BB-17-11. MS-ESI m/z: 459.9 [M+H]⁺, 461.9 [M+H+2]⁺.

### Step 9: Synthesis of Compound BB-17-12

Compound BB-17-11 (5.89 g, 12.79 mmol) was dissolved in N, N-dimethylformamide (70 mL) at room temperature, then potassium carbonate (3.54 g, 25.59 mmol) and methyl iodide (3.63 g, 25.59 mmol, 1.59 mL) were added thereto. The reaction mixture was stirred and reacted at room temperature for 12 hours. Additional potassium carbonate (3.54 g, 25.59 mmol) and methyl iodide (1 mL) were added thereto. The reaction mixture was further stirred and reacted for 1 hour. After the reaction was completed, the reaction mixture was poured into water (200 mL) and extracted with ethyl acetate (200 mL×3). The organic phases were combined, washed sequentially with 10% brine (200 mL×2) and saturated brine (100 mL), dried over anhydrous sodium sulfate, and filtered. The filtrate was concentrated under reduced pressure to obtain a residue. The resulting residue was separated by column chromatography (eluent: petroleum ether/ethyl acetate=1/0 to 4/1, v/v) to obtain compound BB-17-12. MS-ESI m/z: 473.9 [M+H]⁺, 475.9 [M+H+2]⁺. ¹H NMR (400 MHz, CDCl₃) δ: 8.41 (s, 1H), 8.14 (s, 1H), 7.41-7.27 (m, 5H), 4.53 (s, 2H), 3.97 (s, 3H), 3.36 (d, J=6.4 Hz, 2H), 3.12-3.00 (m, 1H), 2.36-2.23 (m, 2H), 2.06-1.99 (m, 2H), 1.84-1.74 (m, 1H), 1.73-1.61 (m, 2H), 1.26-1.13 (m, 2H).

### Step 10: Synthesis of Compound BB-17-13

Compound BB-17-12 (4.08 g, 8.60 mmol) was dissolved in dichloromethane (20 mL) at room temperature under nitrogen atmosphere, then a solution of boron trichloride (1 M, 100 mL) in dichloromethane was added thereto. The reaction mixture was stirred and reacted at room temperature for 1 hour. After the reaction was completed, the reaction mixture was poured into a saturated aqueous solution of sodium bicarbonate (100 mL), and extracted with dichloromethane (50 mL×3). The organic phases were combined, washed with saturated brine (100 mL), dried over anhydrous sodium sulfate, and filtered. The filtrate was concentrated under reduced pressure to obtain compound BB-17-13. MS-ESI m/z: 383.9 [M+H]⁺, 386.0 [M+H+2]⁺. ¹H NMR (400 MHz, CDCl₃) δ: 8.40 (s, 1H), 8.14 (s, 1H), 3.97 (s, 3H), 3.54 (d, J=6.0 Hz, 2H), 3.10-3.00 (m, 1H), 2.36-2.23 (m, 2H), 2.07-1.95 (m, 2H), 1.75-1.55 (m, 4H), 1.28-1.13 (m, 2H).

### Step 11: Synthesis of Compound BB-17-14

Compound BB-17-13 (3.4 g, 8.85 mmol) was dissolved in dioxane (70 mL) at room temperature under nitrogen atmosphere, and then compound BB-17-2 (1.85 g, 9.37 mmol), 4,5-bisdiphenylphosphino-9,9-dimethylxanthene (1.02 g, 1.77 mmol), tris(dibenzylideneacetone)dipalladium (810.18 mg, 884.75 μmol), and cesium carbonate (8.65 g, 26.54 mmol) were added thereto. The reaction mixture was heated to 80° C., and stirred and reacted for 12 hours. After the reaction was completed, the reaction mixture was cooled to room temperature, and filtered. The filter cake was rinsed with tetrahydrofuran (100 mL×3). The filtrate was collected, and concentrated under reduced pressure to obtain a residue. The residue was separated by column chromatography (eluent: petroleum ether/ethyl acetate=1/0 to 11/9, v/v) to obtain compound BB-17-14. MS-ESI m/z: 494.0 [M+H]. ¹H NMR (400 MHz, DMSO_d₆) δ: 12.79 (s, 1H), 9.41 (s, 1H), 8.50 (s, 1H), 8.45 (d, J=7.6 Hz 1H), 8.39 (t, J=7.8 Hz, 1H), 8.21 (d, J=7.2 Hz, 1H), 4.45 (t, J=5.4 Hz, 1H), 3.97 (s, 3H), 3.27 (t, J=5.8 Hz, 2H), 3.11-3.00 (m, 1H), 2.23-2.12 (m, 2H), 1.93-1.82 (m, 2H), 1.62-1.50 (m, 2H), 1.49-1.36 (m, 1H), 1.17-1.03 (m, 2H).

### Step 12: Synthesis of Compound BB-17-15

Compound BB-17-14 (2 g, 4.05 mmol) was dissolved in tetrahydrofuran (40 mL) at room temperature under nitrogen atmosphere. The mixture was cooled to 0° C., and then a solution of methylmagnesium bromide (3 M, 8.11 mL) in tetrahydrofuran was added dropwise thereto. The reaction mixture was stirred and reacted at 0° C. for 2 hours. An additional solution of methylmagnesium bromide (3 M, 1.35 mL) in tetrahydrofuran was added. The mixture was further stirred and reacted for 1 hour. After the reaction was completed, the reaction mixture was poured into saturated ammonium chloride (100 mL) and extracted with ethyl acetate (50 mL×3). The organic phases were combined, dried over anhydrous sodium sulfate, and filtered. The filtrate was concentrated under reduced pressure to obtain a residue. The residue was separated by column chromatography (eluent: petroleum ether/ethyl acetate=I/O to 1/1, v/v) to obtain compound BB-17-15. MS-ESI m/z: 494.0 [M+H]. ¹H NMR (400 MHz, DMSO_d₆) δ:12.55 (s, 1H), 9.06 (s, 1H), 8.47 (d, J=8.0 Hz, 1H), 8.38 (t, J=7.8 Hz, 1H), 8.18 (dd, J=0.8, 7.6 Hz, 1H), 7.89 (s, 1H), 6.07 (s, 1H), 4.45 (t, J=5.2 Hz, 1H), 3.27 (t, J=5.8 Hz, 2H), 3.08-2.98 (m, 1H), 2.20-2.13 (m, 2H), 1.90-1.83 (m, 2H), 1.63 (s, 6H), 1.59-1.50 (m, 2H), 1.49-1.38 (m, 1H), 1.15-1.06 (m, 2H).

### Step 13: Synthesis of Compound BB-17

Compound BB-17-15 (1.63 g, 3.30 mmol) was dissolved in dichloromethane (40 mL) at room temperature under nitrogen atmosphere, then triphenylphosphine (1.04 g, 3.96 mmol) and imidazole (337.25 mg, 4.95 mmol) were added thereto. The mixture was cooled to 0° C., and iodine (1.09 g, 4.29 mmol) was added thereto. The reaction mixture was stirred and reacted at room temperature for 12 hours. After the reaction was completed, the reaction mixture was poured into saturated sodium sulfite (200 mL), and extracted with dichloromethane (100 mL×3). The organic phases were combined, dried over anhydrous sodium sulfate, and filtered. The filtrate was concentrated under reduced pressure to obtain a residue. The residue was first separated by column chromatography (eluent: petroleum ether/ethyl acetate=1/0 to 7/3, v/v), and further separated twice by preparative high performance liquid chromatography (chromatographic column: Phenomenex luna C18 (250*70 mm, 15 m); mobile phase: water (0.2% formic acid)-acetonitrile; B %: 60%-90%, 30 minutes) and by (chromatogram: Phenomenex Luna C18 75*30 mm*3 m; mobile phase: water (0.2% formic acid)-acetonitrile; B %: 45%-85%, 8 minutes) to obtain compound BB-17. MS-ESI m/z: 603.9 [M+H]⁺. ¹H NMR (400 MHz, DMSO_d₆) δ: 12.55 (s, 1H), 9.07 (s, 1H), 8.47 (d, J=7.6 Hz, 1H), 8.38 (t, J=8.0 Hz, 1H), 8.18 (d, J=8.0 Hz, 1H), 7.89 (s, 1H), 6.07 (s, 1H), 3.27 (d, J=6.4 Hz, 2H), 3.08-2.97 (m, 1H), 2.22-2.13 (m, 2H), 2.02-1.92 (m, 2H), 1.71-1.56 (m, 8H), 1.55-1.42 (m, 1H), 1.28-1.10 (m, 2H).

### Reference Example 18: Fragment BB-18

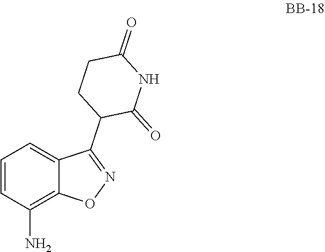 [SMILES: Nc1cccc2c(C3CCC(=O)NC3=O)noc12]

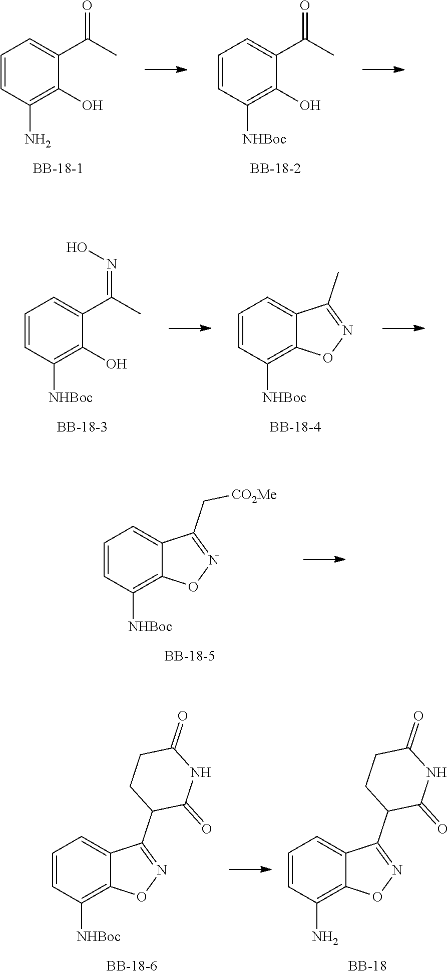

### Step 1: Synthesis of Compound BB-18-2

Compound BB-18-1 (200 g, 1.32 mol) was dissolved in ethanol (1.5 L) at room temperature under nitrogen atmosphere, then di-tert-butyl dicarbonate (346.51 g, 1.59 mol) was added thereto in batches. The reaction mixture was heated to 60° C., and stirred and reacted for 16 hours. After the reaction was completed, the reaction mixture was cooled to room temperature, and concentrated under reduced pressure to remove the solvent. The residue was added with isopropanol (500 mL), cooled to 0° C., stirred for 1 hour, and filtered. The filter cake was rinsed with isopropanol (200 mL). The filtrate was collected and concentrated under reduced pressure to obtain compound BB-18-2. MS-ESI m/z: 196.1 [M+H−56]⁺.

### Step 2: Synthesis of Compound BB-18-3

Compound BB-18-2 (200 g, 795.93 mmol), potassium acetate (97.94 g, 1.19 mol), and hydroxylamine hydrochloride (60.84 g, 875.53 mmol) were dissolved in methanol (1.2 L) at room temperature under nitrogen atmosphere. The reaction mixture was stirred at room temperature for 16 hours, then heated to 50° C. and reacted for 1 hour. Additional hydroxylamine hydrochloride (11.06 g, 159.19 mmol) was added thereto, and the mixture was further reacted for 1 hour. After the reaction was completed, the reaction mixture was cooled to room temperature, added with water (1.5 L) and methyl tert-butyl ether (1.5 L). The mixture was stirred for 5 minutes and the phases were separated. The aqueous phase was extracted with methyl tert-butyl ether (500 mL×2). The organic phases were combined, washed with 10% brine (500 mL×2), dried over anhydrous sodium sulfate, and filtered. The filtrate was concentrated under reduced pressure to remove the solvent to obtain compound BB-18-3. MS-ESI m/z: 211.1 [M+H−56]⁺. ¹H NMR (400 MHz, DMSO_d₆) δ: 12.27 (s, 1H), 11.67 (s, 1H), 7.82 (s, 1H), 7.69 (d, J=7.2 Hz, 1H), 7.23 (d, J=8.0 Hz, 1H), 6.86 (t, J=8.0 Hz, 1H), 2.27 (s, 3H), 1.46 (s, 9H).

### Step 3: Synthesis of Compound BB-18-4

Compound BB-18-3 (100 g, 375.53 mmol) and triethylamine (49.40 g, 488.19 mmol) were dissolved in tetrahydrofuran (1 L) at room temperature under nitrogen atmosphere, and N, N′-carbonyldiimidazole (66.98 g, 413.08 mmol) was slowly added thereto. The reaction mixture was heated to 70° C., and stirred and reacted for 1 hour. After the reaction was completed, the reaction mixture was cooled to room temperature, added with water (1 L), and extracted with a mixed solvent of methyl tert-butyl ether and petroleum ether (1/1, v/v, 500 mL×2). The organic phases were washed with 10% brine (500 mL×2), dried over anhydrous sodium sulfate, and filtered. The filtrate was concentrated under reduced pressure to remove the solvent to obtain compound BB-18-4. MS-ESI m/z: 249.1 [M+H]⁺. ¹H NMR (400 MHz, CDCl₃) δ: 8.16 (s, 1H), 7.30 (s, 1H), 7.00 (s, 1H), 2.59 (s, 3H), 1.57 (s, 9H).

### Step 4: Synthesis of Compound BB-18-5

A solution of lithium diisopropylamide (2 M, 741.11 mL) in tetrahydrofuran was cooled to −68° C. at room temperature under nitrogen atmosphere. Dimethyl carbonate (36.72 g, 407.61 mmol, 34.31 mL) and a solution of compound BB-18-4 (92 g, 370.55 mmol) in tetrahydrofuran (920 mL) were added dropwise thereto, the duration of which is about 30 minutes. The mixture was stirred and reacted for 30 minutes, with temperature controlled at −50° C. to −68° C. After the reaction was completed, the reaction mixture was slowly poured into saturated ammonium chloride (1.5 L), and extracted with ethyl acetate (500 mL×3). The organic phase was washed with saturated brine (500 mL), dried over anhydrous sodium sulfate, and filtered. The filtrate was concentrated under reduced pressure to remove the solvent to obtain compound BB-18-5. MS-ESI m/z: 307.1 [M+H]⁺.

### Step 5: Synthesis of Compound BB-18-6

Compound BB-18-5 (10 g, 32.65 mmol) and acrylamide (2.78 g, 39.18 mmol) were dissolved in tetrahydrofuran (100 mL) at room temperature under nitrogen atmosphere. The mixture was cooled to 0 to 5° C., and a solution of potassium tert-butoxide in tetrahydrofuran (1 M, 48.97 mL) was added dropwise thereto. After the dropwise addition was completed, the mixture was stirred at the same temperature for 2 hours. After the reaction was completed, the reaction mixture was poured into 1 M hydrochloric acid (150 mL), and extracted with ethyl acetate (150 mL×2). The organic phases were combined, washed with saturated brine (150 mL), dried over anhydrous sodium sulfate, and filtered. The filtrate was concentrated under reduced pressure to obtain a crude product. The crude product was added with methyl tert-butyl ether (150 mL). The mixture was stirred at room temperature for 30 minutes, and filtered. The filter cake was rinsed with methyl tert-butyl ether (50 mL), collected, and dried under vacuum to obtain compound BB-18-6. ¹H NMR (400 MHz, DMSO_d₆) δ: 11.10 (s, 1H), 9.55 (s, 1H), 7.69 (d, J=7.6 Hz, 1H), 7.54 (d, J=7.6 Hz, 1H), 7.30 (t, J=8.0 Hz, 1H), 4.60 (dd, J=4.8, 12.0 Hz, 1H), 2.84-2.72 (m, 1H), 2.66-2.57 (m, 1H), 2.57-2.43 (m, 1H), 2.25-2.15 (m, 1H), 1.49 (s, 9H).

Step 6: Synthesis of hydrochloride of compound BB-18

Compound BB-18-6 (9.4 g, 27.22 mmol) was dissolved in ethyl acetate (50 mL) at room temperature, then a solution of hydrochloric acid in ethyl acetate (4 M, 150 mL) was added thereto. The reaction mixture was stirred and reacted at room temperature for 12 hours. After the reaction was completed, the mixture was filtered. The filter cake was rinsed with ethyl acetate (20 mL), and collected to obtain the hydrochloride of compound BB-18. ¹H NMR (400 MHz, DMSO_d₆) δ: 11.09 (s, 1H), 8.32 (s, 2H), 7.31 (d, J=7.6 Hz, 1H), 7.26-7.16 (m, 2H), 4.59 (dd, J=4.8, 12.0 Hz, 1H), 2.83-2.71 (m, 1H), 2.65-2.56 (m, 1H), 2.55-2.42 (m, 1H), 2.25-2.15 (m, 1H).

### Example 1

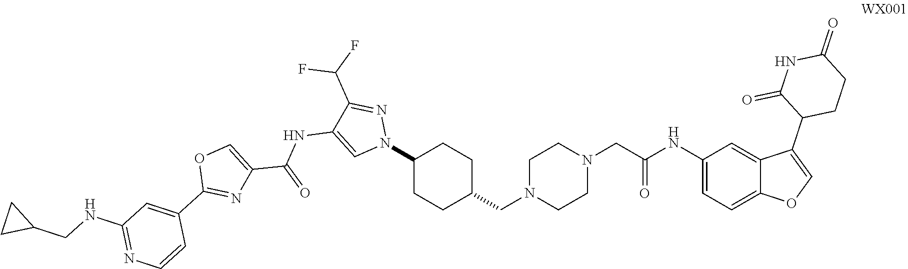 [SMILES: O=C1CCC(c2coc3ccc(NC(=O)CN4CCN(C[C@H]5CC[C@H](n6cc(NC(=O)c7coc(-c8ccnc(NCC9CC9)c8)n7)c(C(F)F)n6)CC5)CC4)cc23)C(=O)N1]

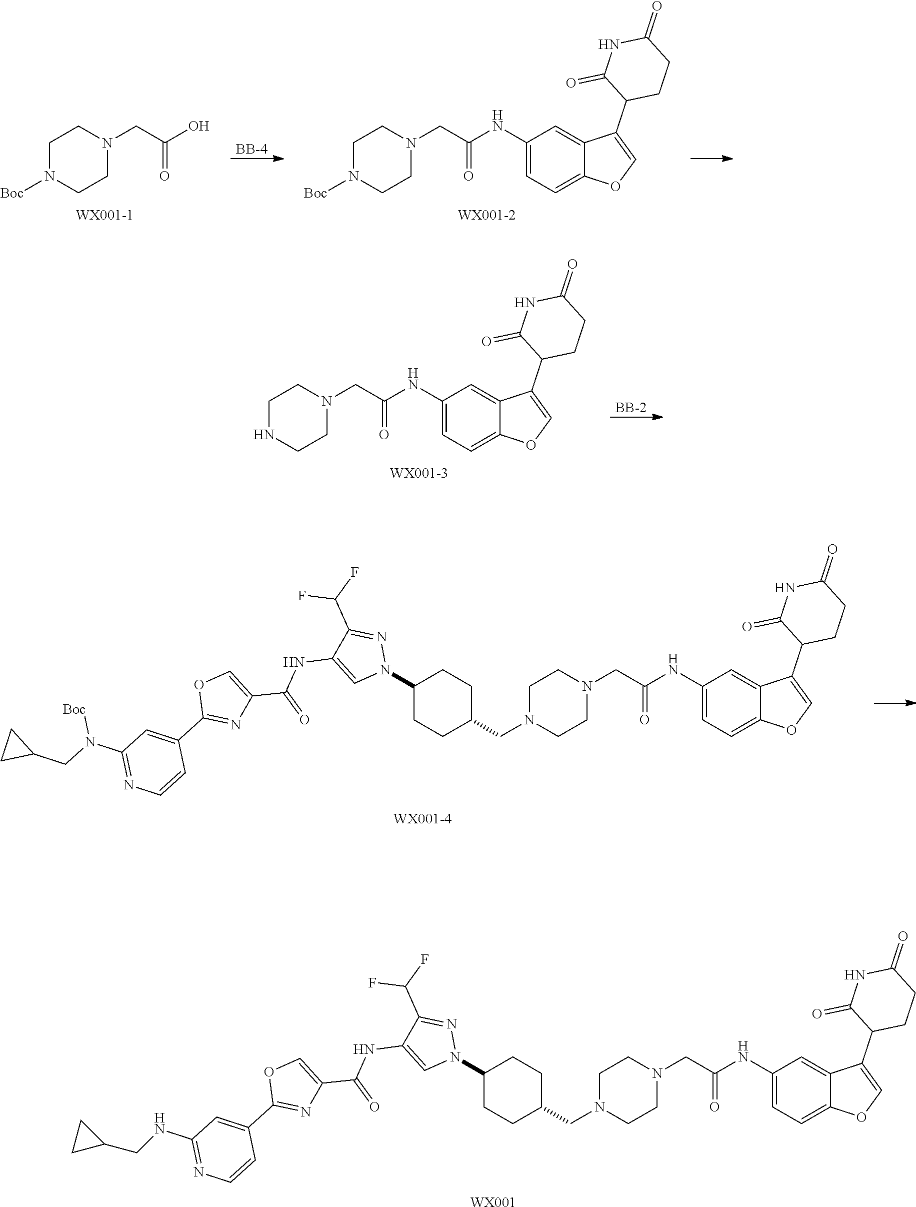

### Step 1: Synthesis of Compound WX001-2

Compound WX001-1 (612.43 mg, 1.85 mmol, purity of 84.92%) and triethylamine (720.96 mg, 7.12 mmol) were dissolved in N, N-dimethylformamide (5 mL) at room temperature, and 2-(7-azabenzotriazol)-N, N, N′, N′-tetramethyluronium hexafluorophosphate (880.46 mg, 2.32 mmol) was added thereto. The reaction mixture was stirred and reacted at room temperature for 1 hour, and the hydrochloride of compound BB-4 (0.5 g, 1.78 mmol) was added thereto. The reaction mixture was further stirred and reacted for 4 hours. After the reaction was completed, methyl tert-butyl ether (20 mL) was added thereto. The mixture was stirred for 5 minutes, and filtered. The filter cake was rinsed with methyl tert-butyl ether (5 mL) and discard. The filtrate was collected. The filtrate was added with water (50 mL) and dichloromethane (100 mL), then the phases were separated. The organic phases were combined, dried over anhydrous sodium sulfate, and filtered. The filtrate was concentrated under reduced pressure to remove the solvent to obtain compound WX001-2, which was used in the next step directly. MS-ESI m/z: 471.3 [M+H]⁺.

### Step 2: Synthesis of Hydrochloride of Compound WX001-3

Compound WX001-2 (0.8 g, 1.70 mmol) was dissolved in dichloromethane (5 mL) at room temperature, then hydrochloric acid/ethyl acetate solution (4 M, 20 mL) was added thereto. The reaction mixture was stirred and reacted at room temperature for 2 hours. After the reaction was completed, the reacted mixture was filtered. The filter cake was rinsed with ethyl acetate (20 mL), and collected to obtain the hydrochloride of compound WX001-3. MS-ESI m/z: 371.1 [M+H]⁺. ¹H NMR (400 MHz, D₂O) δ: 7.78 (s, 1H), 7.64 (d, J=2.0 Hz, 1H), 7.57 (d, J=8.8 Hz, 1H), 7.30 (dd, J=2.0, 8.8 Hz, 1H), 4.21 (dd, J=5.2, 12.4 Hz, 1H), 4.06 (s, 2H), 3.62-3.56 (m, 4H), 3.55-3.50 (m, 4H), 2.90-2.75 (m, 2H), 2.51-2.38 (m, 1H), 2.33-2.24 (m, 1H).

### Step 3: Synthesis of Compound WX001-4

Compound BB-2 (0.1 g, 171.05 μmol) and the hydrochloride of compound WX001-3 (69.60 mg, 171.05 μmol) were dissolved in tetrahydrofuran (4 mL) and N, N-dimethylformamide (0.8 mL) at room temperature under nitrogen atmosphere, and potassium acetate (50.36 mg, 513.16 μmol) and acetic acid (5.14 mg, 85.53 μmol) were added thereto. The reaction mixture was stirred and reacted at room temperature for 2 hours. Sodium triacetoxyborohydride (145.01 mg, 684.21 μmol) was added thereto. The mixture was further stirred and reacted for 10 hours. After the reaction was completed, the reaction mixture was added with a saturated solution of ammonium chloride (10 mL), and extracted with ethyl acetate (20 mL×3). The organic phases were combined, washed with 10% brine (30 mL×2), dried over anhydrous sodium sulfate, and filtered. The filtrate was concentrated under reduced pressure to remove the solvent to obtain compound WX001-4. MS-ESI m/z: 939.5 [M+H]⁺.

### Step 4: Synthesis of Hydrochloride of Compound WX001

Compound WX001-4 (160 mg, 170.39 μmol) was dissolved in dichloromethane (5 mL) and trifluoroacetic acid (5 mL) at room temperature under nitrogen atmosphere. The reaction mixture was stirred and reacted at room temperature for 2 hours. After the reaction was completed, the reaction mixture was concentrated under reduced pressure to remove the solvent. The resulting residue was separated by preparative high performance liquid chromatography (chromatographic column: Phenomenex Luna C18 80×40 mm×3 m; mobile phase: water (0.04% hydrochloric acid)-acetonitrile; acetonitrile %: 13% to 33%, 7 minutes) to obtain the hydrochloride of the target compound WX001. MS-ESI m/z: 839.5 [M+H]⁺. ¹H NMR (400 MHz, DMSO_d₆) δ:10.94 (s, 1H), 9.98 (s, 1H), 9.14 (s, 1H), 8.27-8.15 (m, 1H), 8.06 (d, J=5.2 Hz, 1H), 7.93 (s, 2H), 7.69 (s, 1H), 7.57 (d, J=8.8 Hz, 1H), 7.48 (d, J=8.8 Hz, 1H), 7.37-7.03 (m, 2H), 4.37-4.17 (m, 3H), 4.13 (dd, J=4.4, 12.0 Hz, 2H), 3.41-3.32 (m, 5H), 3.15-3.03 (m, 2H), 2.86-2.73 (m, 2H), 2.69-2.55 (m, 2H), 2.35-2.17 (m, 2H), 2.16-1.99 (m, 5H), 1.98-1.88 (m, 1H), 1.87-1.67 (m, 3H), 1.55 (d, J=7.2 Hz, 1H), 1.27-1.07 (m, 3H), 0.55 (d, J=7.2 Hz, 2H), 0.34 (d, J=4.0 Hz, 2H).

### Example 2

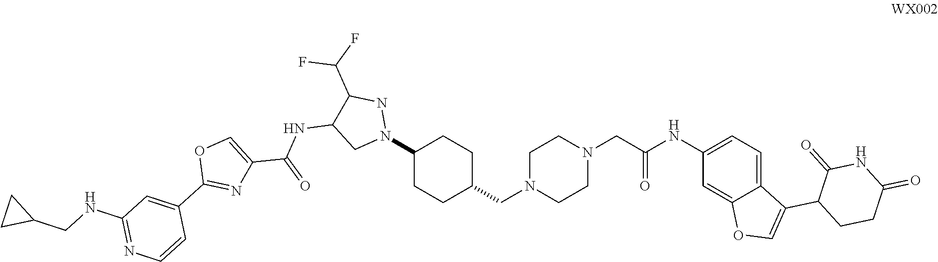 [SMILES: O=C1CCC(c2coc3cc(NC(=O)CN4CCN(C[C@H]5CC[C@H](N6CC(NC(=O)c7coc(-c8ccnc(NCC9CC9)c8)n7)C(C(F)F)N6)CC5)CC4)ccc23)C(=O)N1]

### Synthetic Route:

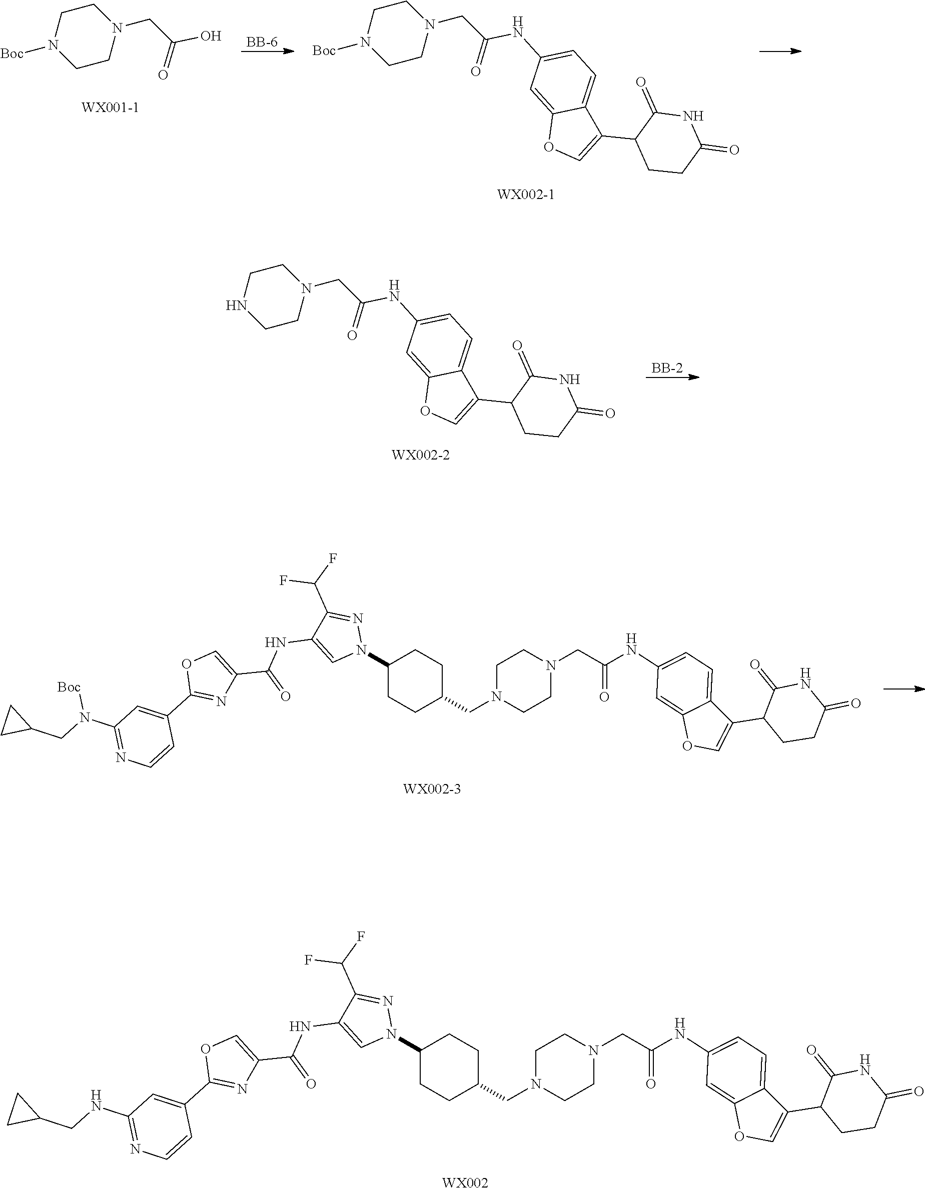

### Step 1: Synthesis of Compound WX002-1

Compound WX001-1 (0.5 g, 1.78 mmol) and triethylamine (632.58 mg, 6.25 mmol) were dissolved in N, N-dimethylformamide (10 mL) at room temperature, and 2-(7-azabenzotriazol)-N, N, N′, N′-tetramethyluronium hexafluorophosphate (812.61 mg, 2.14 mmol) was added thereto. The reaction mixture was stirred and reacted for 1 hour. The hydrochloride of compound BB-6 (522.08 mg, 1.33 mmol) was added thereto. The reaction mixture was further stirred and reacted for 19 hours. After the reaction was completed, the reaction mixture was filtered. The filter cake was washed with N, N-dimethylformamide (5 mL), again washed with tert-butyl methyl ether (20 mL) again, collected, and dried under vacuum to obtain compound WX002-1. MS-ESI m/z: 471.3 [M+H]⁺. ¹H NMR (400 MHz, DMSO_d₆) δ: 10.88 (s, 1H), 9.93 (s, 1H), 8.05 (d, J=1.2 Hz, 1H), 7.83 (s, 1H), 7.49 (d, J=8.4 Hz, 1H), 7.38 (dd, J=1.2, 8.4 Hz, 1H), 4.10 (dd, J=4.8, 12.0 Hz, 1H), 3.43-3.36 (m, 4H), 3.35-3.33 (m, 2H), 3.22-3.16 (m, 2H), 2.79-2.64 (m, 2H), 2.63-2.52 (m, 2H), 2.37-2.24 (m, 1H), 2.16-2.06 (m, 1H), 1.40 (s, 9H).

### Step 2: Synthesis of Hydrochloride of Compound WX002-2

Compound WX002-1 (0.8 g, 1.70 mmol) was dissolved in ethyl acetate (10 mL) at room temperature, then hydrochloric acid/ethyl acetate (4 M, 40 mL) was added thereto. The reaction mixture was stirred and reacted at room temperature for 15 hours. After the reaction was completed, the reaction mixture was filtered. The filter cake was washed with ethyl acetate (5 mL), collected, and dried under vacuum to obtain the hydrochloride of compound WX002-2. MS-ESI m/z: 371.3 [M+H]⁺. ¹H NMR (400 MHz, D₂O) δ: 7.75 (d, J=1.6 Hz, 1H), 7.66 (s, 1H), 7.43 (d, J=8.4 Hz, 1H), 7.19 (dd, J=1.2, 8.4 Hz, 1H), 4.31 (s, 2H), 4.10-4.03 (m, 1H), 3.80-3.72 (m, 4H), 3.71-3.63 (m, 4H), 2.78-2.61 (m, 2H), 2.33-2.20 (m, 1H), 2.18-2.09 (m, 1H).

### Step 3: Synthesis of Compound WX002-3

Compound BB-2 (0.1 g, 171.05 μmol) and the hydrochloride of compound WX002-2 (69.60 mg, 171.05 μmol) were dissolved in tetrahydrofuran (4 mL) and N, N-dimethylformamide (0.8 mL) at room temperature under nitrogen atmosphere. Potassium acetate (50.36 mg, 513.16 μmol) and acetic acid (5.14 mg, 85.53 μmol, 4.89 μL) were added thereto. The reaction mixture was stirred and reacted at room temperature for 2 hours. Sodium triacetoxyborohydride (145.01 mg, 684.21 μmol) was added thereto. The reaction mixture was further stirred and reacted for 10 hours. After the reaction was completed, the reaction mixture was added with a saturated solution of ammonium chloride (10 mL), and extracted with ethyl acetate (20 mL×3). The organic phases were combined, washed with 10% brine (30 mL×2), dried over anhydrous sodium sulfate, and filtered. The filtrate was concentrated under reduced pressure to remove the solvent to obtain compound WX002-3. MS-ESI m/z: 939.5 [M+H]⁺.

### Step 4: Synthesis of Hydrochloride of Compound WX002

Compound WX002-3 (160 mg, 170.39 μmol) was dissolved in dichloromethane (5 mL) and trifluoroacetic acid (5 mL) at room temperature under nitrogen atmosphere. The reaction mixture was stirred and reacted at room temperature for 2 hours. After the reaction was completed, the reaction mixture was concentrated under reduced pressure to remove the solvent. The resulting residue was separated by preparative high performance liquid chromatography (chromatographic column: Phenomenex Luna C18 80×40 mm×3 m; mobile phase: water (0.04% hydrochloric acid)-acetonitrile; acetonitrile %: 13% to 33%, 7 minutes) to obtain the hydrochloride of the target compound WX002. MS-ESI m/z: 839.5 [M+H]⁺. ¹H NMR (400 MHz, DMSO_d₆) δ: 10.91 (s, 1H), 9.94 (s, 1H), 9.13 (s, 1H), 8.19 (s, 1H), 8.10-8.04 (m, 2H), 7.88 (s, 1H), 7.66 (s, 1H), 7.55 (d, J=8.6 Hz, 1H), 7.38 (dd, J=1.4, 8.4 Hz, 1H), 7.34-7.04 (m, 2H), 4.31-4.22 (m, 1H), 4.12 (dd, J=4.6, 11.8H, 2H), 3.50-3.25 (m, 7H), 3.08 (s, 2H), 2.80-2.65 (m, 2H), 2.62-2.53 (m, 2H), 2.38-2.25 (m, 2H), 2.15-2.01 (m, 5H), 1.98-1.88 (m, 1H), 1.87-1.77 (m, 2H), 1.54 (d, J=6.8 Hz, 1H), 1.27-1.10 (m, 3H), 0.60-0.53 (m, 2H), 0.36-0.30 (m, 2H).

### Example 4

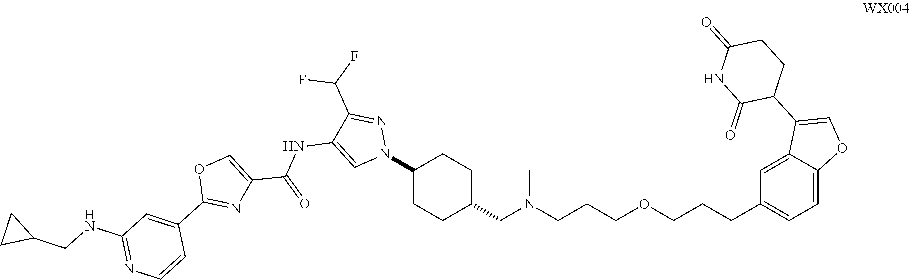 [SMILES: CN(CCCOCCCc1ccc2occ(C3CCC(=O)NC3=O)c2c1)C[C@H]1CC[C@H](n2cc(NC(=O)c3coc(-c4ccnc(NCC5CC5)c4)n3)c(C(F)F)n2)CC1]

### Synthetic Route:

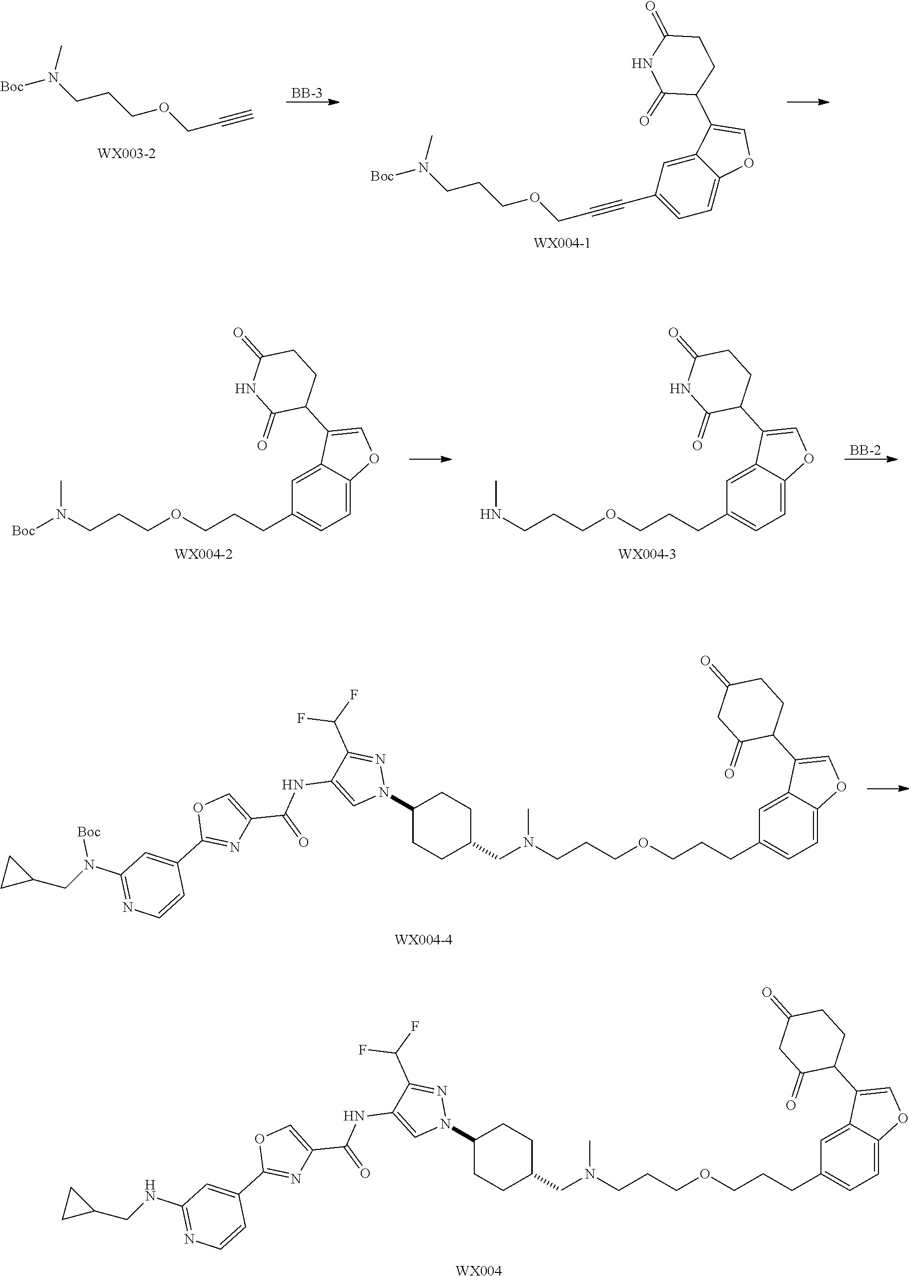

### Step 1: Synthesis of Compound WX004-1

Compound WX003-2 (553.26 mg, 2.43 mmol) was added to N, N-dimethylformamide (10 mL) at room temperature under nitrogen atmosphere. Compound BB-3 (0.5 g, 1.62 mmol), cesium carbonate (2.11 g, 6.49 mmol), cuprous iodide (61.81 mg, 324.54 μmol), and bis(triphenylphosphine)palladium(II) chloride (227.80 mg, 324.54 μmol) were sequentially added thereto. The reaction mixture was heated to 80° C., and stirred and reacted for 5 hours. After the reaction was completed, the reaction mixture was poured into 1 mol/L hydrochloric acid (12 mL), added with water (15 mL), and extracted with ethyl acetate (15 mL×3). The organic phases were combined, washed with saturated brine (25 mL×2), dried over anhydrous sodium sulfate, and filtered. The filtrate was concentrated under reduced pressure to remove the solvent. The resulting residue was separated by column chromatography (eluent: petroleum ether/ethyl acetate=5/1 to 1/1, v/v) to obtain compound WX004-1. MS-ESI m/z: 399.2 [M−55]*. ¹H NMR (400 MHz, DMSO_d₆) δ: 10.90 (s, 1H), 7.97 (s, 1H), 7.74 (s, 1H), 7.59 (d, J=8.4 Hz, 1H), 7.38 (dd, J=1.2, 8.4 Hz, 1H), 4.37 (s, 2H), 4.16 (dd, J=4.8, 12.0 Hz, 1H), 3.51 (t, J=6.4 Hz, 2H), 3.22 (t, J=7.0 Hz, 2H), 2.77 (s, 3H), 2.74-2.66 (m, 1H), 2.61-2.54 (m, 1H), 2.41-2.28 (m, 1H), 2.15-2.05 (m, 1H), 1.78-1.69 (m, 2H), 1.38 (s, 9H).

### Step 2: Synthesis of Compound WX004-2

Compound WX004-1 (0.6 g, 1.32 mmol) was dissolved in tetrahydrofuran (20 mL) at room temperature, then wet palladium on carbon (0.1 g, 1.32 mmol, purity of 10%) and palladium hydroxide/carbon (0.1 g, 1.32 mmol, purity of 10%) were added thereto. The reaction mixture was stirred and reacted at room temperature under hydrogen (15 psi) atmosphere for 12 hours. After the reaction was completed, the reaction mixture was filtered. The filter cake was rinsed with ethyl acetate (30 mL). The filtrate was collected and concentrated under reduced pressure to remove the solvent to obtain compound WX004-2. MS-ESI m/z: 403.2 [M−55]*.

### Step 3: Synthesis of Hydrochloride of Compound WX004-3

Compound WX004-2 (0.1 g, 218.08 μmol) was dissolved in hydrochloric acid/ethyl acetate (4 mol/L, 4 mL) at room temperature. The reaction mixture was stirred and reacted at room temperature for 1 hour. After the reaction was completed, the reaction mixture was filtered. The filter cake was rinsed with ethyl acetate (15 mL), and collected to obtain the hydrochloride of compound WX004-3. MS-ESI m/z: 359.2 [M+H]⁺. ¹H NMR (400 MHz, DMSO_d₆) δ: 10.89 (s, 1H), 8.73 (s, 1H), 7.86 (s, 1H), 7.47 (d, J=8.4 Hz, 1H), 7.39 (s, 1H), 7.15 (dd, J=1.6, 8.4 Hz, 1H), 4.12 (dd, J=4.8, 12.0 Hz, 1H), 3.43 (t, J=6.0 Hz, 2H), 3.38 (t, J=6.6 Hz, 2H), 3.34 (s, 3H), 2.95-2.88 (m, 2H), 2.81-2.74 (m, 1H), 2.73-2.67 (m, 2H), 2.62-2.54 (m, 1H), 2.38-2.27 (m, 1H), 2.14-2.05 (m, 1H), 1.88-1.78 (m, 4H).

### Step 4: Synthesis of Compound WX004-4

Compound BB-2 (0.1 g, 171.05 μmol) and the hydrochloride of compound WX004-3 (67.55 mg, 171.05 μmol) were dissolved in tetrahydrofuran (4 mL) and N, N-dimethylformamide (0.8 mL) at room temperature under nitrogen atmosphere, and potassium acetate (50.36 mg, 513.16 μmol) and acetic acid (5.14 mg, 85.53 μmol) were added thereto. The reaction mixture was stirred and reacted at room temperature for 2 hours. Sodium triacetoxyborohydride (145.01 mg, 684.21 μmol) was added thereto. The mixture was further stirred and reacted for 10 hours. After the reaction was completed, the reaction mixture was added with saturated ammonium chloride solution (10 mL), and extracted with ethyl acetate (3×30 mL). The organic phases were combined, washed with saturated brine (2×30 mL), dried over anhydrous sodium sulfate, and filtered. The filtrate was concentrated under reduced pressure to remove the solvent to obtain compound WX004-4. MS-ESI m/z: 927.6 [M+H]⁺.

### Step 5: Synthesis of Hydrochloride of Compound WX004

Compound WX004-4 (150 mg, 161.80 μmol) was dissolved in dichloromethane (5 mL) and trifluoroacetic acid (5 mL) at room temperature under nitrogen atmosphere. The reaction mixture was stirred and reacted at room temperature for 2 hours. After the reaction was completed, the reaction mixture was concentrated under reduced pressure to remove the solvent. The resulting residue was separated by preparative high performance liquid chromatography (chromatographic column: Phenomenex Luna C18 80×40 mm×3 m; mobile phase: water (0.04% hydrochloric acid)-acetonitrile; acetonitrile %: 17% to 37%, 7 minutes) to obtain the hydrochloride of the target compound WX004. MS-ESI m/z: 827.5 [M+H]⁺. ¹H NMR (400 MHz, DMSO_d₆) δ: 10.90 (s, 1H), 10.12 (s, 1H), 9.95 (s, 1H), 9.15 (s, 1H), 8.18 (d, J=28.8 Hz, 1H), 8.17-8.03 (m, 2H), 7.86 (s, 1H), 7.69 (s, 1H), 7.47 (d, J=8.4 Hz, 1H), 7.40 (s, 1H), 7.34-7.04 (m, 3H), 4.13 (dd, J=5.0, 12.2 Hz, 2H), 3.44 (t, J=5.8 Hz, 2H), 3.41-3.33 (m, 4H), 3.24-3.10 (m, 1H), 3.09-2.97 (m, 2H), 2.96-2.87 (m, 1H), 2.81-2.65 (m, 6H), 2.62-2.55 (m, 1H), 2.39-2.27 (m, 2H), 2.15-2.03 (m, 4H), 2.02-1.89 (m, 3H), 1.87-1.80 (m, 4H), 1.27-1.07 (m, 3H), 0.61-0.51 (m, 2H), 0.39-0.30 (m, 2H).

### Example 7

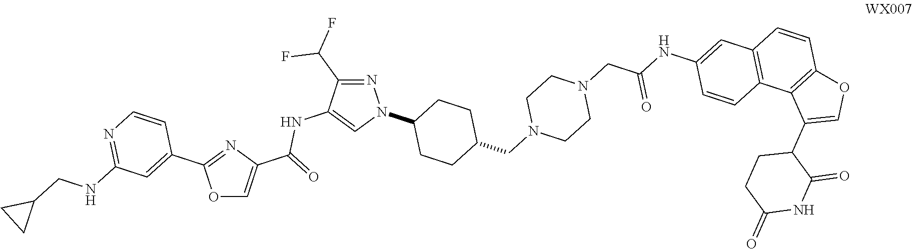

### Synthetic Route:

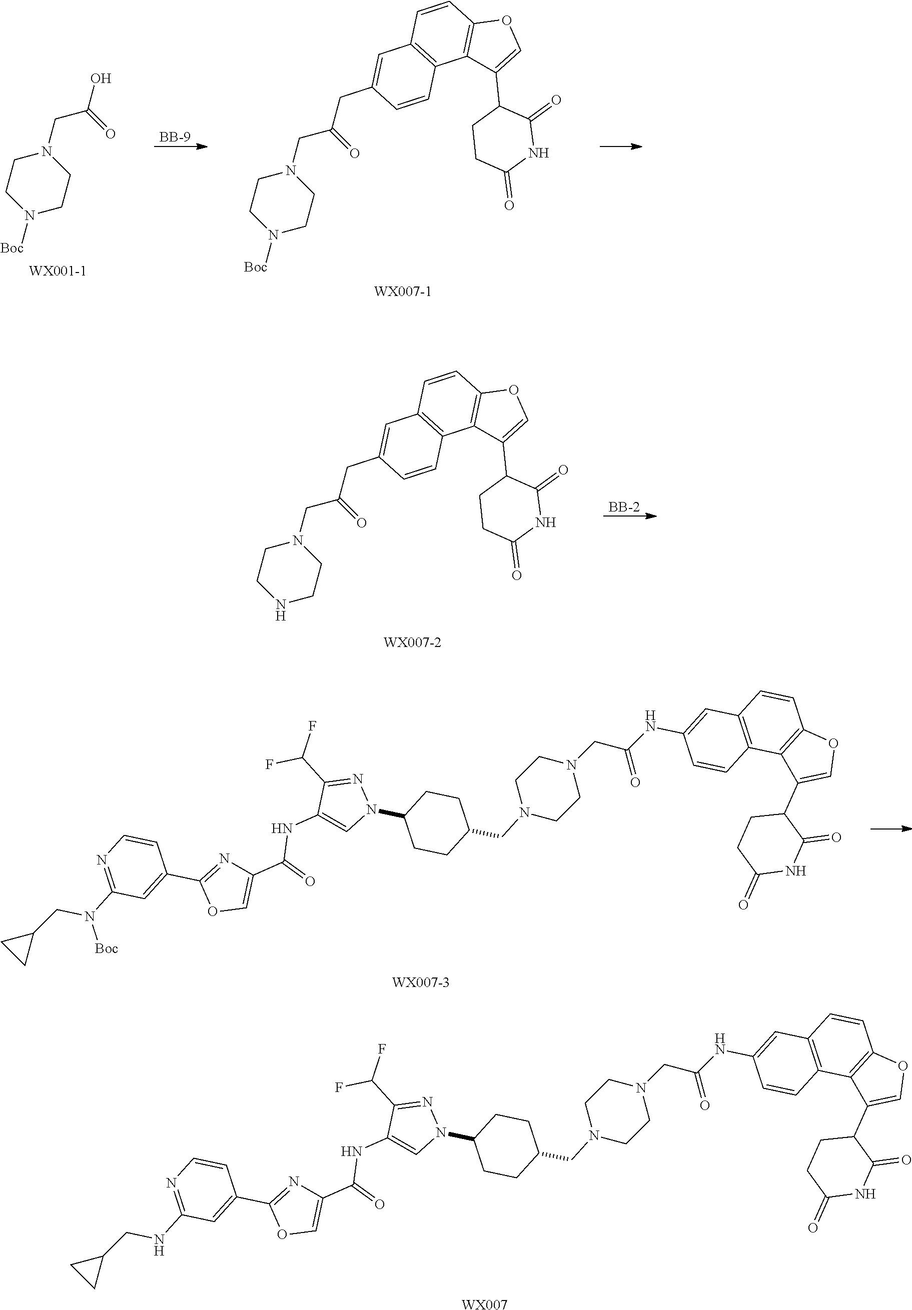

### Step 1: Synthesis of Compound WX007-1

Compound WX001-1 (387.74 mg, 1.59 mmol) was dissolved in N, N-dimethylformamide (10 mL) at room temperature, then 2-(7-azabenzotriazol)-N, N, N′, N′-tetramethyluronium hexafluorophosphate (482.81 mg, 1.27 mmol), N, N-diisopropylethylamine (410.27 mg, 3.17 mmol), and the hydrochloride of compound BB-9 (350 mg, 1.06 mmol) were sequentially added thereto. The reaction mixture was stirred and reacted at room temperature for 12 hours. After the reaction was completed, the reaction mixture was poured into water (50 mL), and extracted with ethyl acetate (10 mL×3). The organic phases were combined, washed with saturated brine (10 mL), dried over anhydrous sodium sulfate, and filtered. The filtrate was concentrated under reduced pressure to remove the solvent. The resulting residue was separated by column chromatography (eluent: petroleum ether/ethyl acetate=5/1 to 1/1, v/v) to obtain compound WX007-1. MS-ESI m/z: 521.1 [M+H]⁺. ¹H NMR (400 MHz, CDCl₃) δ: 9.25 (s, 1H), 8.28 (s, 1H), 8.10 (s, 1H), 7.95 (d, J=8.8 Hz, 1H), 7.77-7.70 (m, 2H), 7.69-7.64 (m, 2H), 4.48 (dd, J=5.0, 8.2 Hz, 1H), 3.61-3.51 (m, 4H), 3.24 (s, 2H), 2.90-2.76 (m, 2H), 2.70-2.59 (m, 4H), 2.56-2.46 (m, 2H), 1.49 (s, 9H).

### Step 2: Synthesis of Hydrochloride of Compound WX007-2

Compound WX007-1 (100 mg, 180.19 μmol, purity of 93.8%) was dissolved in hydrochloric acid/dioxane (4 mol/L, 7.5 mL). The reaction mixture was stirred and reacted at room temperature for 12 hours. After the reaction was completed, the reaction mixture was concentrated under reduced pressure to remove the solvent. The resulting residue was added with ethyl acetate (20 mL), then concentrated under reduced pressure to remove the solvent to obtain the hydrochloride of compound WX007-2. MS-ESI m/z: 421.1 [M+H]⁺.

### Step 3: Synthesis of Compound WX007-3

Compound BB-2 (77 mg, 131.71 μmol) and the hydrochloride of compound WX007-2 (60.18 mg, 131.71 μmol) were dissolved in tetrahydrofuran (5 mL) and N, N-dimethylformamide (1 mL) at room temperature, and then potassium acetate (38.78 mg, 395.13 mol) and acetic acid (3.95 mg, 65.86 μmol) were added thereto. The reaction mixture was stirred and reacted under nitrogen atmosphere for 0.5 hours. Sodium triacetoxyborohydride (83.74 mg, 395.13 μmol) was added thereto. The mixture was further stirred and reacted for 0.5 hours. After the reaction was completed, the reaction mixture was added with a saturated solution of ammonium chloride (20 mL), and extracted with ethyl acetate (20 mL×3). The organic phases were combined, washed with saturated brine (20 mL), dried over anhydrous sodium sulfate, and filtered. The filtrate was concentrated under reduced pressure to remove the solvent to obtain compound WX007-3. MS-ESI m/z: 889.5 [M+H−100]⁺.

### Step 4: Synthesis of Compound WX007

Compound WX007-3 (130 mg, 131.44 μmol) was dissolved in dichloromethane (3 mL) at room temperature and trifluoroacetic acid (3 mL) was added thereto. The reaction mixture was heated to 40° C. and stirred and reacted for 1 hour. After the reaction was completed, the mixture was concentrated under reduced pressure to remove the solvent. The resulting residue was separated by preparative high performance liquid chromatography (chromatographic column: Boston Green ODS 150*30 mm*5 m; mobile phase: water (0.04% hydrochloric acid)-acetonitrile; acetonitrile %: 19%-34%, 10 minutes), and again separated by preparative high performance liquid chromatography (chromatographic column: Welch Xtimate C18 150*25 mm*5 m; mobile phase: water (10 mM ammonium bicarbonate)-acetonitrile; acetonitrile %: 45% to 75%, 11 minutes) to obtain the target compound WX007. MS-ESI m/z: 890.8 [M+H]⁺. ¹H NMR (400 MHz, DMSO_d₆) δ: 10.94 (s, 1H), 9.92 (s, 1H), 9.72 (s, 1H), 8.88 (s, 1H), 8.36 (d, J=2.0 Hz, 1H), 8.17-8.08 (m, 3H), 7.97 (s, 1H), 7.79-7.70 (m, 3H), 7.15-6.96 (m, 4H), 4.63 (dd, J=4.2, 11.8 Hz, 1H), 4.24-4.10 (m, 1H), 3.18-3.14 (m, 3H), 2.94-2.81 (m, 1H), 2.68-2.63 (m, 1H), 2.61-2.54 (m, 4H), 2.30-2.23 (m, 2H), 2.17-2.12 (m, 2H), 2.08-1.95 (m, 4H), 1.93-1.85 (m, 2H), 1.78-1.70 (m, 2H), 1.63-1.55 (m, 1H), 1.22 (s, 3H), 1.09-1.00 (m, 2H), 0.84 (t, J=6.4 Hz, 1H), 0.48-0.40 (m, 2H), 0.24-0.17 (m, 2H).

### Example 8

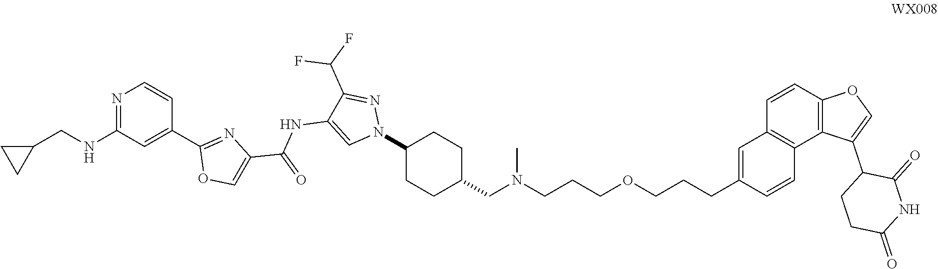 [SMILES: CN(CCCOCCCc1ccc2c(ccc3occ(C4CCC(=O)NC4=O)c32)c1)C[C@H]1CC[C@H](n2cc(NC(=O)c3coc(-c4ccnc(NCC5CC5)c4)n3)c(C(F)F)n2)CC1]

### Synthetic Route:

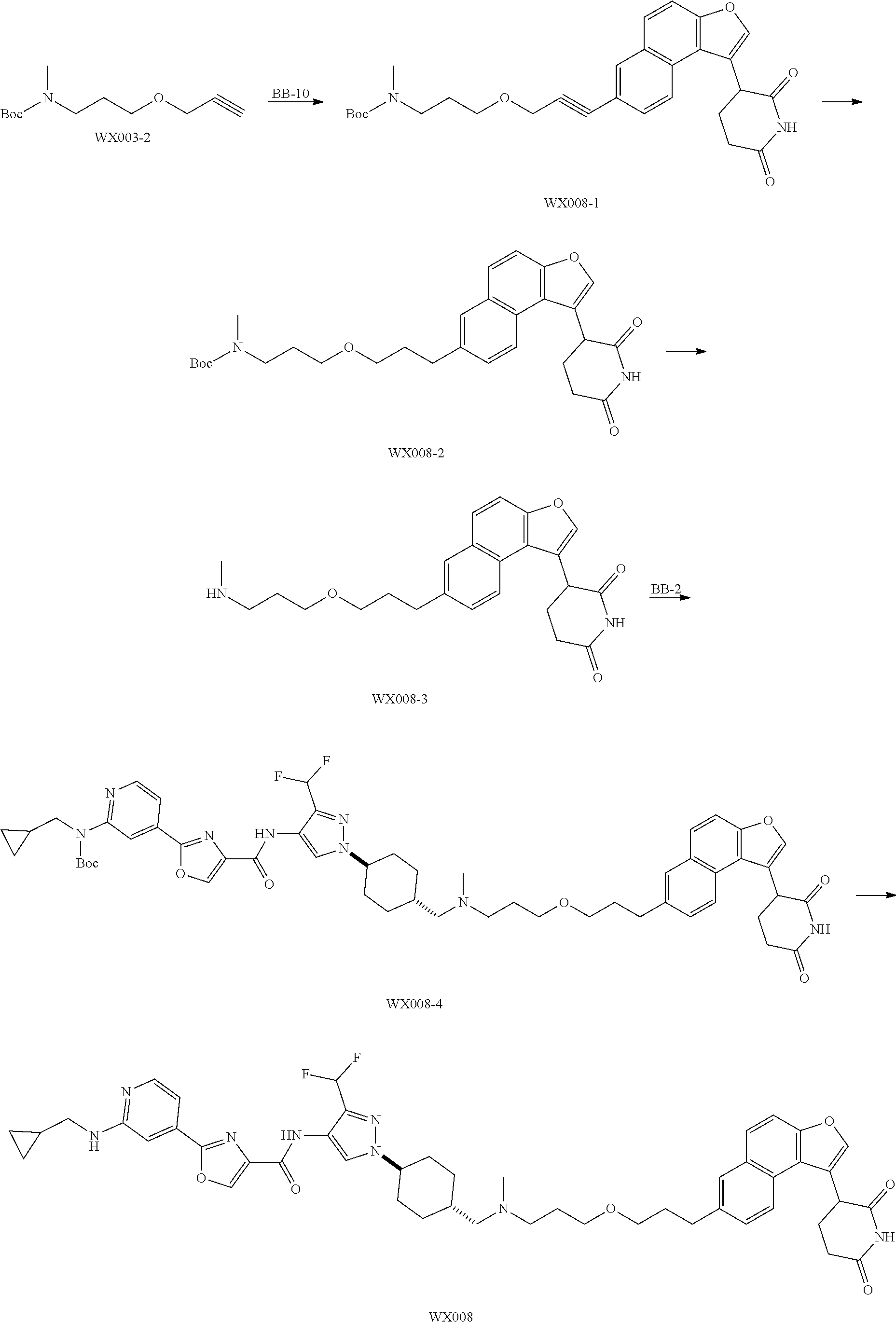

### Step 1: Synthesis of Compound WX008-1

Compound BB-10 (300 mg, 837.55 μmol) was dissolved in N, N-dimethylformamide (10 mL) at room temperature under nitrogen atmosphere, then compound WX003-2 (285.56 mg, 1.26 mmol), cesium carbonate (818.68 mg, 2.51 mmol), cuprous iodide (31.90 mg, 167.51 mol), and bis(triphenylphosphine)palladium(II) chloride (117.58 mg, 167.51 μmol) were sequentially added thereto. The reaction mixture was heated to 80° C., and stirred and reacted for 5 hours. After the reaction was completed, the reaction mixture was cooled to room temperature, poured into a saturated solution of ammonium chloride (100 mL), and extracted with ethyl acetate (10 mL×3). The organic phases were combined, washed with saturated brine (10 mL), dried over anhydrous sodium sulfate, and filtered. The filtrate was concentrated under reduced pressure to remove the solvent. The resulting residue was separated by column chromatography (eluent: petroleum ether/ethyl acetate=5/1 to 1/1, v/v) to obtain compound WX008-1. MS-ESI m/z: 405.2 [M+H−100]⁺. ¹H NMR (400 MHz, CDCl₃) δ: 8.15-8.01 (m, 2H), 7.92 (d, J=8.4 Hz, 1H), 7.73 (d, J=9.2 Hz, 1H), 7.69 (d, J=7.6 Hz, 2H), 7.61 (dd, J=1.6, 8.8 Hz, 1H), 4.50 (dd, J=5.2, 8.8 Hz, 1H), 4.43 (s, 2H), 3.65 (t, J=6.2 Hz, 2H), 3.35 (t, J=6.6 Hz, 2H), 2.89 (s, 3H), 2.85-2.78 (m, 2H), 2.58-2.41 (m, 2H), 1.94-1.83 (m, 2H), 1.47 (s, 9H).

### Step 2: Synthesis of Compound WX008-2

Compound WX008-1 (160 mg, 317.10 μmol) was dissolved in tetrahydrofuran (10 mL) at room temperature, then wet palladium on carbon (0.2 g, 317.10 μmol, purity of 10%) and palladium hydroxide/carbon (0.2 g, 317.10 μmol, purity of 20%) were sequentially added thereto. The reaction mixture was stirred and reacted at room temperature under hydrogen (15 psi) atmosphere for 2 hours. After the reaction was completed, the mixture was filtered. The filtrate was concentrated under reduced pressure to remove the solvent to obtain compound WX008-2. MS-ESI m/z: 409.1 [M+H−100]⁺. ¹H NMR (400 MHz, CDCl₃) δ: 8.09 (s, 1H), 7.92 (d, J=8.4 Hz, 1H), 7.78 (s, 1H), 7.72 (d, J=9.2 Hz, 1H), 7.67-7.63 (m, 2H), 7.45 (d, J=1.4, 8.6 Hz, 1H), 4.52 (dd, J=5.6, 8.0 Hz, 1H), 3.45 (q, J=6.6 Hz, 4H), 3.36-3.28 (m, 2H), 2.92-2.85 (m, 5H), 2.82-2.76 (m, 2H), 2.55-2.46 (m, 2H), 2.04-1.95 (m, 2H), 1.86-1.78 (m, 2H), 1.46 (s, 9H).

### Step 3: Synthesis of Hydrochloride of Compound WX008-3

Compound WX008-2 (100 mg, 196.62 μmol) was dissolved in ethyl acetate (5 mL) at room temperature, then a solution of hydrochloric acid in ethyl acetate (4 M, 5 mL) was added thereto. The reaction mixture was stirred and reacted at room temperature for 12 hours. After the reaction was completed, the mixture was concentrated under reduced pressure to remove the solvent. The resulting residue was added with ethyl acetate (20 mL), then concentrated under reduced pressure to remove the solvent to obtain hydrochloride of compound WX008-3. MS-ESI m/z: 409.2 [M+H]⁺.

### Step 4: Synthesis of Compound WX008-4

Compound BB-2 (91 mg, 147.64 μmol) was dissolved in tetrahydrofuran (5 mL) and N, N-dimethylformamide (1 mL) at room temperature, then the hydrochloride of compound WX008-3 (65.69 mg, 147.64 μmol), potassium acetate (43.47 mg, 442.93 μmol) and acetic acid (8.87 mg, 8.44 μL, 147.64 μmol) were added thereto. The reaction mixture was stirred and reacted for 0.5 hours. Sodium triacetoxyborohydride (93.87 mg, 442.93 μmol) was added thereto. The mixture was further stirred and reacted for 1 hour. After the reaction was completed, the reaction mixture was added with a saturated solution of ammonium chloride (20 mL), and extracted with ethyl acetate (20 mL×3). The organic phases were combined, washed with saturated brine (20 mL), dried over anhydrous sodium sulfate, and filtered. The filtrate was concentrated under reduced pressure to remove the solvent. The resulting residue was separated by column chromatography (eluent: dichloromethane/methanol=1/0 to 20/1, v/v) to obtain compound WX008-4. MS-ESI m/z: 977.6 [M+H]⁺.

### Step 5: Synthesis of Hydrochloride of Compound WX008

Compound WX008-4 (110 mg, 100.36 μmol, purity of 89.15%) was dissolved in dichloromethane (3 mL) at room temperature, then trifluoroacetic acid (3 mL) was added thereto. The reaction mixture was heated to 40° C., and stirred and reacted for 1 hour. After the reaction was completed, the mixture was concentrated under reduced pressure to remove the solvent. The resulting residue was separated by preparative high performance liquid chromatography (chromatographic column: Boston Green ODS 150*30 mm*5 m; mobile phase: water (0.04% hydrochloric acid)-acetonitrile; acetonitrile %: 25% to 45%, 10 minutes) to obtain the hydrochloride of the target compound WX008. MS-ESI m/z: 877.5 [M+H]⁺. ¹H NMR (400 MHz, DMSO_d₆) δ: 10.96 (s, 1H), 9.89 (s, 1H), 9.54 (s, 1H), 9.07 (s, 1H), 8.17 (s, 1H), 8.07 (d, J=6.0 Hz, 2H), 7.98 (s, 1H), 7.84 (s, 1H), 7.77 (dd, J=8.8, 16.0 Hz, 2H), 7.57 (s, 1H), 7.46 (d, J=8.4 Hz, 1H), 7.35-7.00 (m, 2H), 4.64 (dd, J=3.8, 11.8 Hz, 1H), 4.23 (t, J=11.8 Hz, 1H), 3.31-3.24 (m, 2H), 3.20-3.11 (m, 2H), 3.10-2.97 (m, 3H), 2.96-2.86 (m, 2H), 2.82 (t, J=7.4 Hz, 2H), 2.76 (d, J=4.0 Hz, 3H), 2.67-2.59 (m, 1H), 2.42-2.31 (m, 1H), 2.30-2.21 (m, 1H), 2.10-1.74 (m, 12H), 1.27-1.10 (m, 4H), 0.61-0.51 (m, 2H), 0.36-0.27 (m, 2H).

### Example 10

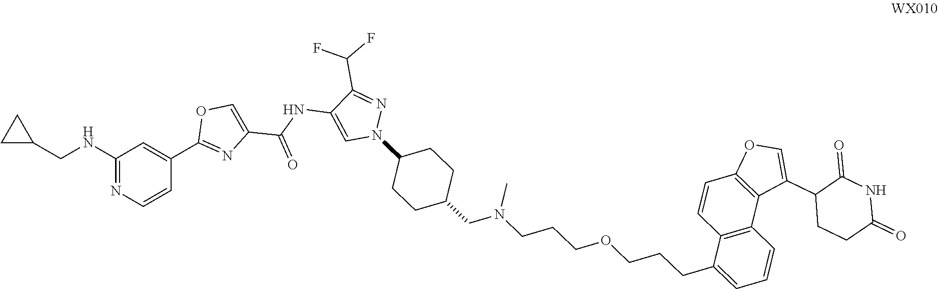

### Synthetic Route:

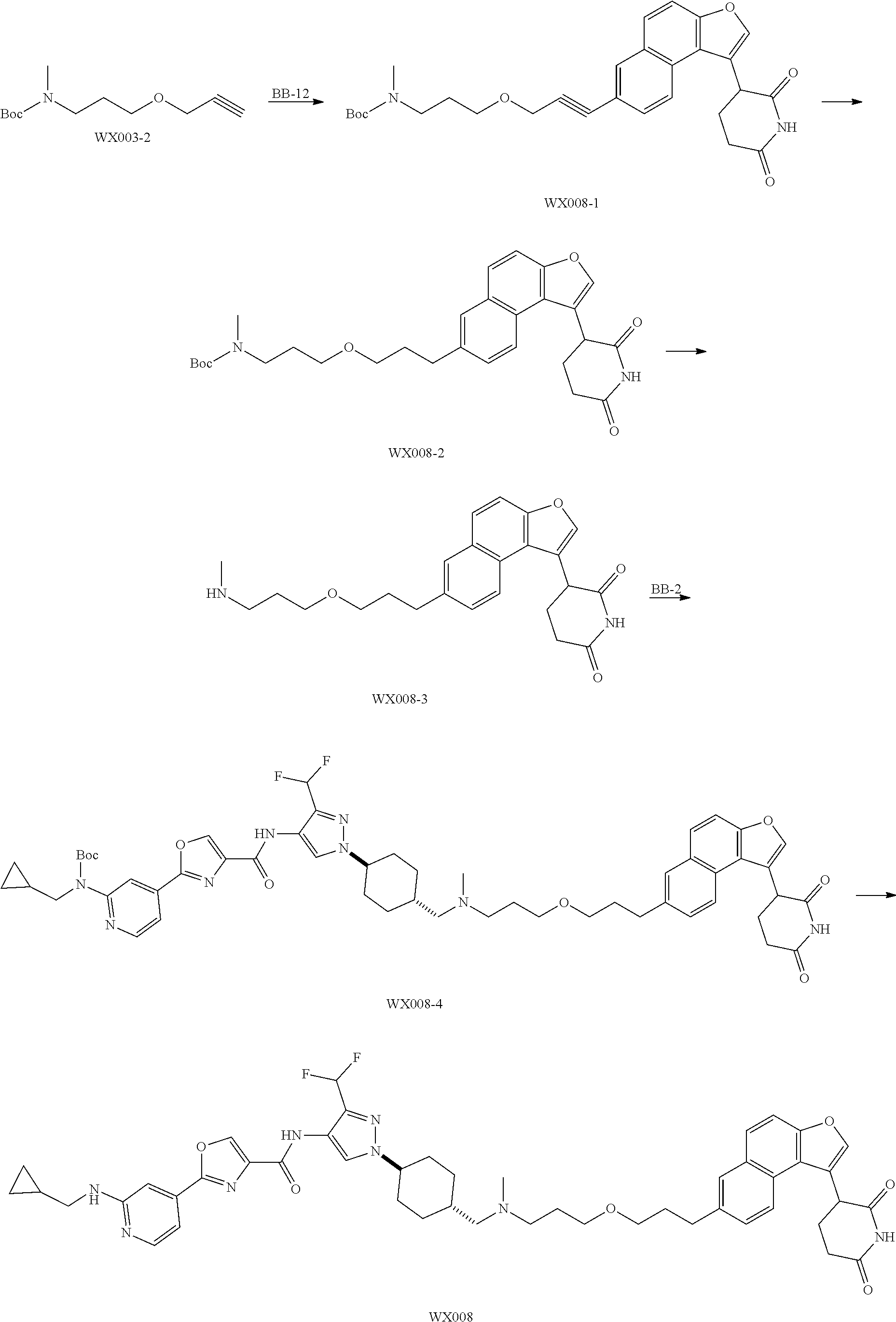

### Step 1: Synthesis of Compound WX010-1

Compound BB-12 (0.1 g, 279.18 μmol), compound WX003-2 (126.92 mg, 558.37 mol), cuprous iodide (10.63 mg, 55.84 μmol), bis(triphenylphosphine)palladium(II) dichloride (39.19 mg, 55.84 μmol), and N, N-diisopropyl ethyl amine (72.17 mg, 558.37 μmol) were dissolved in dimethyl sulfoxide (2 mL) at room temperature under nitrogen atmosphere. The reaction mixture was heated to 85° C., and stirred and reacted for 2.5 hours. After the reaction was completed, the reaction mixture was cooled to room temperature, added with water (2 mL), and extracted with dichloromethane (5 mL×2). The organic phases were combined, washed with saturated brine (5 mL), dried over anhydrous sodium sulfate, and filtered. The filtrate was concentrated under reduced pressure. The resulting residue was purified by a chromatography plate (eluent: petroleum ether/ethyl acetate=1/1, v/v) to obtain compound WX010-1. MS-ESI m/z: 405.2 [M+H−100]⁺. ¹H NMR (400 MHz, CDCl₃) δ: 8.35 (d, J=9.2 Hz, 1H), 8.12 (s, 1H), 7.99 (d, J=8.4 Hz, 1H), 7.76 (d, J=9.2 Hz, 1H), 7.71 (d, J=6.8 Hz, 1H), 7.69 (s, 1H), 7.53 (t, J=8.0 Hz, 1H), 4.54 (s, 2H), 4.50 (dd, J=5.4, 9.0 Hz, 1H), 3.70 (t, J=6.4 Hz, 2H), 3.36 (t, J=6.4 Hz, 2H), 2.89 (s, 3H), 2.85-2.77 (m, 2H), 2.57-2.42 (m, 2H), 1.96-1.86 (m, 2H), 1.46 (s, 9H).

### Step 2: Synthesis of Compound WX010-2

Compound WX010-1 (60 mg, 118.91 μmol) was dissolved in tetrahydrofuran (5 mL) at room temperature, then wet palladium on carbon (10 mg, purity of 10%) and palladium hydroxide/carbon (10 mg, purity of 20%) were sequentially added thereto. The reaction mixture was stirred and reacted at room temperature under hydrogen (15 psi) atmosphere for 15 hours. After the reaction was completed, the mixture was filtered. The filter cake was rinsed with tetrahydrofuran (10 mL×2). The filtrate was collected and concentrated under reduced pressure to obtain compound WX010-2. MS-ESI m/z: 409.2 [M+H−100]⁺.

Step 3: Synthesis of trifluoroacetate of compound WX010-3

Compound WX010-2 (60 mg, 117.97 μmol) was dissolved in dichloromethane (1 mL) at room temperature and trifluoroacetic acid (184.80 mg, 1.62 mmol) was added thereto. The reaction mixture was stirred and reacted at room temperature for 2 hours. After the reaction was completed, the mixture was concentrated under reduced pressure to remove the solvent to obtain the trifluoroacetate of compound WX010-3. MS-ESI m/z: 409.2 [M+H]⁺.

### Step 4: Synthesis of Compound WX010-4

Compound BB-2 (50 mg, 85.53 μmol), the trifluoroacetate of compound WX010-3 (53.63 mg, 102.63 μmol), and potassium acetate (25.18 mg, 256.58 μmol) were dissolved in a mixed solvent of N, N-dimethylformamide (1 mL) and glacial acetic acid (0.1 mL) at room temperature under nitrogen atmosphere. The reaction mixture was stirred and reacted at room temperature for 2 hours. Sodium triacetoxyborohydride (54.38 mg, 256.58 μmol) was added thereto. The reaction mixture was further stirred and reacted for 15 hours. After the reaction was completed, the reaction mixture was added with a saturated solution of ammonium chloride (8 mL), and extracted with ethyl acetate (10 mL×2). The organic phases were combined, washed with saturated brine (10 mL), dried over anhydrous sodium sulfate, and filtered. The filtrate was concentrated under reduced pressure to obtain compound WX010-4. MS-ESI m/z: 977.6 [M+H]⁺.

Step 5: Synthesis of trifluoroacetate of compound WX010

Compound WX010-4 (0.1 g, 102.34 μmol) was dissolved in dichloromethane (2 mL) at room temperature and trifluoroacetic acid (3.08 g, 27.01 mmol) was added dropwise thereto. The reaction mixture was stirred and reacted at room temperature for 4 hours. After the reaction was completed, the mixture was concentrated under reduced pressure to remove the solvent. The resulting crude product was separated by preparative high performance liquid chromatography (chromatographic column: Phenomenex Luna C18 80*30 mm*3 m; mobile phase: water (0.1% trifluoroacetic acid)-acetonitrile; acetonitrile %: 20% to 50%, 8 minutes) to obtain the trifluoroacetate of the target compound WX010. MS-ESI m/z: 877.6 [M+H]⁺. ¹H NMR (400 MHz, MeOD_d₄) δ: 8.81 (s, 1H), 8.16 (d, J=1.6 Hz, 1H), 8.12 (d, J=9.6 Hz, 1H), 8.06 (d, J=8.4 Hz, 1H), 7.99 (d, J=6.4 Hz, 1H), 7.84 (s, 1H), 7.74 (d, J=9.6 Hz, 1H), 7.70 (s, 1H), 7.53 (t, J=7.8 Hz, 1H), 7.45 (d, J=6.4 Hz, 1H), 7.41 (d, J=6.8 Hz, 1H), 6.93 (t, J=54.4 Hz 1H), 4.65 (dd, J=5.0 Hz, 10.2 Hz, 1H), 4.11-4.02 (m, 1H), 3.65-3.53 (m, 4H), 3.31-3.20 (m, 4H), 3.18-3.07 (m, 2H), 3.01-2.88 (m, 2H), 2.87 (s, 3H), 2.79-2.71 (m, 1H), 2.55-2.40 (m, 2H), 2.17-1.75 (m, 12H), 1.29-1.17 (m, 3H), 0.75-0.68 (m, 2H), 0.45-0.38 (m, 2H).

### Example 11

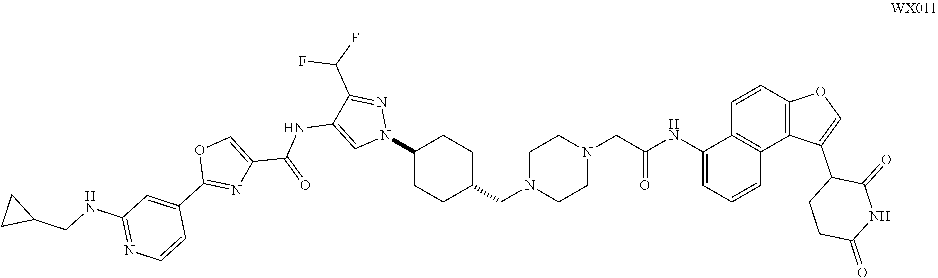 [SMILES: O=C1CCC(c2coc3ccc4c(NC(=O)CN5CCN(C[C@H]6CC[C@H](n7cc(NC(=O)c8coc(-c9ccnc(NCC%10CC%10)c9)n8)c(C(F)F)n7)CC6)CC5)cccc4c23)C(=O)N1]

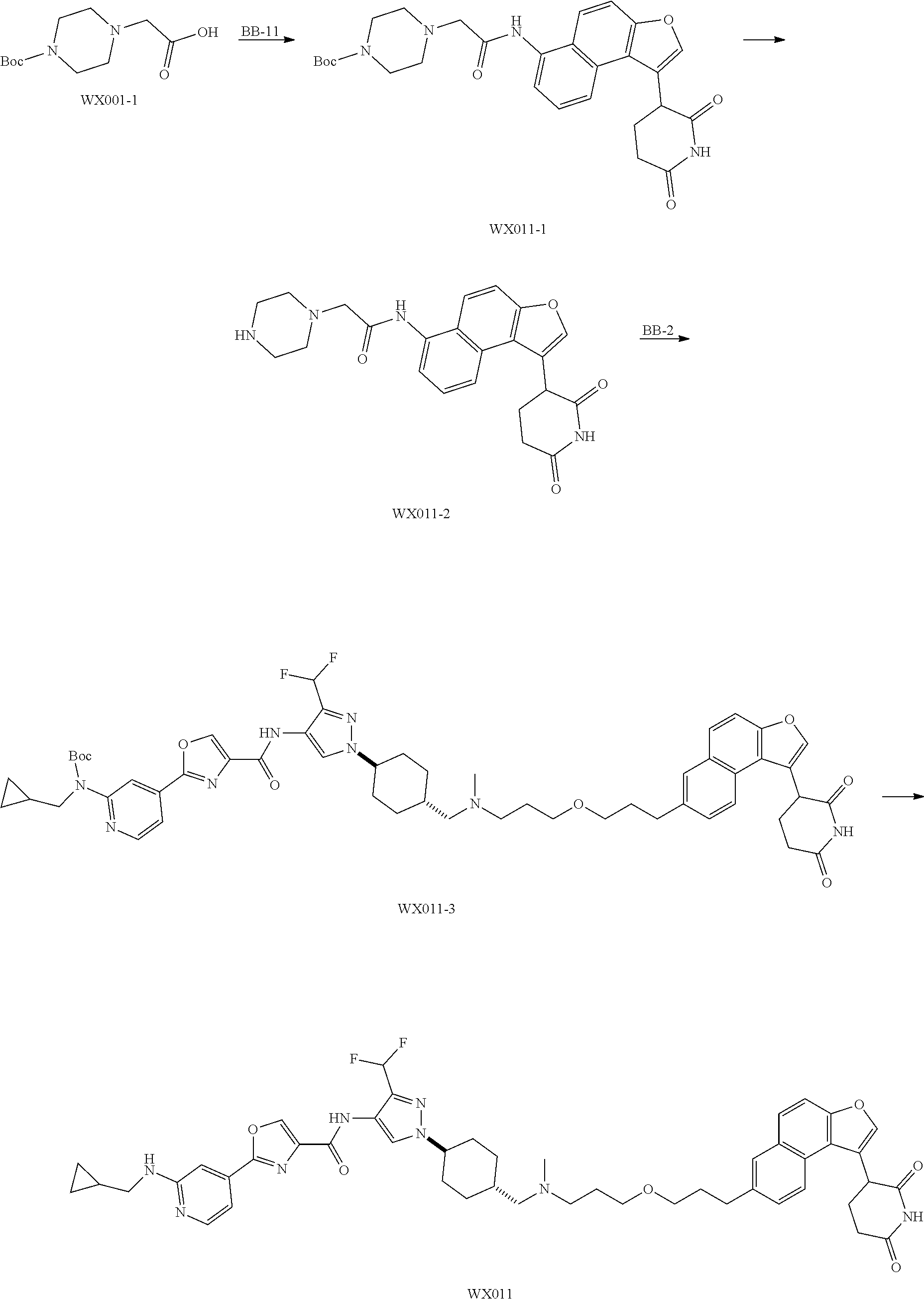

### Step 1: Synthesis of Compound WX011-1

Compound WX001-1 (102.70 mg, 365.82 μmol) was dissolved in: N, N-dimethylformamide (2 mL) at room temperature under nitrogen atmosphere, and 2-(7-azabenzotriazol)-N, N, N, N-tetramethyluronium hexafluorophosphate (164.39 mg, 432.33 mol) and N, N-diisopropylethylamine (214.91 mg, 1.66 mmol) were added thereto. The reaction mixture was stirred and reacted at room temperature for 0.5 hours. The hydrochloride of compound BB-11 (0.11 g, 332.56 μmol) was added thereto. The reaction mixture was stirred and reacted at room temperature for 15 hours. After the reaction was completed, the reaction mixture was added with water (5 mL) and extracted with ethyl acetate (2 mL×2). The organic phases were combined, washed with saturated brine (5 mL), dried over anhydrous sodium sulfate, and filtered. The filtrate was concentrated under reduced pressure to remove the solvent. The resulting residue was purified by a chromatography plate (developing solvent: ethyl acetate) to obtain compound WX011-1. MS-ESI m/z: 521.2 [M+H]⁺.

### Step 2: Synthesis of Hydrochloride of Compound WX011-2

Compound WX011-1 (0.12 g, 230.51 μmol) was dissolved in ethyl acetate (1 mL) at room temperature, and a solution of hydrochloric acid in ethyl acetate (4 M, 6 mL) was added thereto. The reaction mixture was stirred at room temperature for 1 hour. After the reaction was completed, the mixture was concentrated under reduced pressure to remove the solvent to obtain the hydrochloride of compound WX011-2. MS-ESI m/z: 421.2 [M+H]⁺.

### Step 3: Synthesis of Compound WX011-3

Compound BB-2 (50 mg, 85.53 μmol), the hydrochloride of the compound WX011-2 (42.99 mg, 94.08 μmol), and potassium acetate (25.18 mg, 256.58 μmol) were dissolved in a mixed solvent of N, N-dimethylformamide (1 mL) and glacial acetic acid (0.1 mL) at room temperature under nitrogen atmosphere. The reaction mixture was stirred and reacted at room temperature for 2 hours. Sodium triacetoxyborohydride (54.38 mg, 256.58 μmol) was added thereto. The reaction mixture was stirred and reacted at room temperature for 15 hours. After the reaction was completed, the mixture was added with a saturated solution of ammonium chloride (10 mL), and extracted with ethyl acetate (10 mL×2). The organic phases were combined, washed with saturated brine (10 mL), dried over anhydrous sodium sulfate, and filtered. The filtrate was concentrated under reduced pressure to obtain compound WX011-3. MS-ESI m/z: 467.3 [(M−56)/2+H]⁺.

Step 4: Synthesis of trifluoroacetate of compound WX011

Compound WX011-3 (80 mg, 80.88 μmol) was dissolved in dichloromethane (1 mL) at room temperature and trifluoroacetic acid (308.00 mg, 2.70 mmol, 0.2 ml) was added thereto. The reaction mixture was stirred and reacted at room temperature for 5 hours. After the reaction was completed, the mixture was concentrated under reduced pressure to remove the solvent. The resulting residue was separated by preparative high performance liquid chromatography (chromatographic column: Phenomenex Luna C18 100*40 mm*5 m; mobile phase: water (0.1% trifluoroacetic acid)-acetonitrile; acetonitrile %: 25% to 70%, 8 minutes) to obtain the trifluoroacetate of the target compound WX011. MS-ESI m/z: 889.5 [M+H]⁺. ¹H NMR (400 MHz, MeOD_d₄) δ: 8.79 (s, 1H), 8.26 (s, 1H), 8.13 (d, J=7.6 Hz, 1H), 7.97 (d, J=6.8 Hz, 1H), 7.93 (d, J=9.2 Hz, 1H), 7.88 (s, 1H), 7.77 (d, J=9.6 Hz, 1H), 7.70 (s, 1H), 7.67-7.60 (m, 2H), 7.44 (dd, J=0.8 Hz, 6.8 Hz, 1H), 7.09-6.79 (m, 1H), 4.67 (dd, J=5.4 Hz, 10.6 Hz, 1H), 4.31-4.19 (m, 1H), 3.60 (s, 2H), 3.53-3.38 (m, 3H), 3.28 (s, 2H), 3.24-3.18 (m, 2H), 3.16-3.00 (m, 4H), 2.96-2.85 (m, 1H), 2.81-2.72 (m, 1H), 2.56-2.41 (m, 2H), 2.23 (d, J=11.6 Hz, 2H), 2.10-1.65 (m, 6H), 1.43-1.27 (m, 2H), 1.26-1.17 (m, 1H), 0.74-0.67 (m, 2H), 0.43-0.37 (m, 2H).

### Example 13

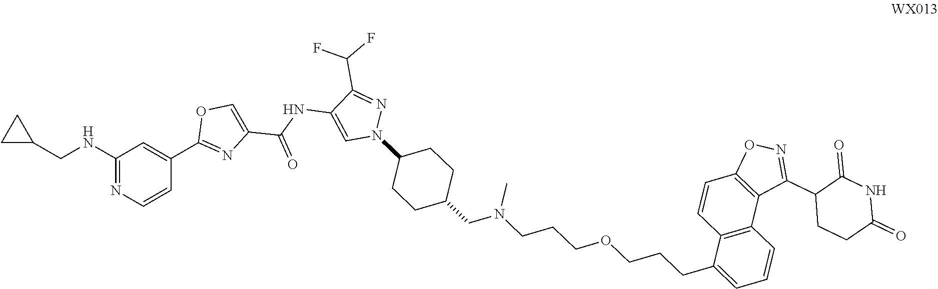

### Synthetic Route:

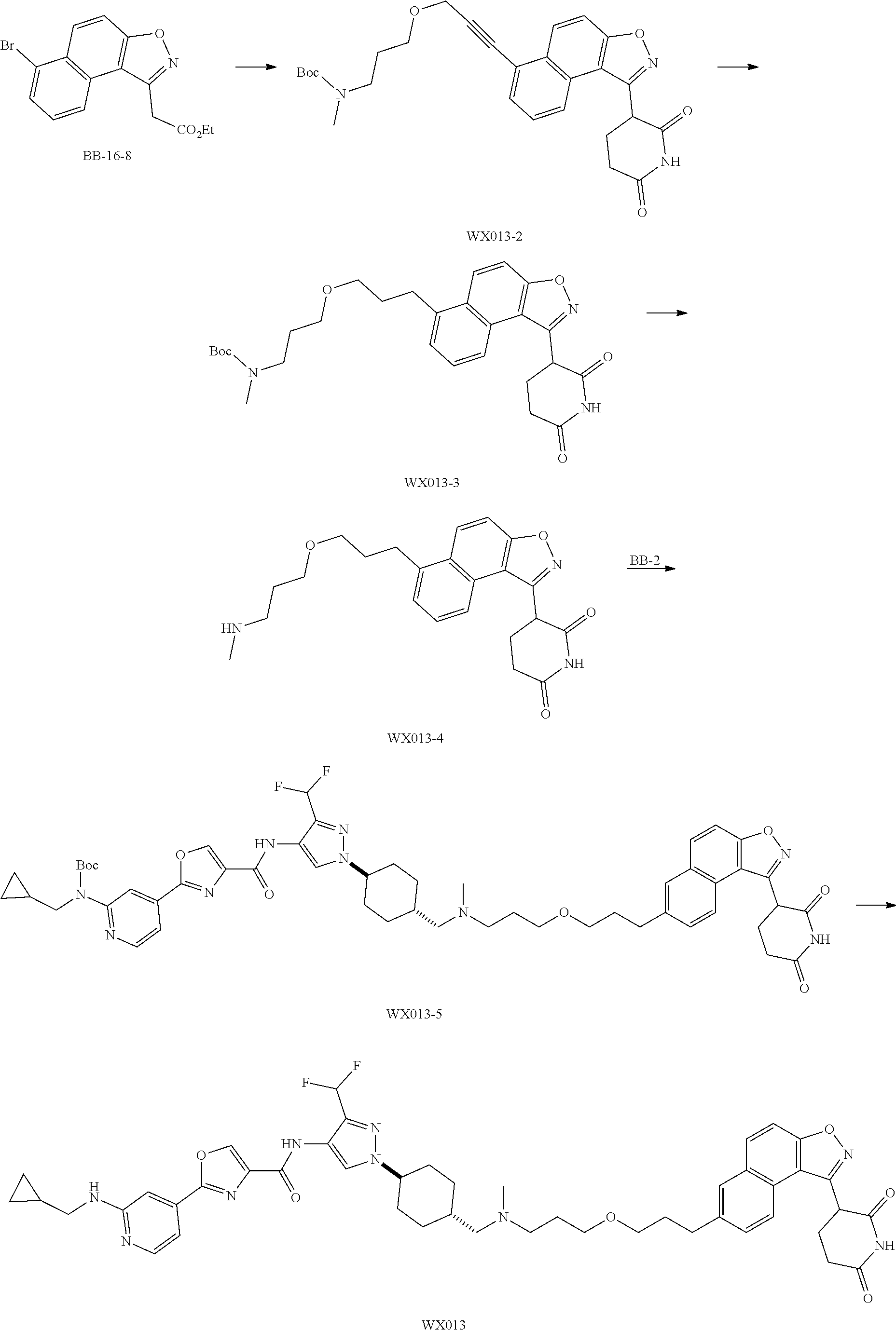

### Step 1: Synthesis of Compound WX013-1

Compound BB-16-8 (1 g, 2.99 mmol) and compound WX003-2 (816.24 mg, 3.59 mmol) were dissolved in dimethyl sulfoxide (10 mL) at room temperature under nitrogen atmosphere. Bis(triphenylphosphine)palladium(II) chloride (420.09 mg, 598.51 μmol), cuprous iodide (113.99 mg, 598.51 μmol), and N, N-diisopropylethylamine (773.51 mg, 5.99 mmol) were added thereto. The reaction mixture was heated to 85° C., and stirred and reacted for 2 hours. After the reaction was completed, the mixture was cooled to room temperature, added with water (30 mL), and extracted with ethyl acetate (3×20 mL). The organic phases were combined, washed with 10% brine (3×40 mL), dried over anhydrous sodium sulfate, and filtered. The filtrate was concentrated under reduced pressure to remove the solvent. The resulting residue was separated by column chromatography (eluent: petroleum ether/ethyl acetate=1/0 to 4/1, v/v) to obtain compound WX013-1. MS-ESI m/z: 381.2 [M+H−100]⁺. ¹H NMR (400 MHz, CDCl₃) δ: 8.54 (d, J=9.2 Hz, 1H), 8.11 (d, J=8.4 Hz, 1H), 7.79 (d, J=9.2 Hz, 1H), 7.75 (dd, J=1.0 Hz, 7.4 Hz, 1H), 7.62 (d, J=8.0 Hz, 1H), 4.54 (s, 2H), 4.34 (s, 2H), 4.22 (q, J=7.2 Hz, 2H), 3.70 (t, J=6.4 Hz, 2H), 3.36 (t, J=6.6 Hz, 2H), 2.89 (s, 3H), 1.95-1.86 (m, 2H), 1.46 (s, 9H), 1.20 (t, J=7.0 Hz, 3H).

### Step 2: Synthesis of Compound WX013-2

Compound WX013-1 (0.8 g, 1.66 mmol) and acrylamide (130.16 mg, 1.83 mmol) were dissolved in tetrahydrofuran (10 mL) at room temperature under nitrogen atmosphere. The mixture was cooled to 0° C., and potassium tert-butoxide (224.16 mg, 2.00 mmol) was added thereto. The reaction mixture was stirred and reacted at room temperature for 1 hour. After the reaction was completed, the reaction mixture was added to 1 N hydrochloric acid to adjust the pH to 3 to 4, added with water (10 mL), and extracted with ethyl acetate (3×20 mL). The organic phases were combined, dried over anhydrous sodium sulfate, and filtered. The filtrate was concentrated under reduced pressure to remove the solvent. The resulting residue was separated by column chromatography (eluent: petroleum ether/ethyl acetate=3/1 to 1/1, v/v) to obtain compound WX013-2. MS-ESI m/z: 406.2 [M+H−100]⁺. ¹H NMR (400 MHz, CDCl₃) δ: 8.59 (d, J=9.2 Hz, 1H), 8.07 (s, 1H), 8.04 (d, J=8.4 Hz, 1H), 7.82 (d, J=9.2 Hz, 1H), 7.77 (dd, J=0.8, 7.2 Hz, 1H), 7.65 (t, J=7.8 Hz, 1H), 4.74 (dd, J=5.0 Hz, 8.2 Hz, 1H), 4.54 (s, 2H), 3.70 (t, J=6.2 Hz, 2H), 3.37 (t, J=6.2 Hz, 2H), 2.99-2.91 (m, 1H), 2.90 (s, 3H), 2.84-2.68 (m, 2H), 2.64-2.53 (m, 1H), 1.96-1.86 (m, 2H), 1.46 (s, 9H).

### Step 3: Synthesis of Compound WX013-3

Wet palladium on carbon (20 mg, purity of 10%) and palladium hydroxide/carbon (20 mg, purity of 20%) were dissolved in tetrahydrofuran (3 mL) at room temperature. Compound WX013-2 (150 mg, 296.70 μmol) and triethylamine (30.02 mg, 296.70 μmol) were added thereto. The reaction mixture was stirred and reacted at room temperature under hydrogen (15 psi) atmosphere for 12 hours. After the reaction was completed, the mixture was filtered through diatomite. The filter cake was rinsed with ethyl acetate (20 mL). The filtrate was concentrated under reduced pressure to obtain compound WX013-3. MS-ESI m/z: 410.2 [M+H−100]⁺. 1H NMR (400 MHz, CDCl3) δ: 8.31 (d, J=9.2 Hz, 1H), 8.12 (s, 1H), 7.91 (d, J=8.4 Hz, 1H), 7.75 (d, J=9.6 Hz, 1H), 7.62 (t, J=7.6 Hz, 1H), 7.45 (d, J=6.8 Hz, 1H), 4.77 (dd, J=5.4 Hz, 7.8 Hz, 1H), 3.52-3.45 (m, 4H), 3.38-3.30 (m, 2H), 3.24 (t, J=7.6 Hz, 2H), 2.91-2.85 (m, 4H), 2.82-2.68 (m, 2H), 2.62-2.52 (m, 1H), 2.05-1.98 (m, 2H), 1.90-1.81 (m, 2H), 1.47 (s, 9H).

Step 4: Synthesis of trifluoroacetate of compound WX013-4

Compound WX013-3 (120 mg, 235.48 μmol) was dissolved in dichloromethane (3 mL) and trifluoroacetic acid (0.5 mL) at room temperature. The reaction mixture was stirred and reacted at room temperature for 1 hour. After the reaction was completed, the mixture was concentrated under reduced pressure to remove the solvent to obtain the trifluoroacetate of compound WX013-4. MS-ESI m/z: 410.3 [M+H]⁺.

### Step 5: Synthesis of Compound WX013-5

Compound BB-2 (0.1 g, 171.05 μmol) and the trifluoroacetate of compound WX013-4 (107.45 mg, 205.26 μmol) were dissolved in N, N-dimethylformamide (3 mL) at room temperature under nitrogen atmosphere. Potassium acetate (50.36 mg, 513.15 μmol) and acetic acid (5.14 mg, 85.53 μmol) were added thereto. The reaction mixture was stirred and reacted at room temperature for 2 hours. Sodium triacetoxyborohydride (145.01 mg, 684.20 mol) was added thereto. The reaction mixture was further stirred and reacted for 1 hour. After the reaction was completed, the mixture was added with 1 N hydrochloric acid solution (1 mL), and concentrated under reduced pressure to remove the solvent. The resulting residue was separated by preparative high performance liquid chromatography (chromatographic column: Phenomenex Luna C18 80*40 mm*3 m; mobile phase: water (0.04% hydrochloric acid)-acetonitrile; acetonitrile %; 40% to 60%, 7 minutes) to obtain compound WX013-5. MS-ESI m/z: 978.5 [M+H]⁺. ¹H NMR (400 MHz, CDCl₃) δ: 9.06 (s, 1H), 8.40 (d, J=5.6 Hz, 2H), 8.41 (s, 1H), 8.33 (s, 1H), 8.29 (t, J=4.8 Hz, 2H), 7.92 (d, J=8.4 Hz, 1H), 7.78 (d, J=9.2 Hz, 1H), 7.65-7.57 (m, 2H), 7.44 (d, J=7.2 Hz, 1H), 7.00-6.65 (m, 1H), 4.82-4.73 (m, 1H), 4.09-4.00 (m, 1H), 3.94 (d, J=6.8 Hz, 2H), 3.52 (t, J=5.6 Hz, 4H), 3.30-3.16 (m, 3H), 2.96-2.85 (m, 2H), 2.81 (s, 3H), 2.78-2.70 (m, 2H), 2.62-2.50 (m, 1H), 2.26-2.12 (m, 4H), 1.93-1.75 (m, 5H), 1.73-1.62 (m, 4H), 1.58 (s, 9H), 1.26-1.15 (m, 3H), 0.47-0.40 (m, 2H), 0.31-0.25 (m, 2H).

### Step 6: Synthesis of Hydrochloride of Compound WX013

Compound WX013-5 (50 mg, 51.12 μmol) was dissolved in dichloromethane (2 mL) and trifluoroacetic acid (2 mL) at room temperature under nitrogen atmosphere. The reaction mixture was stirred and reacted at room temperature for 1.5 hours. After the reaction was completed, the mixture was concentrated under reduced pressure to remove the solvent. The resulting residue was separated by preparative high performance liquid chromatography (chromatographic column: Phenomenex Luna C18 80*40 mm*3 m; mobile phase: water (0.04% hydrochloric acid)-acetonitrile; acetonitrile %: 14% to 34%, 7 minutes) to obtain the hydrochloride of the target compound WX013. MS-ESI m/z: 878.4 [M+H]⁺. ¹H NMR (400 MHz, MeOD_d₄) δ: 8.80 (s, 1H), 8.40 (d, J=9.2 Hz, 1H), 8.17 (s, 1H), 8.11 (d, J=8.4 Hz, 1H), 7.97 (d, J=6.8 Hz, 1H), 7.81 (d, J=9.6 Hz, 1H), 7.72 (d, J=1.2 Hz, 1H), 7.64 (t, J=7.8 Hz, 1H), 7.49 (d, J=6.8 Hz, 1H), 7.45 (dd, J=1.4 Hz, 6.6 Hz, 1H), 6.91 (t, J=54.6 Hz, 1H), 4.97 (dd, J=4.8 Hz, 10.0 Hz, 1H), 4.19-4.07 (m, 1H), 3.62-3.55 (m, 4H), 3.27-3.08 (m, 4H), 3.04-2.94 (m, 1H), 2.89 (s, 3H), 2.87-2.59 (m, 4H), 2.54-2.45 (m, 1H), 2.17-1.77 (m, 13H), 1.30-1.15 (m, 3H), 0.74-0.68 (m, 2H), 0.44-0.38 (m, 2H).

### Example 14

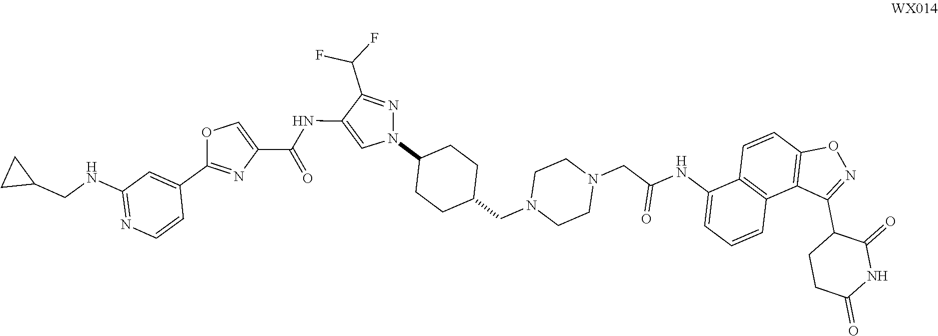 [SMILES: O=C1CCC(c2noc3ccc4c(NC(=O)CN5CCN(C[C@H]6CC[C@H](n7cc(NC(=O)c8coc(-c9ccnc(NCC%10CC%10)c9)n8)c(C(F)F)n7)CC6)CC5)cccc4c23)C(=O)N1]

### Synthetic Route:

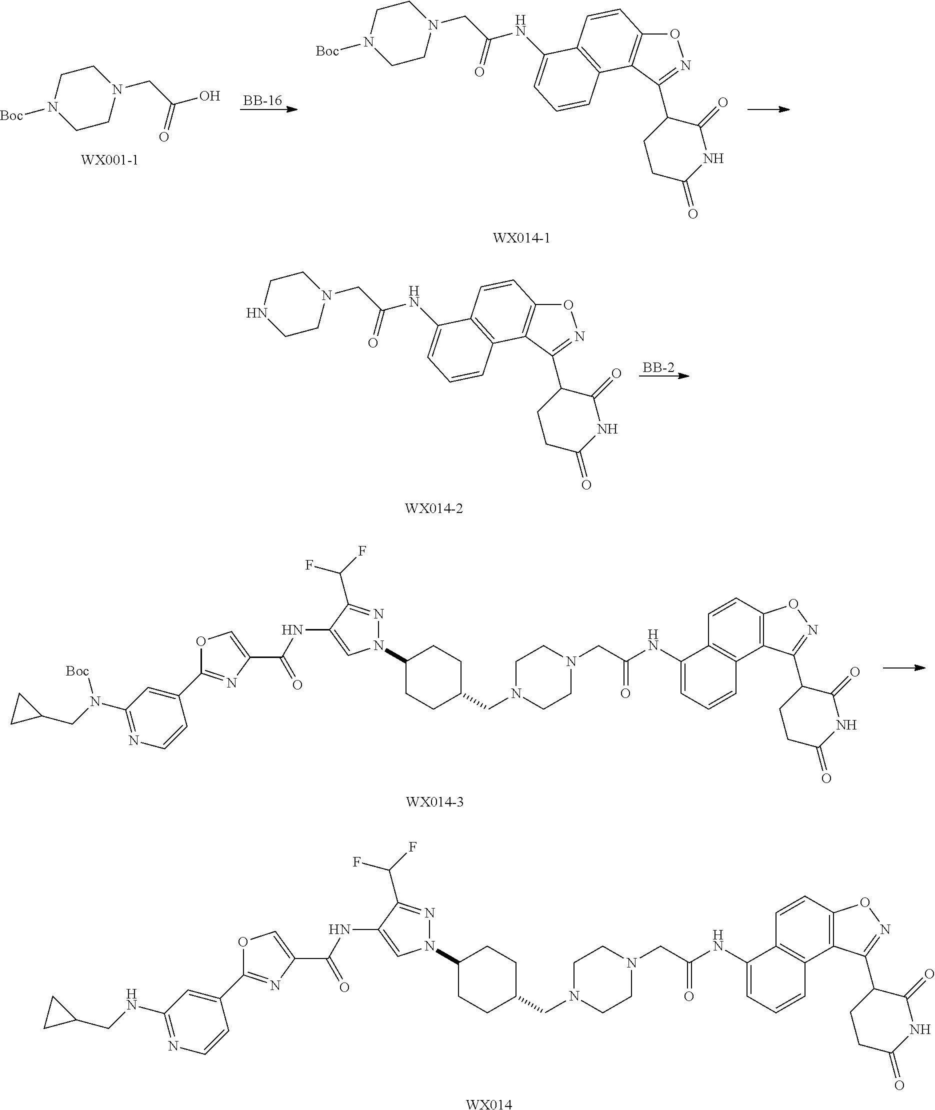

### Step 1: Synthesis of Compound WX014-1

Compound WX001-1 (93.09 mg, 331.57 μmol) was dissolved in N, N-dimethylformamide (2 mL) at room temperature under nitrogen atmosphere. N, N-Diisopropylethylamine (194.78 mg, 1.51 mmol) and 2-(7-azabenzotriazol)-N, N, N′, N′-tetramethyluronium hexafluorophosphate (171.92 mg, 452.14 μmol) were added thereto. The reaction mixture was stirred and reacted at room temperature for 0.5 hours. The hydrochloride of compound BB-16 (0.1 g, 301.43 μmol) was added thereto. The reaction mixture was stirred and reacted at room temperature for 11.5 hours. After the reaction was completed, the mixture was added with water (20 mL), and extracted with ethyl acetate (3×20 mL). The organic phases were combined, washed with 10% brine (2×30 mL), dried over anhydrous sodium sulfate, and filtered. The filtrate was concentrated under reduced pressure to remove the solvent. The resulting residue was separated by column chromatography (eluent: petroleum ether/ethyl acetate=3/1 to 1/1, v/v) to obtain compound WX014-1. MS-ESI m/z: 522.3 [M+H]⁺.

### Step 2: Synthesis of Hydrochloride of Compound WX014-2

Compound WX014-1 (0.1 g, 191.73 μmol) was dissolved in dichloromethane (1 mL) at room temperature, and a solution of hydrochloric acid in ethyl acetate (4 M, 2 mL) was added thereto. The reaction mixture was stirred and reacted at room temperature for 12 hours. After the reaction was completed, the mixture was concentrated under reduced pressure to remove the solvent to obtain the hydrochloride of compound WX014-2. MS-ESI m/z: 422.2 [M+H]⁺. ¹H NMR (400 MHz, DMSO_d₆) δ: 11.15 (s, 1H), 10.75 (s, 1H), 9.58 (s, 1H), 8.36 (d, J=9.2 Hz, 1H), 8.16 (d, J=7.2 Hz, 1H), 8.00 (d, J=9.6 Hz, 1H), 7.76 (t, J=7.8 Hz, 1H), 7.71 (d, J=6.8 Hz, 1H), 5.10 (dd, J=4.6, 11.4 Hz, 1H), 4.35-4.10 (m, 2H), 3.41-3.34 (m, 6H), 2.92-2.80 (m, 2H), 2.70-2.66 (m, 1H), 2.65-2.61 (m, 1H), 2.60-2.56 (m, 1H), 2.44-2.36 (m, 1H).

### Step 3: Synthesis of Compound WX014-3

Compound BB-2 (0.1 g, 171.05 μmol) and the hydrochloride of compound WX014-2 (78.33 mg, 171.05 μmol) were dissolved in tetrahydrofuran (2 mL) and N, N-dimethylformamide (0.4 mL) at room temperature under nitrogen atmosphere, and potassium acetate (50.36 mg, 513.16 μmol) and acetic acid (5.14 mg, 85.53 μmol) were added thereto. The reaction mixture was stirred and reacted at room temperature for 2 hours. Sodium triacetoxyborohydride (145.01 mg, 684.21 μmol) was added thereto. The mixture was further stirred and reacted for 10 hours. After the reaction was completed, the mixture was added with a saturated solution of ammonium chloride (20 mL), and extracted with ethyl acetate (3×20 mL). The organic phases were combined, washed with 10% brine (2×30 mL), dried over anhydrous sodium sulfate, and filtered. The filtrate was concentrated under reduced pressure to remove the solvent to obtain compound WX014-3. MS-ESI m/z: 990.6 [M+H]⁺.

### Step 4: Synthesis of Hydrochloride of Compound WX014

Compound WX014-3 (0.2 g, 202.01 μmol) was dissolved in dichloromethane (7 mL) and trifluoroacetic acid (7 mL) at room temperature under nitrogen atmosphere. The reaction mixture was stirred and reacted at room temperature for 2 hours. After the reaction was completed, the mixture was concentrated under reduced pressure to remove the solvent. The resulting residue was separated by preparative high performance liquid chromatography (chromatographic column: Phenomenex Luna C18 80*40 mm*3 m; mobile phase: water (0.04% hydrochloric acid)-acetonitrile; acetonitrile %: 12% to 32%, 7 minutes) to obtain the hydrochloride of the target compound WX014. MS-ESI m/z: 890.5 [M+H]⁺. ¹H NMR (400 MHz, MeOD_d₄) δ: 8.79 (s, 1H), 8.29 (d, J=9.6 Hz, 1H), 8.26 (s, 1H), 8.19 (dd, J=1.2 Hz, 6.8 Hz, 1H), 7.97 (d, J=6.8 Hz, 1H), 7.86 (d, J=9.2 Hz, 1H), 7.79-7.69 (m, 3H), 7.45 (dd, J=1.2 Hz, 6.8 Hz, 1H), 6.94 (t, J=54.6 Hz, 1H), 5.00 (dd, J=5.0 Hz, 10.2 Hz, 1H), 4.30-4.23 (m, 1H), 4.21 (s, 2H), 3.86-3.54 (m, 8H), 3.20 (d, J=6.4 Hz, 2H), 2.94-2.63 (m, 4H), 2.58-2.47 (m, 1H), 2.22 (d, J=11.6 Hz, 2H), 2.11 (d, J=12.0 Hz, 2H), 2.05-1.88 (m, 3H), 1.42-1.30 (m, 2H), 1.27-1.16 (m, 1H), 0.75-0.68 (m, 2H), 0.45-0.38 (m, 2H).

### Example 15

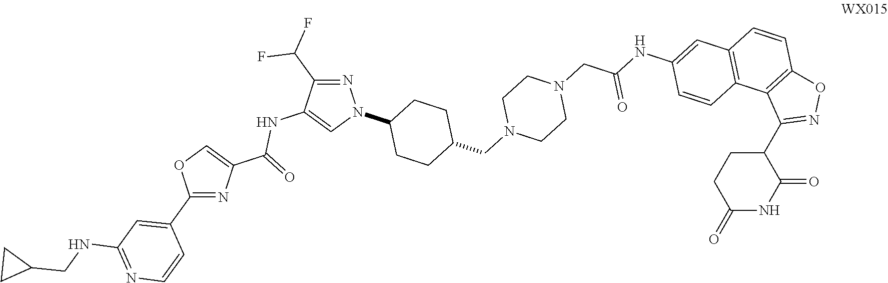

### Synthetic Route:

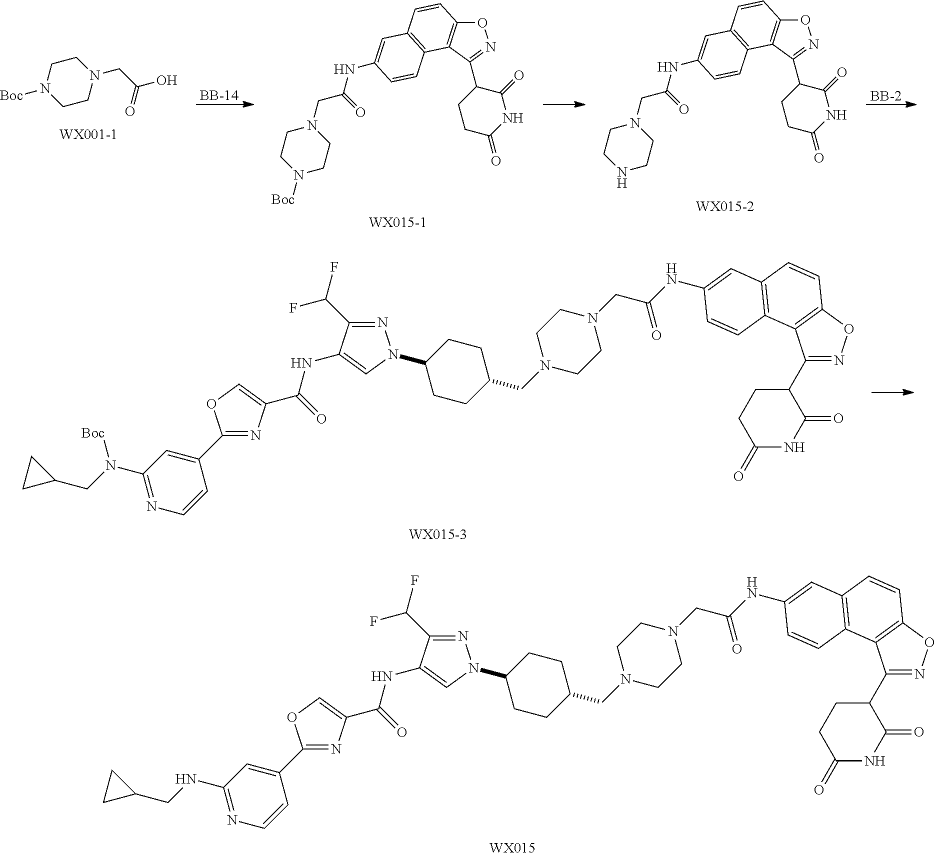

### Step 1: Synthesis of Compound WX015-1

Compound WX001-1 (93.09 mg, 331.57 μmol) was dissolved in N, N-dimethylformamide (2 mL) at room temperature under nitrogen atmosphere. N, N-diisopropylethylamine (194.78 mg, 1.51 mmol) and 2-(7-azabenzotriazol)-N, N, N′, N′-tetramethyluronium hexafluorophosphate (171.92 mg, 452.14 μmol) were added thereto. The reaction mixture was stirred and reacted at room temperature for 0.5 hours. The hydrochloride of compound BB-14 (0.1 g, 301.43 μmol) was added thereto. The reaction mixture was further reacted at room temperature for 1.5 hours. After the reaction was completed, the mixture was added with water (20 mL), and extracted with ethyl acetate (4×20 mL). The organic phases were combined, washed with 10% brine (2×30 mL), dried over anhydrous sodium sulfate, and filtered. The filtrate was concentrated under reduced pressure to remove the solvent to obtain compound WX015-1. MS-ESI m/z: 522.3 [M+H]⁺.

### Step 2: Synthesis of Hydrochloride of Compound WX015-2

Compound WX015-1 (155 mg, 297.18 μmol) was dissolved in dichloromethane (3 mL) at room temperature. A solution of hydrochloric acid in ethyl acetate (4 M, 5 mL) was added thereto. The reaction mixture was stirred and reacted at room temperature for 12 hours, then heated to 40° C., and stirred and reacted for 2 hours. After the reaction was completed, the mixture was concentrated under reduced pressure to remove the solvent to obtain the hydrochloride of compound WX015-2. MS-ESI m/z: 422.2 [M+H]⁺.

### Step 3: Synthesis of Compound WX015-3

Compound BB-2 (0.1 g, 171.05 μmol) and the hydrochloride of compound WX015-2 (78.33 mg, 171.05 μmol) were dissolved in N, N-dimethylformamide (3 mL) at room temperature under nitrogen atmosphere. Potassium acetate (50.36 mg, 513.15 μmol) and acetic acid (5.14 mg, 85.53 μmol) were added thereto. The reaction mixture was stirred and reacted at room temperature for 2 hours. Sodium triacetoxyborohydride (145.01 mg, 684.20 mol) was added thereto. The reaction mixture was further reacted for 10 hours. After the reaction was completed, the mixture was added with a saturated solution of ammonium chloride (20 mL), and extracted with ethyl acetate (4×20 mL). The organic phases were combined, washed with 10% brine (2×30 mL), dried over anhydrous sodium sulfate, and filtered. The filtrate was concentrated under reduced pressure to remove the solvent to obtain compound WX015-3. MS-ESI m/z: 990.6 [M+H]⁺.

### Step 4: Synthesis of Hydrochloride of Compound WX015

Compound WX015-3 (160 mg, 161.61 μmol) was dissolved in dichloromethane (6 mL) and trifluoroacetic acid (6 mL) at room temperature. The reaction mixture was stirred and reacted at room temperature for 2 hours. After the reaction was completed, the mixture was concentrated under reduced pressure to remove the solvent. The resulting residue was separated by preparative high performance liquid chromatography (chromatographic column: Phenomenex Luna C18 80*40 mm*3 m; mobile phase: water (0.04% hydrochloric acid)-acetonitrile; acetonitrile %: 14% to 34%, 7 minutes) to obtain the hydrochloric acid of the target compound WX015. MS-ESI m/z: 890.4 [M+H]⁺. ¹H NMR (400 MHz, MeOD_d₄) δ: 8.79 (s, 1H), 8.42 (d, J=2.0 Hz, 1H), 8.26 (s, 1H), 8.19 (d, J=8.8 Hz, 1H), 8.01 (d, J=9.2 Hz, 1H), 7.96 (d, J=6.8 Hz, 1H), 7.88 (dd, J=2.0 Hz, 8.8 Hz, 1H), 7.77 (d, J=9.2 Hz, 1H), 7.69 (s, 1H), 7.43 (dd, J=1.6 Hz, 6.8 Hz, 1H), 6.93 (t, J=54.4 Hz, 1H), 4.95 (dd, J=5.0 Hz, 10.2 Hz, 1H), 4.32-4.19 (m, 1H), 4.05 (s, 2H), 3.88-3.49 (m, 8H), 3.29 (s, 1H), 3.21 (d, J=6.4 Hz, 2H), 2.95-2.74 (m, 2H), 2.73-2.61 (m, 1H), 2.58-2.45 (m, 1H), 2.22 (d, J=10.4 Hz, 2H), 2.12 (d, J=12.8 Hz, 2H), 2.07-1.70 (m, 4H), 1.45-1.30 (m, 2H), 1.29-1.17 (m, 1H), 0.75-0.67 (m, 2H), 0.44-0.37 (m, 2H).

### Example 16

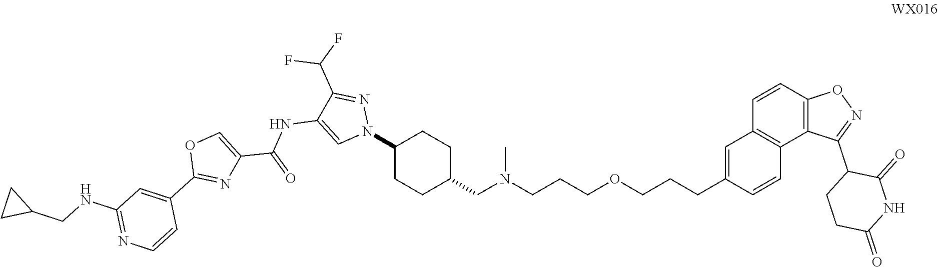 [SMILES: CN(CCCOCCCc1ccc2c(ccc3onc(C4CCC(=O)NC4=O)c32)c1)C[C@H]1CC[C@H](n2cc(NC(=O)c3coc(-c4ccnc(NCC5CC5)c4)n3)c(C(F)F)n2)CC1]

### Synthetic Route:

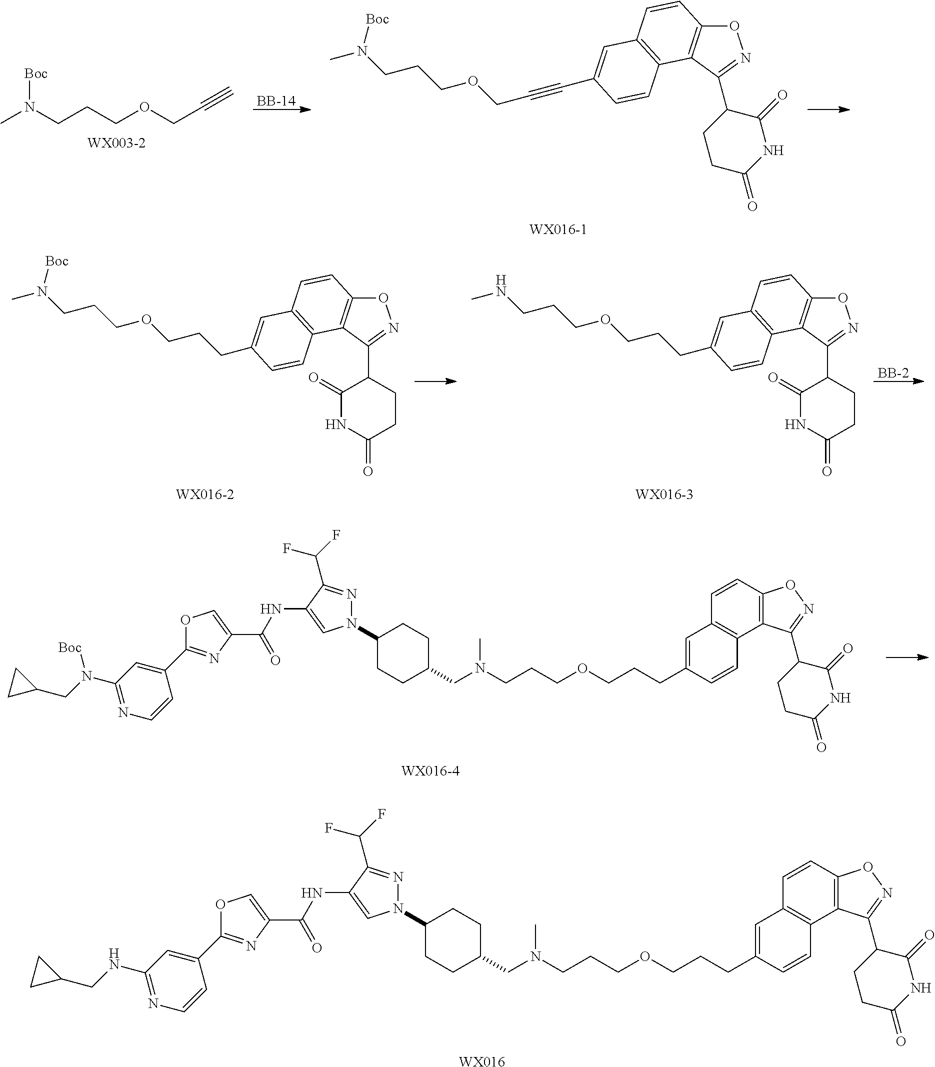

### Step 1: Synthesis of Compound WX016-1

Compound BB-15 (0.2 g, 556.83 μmol) and compound WX003-2 (164.54 mg, 723.88 mol) were dissolved in dimethyl sulfoxide (5 mL) at room temperature under nitrogen atmosphere. Bis(triphenylphosphine)palladium(II) chloride (78.17 mg, 111.37 μmol), cuprous iodide (21.21 mg, 111.37 μmol), and N, N-diisopropylethylamine (143.93 mg, 1.11 mmol) were added thereto. The reaction mixture was heated to 85° C., and stirred and reacted for 2 hours. After the reaction was completed, the mixture was cooled to room temperature, added with water (30 mL), and extracted with ethyl acetate (3×30 mL). The organic phases were combined, washed with 10% brine (3×40 mL), dried over anhydrous sodium sulfate, and filtered. The filtrate was concentrated under reduced pressure to remove the solvent. The resulting residue was separated by column chromatography (eluent: petroleum ether/ethyl acetate=3/1 to 1/1, v/v) to obtain compound WX016-1. MS-ESI m/z: 406.2 [M+H−100]⁺. ¹H NMR (400 MHz, CDCl₃) δ: 8.18 (s, 1H), 8.13 (d, J=1.2 Hz, 1H), 7.97 (t, J=9.2 Hz, 2H), 7.75 (d, J=8.8 Hz, 1H), 7.71 (dd, J=1.6 Hz, 8.4 Hz, 1H), 4.72 (dd, J=5.2 Hz, 8.4 Hz, 1H), 4.43 (s, 2H), 3.65 (t, J=6.4 Hz, 2H), 3.35 (t, J=6.8 Hz, 2H), 2.89 (s, 3H), 2.84-2.68 (m, 3H), 2.65-2.53 (m, 1H), 1.93-1.85 (m, 2H), 1.47 (s, 9H).

### Step 2: Synthesis of Compound WX016-2

Wet palladium on carbon (20 mg, purity of 10%) and palladium hydroxide/carbon (20 mg, purity of 20%) were dissolved in tetrahydrofuran (3 mL) at room temperature. Compound WX016-1 (0.1 g, 197.80 μmol) and triethylamine (20.02 mg, 197.80 μmol) were added thereto. The reaction mixture was stirred and reacted at room temperature under hydrogen (15 psi) atmosphere for 12 hours. After the reaction was completed, the mixture was filtered. The filtrate was concentrated under reduced pressure to remove the solvent. The resulting residue was purified by a chromatography plate (developing solvent: petroleum ether/ethyl acetate=1/1) to obtain compound WX016-2. MS-ESI m/z: 410.2 [M+H−100]⁺. ¹H NMR (400 MHz, CDCl₃) δ: 8.20 (s, 1H), 7.96 (d, J=2.4 Hz, 1H), 7.94 (d, J=2.4 Hz, 1H), 7.82 (s, 1H), 7.70 (d, J=8.8 Hz, 1H), 7.55 (dd, J=1.6 Hz, 8.4 Hz, 1H), 4.75 (dd, J=4.8 Hz, 8.4 Hz, 1H), 3.51-3.41 (m, 4H), 3.36-3.26 (m, 2H), 2.96-2.88 (m, 3H), 2.87 (s, 3H), 2.83-2.66 (m, 2H), 2.63-2.51 (m, 1H), 2.04-1.94 (m, 2H), 1.87-1.77 (m, 2H), 1.46 (s, 9H).

Step 3: Synthesis of trifluoroacetate of compound WX016-3

Compound WX016-2 (70 mg, 137.36 μmol) was dissolved in dichloromethane (3 mL) and trifluoroacetic acid (0.5 mL) at room temperature. The reaction mixture was stirred and reacted at room temperature for 40 minutes. After the reaction was completed, the mixture was concentrated under reduced pressure to remove the solvent to obtain the trifluoroacetate of compound WX016-3. MS-ESI m/z: 410.2 [M+H]⁺.

### Step 4: Synthesis of Compound WX016-4

Compound BB-2 (70 mg, 119.74 μmol) and the trifluoroacetate of compound WX016-3 (68.95 mg, 131.71 μmol) were dissolved in N, N-dimethylformamide (4 mL) at room temperature under nitrogen atmosphere. Potassium acetate (35.25 mg, 359.21 μmol) and acetic acid (3.60 mg, 59.87 μmol) were added thereto. The reaction mixture was stirred and reacted at room temperature for 2 hours. Sodium triacetoxyborohydride (101.51 mg, 478.95 mol, 4 eq) was added thereto. The reaction mixture was further stirred and reacted for 10 hours. After the reaction was completed, the mixture was added with a saturated solution of ammonium chloride (20 mL), and extracted with ethyl acetate (2×20 mL). The organic phases were combined, washed with 10% brine (2×30 mL), dried over anhydrous sodium sulfate, and filtered. The filtrate was concentrated under reduced pressure to remove the solvent to obtain compound WX016-4. MS-ESI m/z: 978.4 [M+H]⁺.

### Step 5: Synthesis of Hydrochloride of Compound WX016

Compound WX016-4 (110 mg, 112.46 μmol) was dissolved in dichloromethane (4 mL) and trifluoroacetic acid (4 mL) at room temperature under nitrogen atmosphere. The reaction mixture was stirred and reacted at room temperature for 2 hours. After the reaction was completed, the mixture was concentrated under reduced pressure to remove the solvent. The resulting residue was separated by preparative high performance liquid chromatography (chromatographic column: Phenomenex Luna C18 80×40 mm×3 m; mobile phase: water (0.04% hydrochloric acid)-acetonitrile; acetonitrile %: 13% to 30%, 7 minutes) to obtain the hydrochloride of the target compound WX016. MS-ESI m/z: 878.5 [M+H]⁺. ¹H NMR (400 MHz, MeOD_d₄) δ: 8.77 (s, 1H), 8.13 (d, J=8.0 Hz, 1H), 8.11 (s, 1H), 8.02 (d, J=9.2 Hz, 1H), 7.99-7.95 (m, 1H), 7.89 (s, 1H), 7.75-7.68 (m, 2H), 7.61 (d, J=8.0 Hz, 1H), 7.46-7.42 (m, 1H), 6.89 (t, J=54.4 Hz, 1H), 4.93 (dd, J=5.2 Hz, 10.4 Hz, 1H), 4.21-4.09 (m, 1H), 3.62-3.53 (m, 3H), 3.17-3.02 (m, 2H), 2.93 (t, J=7.4 Hz, 3H), 2.86 (s, 3H), 2.85-2.72 (m, 2H), 2.70-2.58 (m, 1H), 2.57-2.43 (m, 1H), 2.23-1.76 (m, 13H), 1.34-1.14 (m, 4H), 0.74-0.68 (m, 2H), 0.44-0.39 (m, 2H).

### Example 17

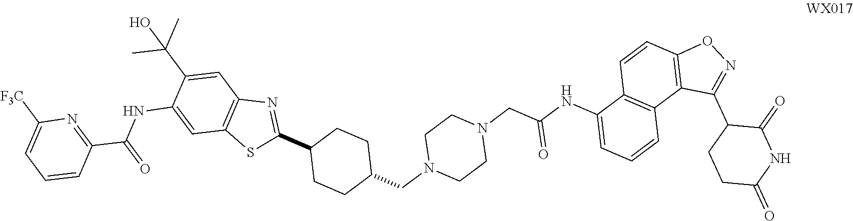

### Synthetic Route:

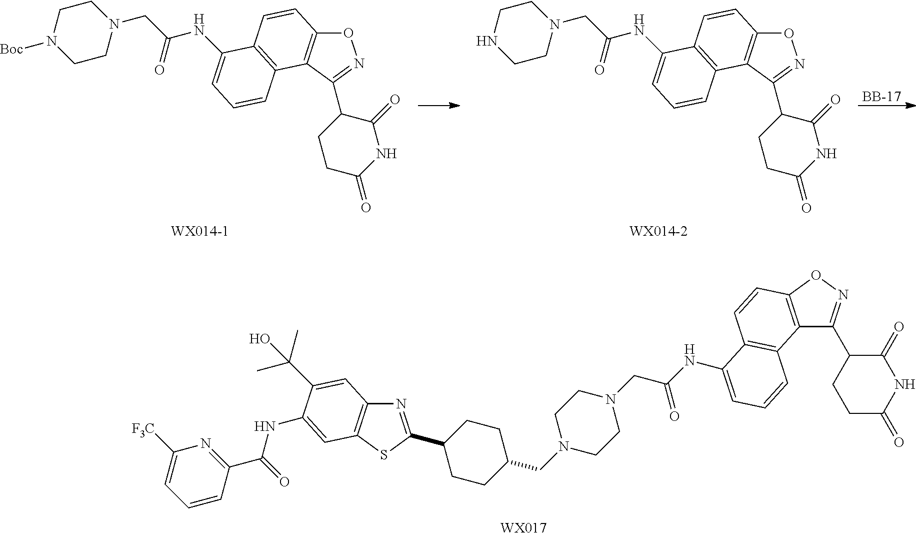

### Step 1: Synthesis of Trifluoroacetate of Compound WX014-2

Compound WX014-1 (100 mg, 191.73 μmol) was dissolved in dichloromethane (1 mL) at room temperature under nitrogen atmosphere, then trifluoroacetic acid (308.00 mg, 2.70 mmol, 0.2 mL) was added thereto. The reaction mixture was stirred and reacted at room temperature for 12 hours. After the reaction was completed, the reaction mixture was concentrated under reduced pressure to obtain the trifluoroacetate of compound WX014-2. MS-ESI m/z: 422.1 [M+H]⁺.

Step 2: Synthesis of formate of compound WX017

The trifluoroacetate of compound WX014-2 (100 mg, 186.75 μmol) was dissolved in acetonitrile (2 mL) at room temperature under nitrogen atmosphere, then N, N-diisopropylethylamine (120.68 mg, 933.76 μmol, 162.64 μL) and compound BB-17 (112 mg, 167.01 μmol, purity of 89.98%) were added thereto. The reaction mixture was heated to 90° C., and stirred and reacted for 12 hours. After the reaction was completed, the reaction mixture was concentrated under reduced pressure to remove the solvent. The residue was separated by preparative high performance liquid chromatography (chromatographic column: Phenomenex Luna C18 75*30 mm*3 m; mobile phase: water (0.2% formic acid)-acetonitrile; acetonitrile %: 30% to 60%, 8 minutes) to obtain the formate of the target compound WX017. MS-ESI m/z: 897.0 [M+H]⁺. ¹H NMR (400 MHz, DMSO_d₆) δ: 12.55 (s, 1H), 11.14 (s, 1H), 10.13 (s, 1H), 9.07 (s, 1H), 8.46 (t, J=7.6 Hz, 1H), 8.38 (t, J=8.0 Hz, 1H), 8.23-8.16 (m, 2H), 8.15-8.08 (m, 1H), 8.01 (d, J=9.2 Hz, 1H), 7.89 (s, 1H), 7.79 (d, J=7.6 Hz, 1H), 7.72 (t, J=8.0 Hz, 1H), 6.08 (s, 1H), 5.09 (dd, J=4.8 Hz, 11.6 Hz, 1H), 3.30-3.23 (m, 2H), 3.13-3.02 (m, 1H), 2.92-2.79 (m, 1H), 2.78-2.51 (m, 10H), 2.45-2.34 (m, 2H), 2.32-2.25 (m, 1H), 2.19 (d, J=10.8 Hz, 2H), 1.94 (d, J=10.8 Hz, 2H), 1.70-1.55 (m, 9H), 1.18-1.02 (m, 2H).

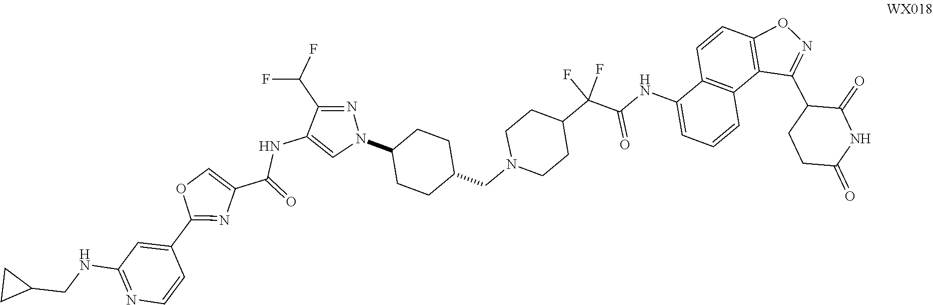

### Synthetic Route:

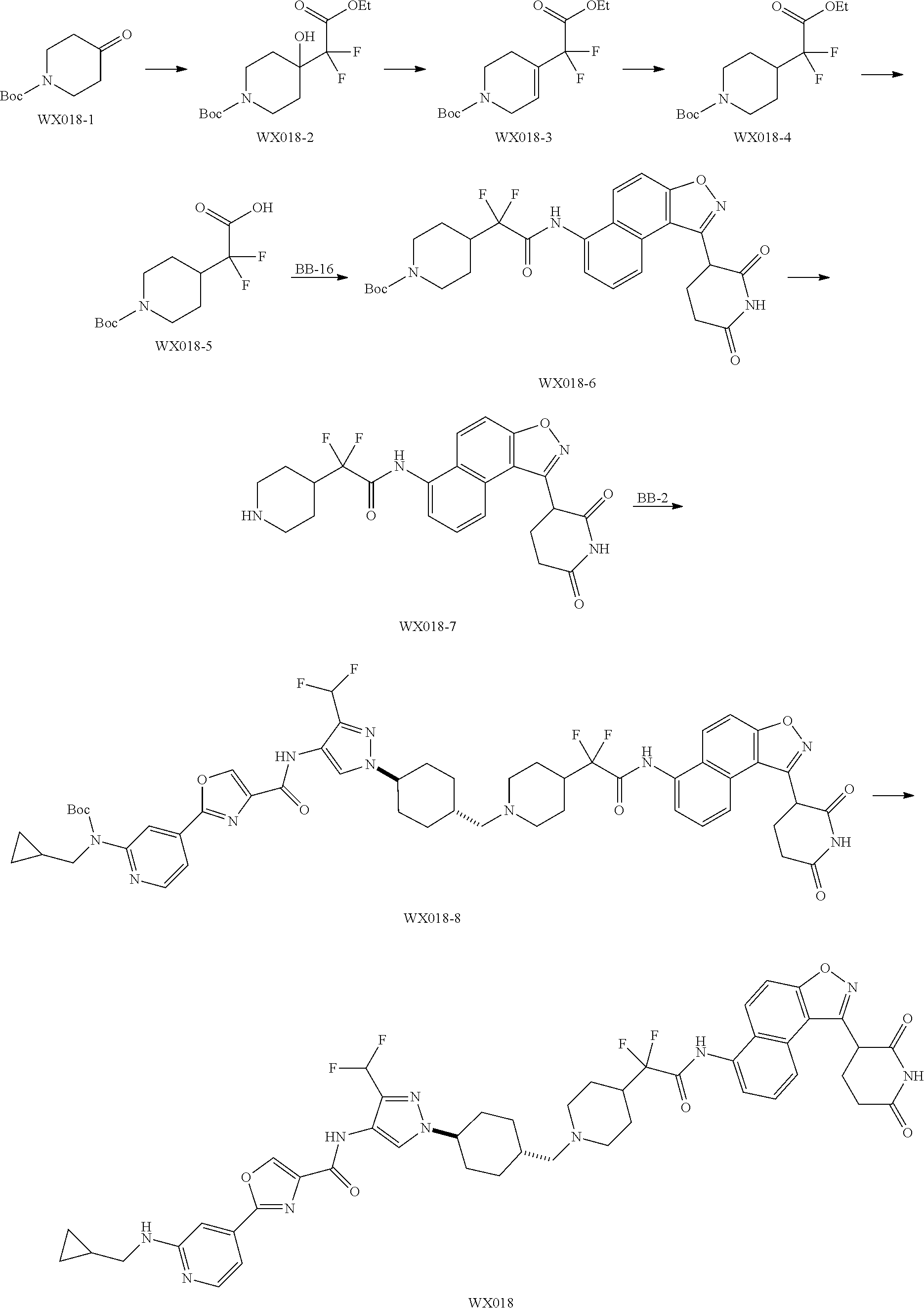

### Step 1: Synthesis of Compound WX018-2

Titanocene dichloride (1.30 g, 5.02 mmol) and zinc powder (13.31 g, 203.55 mmol) were added to a dried reaction flask at room temperature under nitrogen atmosphere. Tetrahydrofuran (100 mL) was added for dissolution. A solution of ethyl bromodifluoroacetate (20.37 g, 100.38 mmol, 12.90 mL) in tetrahydrofuran (20 mL) was added dropwise thereto (first adding 1/10 of the solution, after the reaction was initiated (with a noticeable increase in temperature), then adding the remaining solution dropwise). After the dropwise addition was completed, the reaction mixture was further stirred for 0.5 hours, and filtered. The filtrate was added to a solution of compound WX018-1 (10 g, 50.19 mmol) in tetrahydrofuran (50 mL), and stirred and reacted for 12 hours. After the reaction was completed, the mixture was added with 1 M hydrochloric acid to adjust the pH to 3, then added with water (100 mL), and extracted with ethyl acetate (100 mL×3). The organic phase was washed with saturated brine (50 mL×3), dried over anhydrous sodium sulfate, and filtered. The filtrate was concentrated under reduced pressure. The resulting residue was subjected to column chromatography (eluent: petroleum ether/ethyl acetate=0/1 to I/O, v/v) to obtain compound WX018-2. MS-ESI m/z: 268.0 [M+H−56]⁺. ¹H NMR (400 MHz, CDCl₃) δ: 4.37 (q, J=7.2 Hz, 2H), 4.20-3.92 (m, 2H), 3.17-2.94 (m, 2H), 2.33 (s, 1H), 1.87-1.74 (m, 2H), 1.72-1.65 (m, 2H), 1.47 (s, 9H), 1.38 (t, J=7.2 Hz, 3H).

### Step 2: Synthesis of Compound WX018-3

Compound WX018-2 (1 g, 3.09 mmol), thionyl chloride (2.21 g, 18.56 mmol), and pyridine (1.47 g, 18.56 mmol) were added to dioxane (15 mL) at room temperature under nitrogen atmosphere. The reaction mixture was stirred at room temperature for 1.5 hours, then 4-dimethylaminopyridine (37.78 mg, 309.28 μmol) was added thereto. The reaction mixture was further stirred and reacted for 12 hours. After the reaction was completed, the reaction mixture was added with water (20 mL), and extracted with ethyl acetate (30 mL×3). The organic phase was washed with saturated brine (20 mL×3), dried over anhydrous sodium sulfate, and filtered. The filtrate was concentrated under reduced pressure to remove the solvent. The resulting residue was subjected to column chromatography (eluent: petroleum ether/ethyl acetate=20/1 to 9/1, v/v) to obtain compound WX018-3. MS-ESI m/z: 206.2 [M+H−100]⁺. ¹H NMR (400 MHz, CDCl₃) δ: 6.18 (s, 1H), 4.34 (q, J=7.0 Hz, 2H), 4.02 (d, J=2.0 Hz, 2H), 3.54 (t, J=5.6 Hz, 2H), 2.25 (s, 2H), 1.48 (s, 9H), 1.36 (t, J=7.2 Hz, 3H)

### Step 3: Synthesis of Compound WX018-4

Wet palladium hydroxide (0.5 g, 712.07 μmol, purity of 20%) was added to a hydrogenation flask at room temperature under argon atmosphere, then methanol (20 mL) and compound WX018-3 (1.5 g, 4.91 mmol) were added thereto. The atmosphere was replaced with hydrogen three times, and the reaction mixture was stirred and reacted at room temperature and at 40 psi for 12 hours. After the reaction was completed, the mixture was filtered. The filter cake was rinsed with methanol (50 mL×4). The filtrate was collected and concentrated under reduced pressure to remove the remaining solvent. The resulting residue was subjected to column chromatography (eluent: petroleum ether/ethyl acetate=20/1 to 10/1, v/v) to obtain compound WX018-4. MS-ESI m/z: 252.2 [M+H−56]⁺. 1H NMR (400 MHz, CDCl₃) δ: 4.35 (q, J=7.0 Hz, 2H), 4.30-4.15 (m, 2H), 2.68 (t, J=12.8 Hz, 2H), 2.32-2.14 (m, 1H), 1.73 (d, J=12.8 Hz, 2H), 1.49 (d, J=4.8 Hz, 1H), 1.47 (s, 9H), 1.43 (d, J=4.4 Hz, 1H), 1.37 (t, J=7.0 Hz, 3H).

### Step 4: Synthesis of Compound WX018-5

Compound WX018-4 (0.26 g, 845.99 μmol) was dissolved in a mixed solvent of ethanol (5 mL) and water (2 mL) at room temperature under nitrogen atmosphere, then lithium hydroxide monohydrate (71.00 mg, 1.69 mmol) was added thereto. The reaction mixture was stirred and reacted at room temperature for 12 hours. After the reaction was completed, the mixture was concentrated under reduced pressure. The residue was added with 1 M hydrochloric acid to adjust the pH to 2 to 3, resulting in the precipitation of solid. The mixture was filtered. The filter cake was collected, and dried under vacuum to obtain compound WX018-5. MS-ESI m/z: 224.0 [M+H−56]⁺. ¹H NMR (400 MHz, DMSO_d₆) δ: 4.05-3.94 (m, 2H), 2.80-2.63 (m, 2H), 2.37-2.21 (m, 1H), 1.71-1.61 (m, 2H), 1.39 (s, 9H), 1.27-1.12 (m, 2H).

### Step 5: Synthesis of Compound WX018-6

Compound WX018-5 (101.02 mg, 361.71 μmol) was dissolved in N, N-dimethylformamide (3 mL) at room temperature under nitrogen atmosphere, and 2-(7-azabenzotriazol)-N, N, N′, N′-tetramethyluronium hexafluorophosphate (229.22 mg, 602.86 mol) and N, N-diisopropylethylamine (233.74 mg, 1.81 mmol, 315.01 μL) were added thereto. The reaction mixture was stirred for 0.5 hours. The hydrochloride of compound BB-16 (0.1 g, 301.43 μmol) was added thereto. The reaction mixture was further stirred and reacted at room temperature for 12 hours. After the reaction was completed, the reaction mixture was added with water (20 mL), resulting in the precipitation of solid. The mixture was filtered. The solid was collected, and dried under vacuum to obtain compound WX018-6. MS-ESI m/z: 457.1 [M+H−100]⁺. ¹H NMR (400 MHz, DMSO_d₆) δ: 11.15 (s, 1H), 11.02 (s, 1H), 8.31-8.18 (m, 1H), 8.05 (q, J=9.2 Hz, 2H), 7.83-7.71 (m, 1H), 7.58 (d, J=7.6 Hz, 1H), 5.18-5.04 (m, 1H), 4.15-4.04 (m, 2H), 2.90-2.80 (m, 2H), 2.74-2.61 (m, 4H), 2.44-2.34 (m, 2H), 1.42 (s, 9H), 1.18-1.10 (m, 3H).

### Step 6: Synthesis of Hydrochloride of Compound WX018-7

Compound WX018-6 (110 mg, 197.64 μmol) was dissolved in ethyl acetate (2 mL) at room temperature. A solution of hydrochloric acid in ethyl acetate (3 mL, 4 M) was added thereto. The reaction mixture was stirred and reacted for 2 hours. After the reaction was completed, the mixture was filtered. The filter cake was rinsed with ethyl acetate (5 mL×3), collected, and dried under vacuum to obtain the hydrochloride of compound WX018-7. MS-ESI m/z: 457.2 [M+H]⁺.

### Step 7: Synthesis of Compound WX018-8

The hydrochloride of compound WX018-7 (97 mg, 196.79 μmol) was dissolved in tetrahydrofuran (5 mL) and N, N-dimethylformamide (1 mL) at room temperature under nitrogen atmosphere. Compound BB-2 (115.05 mg, 196.79 μmol), potassium acetate (57.94 mg, 590.38 μmol), and glacial acetic acid (5.91 mg, 98.40 μmol, 5.63 μL) were added thereto. The reaction mixture was stirred for 2 hours, then sodium triacetoxyborohydride (166.83 mg, 787.17 μmol) was added thereto. The reaction mixture was further stirred and reacted for 1 hour. After the reaction was completed, the reaction mixture was added with a saturated aqueous solution of ammonium chloride (20 mL), and extracted with ethyl acetate (30 mL×3). The organic phase was washed with saturated brine (30 mL×2), dried over anhydrous sodium sulfate, and filtered. The filtrate was concentrated under reduced pressure to remove the solvent to obtain compound WX018-8. MS-ESI m/z: 1025.4 [M+H]⁺.

### Step 8: Synthesis of Hydrochloride of Compound WX018

Compound WX018-8 (0.2 g, 195.11 μmol) was dissolved in ethyl acetate (2 mL) at room temperature. A solution of hydrochloric acid in ethyl acetate (4.98 mL, 4 M) was added thereto. The reaction mixture was stirred and reacted for 2 hours. After the reaction was completed, the mixture was filtered. The filter cake was rinsed with ethyl acetate (5 mL×3), and collected. The filter cake was separated by preparative high performance liquid chromatography (chromatographic column: Phenomenex Luna 80*30 mm*3 m; mobile phase: water (0.04% hydrochloric acid)-acetonitrile; acetonitrile %: 10% to 30%, 8 minutes) to obtain the hydrochloride of the target compound WX018. MS-ESI m/z: 925.5 [M+H]⁺. ¹H NMR (400 MHz, MeOD_d₄) δ: 8.80 (s, 1H), 8.30-8.23 (m, 2H), 8.15 (d, J=9.2 Hz, 1H), 7.97 (d, J=6.8 Hz, 1H), 7.89 (d, J=9.6 Hz, 1H), 7.78 (t, J=8.0 Hz, 1H), 7.72-7.68 (m, 1H), 7.68-7.61 (m, 1H), 7.45 (dd, J=1.6 Hz, 6.8 Hz, 1H), 6.94 (t, J=54.6 Hz, 1H), 5.01 (dd, J=5.0 Hz, 10.2 Hz, 1H), 4.32-4.19 (m, 1H), 3.79 (d, J=12.0 Hz, 2H), 3.30-3.27 (m, 1H), 3.24-3.00 (m, 4H), 2.97-2.63 (m, 4H), 2.59-2.46 (m, 1H), 2.34-2.15 (m, 4H), 2.14-1.90 (m, 7H), 1.42-1.27 (m, 3H), 1.26-1.17 (m, 1H), 0.75-0.66 (m, 2H), 0.45-0.36 (m, 2H).

### Example 19

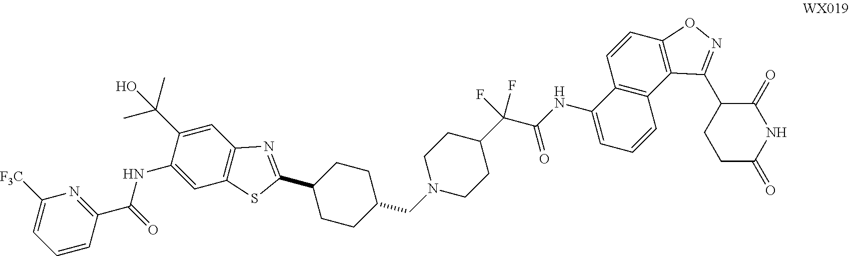 [SMILES: CC(C)(O)c1cc2nc([C@H]3CC[C@H](CN4CCC(C(F)(F)C(=O)Nc5cccc6c5ccc5onc(C7CCC(=O)NC7=O)c56)CC4)CC3)sc2cc1NC(=O)c1cccc(C(F)(F)F)n1]

### Synthetic Route:

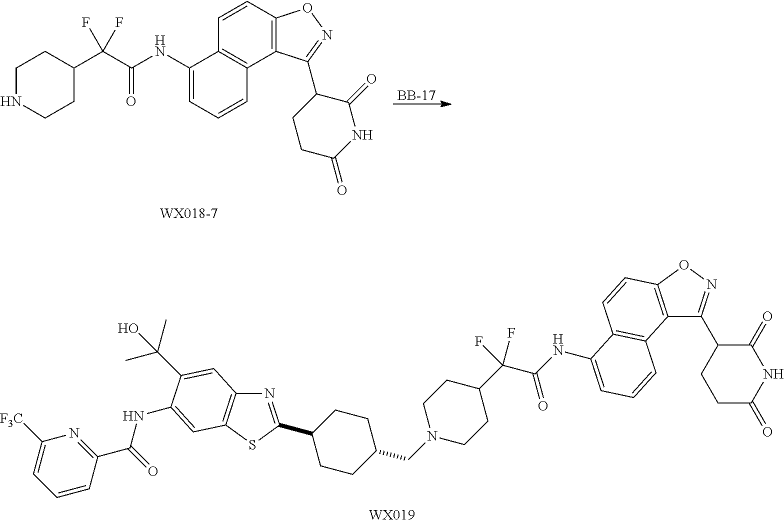

### Synthesis of Formate of Compound WX019

Compound BB-17 (0.1 g, 165.72 μmol) and the trifluoroacetate of compound WX018-7 (0.15 g, 262.94 μmol) were dissolved in acetonitrile (3 mL) at room temperature under nitrogen atmosphere, and N, N-diisopropylethylamine (214.18 mg, 1.66 mmol, 288.65 μL) was added thereto. The reaction mixture was heated to 90° C., and stirred and reacted for 14 hours. After the reaction was completed, the reaction mixture was concentrated under reduced pressure to remove the solvent. The resulting residue was separated by preparative high performance liquid chromatography (chromatographic column: Phenomenex Luna C18 200*40 mm*10 m; mobile phase: water (0.2% formic acid)-acetonitrile; acetonitrile %: 20% to 50%, 8 minutes) to obtain the formate of the target compound WX019. MS-ESI m/z: 932.3 [M+H]⁺. ¹H NMR (400 MHz, DMSO_d₆) δ: 12.55 (s, 1H), 11.15 (s, 1H), 10.99 (s, 1H), 9.07 (s, 1H), 8.47 (d, J=8.0 Hz, 1H), 8.38 (t, J=7.8 Hz, 1H), 8.27-8.22 (m, 1H), 8.19 (d, J=8.8 Hz, 1H), 8.06 (q, J=9.6 Hz, 2H), 7.89 (s, 1H), 7.77 (t, J=8.0 Hz, 1H), 7.58 (d, J=7.2 Hz, 1H), 6.07 (s, 1H), 5.11 (dd, J=4.2 Hz, 11.4 Hz, 1H), 3.13-2.96 (m, 3H), 2.92-2.80 (m, 1H), 2.71-2.57 (m, 2H), 2.48-2.24 (m, 3H), 2.22-2.12 (m, 4H), 1.99-1.88 (m, 4H), 1.86-1.77 (m, 2H), 1.63 (s, 6H), 1.62-1.51 (m, 4H), 1.18-1.01 (m, 2H).

### Example 20

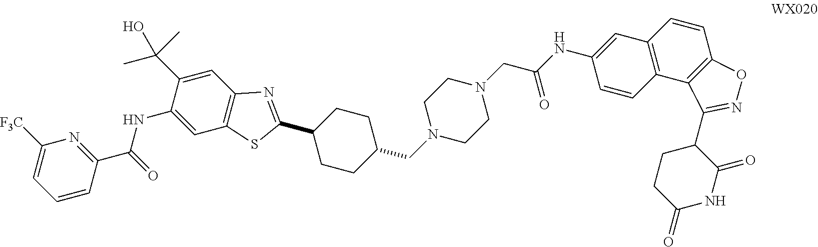 [SMILES: CC(C)(O)c1cc2nc([C@H]3CC[C@H](CN4CCN(CC(=O)Nc5ccc6c(ccc7onc(C8CCC(=O)NC8=O)c76)c5)CC4)CC3)sc2cc1NC(=O)c1cccc(C(F)(F)F)n1]

### Synthetic Route:

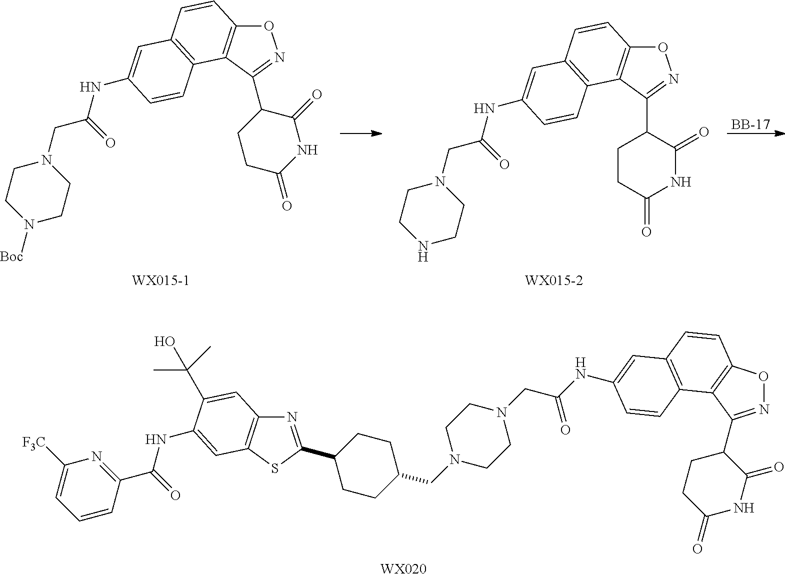

Step 1: Synthesis of trifluoroacetate of compound WX015-2

Compound WX015-1 (0.1 g, 191.73 μmol) was dissolved in dichloromethane (3 mL) at room temperature under nitrogen atmosphere, and trifluoroacetic acid (459.10 mg, 4.03 mmol, 298.11 μL) was added thereto. The reaction mixture was stirred and reacted at room temperature for 4 hours. After the reaction was completed, the reaction mixture was concentrated under reduced pressure to obtain the trifluoroacetate of compound WX015-2. MS-ESI m/z: 422.1 [M+H]⁺.

Step 2: Synthesis of formate of compound WX020

Compound BB-17 (0.075 g, 124.29 μmol) and the trifluoroacetate of compound WX015-2 (0.125 g, 185.93 μmol) were dissolved in acetonitrile (3 mL) at room temperature under nitrogen atmosphere, and N, N-diisopropylethylamine (160.63 mg, 1.24 mmol, 216.49 μL) was added thereto. The reaction mixture was heated to 90° C., and stirred and reacted for 36 hours. After the reaction was completed, the reaction mixture was concentrated under reduced pressure to remove the solvent. The resulting residue was separated by preparative high performance liquid chromatography (chromatographic column: Phenomenex Luna C18 75*30 mm*3 m; mobile phase: water (0.2% formic acid)-acetonitrile; acetonitrile %: 10% to 45%, 8 minutes) to obtain the formate of the target compound WX020. MS-ESI m/z: 897.4 [M+H]⁺. ¹H NMR (400 MHz, DMSO_d₆) δ: 12.54 (s, 1H), 11.13 (s, 1H), 10.01 (s, 1H), 9.06 (s, 1H), 8.50 (d, J=2.0 Hz, 1H), 8.46 (d, J=7.6 Hz, 1H), 8.38 (t, J=7.8 Hz, 1H), 8.18 (d, J=7.6 Hz, 2H), 8.12 (d, J=9.2 Hz, 1H), 7.95-7.86 (m, 3H), 6.07 (s, 1H), 5.03 (dd, J=4.6 Hz, 11.4 Hz, 1H), 3.20 (s, 2H), 3.12-2.99 (m, 1H), 2.91-2.76 (m, 2H), 2.70-2.62 (m, 3H), 2.58-2.53 (m, 4H), 2.43-2.31 (m, 3H), 2.22-2.12 (m, 4H), 1.97-1.85 (m, 2H), 1.72-1.49 (m, 10H), 1.16-0.99 (m, 2H).

### Example 21

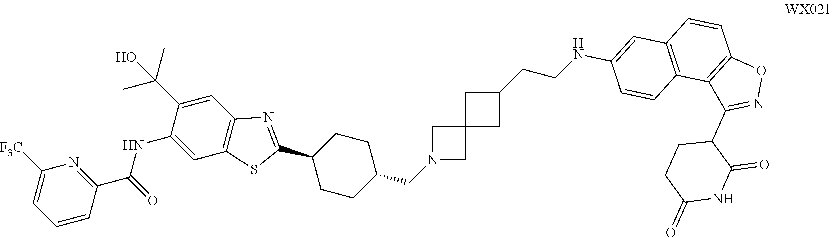 [SMILES: CC(C)(O)c1cc2nc([C@H]3CC[C@H](CN4CC5(CC(CCNc6ccc7c(ccc8onc(C9CCC(=O)NC9=O)c87)c6)C5)C4)CC3)sc2cc1NC(=O)c1cccc(C(F)(F)F)n1]

### Synthetic Route:

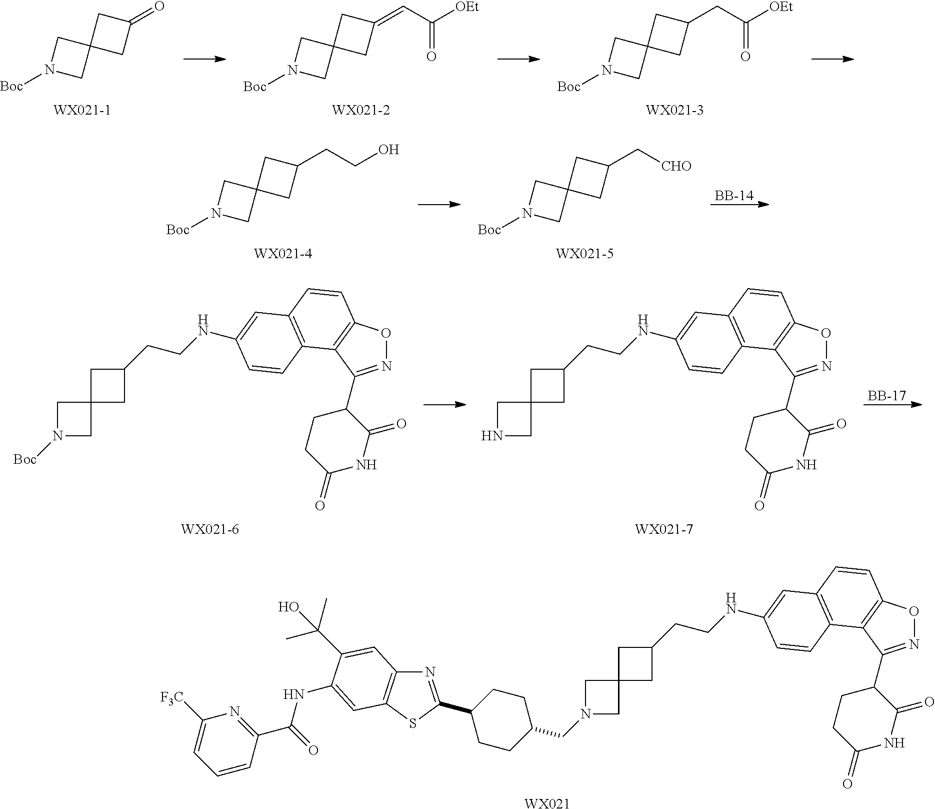

### Step 1: Synthesis of Compound WX021-2

Compound WX021-1 (30 g, 142.01 mmol) and (ethoxycarbonylmethylene)triphenylphosphorane (54.42 g, 156.21 mmol) were dissolved in toluene (450 mL) at room temperature under nitrogen atmosphere. The reaction mixture was heated to 80° C., and stirred and reacted for 12 hours. After the reaction was completed, the reaction mixture was cooled to room temperature and washed with water (3×200 mL). The organic phase was dried over anhydrous sodium sulfate, and filtered. The filtrate was concentrated under reduced pressure to obtain a yellow residue. The residue was separated by column chromatography (eluent: petroleum ether/ethyl acetate=1/0 to 4/1, v/v) to obtain compound WX021-2. MS-ESI m/z: 226.2 [M+H−56]⁺. ¹H NMR (400 MHz, CDCl₃) δ: 5.67-5.63 (m, 1H), 4.14 (q, J=7.2 Hz, 2H), 4.05-3.87 (m, 4H), 3.29 (d, J=2.4 Hz, 2H), 3.00 (s, 2H), 1.43 (s, 9H), 1.27 (t, J=7.2 Hz, 3H).

### Step 2: Synthesis of Compound WX021-3

Compound WX021-2 (37 g, 131.51 mmol) was dissolved in ethanol (400 mL) at room temperature under nitrogen atmosphere, then palladium on carbon (3.8 g, purity of 10%) was added thereto. The atmosphere was replaced with hydrogen three times, and the reaction mixture was stirred and reacted at room temperature and at 25 psi for 12 hours. After the reaction was completed, the mixture was filtered. The filter cake was rinsed with ethyl acetate (700 mL). The filtrate was concentrated under reduced pressure to obtain compound WX021-3. MS-ESI m/z: 228.2 [M+H−56]⁺. ¹H NMR (400 MHz, CDCl₃) δ: 4.11 (q, J=7.0 Hz, 2H), 3.93 (s, 2H), 3.80 (s, 2H), 2.60-2.45 (m, 1H), 2.40-2.29 (m, 4H), 1.91-1.82 (m, 2H), 1.43 (s, 9H), 1.24 (t, J=7.0 Hz, 3H).

### Step 3: Synthesis of Compound WX021-4

Lithium borohydride (2.59 g, 118.90 mmol) was dissolved in tetrahydrofuran (100 mL) at room temperature under nitrogen atmosphere. A solution of compound WX021-3 (10 g, 35.29 mmol) in tetrahydrofuran (20 mL) was added thereto. The reaction mixture was heated to 30° C., and methanol (20 mL) was slowly added dropwise thereto. After the dropwise addition was completed, the reaction mixture was further stirred and reacted for 12 hours. After the reaction was completed, the reaction mixture was poured into water (300 L), and extracted with ethyl acetate (3×300 mL). The organic phase was washed with saturated brine (400 mL), dried over anhydrous sodium sulfate, and filtered. The filtrate was concentrated under reduced pressure to obtain compound WX021-4. ¹H NMR (400 MHz, CDCl₃) δ: 3.92 (s, 2H), 3.80 (s, 2H), 3.57 (t, J=6.6 Hz, 2H), 2.33-2.18 (m, 3H), 1.84-1.75 (m, 2H), 1.63 (q, J=6.8 Hz, 2H), 1.43 (s, 9H).

### Step 4: Synthesis of Compound WX021-5

Compound WX021-4 (5.3 g, 21.96 mmol) was dissolved in dichloromethane (70 mL) at room temperature under nitrogen atmosphere, then Dess-Martin periodinane (12.11 g, 28.55 mmol) was added thereto. The reaction mixture was stirred and reacted for 1.5 hours. After the reaction was completed, the reaction mixture was concentrated under reduced pressure. The residue was separated by column chromatography (eluent: petroleum ether/ethyl acetate=I/O to 4/1, v/v) to obtain compound WX021-5. ¹H NMR (400 MHz, CDCl₃) δ: 9.69 (s, 1H), 3.95 (s, 2H), 3.81 (s, 2H), 2.67-2.51 (m, 3H), 2.45-2.32 (m, 2H), 1.92-1.81 (m, 2H), 1.42 (s, 9H).

### Step 5: Synthesis of Compound WX021-6

Compound WX021-5 (0.5 g, 2.09 mmol) and the hydrochloride of compound BB-14 (0.6 g, 1.81 mmol) were dissolved in tetrahydrofuran (15 mL) at room temperature under nitrogen atmosphere, and potassium acetate (709.99 mg, 7.23 mmol) and acetic acid (217.22 mg, 3.62 mmol) were added thereto. The reaction mixture was stirred for 2 hours. Sodium triacetoxyborohydride (1.53 g, 7.23 mmol) was added thereto. The reaction mixture was further stirred for 1 hour. After the reaction was completed, the reaction mixture was added with water (20 mL) and extracted with ethyl acetate (20 mL×4). The organic phases were combined, washed with saturated brine (80 mL×3), dried over anhydrous sodium sulfate, and filtered. The filtrate was concentrated under reduced pressure to remove the solvent. The resulting residue was separated by column chromatography (eluent: petroleum ether/ethyl acetate=2/1 to 1/1, v/v) to obtain compound WX021-6. MS-ESI m/z: 419.2 [M+H−100]⁺. ¹H NMR (400 MHz, CDCl₃) δ: 8.18 (s, 1H), 7.80 (d, J=3.2 Hz, 1H), 7.77 (d, J=3.6 Hz, 1H), 7.61 (d, J=9.2 Hz, 1H), 7.03 (dd, J=2.4 Hz, 8.8 Hz, 1H), 7.00 (d, J=2.4 Hz, 1H), 4.69 (dd, J=5.2 Hz, 8.0 Hz, 1H), 3.95 (s, 2H), 3.83 (s, 2H), 3.16 (t, J=7.0 Hz, 2H), 2.94-2.84 (m, 1H), 2.80-2.63 (m, 2H), 2.58-2.48 (m, 1H), 2.40-2.25 (m, 3H), 1.91-1.83 (m, 2H), 1.77 (q, J=7.0 Hz, 2H), 1.44 (s, 9H).

Step 6: Synthesis of trifluoroacetate of compound WX021-7

Compound WX021-6 (0.18 g, 347.09 μmol) was dissolved in dichloromethane (6 mL) at room temperature under nitrogen atmosphere, and trifluoroacetic acid (893.20 mg, 7.83 mmol, 0.58 mL) was added thereto. The reaction mixture was stirred and reacted at room temperature for 3 hours. After the reaction was completed, the reaction mixture was concentrated under reduced pressure to remove the solvent to obtain the trifluoroacetate of compound WX021-7. MS-ESI m/z: 419.1 [M+H]⁺.

### Step 7: Synthesis of Compound WX021

Compound BB-17 (0.1 g, 165.72 μmol) and the trifluoroacetate of compound WX021-7 (125 mg, 174.84 μmol) was dissolved in acetonitrile (3 mL) at room temperature under nitrogen atmosphere, and N, N-diisopropylethylamine (257.01 mg, 1.99 mmol, 346.38 μL) was added thereto. The reaction mixture was heated to 90° C., and stirred and reacted for 30 hours. After the reaction was completed, the reaction mixture was concentrated under reduced pressure to remove the solvent. The resulting residue was first separated by preparative high performance liquid chromatography (chromatographic column: Waters Xbridge BEH C18 100*30 mm*10 m; mobile phase: water (10 mM ammonium bicarbonate)-acetonitrile; acetonitrile %: 50% to 70%, 8 minutes), then separated by thin-layer chromatography (developing solvent: dichloromethane/methanol=5/1, v/v) to obtain the target compound WX021. MS-ESI m/z: 894.4 [M+H]⁺. ¹H NMR (400 MHz, DMSO_d₆) δ: 12.54 (s, 1H), 11.11 (s, 1H), 9.06 (s, 1H), 8.46 (d, J=7.6 Hz, 1H), 8.38 (t, J=7.8 Hz, 1H), 8.19 (d, J=8.0 Hz, 1H), 7.94-7.86 (m, 3H), 7.71 (d, J=9.2 Hz, 1H), 7.12 (dd, J=2.2 Hz, 9.0 Hz, 1H), 6.99 (d, J=2.4 Hz, 1H), 6.08 (s, 1H), 5.92 (t, J=5.4 Hz, 1H), 4.93 (dd, J=4.8 Hz, 11.2 Hz, 1H), 3.31-3.21 (m, 4H), 3.07-3.00 (m, 3H), 2.87-2.78 (m, 1H), 2.69-2.52 (m, 3H), 2.42-2.18 (m, 5H), 2.16-2.11 (m, 2H), 1.88-1.81 (m, 3H), 1.73-1.66 (m, 2H), 1.63 (s, 6H), 1.59-1.41 (m, 3H), 1.29-1.20 (m, 1H), 1.18-1.03 (m, 2H).

### Example 23

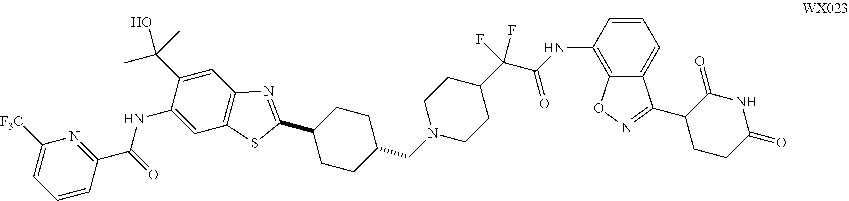 [SMILES: CC(C)(O)c1cc2nc([C@H]3CC[C@H](CN4CCC(C(F)(F)C(=O)Nc5cccc6c(C7CCC(=O)NC7=O)noc56)CC4)CC3)sc2cc1NC(=O)c1cccc(C(F)(F)F)n1]

### Synthetic Route:

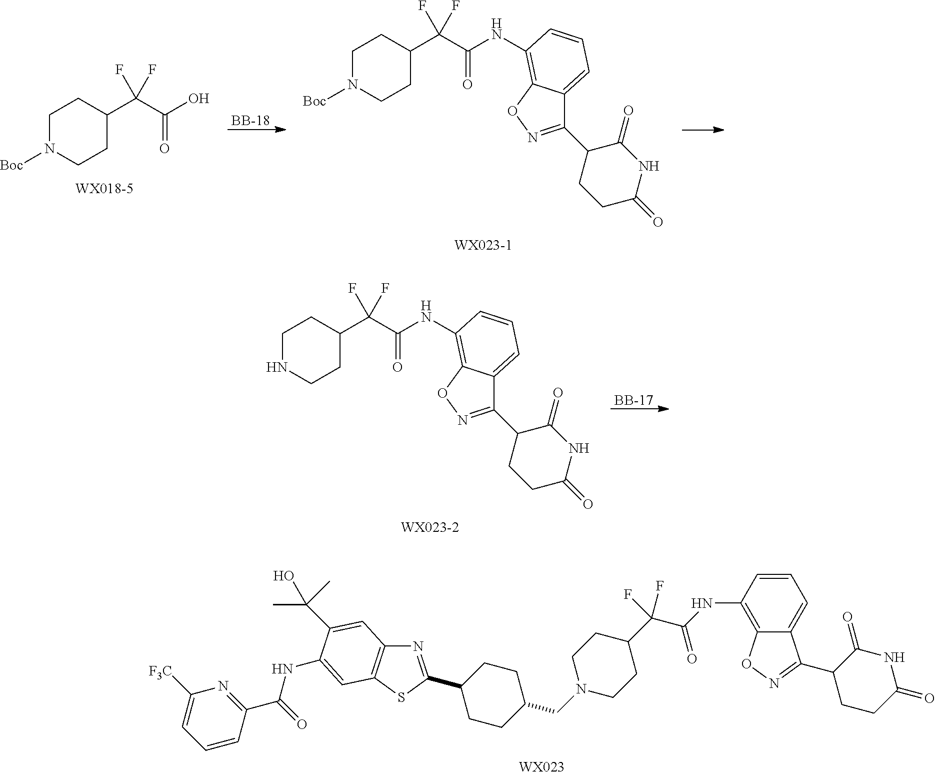

### Step 1: Synthesis of Compound WX023-1

Compound WX018-5 (600 mg, 2.15 mmol) was dissolved in N, N-dimethylformamide (6 mL) at room temperature under nitrogen atmosphere, then N, N-diisopropylethylamine (1.11 g, 8.59 mmol, 1.50 mL) and 2-(7-azabenzotriazol)-N, N, N′, N′-tetramethyluronium hexafluorophosphate (1.09 g, 2.86 mmol) were added thereto. The reaction mixture was stirred for 0.5 hours. The hydrochloride of compound BB-18 (403.46 mg, 1.43 mmol) was added thereto. The reaction mixture was further stirred for 12 hours. After the reaction was completed, the reaction mixture was added with ethyl acetate (30 mL) and 10% brine (15 mL). The organic phase was separated out. The aqueous phase was extracted with ethyl acetate (20 mL). The organic phases were combined, washed with saturated brine (20 mL), dried over anhydrous sodium sulfate, and filtered. The filtrate was concentrated under reduced pressure to obtain a crude product. The crude product was separated by column chromatography (eluent: petroleum ether/ethyl acetate=1/0 to 0/1, v/v) to obtain compound WX023-1. MS-ESI m/z: 451.2 [M+H−56]⁺. ¹H NMR (400 MHz, DMSO_d₆) δ: 11.13 (s, 1H), 10.99 (s, 1H), 7.80 (d, J=7.6 Hz, 1H), 7.62 (d, J=7.6 Hz, 1H), 7.42 (t, J=7.8 Hz, 1H), 4.65 (dd, J=5.2 Hz, 12.0 Hz, 1H), 4.13-4.02 (m, 2H), 2.84-2.75 (m, 3H), 2.68-2.54 (m, 2H), 2.28-2.18 (m, 1H), 1.89-1.75 (m, 2H), 1.41 (s, 9H), 1.38-1.22 (m, 3H).

Step 2: Synthesis of trifluoroacetate of compound WX023-2

Compound WX023-1 (600 mg, 1.18 mmol) was dissolved in dichloromethane (5 mL) at room temperature under nitrogen atmosphere, and trifluoroacetic acid (1.85 g, 16.21 mmol, 1.20 mL) was added thereto. The reaction mixture was stirred and reacted at room temperature for 2 hours. After the reaction was completed, the reaction mixture was concentrated under reduced pressure to obtain the trifluoroacetate of compound WX023-2. MS-ESI m/z: 407.1 [M+H]⁺.

Step 3: Synthesis of formate of compound WX023

Compound BB-17 (150 mg, 248.58 μmol) was added to acetonitrile (2 mL) at room temperature under nitrogen atmosphere, and N, N-diisopropylethylamine (149.01 mg, 1.15 mmol) was added thereto. The mixture was stirred and reacted for 10 minutes, then the trifluoroacetate of compound WX023-2 (200 mg, 384.32 μmol) was added thereto. The reaction mixture was heated to 85° C., and stirred and reacted for 6 hours. After the reaction was completed, the reaction mixture was concentrated under reduced pressure to obtain a crude product. The crude product was separated by preparative high performance liquid chromatography (chromatographic column: Phenomenex Luna C18 75*30 mm*3 m; mobile phase: water (0.2% formic acid)-acetonitrile; acetonitrile %: 20% to 50%, 8 minutes) to obtain the formate of the target compound WX023. MS-ESI m/z: 882.9 [M+H]⁺. ¹H NMR (400 MHz, DMSO_d₆) δ: 12.56 (s, 1H), 11.13 (s, 1H), 11.00 (s, 1H), 9.08 (s, 1H), 8.47 (d, J=8.0 Hz, 1H), 8.39 (t, J=7.8 Hz, 1H), 8.19 (d, J=8.0 Hz, 1H), 8.14 (s, 1H), 7.90 (s, 1H), 7.80 (d, J=8.0 Hz, 1H), 7.63 (d, J=7.6 Hz, 1H), 7.42 (t, J=7.8 Hz, 1H), 6.08 (s, 1H), 4.65 (dd, J=5.2 Hz, 12.0 Hz, 1H), 3.19-3.00 (m, 3H), 2.87-2.72 (m, 1H), 2.68-2.53 (m, 2H), 2.41-2.28 (m, 3H), 2.27-2.07 (m, 5H), 1.93 (d, J=11.6 Hz, 2H), 1.84 (d, J=11.6 Hz, 2H), 1.76-1.51 (m, 11H), 1.18-1.03 (m, 2H).

### Example 24

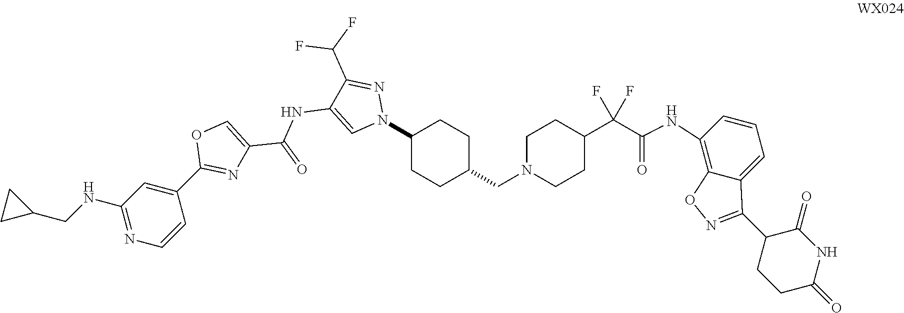 [SMILES: O=C1CCC(c2noc3c(NC(=O)C(F)(F)C4CCN(C[C@H]5CC[C@H](n6cc(NC(=O)c7coc(-c8ccnc(NCC9CC9)c8)n7)c(C(F)F)n6)CC5)CC4)cccc23)C(=O)N1]

### Synthetic Route:

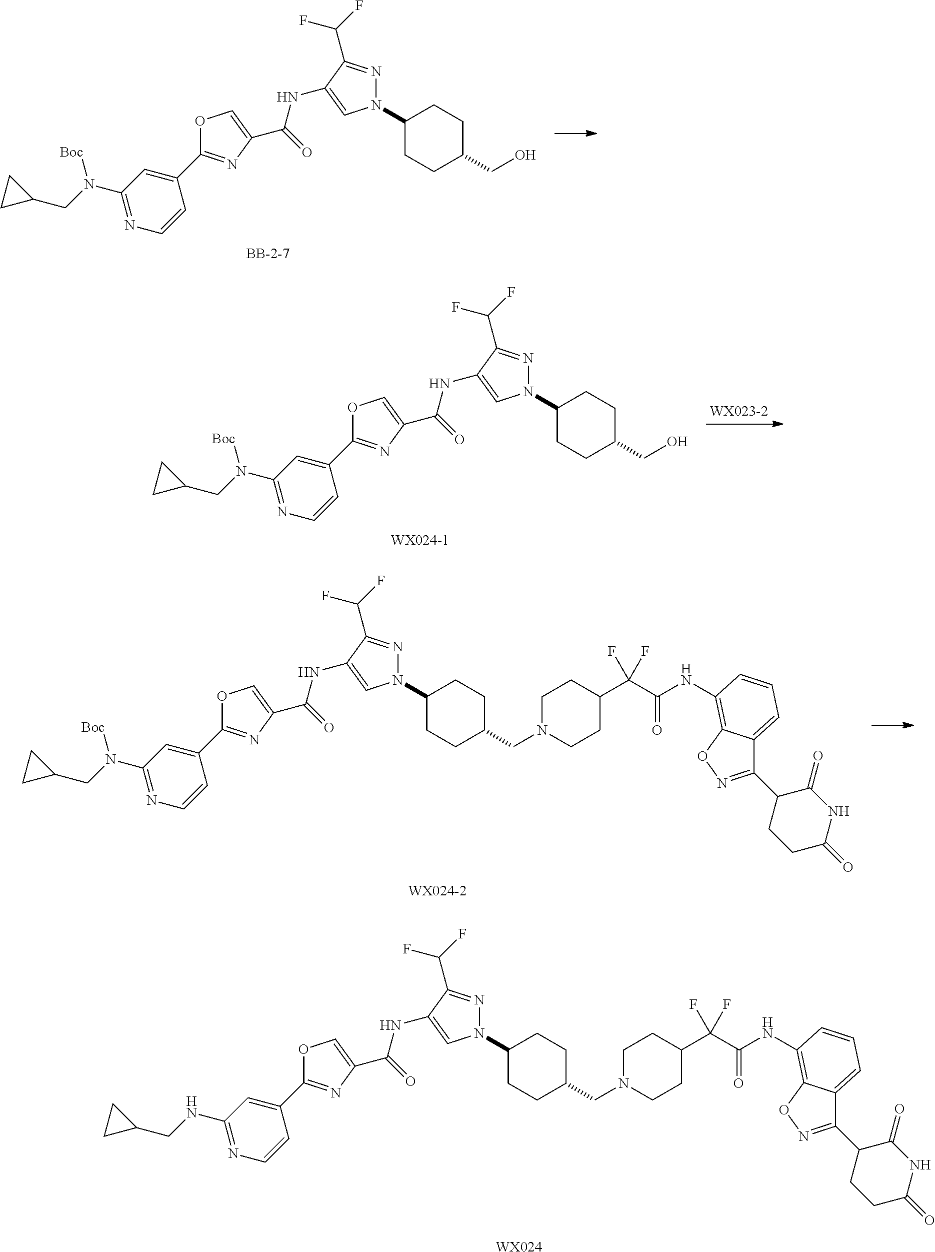

### Step 1: Synthesis of Compound WX024-1

Compound BB-2-7 (2 g, 3.41 mmol) was dissolved in dichloromethane (40 mL) at room temperature under nitrogen atmosphere, then triphenylphosphine (1.07 g, 4.09 mmol) and imidazole (348.15 mg, 5.11 mmol) were added thereto. The mixture was cooled in an ice-water bath to 0° C., and iodine (1.12 g, 4.43 mmol) was added thereto. The reaction mixture was stirred and reacted at room temperature for 12 hours. After the reaction was completed, the reaction mixture was poured into a saturated solution of sodium sulfite (50 mL), and extracted with dichloromethane (80 mL×3). The organic phases were combined, washed with saturated brine (50 mL×2), dried over anhydrous sodium sulfate, and filtered. The filtrate was concentrated under reduced pressure to remove the solvent. The resulting residue was separated by column chromatography (eluent: petroleum ether/ethyl acetate=5/1 to 3/1, v/v) to obtain compound WX024-1. MS-ESI m/z: 697.0 [M+H]⁺. ¹H NMR (400 MHz, CDCl₃) δ: 9.07 (s, 1H), 8.52 (d, J=4.8 Hz, 1H), 8.37 (s, 1H), 8.33 (s, 1H), 8.32 (s, 1H), 7.64 (dd, J=1.2 Hz, 5.2 Hz, 1H), 6.83 (t, J=54.8 Hz, 1H), 4.16-4.03 (m, 1H), 3.95 (d, J=7.2 Hz, 2H), 3.17 (d, J=6.0 Hz, 2H), 2.28-2.18 (m, 2H), 2.15-2.07 (m, 2H), 1.93-1.80 (m, 2H), 1.60-1.54 (m, 10H), 1.32-1.17 (m, 3H), 0.48-0.40 (m, 2H), 0.31-0.25 (m, 2H).

### Step 2: Synthesis of Compound WX024-2

Compound WX024-1 (196.30 mg, 281.83 μmol) was added to acetonitrile (2 mL) at room temperature under nitrogen atmosphere, and N, N-diisopropylethylamine (218.54 mg, 1.69 mmol) was added thereto. The mixture was stirred and reacted for 10 minutes, then the trifluoroacetate of compound WX023-2 (220 mg, 422.75 μmol) was added thereto. The reaction mixture was heated to 85° C., and stirred and reacted for 6 hours. After the reaction was completed, the reaction mixture was cooled to room temperature, added with ethyl acetate (20 mL) and water (10 mL) to separate the organic phase. The aqueous phase was extracted with ethyl acetate (15 mL×2). The organic phases were combined, dried over anhydrous sodium sulfate, and filtered, The filtrate was concentrated under reduced pressure to obtain compound WX024-2. MS-ESI m/z: 975.3 [M+H]⁺.

Step 3: Synthesis of formate of compound WX024

Compound WX024-2 (270 mg, 276.92 μmol) was dissolved in dichloromethane (5 mL) at room temperature under nitrogen atmosphere, and trifluoroacetic acid (770.00 mg, 6.75 mmol, 0.5 mL) was added thereto. The reaction mixture was stirred and reacted for 5 hours. After the reaction was completed, the reaction mixture was concentrated under reduced pressure to obtain a crude product. The crude product was separated by preparative high performance liquid chromatography (chromatographic column: Phenomenex Luna C18 75*30 mm*3 m; mobile phase: water (0.2% formic acid)-acetonitrile; acetonitrile %: 1% to 30%, 8 minutes) to obtain the formate of the target compound WX024. MS-ESI m/z: 875.5 [M+H]+¹H NMR (400 MHz, DMSO_d₆) δ: 11.12 (s, 1H), 10.97 (s, 1H), 9.69 (s, 1H), 8.92 (s, 1H), 8.20-8.12 (m, 2H), 7.79 (d, J=8.0 Hz, 1H), 7.61 (d, J=7.6 Hz, 1H), 7.41 (t, J=7.8 Hz, 1H), 7.32-6.99 (m, 4H), 4.64 (dd, J=4.8 Hz, 12.0 Hz, 1H), 4.27-4.14 (m, 1H), 3.18 (t, J=6.2 Hz, 2H), 3.01 (d, J=10.8 Hz, 2H), 2.86-2.73 (m, 1H), 2.66-2.52 (m, 2H), 2.36-2.14 (m, 4H), 2.10-1.87 (m, 6H), 1.85-1.70 (m, 4H), 1.68-1.49 (m, 3H), 1.15-0.99 (m, 3H), 0.50-0.42 (m, 2H), 0.26-0.18 (m, 2H).

### Example 25

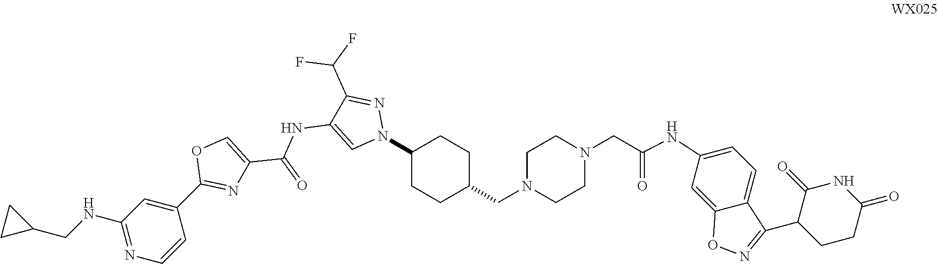 [SMILES: O=C1CCC(c2noc3cc(NC(=O)CN4CCN(C[C@H]5CC[C@H](n6cc(NC(=O)c7coc(-c8ccnc(NCC9CC9)c8)n7)c(C(F)F)n6)CC5)CC4)ccc23)C(=O)N1]

### Synthetic Route:

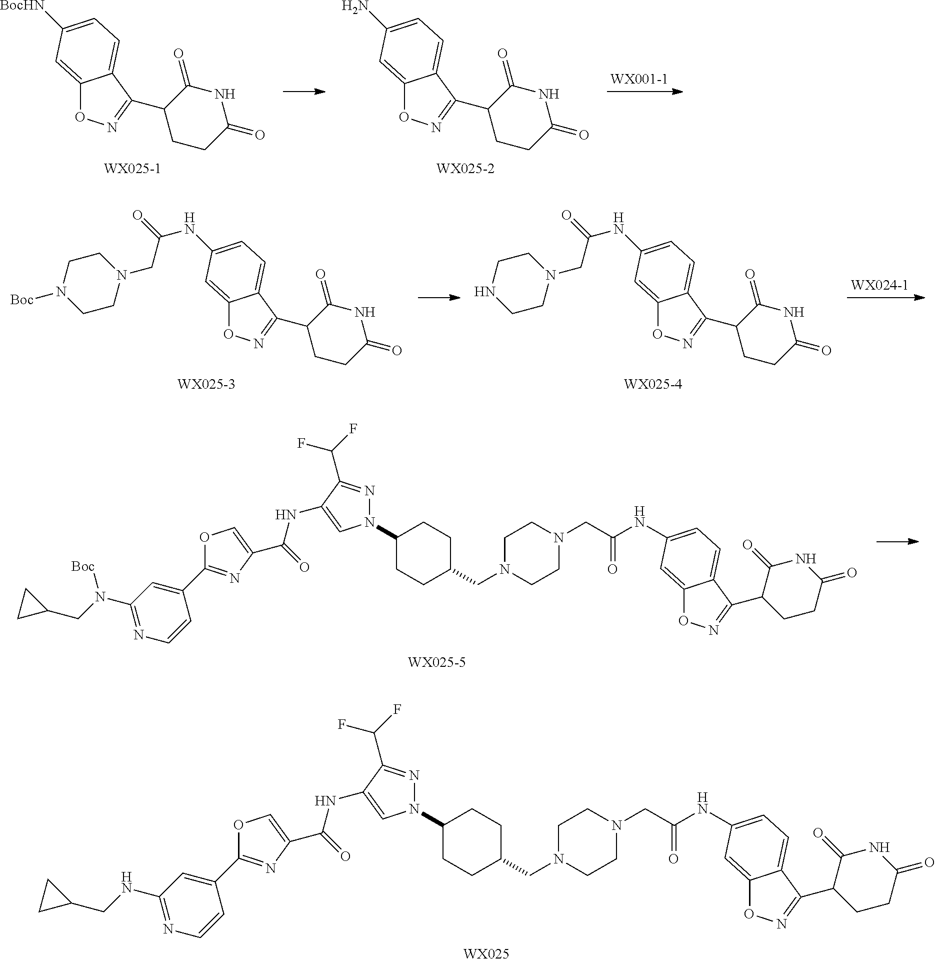

### Step 1: Synthesis of Hydrochloride of Compound WX025-2

Compound WX025-1 (700 mg, 2.03 mmol) was added to a solution of hydrochloric acid in ethyl acetate (4 M, 10 mL) at room temperature under nitrogen atmosphere. The mixture was stirred and reacted for 1 hour. After the reaction was completed, the reaction mixture was concentrated under reduced pressure to obtain the hydrochloride of the compound WX025-2. MS-ESI m/z: 246.2 [M+H]⁺. ¹H NMR (400 MHz, DMSO_d₆) δ: 11.04 (s, 1H), 7.47 (d, J=8.4 Hz, 1H), 6.77 (s, 1H), 6.72 (dd, J=1.4 Hz, 8.6 Hz, 1H), 4.41 (dd, J=5.0 Hz, 11.4 Hz, 1H), 2.79-2.68 (m, 1H), 2.62-2.54 (m, 1H), 2.47-2.35 (m, 1H), 2.21-2.10 (m, 1H).

### Step 2: Synthesis of Compound WX025-3

Compound WX001-1 (728.45 mg, 2.98 mmol) was dissolved in N, N-dimethylformamide (15 mL), then 2-(7-azabenzotriazol)-N, N, N′, N′-tetramethyluronium hexafluorophosphate (1.89 g, 4.97 mmol) and N, N-diisopropylethylamine (1.93 g, 14.91 mmol, 2.60 mL) were added thereto, stirred and reacted for 30 minutes, then the hydrochloride of compound WX025-2 (0.7 g, 2.48 mmol) was added thereto. The reaction mixture was stirred and reacted for another 4.5 hours. After the reaction was completed, the reaction mixture was added with water (10 mL) and ethyl acetate (30 mL), stirred at room temperature for 15 minutes, and filtered. The filter cake was collected. The filtrate was extracted with ethyl acetate (30 mL×3). The organic phases were combined, washed with saturated brine (30 mL), dried over anhydrous sodium sulfate, and filtered. The filtrate was concentrated under reduced pressure to obtain a crude product. The crude product was separated by column chromatography (eluent: petroleum ether/ethyl acetate=1/0 to 0/1, v/v). The crude product obtained was added with ethyl acetate (10 mL), stirred at room temperature for 1 hour, and filtered. The filter cake was collected, and dried under vacuum to obtain compound WX025-3. MS-ESI m/z: 472.3 [M+H]⁺. ¹H NMR (400 MHz, DMSO_d₆) δ: 11.09 (s, 1H), 10.26 (s, 1H), 8.23 (s, 1H), 7.77 (d, J=8.4 Hz, 1H), 7.51 (d, J=8.8 Hz, 1H), 4.55 (dd, J=4.6 Hz, 11.8 Hz, 1H), 3.43-3.35 (m, 4H), 3.24 (s, 2H), 2.82-2.71 (m, 1H), 2.64-2.56 (m, 1H), 2.49-2.42 (m, 5H), 2.27-2.12 (m, 1H), 1.40 (s, 9H).

Step 3: Synthesis of trifluoroacetate of compound WX025-4

Compound WX025-3 (400 mg, 848.35 μmol) was dissolved in dichloromethane (4 mL) at room temperature under nitrogen atmosphere, and trifluoroacetic acid (1.23 g, 10.80 mmol, 0.8 mL) was added thereto. The reaction mixture was stirred and reacted for 1 hour. After the reaction was completed, the reaction mixture was concentrated under reduced pressure to obtain the trifluoroacetate of compound WX025-4. MS-ESI m/z: 372.3 [M+H]⁺.

### Step 4: Synthesis of Compound WX025-5

The trifluoroacetate of compound WX025-4 (252.98 mg, 521.16 μmol) was added to acetonitrile (7 mL) at room temperature under nitrogen atmosphere, and N, N-diisopropylethylamine (367.39 mg, 2.84 mmol) was added thereto. The reaction mixture was stirred for 10 minutes, then compound WX024-1 (330 mg, 473.78 μmol) was added thereto. The reaction mixture was heated to 85° C., and stirred and reacted for 6 hours. After the reaction was completed, the reaction mixture was cooled to room temperature, then poured into a saturated solution of ammonium chloride (10 mL), and extracted with ethyl acetate (10 mL×3). The organic phases were combined, washed with saturated brine (20 mL), dried over anhydrous sodium sulfate, and filtered. The filtrate was concentrated under reduced pressure to obtain a crude product. The crude product was purified by column chromatography (eluent: petroleum ether/ethyl acetate=1/0 to 5/1, v/v) to obtain compound WX025-5. MS-ESI m/z: 940.6 [M+H]⁺.

### Step 5: Synthesis of Hydrochloride of Compound WX025

Compound WX025-5 (300 mg, 319.15 μmol) was dissolved in ethyl acetate (1 mL) at room temperature under nitrogen atmosphere. A solution of hydrochloric acid in ethyl acetate (4 M, 3.00 mL) was added thereto. The reaction mixture was stirred and reacted for 2 hours. After the reaction was completed, the reaction mixture was filtered, and rinsed with ethyl acetate (2 mL×2). The filter cake was collected to obtain a crude product. The crude product was added with a mixed solution of acetonitrile (1 mL) and water (1 mL). The mixture was stirred at room temperature for 0.5 hours, and filtered. The filter cake was collected, and dried under vacuum to obtain the hydrochloride of the target compound WX025. MS-ESI m/z: 840.5 [M+H]⁺. ¹H NMR (400 MHz, DMSO_d₆) δ: 11.11 (s, 1H), 9.91 (s, 1H), 9.13 (s, 1H), 8.23 (d, J=1.2 Hz, 1H), 8.20 (s, 1H), 8.09 (d, J=6.8 Hz, 1H), 7.82 (d, J=8.4 Hz, 1H), 7.64 (s, 1H), 7.51 (dd, J=1.2 Hz, 8.8 Hz, 1H), 7.36-7.05 (m, 2H), 4.58 (dd, J=4.8 Hz, 12.0 Hz, 1H), 4.34-4.22 (m, 1H), 3.99-3.84 (m, 2H), 3.78-3.70 (m 2H), 3.47-3.37 (m, 4H), 3.36-3.28 (m, 4H), 3.12-2.98 (m, 2H), 2.87-2.73 (m, 1H), 2.66-2.58 (m, 1H), 2.26-2.17 (m, 1H), 2.13-2.01 (m, 4H), 1.98-1.88 (m, 1H), 1.87-1.75 (m, 2H), 1.32-1.09 (m, 3H), 0.61-0.54 (m, 2H), 0.37-0.31 (m, 2H).

### Example 26

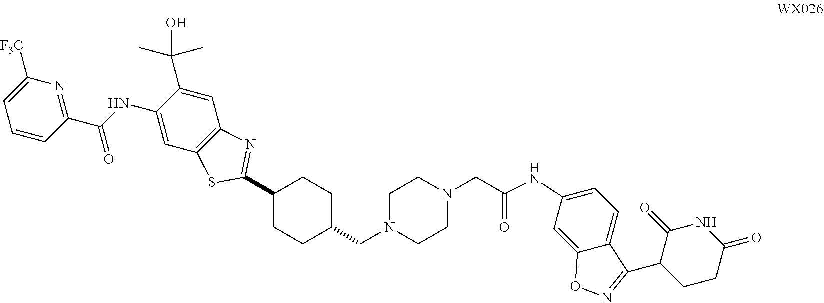 [SMILES: CC(C)(O)c1cc2nc([C@H]3CC[C@H](CN4CCN(CC(=O)Nc5ccc6c(C7CCC(=O)NC7=O)noc6c5)CC4)CC3)sc2cc1NC(=O)c1cccc(C(F)(F)F)n1]

### Synthetic Route:

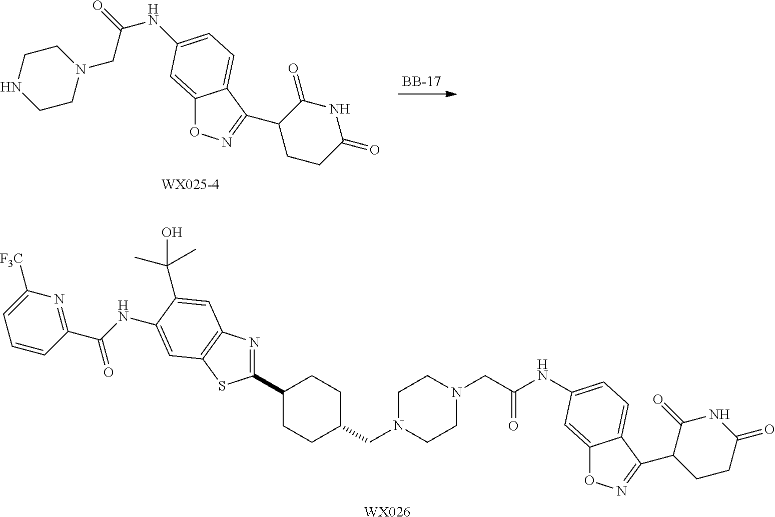

### Synthesis of Formate of Compound WX026

The trifluoroacetate of compound WX025-4 (125.49 mg, 258.52 μmol) was added to acetonitrile (2 mL) at room temperature under nitrogen atmosphere, and N, N-diisopropylethylamine (133.64 mg, 1.03 mmol) was added thereto. The reaction mixture was stirred and reacted for 10 minutes, then compound BB-17 (104.00 mg, 172.35 μmol) was added thereto. The reaction mixture was heated to 85° C., and stirred and reacted for 6 hours. After the reaction was completed, the reaction mixture was concentrated under reduced pressure to obtain a crude product. The crude product was separated by preparative high performance liquid chromatography (chromatographic column: Phenomenex Luna C18 75*30 mm*3 m; mobile phase: water (0.2% formic acid)-acetonitrile; acetonitrile %: 30% to 70%, 8 minutes) to obtain the formate of the target compound WX026. MS-ESI m/z: 847.0 [M+H]⁺. ¹H NMR (400 MHz, DMSO_d₆) δ: 12.55 (s, 1H), 11.09 (s, 1H), 10.16 (s, 1H), 9.07 (s, 1H), 8.47 (d, J=7.6 Hz, 1H), 8.39 (t, J=7.8 Hz, 1H), 8.23 (s, 1H), 8.19 (dd, J=0.6 Hz, 7.8 Hz, 1H), 7.90 (s, 1H), 7.78 (d, J=8.8 Hz, 1H), 7.49 (dd, J=1.4 Hz, 8.6 Hz, 1H), 6.08 (s, 1H), 4.55 (dd, J=5.0 Hz, 11.8 Hz, 1H), 3.30 (s, 2H), 3.22 (s, 2H), 3.11-3.01 (m, 1H), 2.82-2.72 (m, 1H), 2.66-2.55 (m, 5H), 2.47-2.41 (m, 2H), 2.28-2.13 (m, 5H), 1.98-1.87 (m, 2H), 1.72-1.50 (m, 10H), 1.17-1.00 (m, 2H).

### Example 28

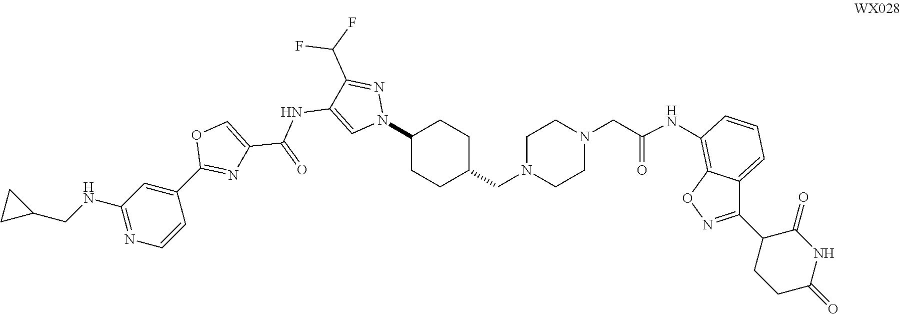 [SMILES: O=C1CCC(c2noc3c(NC(=O)CN4CCN(C[C@H]5CC[C@H](n6cc(NC(=O)c7coc(-c8ccnc(NCC9CC9)c8)n7)c(C(F)F)n6)CC5)CC4)cccc23)C(=O)N1]

### Synthetic Route:

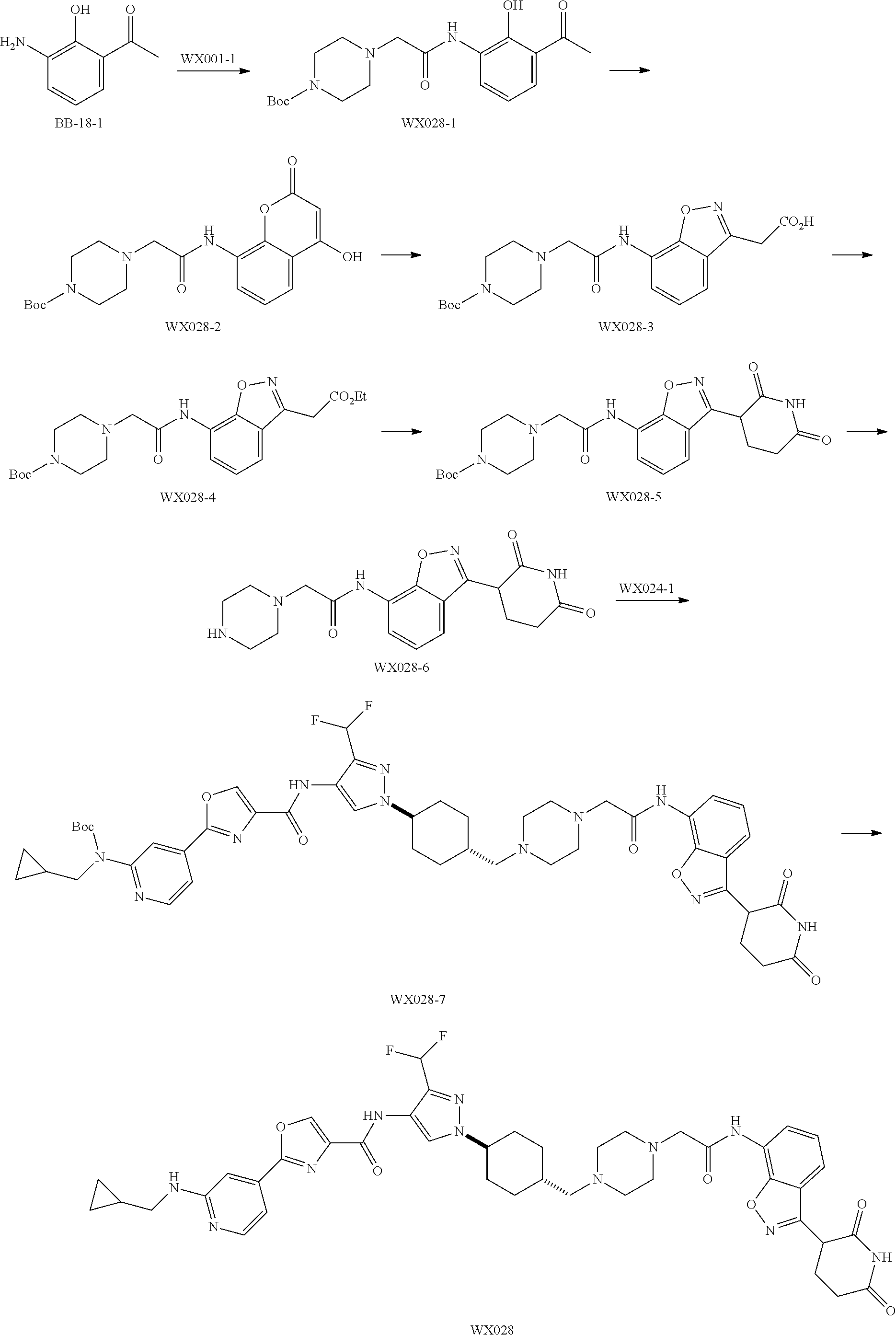

### Step 1: Synthesis of Compound WX028-1

Compound WX001-1 (9.7 g, 39.69 mmol) was dissolved in N, N-dimethylformamide (50 mL) at room temperature under nitrogen atmosphere, then N, N-diisopropylethylamine (8.55 g, 66.15 mmol) and 2-(7-azabenzotriazol)-N, N, N′, N′-tetramethyluronium hexafluorophosphate (16.35 g, 43.00 mmol) were sequentially added thereto. The reaction mixture was stirred and reacted at room temperature for 0.5 hours, then compound BB-18-1 (5 g, 33.08 mmol) was added thereto. The reaction mixture was further stirred and reacted at room temperature for 2 hours. After the reaction was completed, the reaction mixture was added with water (200 mL) and extracted with ethyl acetate (50 mL×3). The organic phases were combined, washed with saturated brine (100 mL), dried over anhydrous sodium sulfate, and filtered. The filtrate was concentrated under reduced pressure to remove the solvent. The resulting residue was separated by column chromatography (eluent: petroleum ether/ethyl acetate=2/1, v/v) to obtain compound WX028-1. MS-ESI m/z: 378.3 [M+H]⁺.

### Step 2: Synthesis of Compound WX028-2

Compound WX028-1 (4 g, 10.60 mmol) and dimethyl carbonate (3.82 g, 42.39 mmol, 3.57 mL) were dissolved in tetrahydrofuran (50 mL) at room temperature under nitrogen atmosphere, and potassium tert-butoxide (7.14 g, 63.59 mmol) was added thereto. The reaction mixture was heated to 90° C., and stirred and reacted for 4 hours. After the reaction was completed, the mixture was concentrated under reduced pressure to remove the solvent, added with ice water (50 mL) for dissolution, and extracted with methyl tert-butyl ether (50 mL×2). The aqueous phase was collected, added with 6 M hydrochloric acid to adjust the pH to 7. The aqueous phase was concentrated under reduced pressure to obtain compound WX028-2. MS-ESI m/z: 404.2 [M+H]⁺. ¹H NMR (400 MHz, D₂O) δ: 7.78 (d, J=7.6 Hz, 1H), 7.60 (d, J=7.6 Hz, 1H), 7.19 (t, J=7.8 Hz, 1H), 5.17 (s, 1H), 3.58-3.45 (m, 4H), 3.27 (s, 2H), 2.62-2.50 (m, 4H), 1.42 (s, 9H).

### Step 3: Synthesis of Compound WX028-3

Compound WX028-2 (4.2 g, 10.41 mmol) was dissolved in ethanol (80 mL) at room temperature, then sodium acetate (3.42 g, 41.64 mmol) and hydroxylamine hydrochloride (2.53 g, 36.44 mmol) were added thereto. The reaction mixture was heated to 80° C., and stirred and reacted for 5 hours. After the reaction was completed, the reaction mixture was cooled to room temperature, and filtered. The filter cake was rinsed with dichloromethane (50 mL×2). The filtrate was concentrated to remove most of the solvent, added with water (100 mL), and extracted with dichloromethane (50 mL×3). The organic phases were combined, washed with saturated brine (100 mL), dried over anhydrous sodium sulfate, and filtered. The filtrate was concentrated under reduced pressure to obtain compound WX028-3. MS-ESI m/z: 419.1 [M+H]⁺.

### Step 4: Synthesis of Compound WX028-4

Compound WX028-3 (2.5 g, 5.97 mmol) was dissolved in dichloromethane (40 mL) at 30° C., 4-dimethylaminopyridine (1.09 g, 8.96 mmol) and di-tert-butyl carbonate (2 g, 9.16 mmol, 2.11 mL) were added thereto. After the reaction mixture was stirred and reacted at 30° C. for 1.5 hours, anhydrous ethanol (2.75 g, 59.75 mmol) was added thereto. The mixture was further reacted for 1 hour. After the reaction was completed, the reaction mixture was poured into water (50 mL), and the phases were separated. The aqueous was again extracted with dichloromethane (30 mL). The organic phases were combined, dried over anhydrous sodium sulfate, and filtered. The filtrate was concentrated under reduced pressure to obtain a crude product. The crude product was purified by column chromatography (eluent: ethyl acetate/petroleum ether=1/5) to obtain compound WX028-4. MS-ESI m/z: 447.3 [M+H]⁺. ¹H NMR (400 MHz, CDCl₃) δ: 9.79 (s, 1H), 8.46 (d, J=7.6 Hz, 1H), 7.41 (d, J=7.6 Hz, 1H), 7.32 (t, J=8.0 Hz, 1H), 4.22 (q, J=7.0 Hz, 2H), 4.04 (s, 2H), 3.63-3.54 (m, 4H), 3.26 (s, 2H), 2.69-2.58 (m, 4H), 1.48 (s, 9H), 1.26 (t, J=7.2 Hz, 3H).

### Step 5: Synthesis of Compound WX028-5

Compound WX028-4 (0.92 g, 2.06 mmol) and acrylamide (175.75 mg, 2.47 mmol) were dissolved in tetrahydrofuran (20 mL) at room temperature under nitrogen atmosphere. The mixture was cooled to 0° C., and a solution of potassium tert-butoxide in tetrahydrofuran (1 M, 3.09 mL) was added dropwise thereto. The reaction mixture was stirred and reacted at 0° C. for 1.5 hours. After the reaction was completed, the reaction mixture was added with 0.05 M hydrochloric acid (70 mL), and extracted with ethyl acetate (40 mL×2). The organic phases were combined, washed with saturated brine (40 mL), dried over anhydrous sodium sulfate, and filtered. The filtrate was concentrated under reduced pressure to obtain a crude product. The crude product was purified by column chromatography (eluent: petroleum ether/ethyl acetate=5/1 to 1/1) to obtain compound WX028-5. MS-ESI m/z: 472.2 [M+H]⁺. ¹H NMR (400 MHz, CDCl₃) δ: 9.83 (s, 1H), 8.49 (d, J=6.4 Hz, 1H), 8.10 (s, 1H), 7.42 (d, J=7.6 Hz, 1H), 7.35 (t, J=7.8 Hz, 1H), 4.34 (dd, J=5.0 Hz, 9.0 Hz, 1H), 3.66-3.52 (m, 4H), 3.27 (s, 2H), 3.06-2.95 (m, 1H), 2.83-2.73 (m, 1H), 2.71-2.56 (m, 5H), 2.53-2.43 (m, 1H), 1.49 (s, 9H).

### Step 6: Synthesis of Trifluoroacetate of Compound WX028-6

Compound WX028-5 (3 g, 6.36 mmol) was dissolved in dichloromethane (40 mL) at room temperature, and trifluoroacetic acid (9.24 g, 81.04 mmol) was added dropwise thereto. The reaction mixture was stirred at room temperature for 4 hours. After the reaction was completed, the mixture was concentrated under reduced pressure to remove the solvent to obtain the trifluoroacetate of compound WX028-6. MS-ESI m/z: 372.0 [M+H]⁺.

### Step 7: Synthesis of Compound WX028-7

Compound WX024-1 (0.2 g, 287.14 μmol) was dissolved in acetonitrile (4 mL) at room temperature under nitrogen atmosphere. The trifluoroacetate of compound WX028-6 (225.35 mg, 315.85 μmol) and N, N-diisopropylethylamine (259.78 mg, 2.01 mmol) were added thereto. The reaction mixture was heated to 90° C., and stirred and reacted for 12 hours. After the reaction was completed, the reaction mixture was cooled to room temperature, poured into water (30 mL), and extracted with ethyl acetate (50 mL×3). The organic phase was washed with saturated brine (50 mL×3), dried over anhydrous sodium sulfate, and filtered. The filtrate was concentrated under reduced pressure to remove the remaining solvent. The resulting residue was added with methyl tert-butyl ether (3 mL), stirred at room temperature for 0.5 hours, and filtered. The filter cake was rinsed with methyl tert-butyl ether (3 mL×2), collected, and dried under vacuum to obtain compound WX028-7. MS-ESI m/z: 940.5 [M+H]⁺.

### Step 8: Synthesis of Hydrochloride of Compound WX028

Compound WX028-7 (0.21 g, 223.40 μmol) was dissolved in ethyl acetate (2 mL) at room temperature, and hydrochloric acid/ethyl acetate (5 mL, 4 M) was added thereto. The reaction mixture was stirred and reacted at room temperature for 1 hour. After the reaction was completed, the reaction mixture was filtered. The filter cake was rinsed with ethyl acetate (3 mL×3), and collected. The filter cake was separated by preparative high performance liquid chromatography (chromatographic column: Phenomenex Luna 80*30 mm*3 m; mobile phase: water (0.04% hydrochloric acid))-acetonitrile; acetonitrile %: 1% to 30%, 8 minutes) to obtain the hydrochloride of the target compound WX028. MS-ESI m/z: 840.4 [M+H]⁺. ¹H NMR (400 MHz, MeOD_d₄) δ: 8.80 (s, 1H), 8.26 (s, 1H), 8.13 (d, J=7.6 Hz, 1H), 7.97 (d, J=7.2 Hz, 1H), 7.71 (d, J=0.8 Hz, 1H), 7.63 (d, J=8.0 Hz, 1H), 7.45 (dd, J=1.4 Hz, 7.0 Hz, 1H), 7.39 (t, J=7.8 Hz, 1H), 6.93 (t, J=54.4 Hz, 1H), 4.56 (dd, J=5.0, 11.0 Hz, 1H), 4.32-4.20 (m, 1H), 4.09-3.97 (m, 2H), 3.87-3.41 (m, 8H), 3.29 (s, 2H), 3.25-3.16 (m, 2H), 2.86-2.77 (m, 2H), 2.65-2.50 (m, 1H), 2.43-2.34 (m, 1H), 2.23 (d, J=12.0 Hz, 2H), 2.11 (d, J=13.2 Hz, 2H), 2.06-1.88 (m, 3H), 1.45-1.30 (m, 2H), 1.28-1.14 (m, 1H), 0.77-0.64 (m, 2H), 0.46-0.35 (m, 2H).

### Example 29

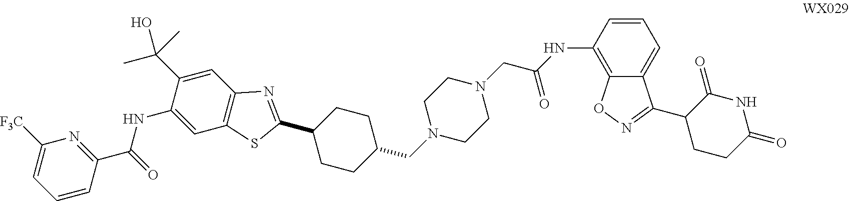 [SMILES: CC(C)(O)c1cc2nc([C@H]3CC[C@H](CN4CCN(CC(=O)Nc5cccc6c(C7CCC(=O)NC7=O)noc56)CC4)CC3)sc2cc1NC(=O)c1cccc(C(F)(F)F)n1]

### Synthetic Route:

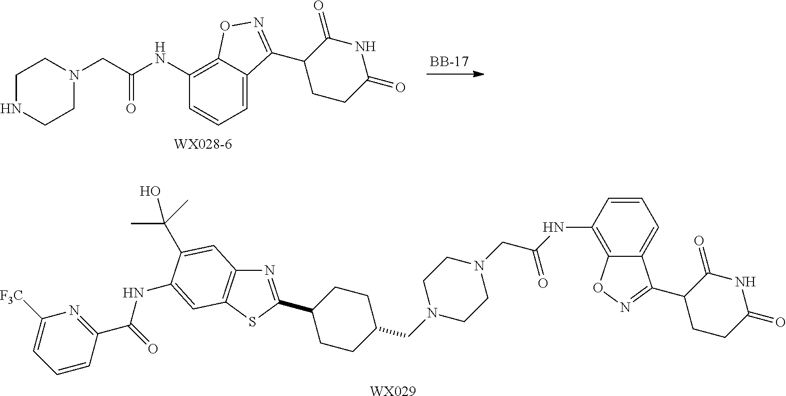

### Step 1: Synthesis of Formate of Compound WX029

The trifluoroacetate of compound WX028-6 (170 mg, 238.28 μmol) was dissolved in acetonitrile (2 mL) at room temperature under nitrogen atmosphere, then N, N-diisopropylethylamine (153.97 mg, 1.19 mmol, 207.51 μL) and compound BB-17 (143.78 mg, 238.28 mol) were added thereto. The reaction mixture was heated to 90° C., and stirred and reacted for 12 hours. After the reaction was completed, the reaction mixture was concentrated under reduced pressure to remove the solvent. The resulting residue was separated by preparative high performance liquid chromatography (chromatographic column: Phenomenex Luna C18 75*30 mm*3 m; mobile phase: water (0.2% formic acid)-acetonitrile; acetonitrile %: 20% to 50%, 8 minutes) to obtain the formate of the target compound WX029. MS-ESI m/z: 847.3 [M+H]⁺. ¹H NMR (400 MHz, DMSO_d₆) δ: 12.55 (s, 1H), 11.12 (s, 1H), 10.02 (s, 1H), 9.07 (s, 1H), 8.46 (J=7.6 Hz, 1H), 8.38 (t, J=8.0 Hz, 1H), 8.18 (dd, J=0.6, 7.8 Hz, 1H), 8.15 (s, 1H), 8.10 (d, J=7.6 Hz, 1H), 7.89 (s, 1H), 7.59 (dd, J=0.8 Hz, 8.0 Hz, 1H), 7.36 (t, J=7.8 Hz, 1H), 6.07 (s, 1H), 4.63 (dd, J=4.8 Hz, 12.0 Hz, 1H), 3.25 (s, 2H), 3.11-2.99 (m, 1H), 2.85-2.72 (m, 1H), 2.70-2.51 (m, 8H), 2.48-2.43 (m, 2H), 2.28-2.13 (m, 5H), 1.93 (d, J=11.2 Hz, 2H), 1.71-1.52 (m, 9H), 1.17-0.99 (m, 2H).

### Example 30 and Example 31

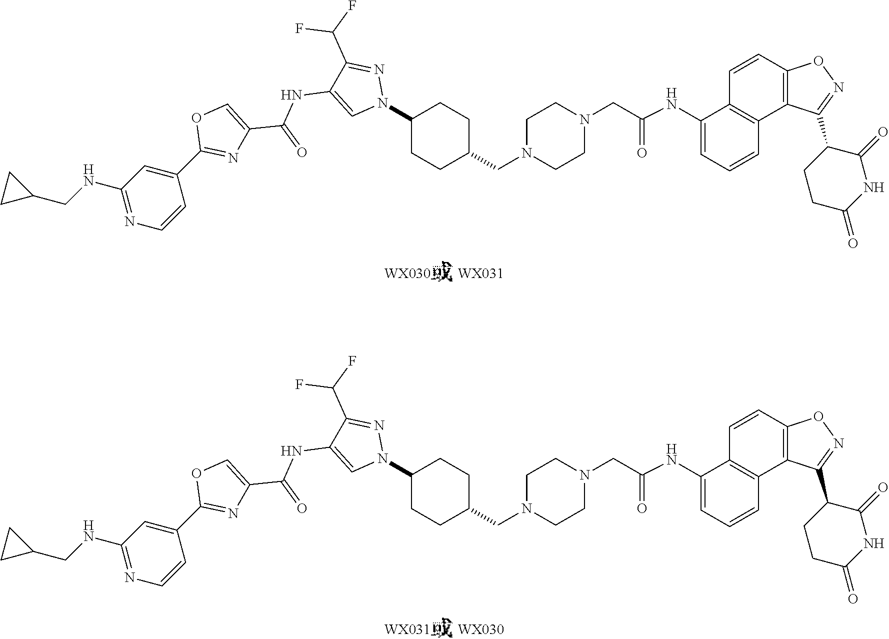

### Synthetic Route:

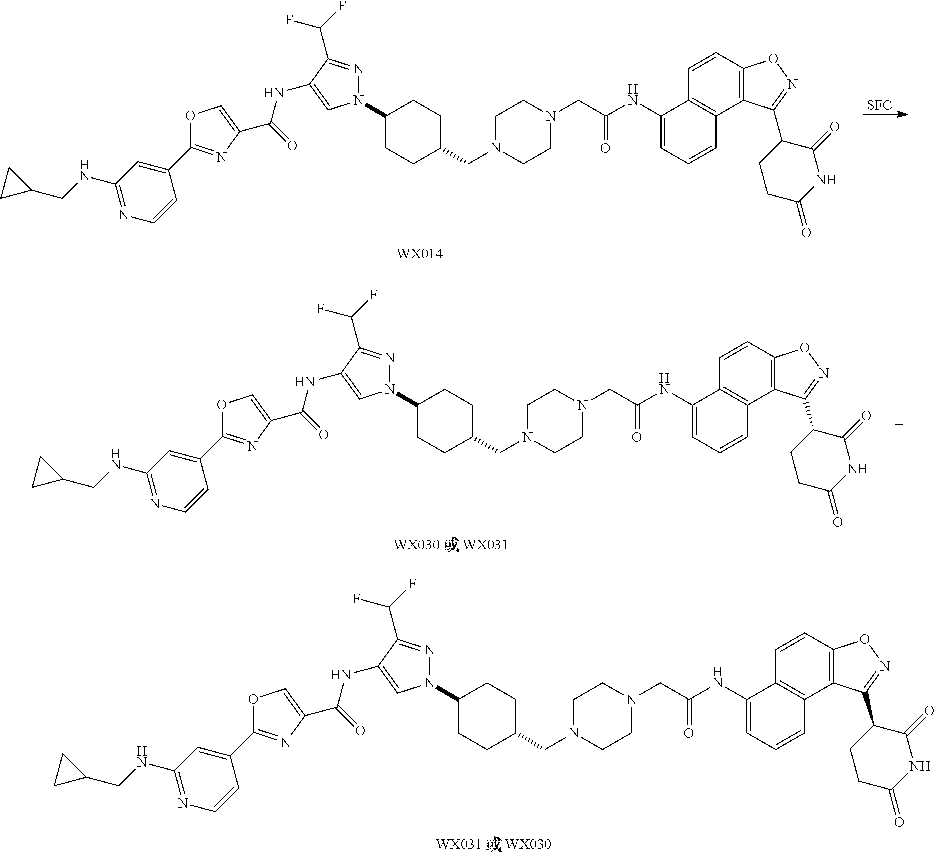

### Synthesis of Hydrochloride of Compound WX030 or Hydrochloride of Compound WX031

The hydrochloride of compound WX014 (480 mg, 518.13 μmol) was separated by supercritical fluid chromatography (separation conditions, chromatographic column: REGIS(S,S)WHELK-O1 (250 mm*25 mm, 10 m); mobile phase: A: CO₂; B: EtOH/ACN (0.1% IPAm, v/v); B %: 65%-65%, 15 minutes) and the samples with a retention time of 1.576 minutes were collected and then separated by preparative high performance liquid chromatography (chromatographic column: Phenomenex Luna 80*30 mm*3 m; mobile phase: water (0.04% hydrochloric acid)-acetonitrile; acetonitrile %: 1%-30%, 8 minutes) to obtain the hydrochloride of the target compound WX030 (ee %: 99.12%). The samples with a retention time of 2.770 minutes were collected, then separated by preparative high performance liquid chromatography (chromatographic column: Phenomenex Luna 80*30 mm*3 m; mobile phase: water (0.04% hydrochloric acid))-acetonitrile; acetonitrile %: 1% to 30%, 8 minutes) to obtain the hydrochloride of the target compound WX031 (ee %: 96.36%). The SFC analysis method corresponding to the retention time as described above: chromatographic column: (S,S)-WHELK-01, 50×4.6 mm I.D., 3.5 m; mobile phase: A: CO₂; B: EtOH:ACN=1:1 (0.1% IPAm, v/v), 4 min.

Synthesis of hydrochloride of compound WX030: ¹H NMR (400 MHz, MeOD_d₄) δ: 11.15 (s, 1H), 10.62 (s, 1H), 9.88 (s, 1H), 9.11 (s, 1H), 8.34 (d, J=9.2 Hz, 1H), 8.20 (s, 1H), 8.18-8.12 (m, 1H), 8.08 (d, J=6.4 Hz, 1H), 8.00 (d, J=9.6 Hz, 1H), 7.80-7.70 (m, 2H), 7.66-7.54 (m, 1H), 7.36-7.03 (m, 2H), 5.10 (dd, J=4.8 Hz, 11.2 Hz, 1H), 4.38-4.20 (m, 2H), 4.18-3.27 (m, 12H), 3.18-2.98 (m, 2H), 2.93-2.80 (m, 1H), 2.72-2.54 (m, 2H), 2.45-2.35 (m, 1H), 2.17-1.99 (m, 4H), 1.97-1.66 (m, 3H), 1.33-1.06 (m, 3H), 0.63-0.48 (m, 2H), 0.38-0.27 (m, 2H).

Synthesis of hydrochloride of compound WX031: ¹H NMR (400 MHz, MeOD_d₄) δ: 11.15 (s, 1H), 10.44 (s, 1H), 9.86 (s, 1H), 9.09 (s, 1H), 8.31 (d, J=9.2 Hz, 1H), 8.20 (s, 1H), 8.18-8.12 (m, 1H), 8.09 (d, J=6.4 Hz, 1H), 8.00 (d, J=9.2 Hz, 1H), 7.79-7.69 (m, 2H), 7.61-7.47 (m, 1H), 7.34-7.02 (m, 2H), 5.10 (dd, J=4.8 Hz, 11.2 Hz, 1H), 4.40-4.21 (m, 2H), 4.18-3.27 (m, 12H), 3.15-2.95 (m, 2H), 2.94-2.79 (m, 1H), 2.71-2.58 (m, 2H), 2.45-2.35 (m, 1H), 2.13-1.62 (m, 7H), 1.32-1.06 (m, 3H), 0.61-0.50 (m, 2H), 0.38-0.24 (m, 2H).

### Bioassay

### Experimental Example 1: Evaluation of Target Protein Degradation Effect in K562 IRAK4-HiBiT Cells

Experimental objective: The experiment aims to evaluate the degradation effect of the test compounds on the target protein IRAK4 in K562 IRAK4-HiBiT cells.

### Experimental Materials

### 1. Cells and Culture Medium

Cells: K562 IRAK4-HiBiT cells
Culture medium: RPMI 1640+10% fetal bovine serum+2 mM glutamine+1 mM sodium pyruvate+penicillin/streptomycin.
Positive control: 1000 nM; negative control: 0.1% DMSO.

TABLE 1
reagents and consumables.

| Reagents and consumables | Manufacturers | Cat. No. |
|---|---|---|
| RPMI 1640 | Gibco | Cat # 31800 |
| Fetal bovine serum (FBS) | Biosera | #FB-1058/500 |
| Penicillin/streptomycin | Biosera | #LM-A4118 |
| DPBS | Invitrogen | #14190 |
| 0.25% Trypsin/EDTA | Invitrogen | #25200 |
| solution | | |
| Glutamine | Invitrogen | Cat # 35050061 |
| Sodium pyruvate | Invitrogen | Cat # 11360070 |
| Nano-Glo ® HiBiT assay | Promega | promega# N3040 |
| kit | | |
| 384-well white plate, flat- | Corning | Cat # 3570 |
| bottom | | |
| 384_LDV compound plate | Labcyte | Cat# LP-0200 |

TABLE 2
Instruments

| Instrument name | Manufacturers | Instrument type |
|---|---|---|
| ECHO liquid handler | Labcyte | Echo 550 |
| Bravo automated liquid | Agilent | 16050-101 |
| handling workstation | | |
| Envision plate reader | Perkin Elmer | 2104 |
| Autosampler | Thermofisher | Multidrop Combi |
| Cell counter | Thermo | Countess II FL |

### Experimental Scheme

### Day 1

### 1. Preparation of Compounds

(1). The powder of the compound to be tested was dissolved in DMSO to a storage concentration of 10 mM. A total of 9 μL of the 10 mM of the compound to be tested was manually pipetted into the 1st and 13th columns of the LDV plate using a pipettor;
(2). A volume of 6 μL of DMSO was added to columns 2-12 and 14-24 using a Multidrop Combi;
(3). A 3-fold dilution of the test compounds (3 μL+6 μL) was performed using Bravo, for columns 1-11 and 13-23;
(4). According to the plate layout, 25 nL of compound solution (columns 1-24 of the LDV plate) was transferred to the experimental plate using Echo;
(5) 25 nL of the 1 mM positive control solution was transferred to the assay plate as a 100% degradation control (i.e. LC, HPE), and 25 nL of DMSO was transferred to the assay plate as a 0% control (i.e. HC, ZPE).

### 2. Cell Plating

(1) The culture medium was discarded, and the cells were washed once with DPBS, digested with trypsin, counted, and prepared into a cell suspension of 2×10⁻⁵ cells/mL.
(2) Using the MultiDrop Combi at a medium speed, 25 μL/well of the cell suspension was added to the experimental plate containing the test compounds;
(3) The experimental plate containing cells was returned to the incubator and cultured for 16 to 18 hours at 37° C. and 5% CO₂.

### Day 2

(1). Using the MultiDrop Combi at a high speed, 25 μL/well of the assay reagent (NanoGlo lytic solution+substrate+LgBit protein) was added to the assay plate, followed by shaking for 10 minutes;
(2) The plate was centrifuged at 2000 rpm for 1 minute to remove any bubbles;
(3) Readings were obtained by using an Envision, US Luminescence reader.

### 3. Data Analysis

To calculate the degradation rate (DR) of the test compounds, the following formula is used: IR (%)=(RLU vehicle control−RLU compound)/(RLU vehicle control−RLU positive control)×100%. The solvent control refers to the blank control. The degradation rates of compounds at different concentrations were calculated in Excel, and then XLFit software was used to plot inhibition curves and calculate relevant parameters, including minimum degradation rate, maximum degradation rate, and DC₅₀.

The test results are shown in Table 3.

TABLE 3
Degradation effect of compounds of the present disclosure
on target proteins in K562 IRAK4-HiBiT cells

| | | Maximum |
|---|---|---|
| | | degradation rate |
| Compound No. | DC₅₀ (nM) | (%) |
| Hydrochloride of WX001 | 76.43 | 54.34 |
| Hydrochloride of WX002 | 13.54 | 100.04 |
| Hydrochloride of WX013 | 48.70 | 101.24 |
| Hydrochloride of WX014 | 4.87 | 108.46 |
| Trifluoroacetate of | 50.24 | 81.68 |
| WX010 | | |
| Trifluoroacetate of | 9.60 | 104.57 |
| WX011 | | |
| WX007 | 35.09 | 97.41 |
| Hydrochloride of WX008 | 121.69 | 82.20 |
| Hydrochloride of WX015 | 47.08 | 100.00 |
| Hydrochloride of WX016 | 70.73 | 93.12 |
| Formate of WX017 | 4.00 | 73.88 |
| Hydrochloride of WX018 | 7.91 | 119.43 |
| Formate of WX019 | 15.08 | 92.05 |
| Formate of WX020 | 29.86 | 75.09 |
| Formate of WX023 | 7.16 | 114.38 |
| Formate of WX024 | 1.75 | 117.09 |
| Hydrochloride of WX025 | 2.53 | 105.26 |
| Formate of WX026 | 0.78 | 80.30 |
| Hydrochloride of WX028 | 2.11 | 1113.90 |
| Formate of WX029 | 4.26 | 111.86 |
| Hydrochloride of WX030 | 4.18 | 110.35 |
| Hydrochloride of WX031 | 2.91 | 107.77 |

Conclusion: the compounds of the present disclosure exhibit excellent target protein degradation effects in K562 IRAK4-HiBiT cells.

### Experiment Example 2: Analysis of IKZF1 and IKZF3 Protein Expression Levels in MM.1S Cells Using in Cell Western

Experimental objective: The experiment aims to assess the degradation effect of the test compounds on IKZF1 and 1KZF3 proteins in MM.1S cells by evaluating the impact of the test compounds on the expression levels of IKZF1 and IZKZF3 proteins in MM.1S cells.

### Experimental Materials

Cell lines: MMN.1 S cell (sourced from ATCC; Cat. No CRL-2974)
Negative control: 0.100 DMSO.

TABLE 4
Reagents and consumables.

| Reagents and consumables | Supplier | Cat. No. |
|---|---|---|
| 96-well black-bottomed | Jing An Biological | J09603 |
| microplate | | |
| D-PBS buffer | Bioleaper | P220610030051 |
| Tissue fixative | Meilumbio | MA0192-Jan-28G |
| TritonX-100 Solution (10% | Beyotime | ST797-500ML |
| sterile) | | |
| Intercept Blocking Buffer | LI-COR | 211114 |
| Ikaros (D6N9Y) Rabbit mAb | Cell Signaling | 14859S |
| Aiolos (D1C1E) Rabbit mAb | Cell Signaling | 15103S |
| GAPDH mouse mAb | proteintech | 60004-1-Ig |
| Tween 20 | Sinopharm Chemical | 30189328 |
| | Reagent Co., Ltd | |
| IRDye 800CW Goat anti- | LI-COR | D00812-08 |
| mouse | | |
| IRDye 680RD Goat anti- | LI-COR | D00891-05 |
| Rabbit | | |

TABLE 5
Instruments

| Instrument name | Supplier | Instrument type |
|---|---|---|
| Dual-color infrared laser | LI-COR | Odyssey-CLx |
| imager | | |
| Decolorization shaker | QILNBEIER | TS-1000 |
| refrigerator | HAIER | BCD-256KT |

### Experimental Scheme

1) Log phase MM.1 S cells were plated in a 96-well plate at 1.2×10⁵ cells per well and cultured overnight;
2) The next day, compounds were added with a starting concentration of 300 nM, diluted threefold across three replicate wells for 10 concentration gradients (including DMSO), and the cells were incubated for 24 hours in an incubator;
3) The cells were centrifuged, and the supernatant was carefully removed. 150 μL of 4% paraformaldehyde fixative was added along the wall of the wells, avoiding contact with the bottom cells, and the cells were incubated at room temperature for 20 minutes;
4) Lysis buffer was prepared by mixing 0.5 mL of 10% Triton X-100 with 49.5 mL of PBS until homogenous;
5) 200 μL of lysis buffer was added along the well walls, again avoiding the bottom cells, and incubated on a shaker at room temperature for 5 minutes;
6) The wells were washed four times;
7) 150 μL of Licor INERCEPT Blocking Buffer was added along the well walls, avoiding the bottom cells, and the cells were incubated on a shaker at room temperature for 1.5 hours;
8) Ikaros (D6N9Y) Rabbit mAb, Aiolos (D1C1E) Rabbit mAb and GAPDH mouse mAb (proteintech, 60004-1-Ig) were diluted at a 1:100 ratio using antibody dilution buffer;
9) 50 μL of mixed antibody was added to each well with three replicate wells, and the cells were incubated overnight on a shaker at 4° C.;
10) PBST (PBS with 0.1% Tween 20) was prepared;
11) The primary antibodies were removed, and 200 μL of PBST was added along the well walls, avoiding the bottom cells, and the cells were incubated on a shaker at room temperature for 5 minutes;
12) The wells were washed four times;
13) Secondary antibody dilution buffer was prepared by adding Tween 20 to Licor INTERCEPT Blocking Buffer to a final concentration of 0.2%;
14) Fluorescent secondary antibodies were diluted in the dark at a 1:800 ratio. To 400 μL of secondary antibody dilution solution, 0.5 μL of each IRDye 800CW and IRDye 680CW (corresponding to the primary antibodies) was added.
15) 50 μL of the diluted fluorescent secondary antibodies was added to each well and the cells were incubated on a shaker at room temperature in the dark for 60 minutes;
16) The secondary antibodies were removed, and 200 μL of PBST was added along the well walls, avoiding the bottom cells, and the cells were incubated on a shaker at room temperature in the dark for 5 minutes;
17) The wells were washed four times; immediately after washing, the Odyssey Gel Imaging System was used for detection at dual wavelengths of 700 nm and 800 nm.

### 3. Data Analysis

Using GraphPad Prism 6 software, inhibition rate data was entered to fit the curve and calculate the DC₅₀ value.

Protein inhibition rate=(1−RLs/RLv)*100%
RR (Raw Ratio)=700 nm/800 nm
RLs=RR of sample-treated cells
RLv=RR of solvent-treated cells

The test results are shown in Table 6.

TABLE 6
Degradation effects of compounds of the present disclosure
on IKZF1 and IKZF3 proteins in MM.1S cells

| | | | Maximum |
|---|---|---|---|
| | | | degradation rate |
| Compound No. | Protein | DC₅₀ (nM) | Dmax (%) |
| Hydrochloride of | IKZF1 | 23.40 | 72.32 |
| WX014 | IKZF3 | 20.64 | 73.46 |
| Hydrochloride of | IKZF1 | 14.50 | 88.81 |
| WX018 | IKZF3 | 8.34 | 96.49 |
| Hydrochloride of | IKZF1 | 27.38 | 71.41 |
| WX030 | | | |
| Hydrochloride of | IKZF1 | 20.74 | 71.89 |
| WX031 | | | |
| Hydrochloride of | IKZF1 | 4.66 | 81.43 |
| WX008 | | | |
| Hydrochloride of | IKZF1 | 7.70 | 81.84 |
| WX015 | | | |
| Hydrochloride of | IKZF1 | 6.02 | 81.63 |
| WX016 | | | |
| Formate of WX017 | IKZF1 | 15.68 | 62.94 |
| Formate of WX020 | IKZF1 | 3.77 | 85.16 |
| WX021 | IKZF1 | 2.66 | 87.59 |
| Formate of WX023 | IKZF1 | 31.33 | 57.20 |
| Formate of WX024 | IKZF1 | 35.17 | 60.75 |
| Hydrochloride of | IKZF1 | 14.39 | 72.20 |
| WX025 | | | |
| Formate of WX026 | IKZF1 | 5.64 | 84.43 |
| Hydrochloride of | IKZF1 | 26.71 | 49.69 |
| WX028 | | | |
| Formate of WX029 | IKZF1 | 38.83 | 61.76 |

### Conclusion

The compounds of the present disclosure exhibit excellent target protein degradation effects on IZKZF1 and IZKZF3 proteins in MMN.1 S cells.

### Experiment Example 3: Evaluation of Antiproliferative Effects in Lymphoma Cell Lines OCI-LY10 and TMD-8

Experimental purpose: The experiment aims to evaluate the inhibitory effects of the test compounds on cell proliferation in the diffuse large B-cell lymphoma (DLBCL) cell lines OCI-LY10 and TMD-8.

### Experimental Materials

TABLE 7
Cell line and culture method

| | | Growth | |
|---|---|---|---|
| Cell line | Tumor type | characteristics | Culture medium |
| OCI-LY10 | Lymphoma | Suspension | IMDM + 20% |
| | | | FBS + 55 μM |
| | | | β-mercaptoethanol |
| TMD-8 | Lymphoma | Suspension | MEM + 10% FBS |

TABLE 8
Culture media and reagents

| Culture media and | | |
|---|---|---|
| reagents | Manufacturers | Cat. No. |
| Dulbecco's PBS | Hyclone | SH30256.01 |
| Fetal bovine serum | GIBCO | 10099-141 |
| (FBS) | | |
| Antibiotic-antimycotic | GIBCO | 15240-062 |
| 0.25% Trypsin | GIBCO | 25200072 |
| DMSO | SIGMA | D2650 |
| β-mercaptoethanol | SIGMA | 60-24-2 |
| MEM | GIBCO | 11095-080 |
| IMDM | GIBCO | 12440-053 |

### 1. Multi-Well Plate

Greiner CELLSTAR® 96-well plate, flat-bottom white plate (with lid and clear bottom), #3610.

### 2. Reagents and Instruments for Cell Viability Assay

(1) Promega CellTiter-Glo luminescent cell viability assay kit (Promega-G7573).
(2) 2104 EnVision® plate reader, PerkinElmer.

### Experimental Scheme

### 1. Cell Culture

The tumor cell lines were cultured in a 37° C., 5% CO₂ incubator under the above culture conditions. Regular passage, and cells in logarithmic growth phase were taken for plating.

### 2. Cell Plating

(1) Cells were stained with trypan blue and viable cells were counted.
(2) The cell concentration was adjusted to an appropriate concentration.

TABLE 9
Cell line and seeding density

| | Seeding density |
|---|---|
| Cell line | (cells/well) |
| OCI-LY10 | 5000 |
| TMD-8 | 5000 |

(3) 100 μL of cell suspension per well was added to the culture plate as shown above.
(4) The culture plate was cultured overnight in a 37° C., 5% CO₂ incubator with 100% relative humidity.

### 3. Preparation of Compound Storage Plate

Preparation of stock solution storage plate with 1000 times the initial concentration of the compound: The compound was serially diluted with DMSO from the highest concentration to the lowest concentration. The solution was prepared as needed each time.

4. Preparation of 1000× Compound Working Solution and Cell Treatment with Compounds
(1) Preparation of the working solution with 5 times the initial concentration of the compound: For 3-fold dilution of the compound, 30 μL of the compound from a stock solution at 1000-fold concentration was taken, and 20 μL of DMSO was added to the subsequent wells. Then, 10 μL was transferred from one concentration to the next in sequence. For 5-fold dilution of the compound, 30 μL of the compound from a stock solution at 1000-fold concentration was taken, and 24 μL of DMSO was added to the subsequent wells. Then, 6 μL was transferred from one concentration to the next in sequence. 20 μL of DMSO was added to the vehicle control. The 1000-fold concentrated compound was diluted 200 times with culture medium, i.e., 1 μL of the 1000-fold diluted compound is added to 199 μL of culture medium, and the mixture was homogenized using a pipette.
(2) Compound addition: 25 μL of the 5-fold compound solution was added to the cell culture plate.
(3) The 96-well cell plate was returned to the incubator to culture OCI-LY10 (with either 3-fold or 5-fold dilution, incubated with compounds added for 5 days) and TMD-8 (with either 3-fold or 5-fold dilution, incubated with compounds added for 5 days).

### 5. CellTiter-Glo Luminescent Cell Viability Assay

The following steps were performed according to the instructions of Promega CellTiter-Glo luminescent cell viability assay kit (Promega-G7573).
(1) CellTiter-Glo buffer was melted and brought to room temperature.
(2) CellTiter-Glo substrate was brought to room temperature.
(3) CellTiter-Glo working solution was prepared by adding 10 mL of CellTiter-Glo buffer to a vial of CellTiter-Glo substrate to dissolve the substrate.
(4) The mixture was slowly vortexed and shaken to be fully dissolved.
(5) The cell culture plate was taken out and left for 30 minutes to equilibrate to room temperature.
(6) 60 μL of CellTiter-Go working solution was added per well (equal to half the volume of cell culture medium per well). The cell plate was wrapped in aluminum foil to be protected from light.
(7) The culture plate was shaken on an orbital shaker for 2 minutes to induce cell lysis.
(8) The culture plate was left at room temperature for 10 minutes to stabilize the luminescent signal.
(9) The luminescent signal was detected on a 2104 EnVision plate reader.

### 6. Data Analysis

The inhibition rate (IIR) of the test compound was calculated using the following formula: IR (%)=(1−RLU compound/RLU vehicle control)*100%. The inhibition rates of compounds at different concentrations were calculated in Excel, and then GraphPad Prism software was used to plot inhibition curves and calculate relevant parameters, including minimum inhibition rate, maximum inhibition rate, and IC₅₀.

The test results are shown in Table 10.

TABLE 10
Inhibitory effect of compounds of the present disclosure
on cell proliferation in OCI-LY10 and TMD-8 cell lines

| | OCI-LY10 | | TMD-8 | |
|---|---|---|---|---|
| | | Maximum | | Maximum |
| | IC₅₀ | inhibition | IC₅₀ | inhibition |
| Compound No. | (nM) | rate (%) | (nM) | rate (%) |
| Hydrochloride of | 192.20 | 63.27 | 92.0 | 83.31 |
| WX002 | | | | |
| Hydrochloride of | 13.66 | 94.54 | 10.34 | 89.86 |
| WX014 | | | | |
| Trifluoroacetate of | 17.02 | 93.42 | 17.03 | 94.65 |
| WX011 | | | | |
| Hydrochloride of | 6.99 | 99.92 | 12.85 | 99.53 |
| WX018 | | | | |
| Hydrochloride of | 85.25 | 99.01 | / | / |
| WX028 | | | | |
| Hydrochloride of | 24.39 | 97.39 | / | / |
| WX025 | | | | |
| Formate of WX020 | 24.97 | 94.96 | / | / |
| Formate of WX026 | / | / | 8.65 | 99.92 |

“/” indicates not tested.

Conclusion: The compounds of the present disclosure exhibit excellent inhibitory effect on cell proliferation in both lymphoma cell lines OCI-LY10 and TMD-8.

### Experiment Example 4: Evaluation of Antiproliferative Effects in Lymphoma Cell Line SU-DHL-2

Experimental purpose: The experiment aims to evaluate the inhibitory effects of the test compounds on cell proliferation in the lymphoma cell line SU-DHL-2.

TABLE 11
Cell line and culture method

| | Tumor | Growth | |
|---|---|---|---|
| Cell line | type | characteristics | Culture method |
| SU- | Lymphoma | Suspension | RPMI1640 + 10% |
| DHL-2 | | | FBS (EXCELL) + 1% |
| | | | penicillin/streptomycin |

TABLE 12
Culture media and reagents

| Culture media and | | |
|---|---|---|
| reagents | Manufacturers | Cat. No. |
| RPMI 1640 | GIBCO | 22400-089 |
| Dulbecco's PBS | Thermo | SH30028.02B |
| Fetal bovine serum | Hyclone | 11H233 |
| (FBS) | | |
| Antibiotic-antimycotic | GIBCO | 15240-062 |
| DMSO | SIGMA | D2650 |

### 1. Multi-Well Plate

Greiner CELLSTAR 384-well plate, flat-bottom black plate (with lid), #781090.

### 2. Reagents and Instruments for Cell Viability Assay

(1) Promega CellTiter-Glo luminescent cell viability assay kit (Promega-G7573).
(2) 2104 EnVision® plate reader, PerkinElmer.

### Experimental Scheme

### 1. Cell Culture

The tumor cell lines were cultured in a 37° C., 5% CO₂ incubator under the above culture conditions. Regular passage, and cells in logarithmic growth phase were taken for plating.

### 2. Cell Plating

(1) Cells were stained with trypan blue and viable cells were counted.
(2) The cell concentration was adjusted to an appropriate concentration.

TABLE 13
Cell line and seeding density

| | Seeding density |
|---|---|
| Cell line | (cells/well) |
| SU-DHL-2 | 1500 |

(3) 50 μL of cell suspension per well was added to the culture plate as shown in the figure above.
(4) The culture plate was cultured overnight in a 37° C., 5% CO₂ incubator with 100% relative humidity.

### 3. Preparation of Compound Storage Plate

Compounds were added using the Echo655 instrument. The volume of compound added was 50 nL, with a final DMSO concentration of 0.1%. The culture plate was centrifuged at 1000 rpm for 1 minute, then incubated for 4 days in an incubator at 37° C., 5% CO2, and with 100% relative humidity.

### 4. CellTiter-Glo Luminescent Cell Viability Assay

The following steps were performed according to the instructions of Promega CellTiter-Glo luminescent cell viability assay kit (Promega-G7573).
(1) CellTiter-Glo buffer was melted and brought to room temperature.
(2) CellTiter-Glo substrate was brought to room temperature.
(3) CellTiter-Glo working solution was prepared by adding CellTiter-Glo buffer to a vial of CellTiter-Glo substrate to dissolve the substrate;
(4) The mixture was slowly vortexed and shaken to be fully dissolved.
(5) The cell culture plate was taken out and left for 30 minutes to equilibrate to room temperature.
(6) 25 μL of CellTiter-Glo working solution was added per well (equal to half the volume of cell culture medium per well). The cell plate was wrapped in aluminum foil to be protected from light.
(7) The culture plate was shaken on an orbital shaker for 2 minutes to induce cell lysis.
(8) The culture plate was left at room temperature for 10 minutes to stabilize the luminescent signal.
(9) The luminescent signal was detected on a 2104 EnVision plate reader.

### 5. Data Analysis

The inhibition rate (IR) of the test compound was calculated using the following formula: IR (%)=(1−(RLU compound/RLU blank control)/(RLU vehicle control−RLU blank control)×100%. The inhibition rates of compounds at different concentrations were calculated in Excel, and then GraphPad Prism software was used to plot inhibition curves and calculate relevant parameters, including minimum inhibition rate, maximum inhibition rate, and IC₅₀.

TABLE 14
Inhibitory effect of compounds of the present disclosure
on cell proliferation in SU-DHL-2 cell line

| | SU-DHL-2 IC₅₀ | Maximum inhibition |
|---|---|---|
| Compound No. | (nM) | rate (%) |
| Hydrochloride of | 11.74 | 100.27 |
| WX018 | | |
| Hydrochloride of | 64.52 | 99.05 |
| WX004 | | |
| Formate of WX026 | 0.91 | 100.08 |
| Hydrochloride of | 28.70 | 93.55 |
| WX014 | | |
| Trifluoroacetate of | 25.20 | 99.89 |
| WX011 | | |

### Conclusion

The compounds of the present disclosure exhibit excellent inhibitory effect on cell proliferation in lymphoma cell line SU-DHL-2.

### Experimental Example 5: Pharmacokinetic Evaluation of Compounds in Rats

### Experimental Purpose

In this study, C₅₇BL/6N or CD-1 male mice were selected as test animals, and LC/MS/MS method was used to quantify plasma concentrations of the test compound intravenously injected or gavage to mice at different time points, so as to evaluate the pharmacokinetic profile of the compounds of the present disclosure in mice.

### Experimental Materials

C57BL/6N mice (male, 20 to 30 g, 7 to 10 weeks old, Beijing VitalRiver) or CD-1 mice (male, 20 to 35 g, 7 to 10 weeks old, Beijing VitalRiver).

### Experimental Operation A

The test compounds, either as clear or suspension solutions, were administered intravenously through the tail vein to C57BL/6N mice or CD-1 mice (either fasted overnight or fed) (vehicle: 10% DMSO/10% solutol/80% H₂O or 5% DMSO/10% solutol/85% H₂O), or intragastrically administered to C57BL/6N mice or CD-1 mice (either fasted overnight or fed). For intravenous injection, blood 50 μL was collected via the cheek vein puncture at 0 h (pre-administration), 0.083, 0.25, 0.5, 1, 2, 4, 6, 8, and 24 h after administration, and placed in an anticoagulant tube added with heparin sodium. The mixture was thoroughly vortexed and mixed, and centrifuged at 6000 g for 3 minutes at 2-8° C. For oral administration, blood was collected via the cheek vein puncture at 0 h (pre-administration), 0.083, 0.25, 0.5, 1, 2, 4, 6, 8, and 24 h after administration, and placed in an anticoagulant tube added with heparin sodium. The mixture was thoroughly vortexed and mixed, and centrifuged at 6000 g for 3 minutes at 2-8° C. Plasma concentrations were determined by LC-MS/MS method, and relevant pharmacokinetic parameters were calculated using Phoenix WinNonlin8.2.0 pharmacokinetic software with non-compartmental model linear-log trapezoidal method.

### Experimental Operation B

The test compounds, as clear solutions, were administered intravenously through the tail vein to CD-1 mice (fasted overnight) (vehicle: 10% DMSO/10% solutol/80% H₂O or 5% DMSO/10% solutol/85% H₂O), or intragastrically administered to CD-1 mice (fasted overnight). For intravenous injection, blood was collected via the saphenous vein puncture at 0 h (pre-administration), 0.083, 0.25, 0.5, 1, 2, 4, 8, and 24 h after administration, and placed in a EDTA-K2 anticoagulant tube. The mixture was thoroughly vortexed and mixed, and centrifuged at 3200 g for 10 minutes at 4° C. to obtain plasma. For oral administration, blood was collected via the saphenous vein puncture at 0 h (pre-administration), 0.083, 0.25, 0.5, 1, 2, 4, 8, and 24 h after administration, and placed in a EDTA-K2 anticoagulant tube. The mixture was thoroughly vortexed and mixed, and centrifuged at 3200 g for 10 minutes at 4° C. to obtain plasma. Plasma concentrations were determined by LC-MS/MS method, and relevant pharmacokinetic parameters were calculated using Phoenix WinNonlin 6.3 pharmacokinetic software with non-compartmental model linear-log trapezoidal method.

The test results are shown in Table 15.

TABLE 15
Pharmacokinetic parameters of compounds of the present disclosure in mice

| | Pharmacokinetic parameters in mice | | | | | | | | |
|---|---|---|---|---|---|---|---|---|---|
| | Intravenous injection (2 mg/kg) | | | | | Intragastric administration (10 mg/kg) | | | |
| | | | | | Area | | | Area | |
| | | | | | under | | | under | |
| | | Plasma | | | drug- | | | drug- | |
| | | clearance | | Apparent | time | | Time to | time | |
| | | rate | Elimination | volume of | curve | Peak | peak | curve | Bioavail |
| Compound | Experimental | (mL/min/ | half-life | distribution | (0-inf, | concentration | concentration | (0-inf, | ability F |
| No. | operation: | kg) | (h) | (L/kg) | μM · h) | (μM) | (h) | μM · h) | (%) |
| Hydrochloride | B | / | / | | / | 0.64 | 1.00 | 2.00 | / |
| of WX002 | | | | | | | | | |
| (fasted overnight) | | | | | | | | | |
| Hydrochloride | A | 7.0 | 4.22 | 1.6 | 5.40 | 0.54 | 1.00 | 4.47 | 18.90 |
| of WX014 (fed) | | | | | | | | | |
| Trifluoroacetate | A | 2.76 | 9.45 | 1.8 | 15.31 | 0.45 | 1.67 | 5.00 | 6.53 |
| of WX011 | | | | | | | | | |
| (fasted overnight) | | | | | | | | | |
| Hydrochloride | B | 11.8 | 5.22 | 4.39 | 3.06 | 0.09 | 6.00 | 1.01 | 6.59 |
| of WX018 | | | | | | | | | |
| (fasted overnight) | | | | | | | | | |
| Formate of WX019 | A | / | / | / | / | 0.34 | 3.33 | 5.10 | / |
| (fasted overnight) | | | | | | | | | |
| Formate of WX017 | B | 12.2 | 2.32 | 2.59 | 3.17 | 0.65 | 4.00 | 5.07 | 31.9 |
| (fasted overnight) | | | | | | | | | |

“/” indicates not tested.

Conclusion: the compounds of the present disclosure have high plasma system exposure (AUC_{0-inf}) when administered orally. The compounds of the present disclosure have superior pharmacokinetic properties in rodents like mice.

### Experimental Example 7: Pharmacokinetic Evaluation of Compounds in Beagles

### Experimental Objective

In this study, male beagles were selected as test animals, and LC/MS/MS method was used to quantify plasma concentrations of the test compound intravenously injected or intragastrically administered to beagles at different time points, so as to evaluate the pharmacokinetic profile of the compounds of the present disclosure in beagles.

### Experimental Materials

Beagle (male, 7 to 10 kg, Beijing Marshall Biotechnology Co., Ltd.).

### Experimental Operation

A clear solution of the test compound was slowly injected into beagle via the peripheral vein (fed) (vehicle: 500 DMSO/10% solutol/85% H₂O or 5% DMSO/10% solutol/85% H₂O), or intragastrically administered to beagles (fed). For intravenous injection, 0.5 mL blood was collected via the saphenous vein puncture at 0.083, 0.25, 0.5, 1, 2, 4, 6, 8, and 24 h after administration, and placed in a EDTA-2K anticoagulant tube. The mixture was centrifuged at 3200 g for 10 minutes at 2 to 8° C., and the supernatant was separated. For oral administration, 0.5 mL blood was collected via the saphenous vein puncture at 0.083, 0.25, 0.5, 1, 2, 4, 6, 8, and 24 h after administration, and placed in a EDTA-2K anticoagulant tube. The mixture was centrifuged at 3200 g for 10 minutes at 2 to 8° C., and the supernatant was separated. Plasma drug concentrations were determined by LC-MS/MS method, and relevant pharmacokinetic parameters were calculated using Phoenix WinNonlin 6.3 pharmacokinetic software with non-compartmental model linear-log trapezoidal method.

The test results are shown in Table 16.

TABLE 16
Pharmacokinetic parameters of compounds of the present disclosure in beagles

| | Pharmacokinetic parameters of beagles | | | | | | | |
|---|---|---|---|---|---|---|---|---|
| | Intravenous injection (0.5 mg/kg) | | | | Intragastric administration (5 mg/kg) | | | |
| | | | | Area | | | Area | |
| | | | | under | | | under | |
| | Plasma | | | drug- | | | drug- | |
| | clearance | | Apparent | time | | | time | |
| | rate | Elimination | volume of | curve | Peak | Time to peak | curve | Bioavail |
| Compound | (mL/min/ | half-life | distribution | (0-inf, | concentration | concentration | (0-inf, | ability F |
| No. | kg) | (h) | (L/kg) | μM · h) | (μM) | (h) | μM · h) | (%) |
| Hydrochloride | 21.3 | 5.03 | 5.83 | 0.44 | 0.41 | 4.00 | 3.20 | 72.6 |
| of WX014 | | | | | | | | |

### Conclusion

The compounds of the present disclosure have relatively high plasma system exposure (AUC_{0-inf}) when administered orally. The compounds of the present disclosure have superior pharmacokinetic properties in non-rodents like beagles.

### Experimental Example 8: In Vivo Drug Efficacy Evaluation of Compounds in SCID Mouse Xenograft Model of Human B-Cell Lymphoma OCI-LY10 Cell

### Experimental Objective

The study evaluates the antitumor effect of the test compounds using a SID mouse xenograft model of human B-cell lymphoma OCI-LY10 cells.

### Experimental Materials

1. Experimental animals: SCID mice, female, 6 to 8 weeks old, weighing 17 to 20 g. Beijing Vital River Laboratory Animal Technology Co., Ltd.
2. Cell line: The human B-cell lymphoma OCI-LY10 cell line was purchased from Nanjing Cobioer Biosciences Co. Ltd (Cat. No CBP60558).

TABLE 17
Reagent information

| Name | Manufacturer | Batch number | Storage condition | |
|---|---|---|---|---|
| IMDM | GIBCO | 2323369 | 4° | C. |
| culture medium | Corporation | | | |
| | (US) | | | |
| Fetal bovine serum | GIBCO | 2305262RP | −20° | C. |
| (FBS) | Corporation | | | |
| | (US) | | | |
| Matrix glue | CORNING | 2062001 | −20° | C. |
| (Matrigel) | | | | |

TABLE 18
Instruments

| Name | Manufacturer | Type |
|---|---|---|
| SHJ series | Shanghai Shangjing | CA-1390-1 |
| clean workbench | Purifying | |
| | Equipment Co., Ltd. | |
| CO₂ water-jacketed | Thermo Scientific Forma | 3111 |
| cell culture incubator | | |
| Inverted microscope | Olympus | CKX41SF |
| Electric aspirator | Shanghai MEDICAL | YX930D |
| | Instruments | |
| | (GROUP) Co., Ltd. | |
| Eppendorf AG | Eppendorf | 5811XG639987 |
| centrifuge | | |
| Hundredth balance scale | Shanghai Minqiao Precise | SL502N |
| | Instrument Co., Ltd. | |
| Thousandth balance scale | Shanghai Jinghai | JA2003N |
| | Instrument | |
| | Co., Ltd. | |
| Electronic digital caliper | SHAHE | (0 to 150) mm |

### Model Building:

OCI-LY10 cells were cultured in IMDM medium containing 20% FBS and maintained in a 37° C. incubator with 5% CO₂ and saturated humidity. Log phase OCI-LY10 cells were collected, resuspended in IMDM basic medium, and mixed 1:1 with Matrigel. The cell concentration was adjusted to 4×10⁷/mL. Under sterile conditions, 0.1 mL of the cell suspension was inoculated subcutaneously into the right flank of SCID mice, with an inoculation concentration of 4×10⁶/0.1 mL/mouse.

### Experimental Scheme

For the pharmacological experiment, once the tumors reached a certain size, animals with tumors that were either too large, too small, or irregularly shaped were eliminated. Animals with tumor volumes between 167.65 and 231.29 mm³ were selected. Based on tumor volume, the animals were divided into groups using a random grouping method, with each group consisting of six mice, and the average tumor volume was approximately 201.15 mm³. The day of grouping was designated as Day 0, and dosing began according to the body weight of the animals. The pharmacological study period lasted for 28 days, with dosing once a day and a 24-hour interval between doses. Intragastric administration was used for administration. During the experiment, the body weight and tumor size of the animals were measured twice a week. Clinical symptoms were observed and recorded daily.

The test compounds were administered at doses of 10 mg/kg, 30 mg/kg, and 100 mg/kg, with a solvent composition of 10% DMSO/10% Solutol/80% H₂O. The formula for calculating tumor volume (TV) was: 1/2×a×b², where a and b were the measured length and width of the tumor, respectively. The formula for calculating the Tumor Growth Inhibition (TGI) rate was: TGI (%)=[(1−(average tumor volume at the end of administration in a treatment group−average tumor volume at the beginning of administration in this treatment group))/(average tumor volume at the end of treatment in the vehicle control group−average tumor volume at the beginning of treatment in the vehicle control group)]×100%. Relative Tumor Proliferation rate T/C (%): the calculation formula was as follows: T/C %=T_{RTV}/C_{RTV}×100% (T_{RTV}: average RTV of the treatment group; C_{RTV}: average RTV of the negative control group). According to the results of tumor measurement, the relative tumor volume (RTV) was calculated, and the calculation formula was RTV=Vt/V₀, where V₀ was the tumor volume measured at the time of group administration (i.e. Day 0), V_{T} was the tumor volume at a certain measurement, and T_{RTV} and C_{RTV} were taken from the same day's data.

### Data Analysis:

In this study, experimental data were presented in Mean±SEM.

Statistical analysis was performed using IBM SPSS Statistics software based on the RTV data at the end of the experiment. For comparisons between two groups, a T test was used. For comparisons among three or more groups, one-way ANOVA was utilized. If the variance was homogenous (no significant difference in F value), Tukey's method was applied for analysis. If the variance was heterogeneous (significant difference in F value), the Games-Howell method was used for testing. A p-value of T 0.05 was considered statistically significant.

Experimental results: The test results were shown in Table 19 and 20.

TABLE 19
Evaluation of antitumor efficacy of compounds of the present disclosure in
subcutaneous xenograft tumor model of human B-cell lymphoma OCI-LY10 cells

| | | Tumor | Tumor | Relative | | |
|---|---|---|---|---|---|---|
| | | volume | volume | tumor | | |
| | | (mm³)^{a} | (mm³)^{a} | volume^{a} | T/C (%) | TGI (%) |
| Group | Dose | (Day 0) | (Day 28) | (Day 28) | (Day 28) | (Day 28) |
| vehicle control | 0 | 201.76 ± | 1422.84 ± | 7.11 ± | — | — |
| | mg/kg | 7.08 | 89.14 | 0.56 | | |
| Hydrochloride of | 10 | 202.43 ± | 685.61 ± | 3.37 ± | 47.40 | 60.43 |
| WX014 | mg/kg | 8.41 | 75.95 | 0.34 | | |
| Hydrochloride of | 30 | 199.80 ± | 322.68 ± | 1.64 ± | 23.07 | 89.94 |
| WX014 | mg/kg | 8.02 | 20.47 | 0.17 | | |
| Hydrochloride of | 100 | 199.75 ± | 188.01 ± | 0.91 ± | 12.80 | 100.96 |
| WX014 | mg/kg | 7.96 | 35.59 | 0.15 | | |

Note:
^{a}Average ± SEM.

TABLE 20
p-Values of compounds of the present disclosure for
comparison of relative tumor volumes among groups
in xenograft model of human B-Cell lymphoma OCI-LY10

| | | Hydro- | Hydro- | Hydro- |
|---|---|---|---|---|
| | | chloride | chloride | chloride |
| | | of | of | of |
| | vehicle | WX014 | WX014 | WX014 |
| Group | control | 10 mg/kg | 30 mg/kg | 100 mg/kg |
| vehicle | N/A | 0.003 | 0.001 | <0.001 |
| control | | | | |
| Hydro- | 0.003 | N/A | 0.018 | 0.003 |
| chloride | | | | |
| of WX014 | | | | |
| 10 mg/kg | | | | |
| Hydro- | 0.001 | 0.018 | N/A | 0.077 |
| chloride | | | | |
| of WX014 | | | | |
| 30 mg/kg | | | | |
| Hydro- | <0.001 | 0.003 | 0.077 | N/A |
| chloride | | | | |
| of WX014 | | | | |
| 100 mg/kg | | | | |

Note:
P value was analyzed by IBM SPSS Statistics software.

### Conclusion

The compounds of the present disclosure exhibit remarkable tumor-suppressing effects and is dose-dependent in a SCID mouse xenograft tumor model using human B-cell lymphoma OCI-LY10 cells.

### Experiment Example 9: In Vivo Drug Efficacy Study of Compounds in CB17 SCID Mouse Model with Subcutaneous Xenograft Tumors of Human Lymphoma SU-DHL-2 Cells

### Experimental Objective

The study evaluates the antitumor effect of the test compounds using a CB17 SCID mouse model with subcutaneous xenograft tumors of SU-DHL-2 human lymphoma cells.

### Experimental Materials

1. Experimental animals: CB17 SCID mice, female, 6 to 8 weeks old, weighing 18 to 22 g. Beijing Vital River Laboratory Animal Technology Co., Ltd.
2. Cell line: human lymphoma SU-DHL-2 cells (Cat. No ATCC-CRL-2956).

TABLE 21
Reagent information

| | | | Batch | Period of | Storage | |
|---|---|---|---|---|---|---|
| Name | Manufacturer | Cat. No. | number | validity | condition | |
| 1640 | gibco | 22400089 | 2462009 | 2023 Feb. 28 | 4° | C. |
| culture | | | | | | |
| medium | | | | | | |
| Matrix gel | Corning | 354234 | 2013002 | 2022 Oct. 19 | −20° | C. |
| 100 × | meilunbio | MA0110 | MA0110-Apr-15H | 2023 Apr. 14 | −20° | C. |
| Double | | | | | | |
| antibody | | | | | | |
| (penicillin, | | | | | | |
| streptomycin) | | | | | | |
| PBS | HyClone | SH30256.01 | AG29791799 | 2023 Aug. 31 | 4° | C. |
| Fetal | ExCell Bio | FND500 | 11H233 | 2023 August | −20° | C. |
| bovine | | | | | | |
| serum | | | | | | |

TABLE 22
Instruments

| Name | Manufacturer | Type |
|---|---|---|
| Biological safety | Suzhou Antai Air | BSC-1604IIA2 |
| cabinet | Tech Co., Ltd. | |
| Vernier scale | Mitutoyo | CD-6″ASX |
| Electronic | Changzhou | EL-2KJ |
| balance scale | Tianzhiping Instrument | |
| | Equipment Co., Ltd | |
| Vortex mixer | HaiMen City Qilin | XW-80A |
| | Medical Instrument Factory | |
| Pure water system | Millipore | MILLI- |
| | | Q ® Direct8 |

Cell culture: Human lymphoma SU-DHL-2 cells (ATCC-CRL-2956) were cultured in suspension in vitro. The culture conditions were RPMI 1640 medium with 1000 inactivated fetal bovine serum, 100 U/mL penicillin and 100 μg/mL streptomycin, and cultured in a 37° C., 500 CO₂ incubator. Routine passaging was performed twice a week. When the cell saturation reached 800%-90%0 and the quantity met the requirements, the cells were collected, counted, and seeded.

Tumor cell seeding and grouping: 0.2 mL (10×10⁶ cells) of SU-DHL-2 cells, in a PBS:Matrigel=1:1 mixture, were subcutaneously inoculated into the right dorsal flank of each mouse. When the average tumor volume reached approximately 139 mm³, the animals were divided into groups for drug administration. The day of grouping was recorded as Day 0, and dosing began according to the body weight of the animals.

### Experimental Scheme

The pharmacological experiment consisted of a dosing cycle of seven days, with medication administered once daily at a 24-hour interval. The test compounds were given intragastrically, with a total of three dosing cycles. During the experiment, the body weight and tumor size of the animals were measured twice a week, and clinical symptoms were observed and recorded daily.

The dosing for the test compounds were 10 mg/kg, 30 mg/kg, and 100 mg/kg, with a solvent composition of 10% DMSO/10% Solutol/80% water. The formula for calculating tumor volume (TV) was: 1/2×a×b², where a and b were the measured length and width of the tumor, respectively. The formula for calculating the Tumor Growth Inhibition (TGI) rate was: TGI (%)=[(1−(average tumor volume at the end of administration in a treatment group average tumor volume at the beginning of administration in this treatment group))/(average tumor volume at the end of treatment in the vehicle control group−average tumor volume at the beginning of treatment in the vehicle control group)]×100%. Relative Tumor Proliferation rate T/C (%): the calculation formula was as follows: T/C %=T_{RTV}/C_{RTV}×100% (T_{RTV}: average RTV of the treatment group; C_{RTV}: average RTV of the negative control group). According to the results of tumor measurement, the relative tumor volume (RTV) was calculated, and the calculation formula was RTV=Vₜ/V₀, where V₀ was the tumor volume measured at the time of group administration (i.e. Day 0), VT was the tumor volume at a certain measurement, and T_{RTV} and C_{RTV} were taken from the same day's data.

### Data Analysis:

Statistical analysis is based on the data of RTV at the end of the experiment and analyzed by SPSS software. For comparisons between two groups, a T test was used. For comparisons among three or more groups, one-way ANOVA was utilized. If the variance was homogenous (no significant difference in F value), Tukey's method was applied for analysis. If the variance was heterogeneous (significant difference in F value), the Games-Howell method was used for testing. A p-value of <0.05 was considered statistically significant.

Experimental results: The test results were shown in Table 23 and 24.

TABLE 23
Evaluation of antitumor efficacy of compounds of the present disclosure
in subcutaneous xenograft tumor model of lymphoma SU-DHL-2 cells

| | | Tumor | Tumor | Relative | | |
|---|---|---|---|---|---|---|
| | | volume | volume | tumor | T/C | TGI |
| | | (mm³)^{a} | (mm³)^{a} | volume^{a} | (%) | (%) |
| Group | Dose | (Day 0) | (Day 21) | (on day 21) | (Day 21) | (Day 21) |
| Vehicle control | 0 | 139 ± | 1883 ± | 13.68 | — | — |
| | mg/kg | 6 | 141 | | | |
| Hydrochloride | 10 | 139 ± | 800 ± | 5.79 | 42.33 | 62.13 |
| of WX014 | mg/kg | 6 | 48 | | | |
| Hydrochloride | 30 | 139 ± | 813 ± | 5.78 | 42.26 | 61.41 |
| of WX014 | mg/kg | 6 | 99 | | | |
| Hydrochloride | 100 | 139 ± | 626 ± | 4.55 | 33.23 | 72.07 |
| of WX014 | mg/kg | 5 | 51 | | | |

Note:
^{a}Average ± SEM.

TABLE 24
p-Values of compounds of the present disclosure for comparison
of relative tumor volumes among groups in subcutaneous
xenograft tumor model of lymphoma SU-DHL-2

| | | Hydro- | Hydro- | Hydro- |
|---|---|---|---|---|
| | | chloride | chloride | chloride |
| | | of | of | of |
| | Vehicle | WX014 | WX014 | WX014 |
| Group | control | 10 mg/kg | 30 mg/kg | 100 mg/kg |
| Vehicle | N/A | 0.001 | 0.001 | <0.001 |
| control | | | | |
| Hydro- | 0.001 | N/A | 1.000 | 0.268 |
| chloride | | | | |
| of WX014 | | | | |
| 10 mg/kg | | | | |
| Hydro- | 0.001 | 1.000 | N/A | 0.507 |
| chloride | | | | |
| of WX014 | | | | |
| 30 mg/kg | | | | |
| Hydro- | <0.001 | 0.268 | 0.507 | N/A |
| chloride | | | | |
| of WX014 | | | | |
| 100 mg/kg | | | | |

Note:
p-Values for the analysis of relative tumor volume (RTV) are derived using one-way ANOVA. In case of heterogeneity of variances, the Games-Howell test is applied for analysis.

### Conclusion

The compounds of the present disclosure have remarkable tumor-suppressing effects in a CB17 SCID mouse model with subcutaneous xenograft tumors of human lymphoma SU-DHL-2 cells.

1. A compound of formula (II), a stereoisomer thereof, or a pharmaceutically acceptable salt thereof, 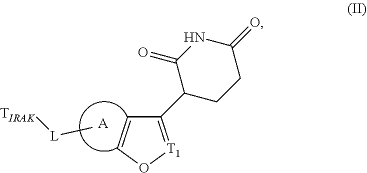
wherein
T_{IRAK} is selected from 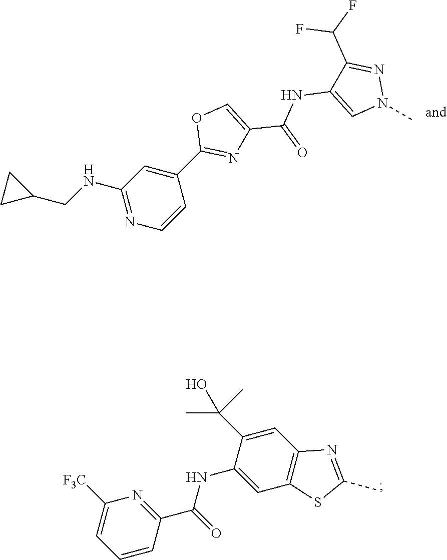
L is selected from C₂₋₁₀ alkylene, wherein any 2, 3 or 4 CH₂ on the C₂₋₁₀ alkylene are each independently substituted by Ra, and each C₂₋₁₀ alkylene is independently and optionally substituted by 1, 2, 3, 4, 5, or 6 halogens;
each Ra is independently selected from —N(R)—, —O—, —C(O)NH—, —C₃₋₆ cycloalkyl-, and -4- to 8-membered heterocycloalkyl-;
R is selected from H and C₁₋₄ alkyl;
T₁ is selected from CH and N;
ring A is selected from C₆₋₁₀ aryl and 5- to 10-membered heteroaryl.

2. The compound, the stereoisomer thereof, or the pharmaceutically acceptable salt thereof according to claim 1, wherein Ra is independently selected from —NH—, —N(CH₃)—, —O—, —C(O)NH—, -cyclopropyl-, -cyclobutyl-, -cyclopentyl-, -cyclohexyl-, -piperidyl-, -piperazinyl-, -azaspiro[3.3]heptyl-, -diazspiro[3.3]heptyl-, and -azabicyclic[3.1.0]hexyl-.

3. The compound, the stereoisomer thereof, or the pharmaceutically acceptable salt thereof according to claim 2, wherein each Ra is independently selected from —NH—, —N(CH₃)—, —O—, —C(O)NH—,

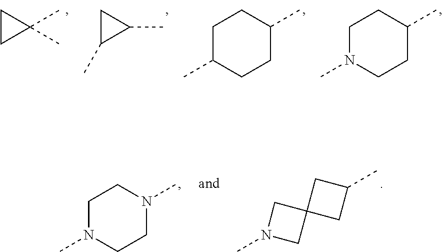

4. The compound, the stereoisomer thereof, or the pharmaceutically acceptable salt thereof according to claim 1, wherein L is selected from C₄₋₁₀ alkylene, any 3 CH₂ on the C₄₋₁₀ alkylene are each independently substituted by Ra, and each C₄₋₁₀ alkylene is independently and optionally substituted by 1, 2, 3, 4, 5 or 6 halogens.

5. The compound, the stereoisomer thereof, or the pharmaceutically acceptable salt thereof according to claim 4, wherein L is selected from —C₃₋₆ cycloalkyl-CH₂—Ra—C₁₋₃ alkylene-Ra—C₀₋₃ alkylene-, —C₃₋₆ cycloalkyl-C₁₋₃ alkylene-4- to 8-membered heterocycloalkyl-C₁₋₃ alkylene-Ra—, and —C₃₋₆ cycloalkyl-C₁₋₃ alkylene-4- to 8-membered heterocycloalkyl-C₁₋₃ alkylene-, and each C₁₋₃ alkylene is independently and optionally substituted by 1 or 2 halogen atoms.

6. The compound, the stereoisomer thereof, or the pharmaceutically acceptable salt thereof according to claim 5, wherein L is selected from -cyclohexyl-CH₂—N(R)—C₁₋₃ alkylene-O—C₁₋₃ alkylene-, -cyclohexyl-CH₂-4- to 8-membered heterocycloalkyl-C₁₋₃ alkylene-N(R)—, -cyclohexyl-CH₂-piperidyl-C₁₋₃ alkylene-C(O)NH—, -cyclohexyl-CH₂-piperidyl-CF₂—C(O)NH—, -cyclohexyl-CH₂-piperazinyl-C₁₋₃ alkylene-C(O)NH—, and -cyclohexyl-CH₂-piperazinyl-C₁₋₃ alkylene-.

7. The compound, the stereoisomer thereof, or the pharmaceutically acceptable salt thereof according to claim 6, wherein L is selected from

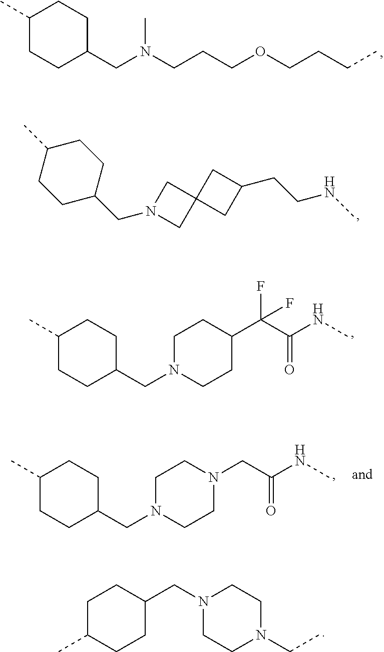

8. The compound, the stereoisomer thereof, or the pharmaceutically acceptable salt thereof according to claim 1, wherein ring A is selected from phenyl or naphthyl.

9. The compound, the stereoisomer thereof, or the pharmaceutically acceptable salt thereof according to claim 1, wherein the structural moiety 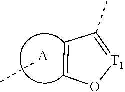
is selected from

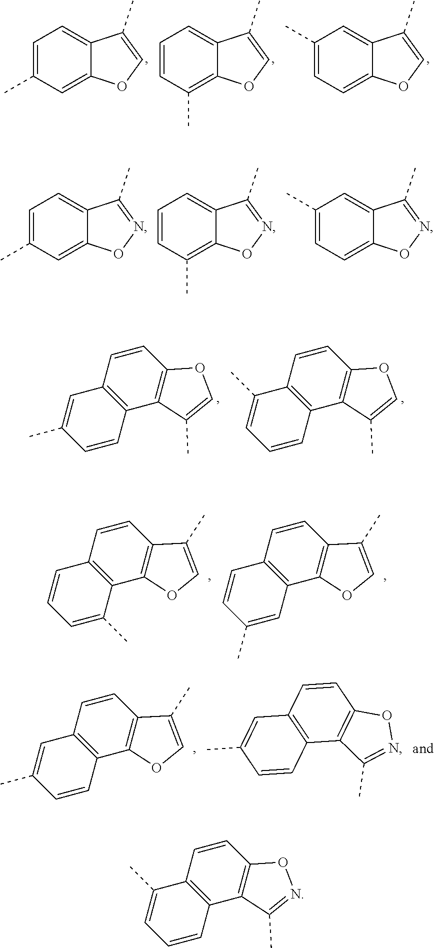

10. The compound, the stereoisomer thereof, or the pharmaceutically acceptable salt thereof according to claim 1, selected from the structures of formulas (II-1), (II-2), (II-3), and (II-4),

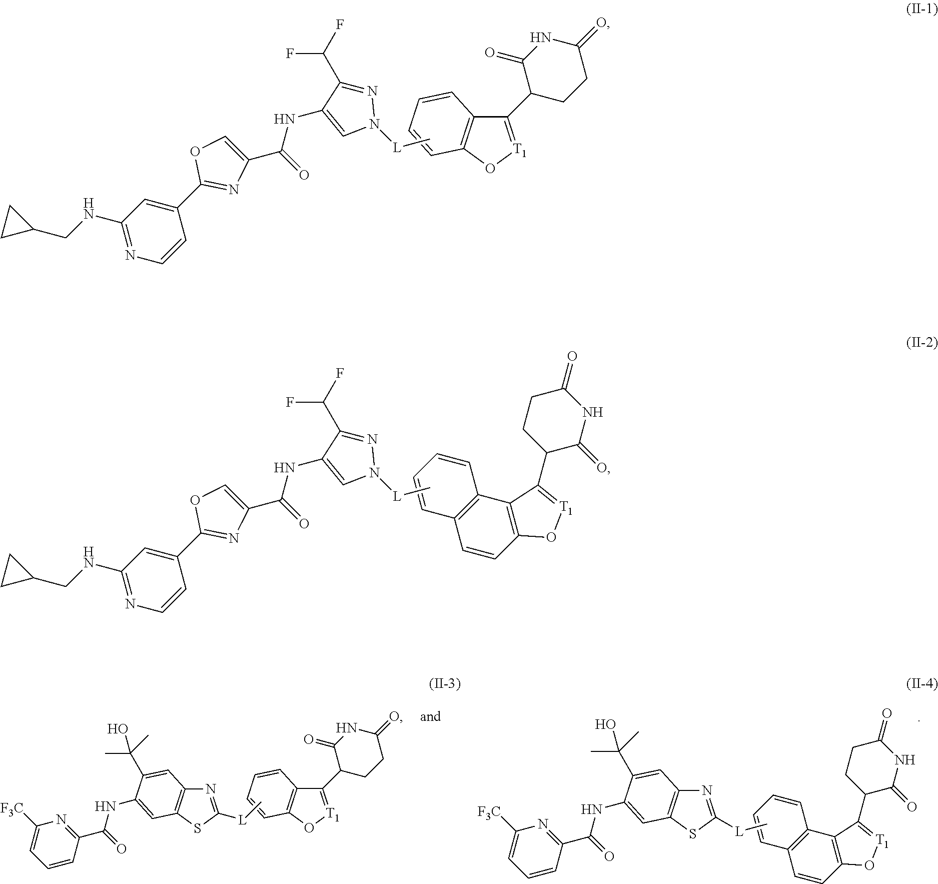

11. The compound, the stereoisomer thereof, or the pharmaceutically acceptable salt thereof according to claim 1, selected from the structures of formulas (II-1a), (II-2a), (II-3a), and (II-4a), 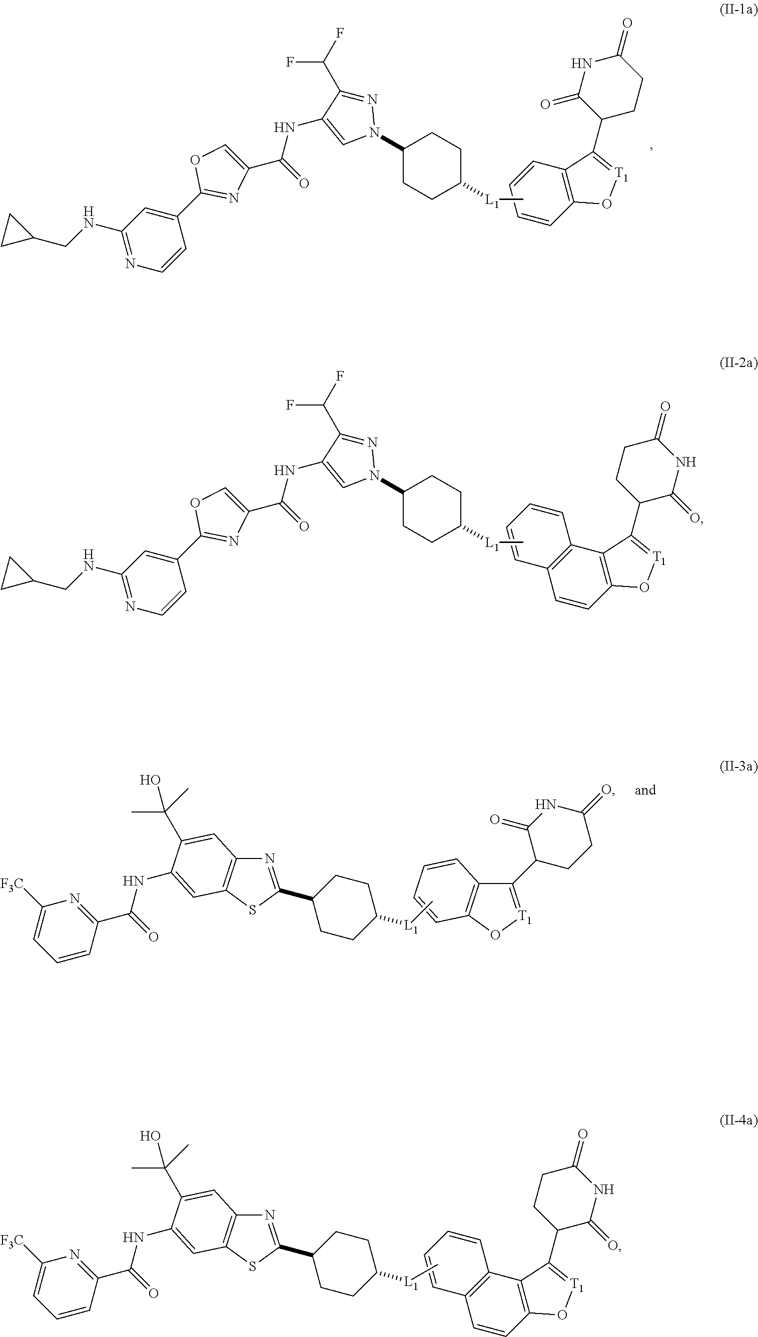
wherein
L₁ is selected from —C₁₋₃ alkylene-Ra—C₁₋₃ alkylene-Ra—C₀₋₃ alkylene-, —C₁₋₃ alkylene-4- to 8-membered heterocycloalkyl-C₁₋₃ alkylene-Ra—, and —C₁₋₃ alkylene-4- to 8-membered heterocycloalkyl-C₁₋₃ alkylene-, and each C₁₋₃ alkylene is independently and optionally substituted by 1 or 2 halogen atoms.

12. A compound, a stereoisomer thereof, or a pharmaceutically acceptable salt thereof as shown below,

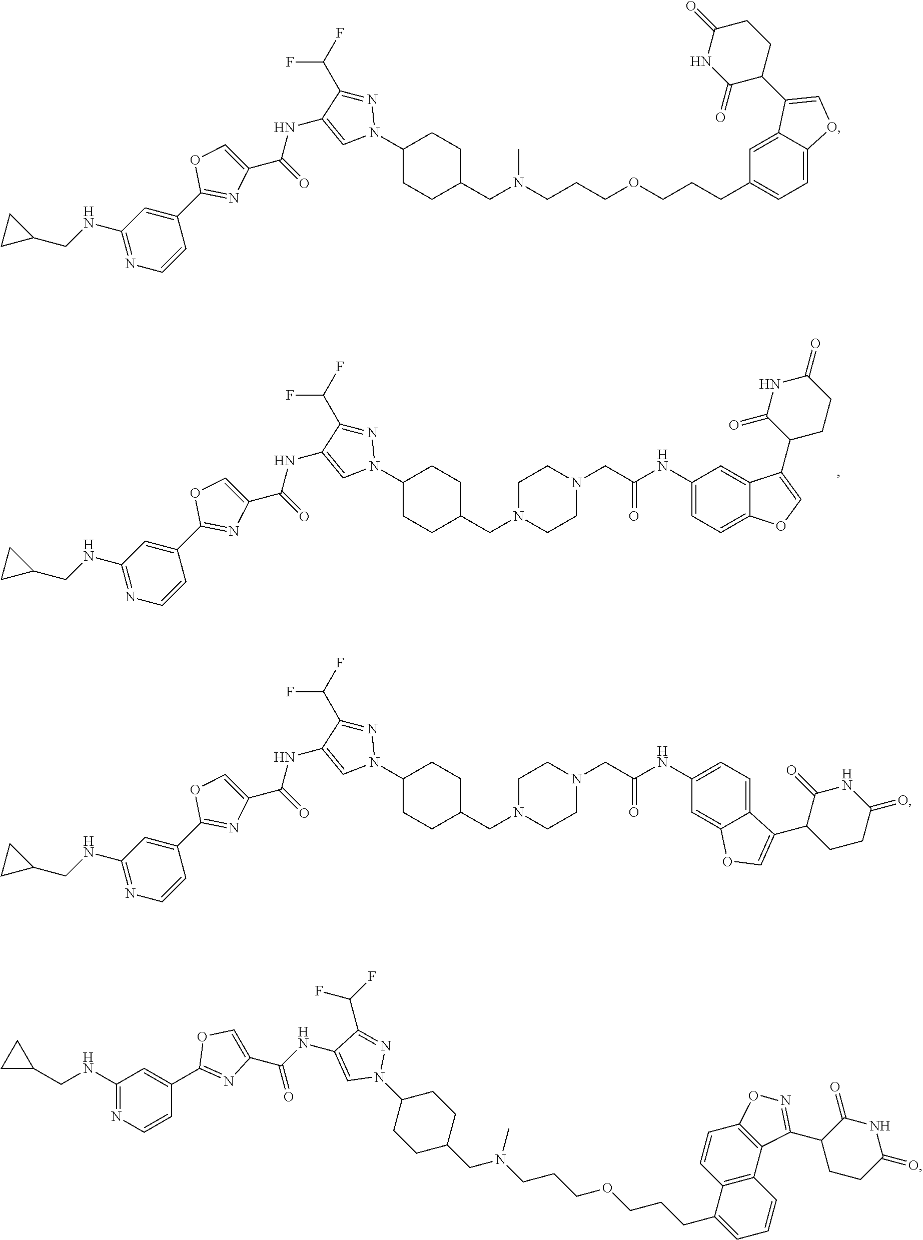 [SMILES: CN(CCCOCCCc1ccc2occ(C3CCC(=O)NC3=O)c2c1)CC1CCC(n2cc(NC(=O)c3coc(-c4ccnc(NCC5CC5)c4)n3)c(C(F)F)n2)CC1 ; O=C1CCC(c2coc3ccc(NC(=O)CN4CCN(CC5CCC(n6cc(NC(=O)c7coc(-c8ccnc(NCC9CC9)c8)n7)c(C(F)F)n6)CC5)CC4)cc23)C(=O)N1 ; O=C1CCC(c2coc3cc(NC(=O)CN4CCN(CC5CCC(n6cc(NC(=O)c7coc(-c8ccnc(NCC9CC9)c8)n7)c(C(F)F)n6)CC5)CC4)ccc23)C(=O)N1 ; CN(CCCOCCCc1cccc2c1ccc1onc(C3CCC(=O)NC3=O)c12)CC1CCC(n2cc(NC(=O)c3coc(-c4ccnc(NCC5CC5)c4)n3)c(C(F)F)n2)CC1]

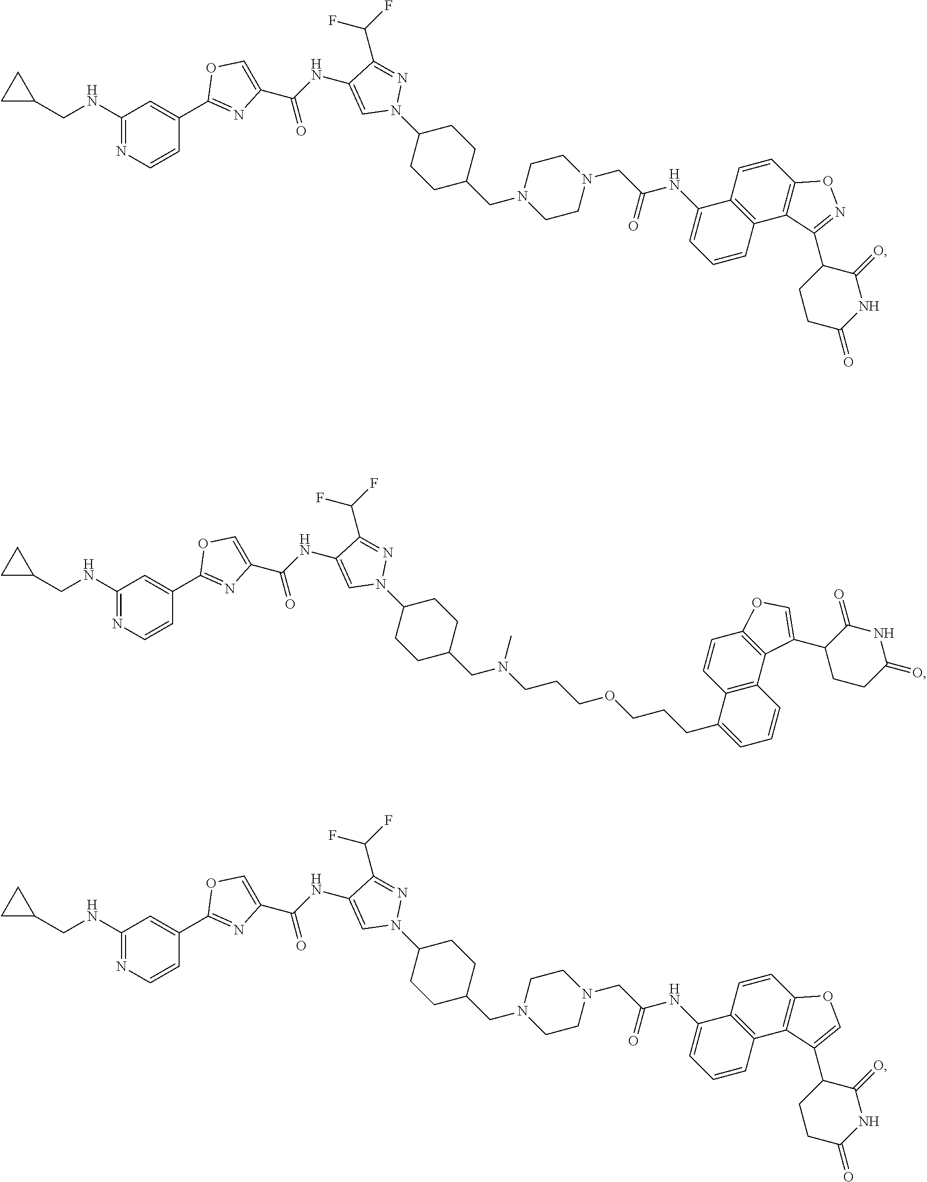 [SMILES: O=C1CCC(c2noc3ccc4c(NC(=O)CN5CCN(CC6CCC(n7cc(NC(=O)c8coc(-c9ccnc(NCC%10CC%10)c9)n8)c(C(F)F)n7)CC6)CC5)cccc4c23)C(=O)N1 ; CN(CCCOCCCc1cccc2c1ccc1occ(C3CCC(=O)NC3=O)c12)CC1CCC(n2cc(NC(=O)c3coc(-c4ccnc(NCC5CC5)c4)n3)c(C(F)F)n2)CC1 ; O=C1CCC(c2coc3ccc4c(NC(=O)CN5CCN(CC6CCC(n7cc(NC(=O)c8coc(-c9ccnc(NCC%10CC%10)c9)n8)c(C(F)F)n7)CC6)CC5)cccc4c23)C(=O)N1]

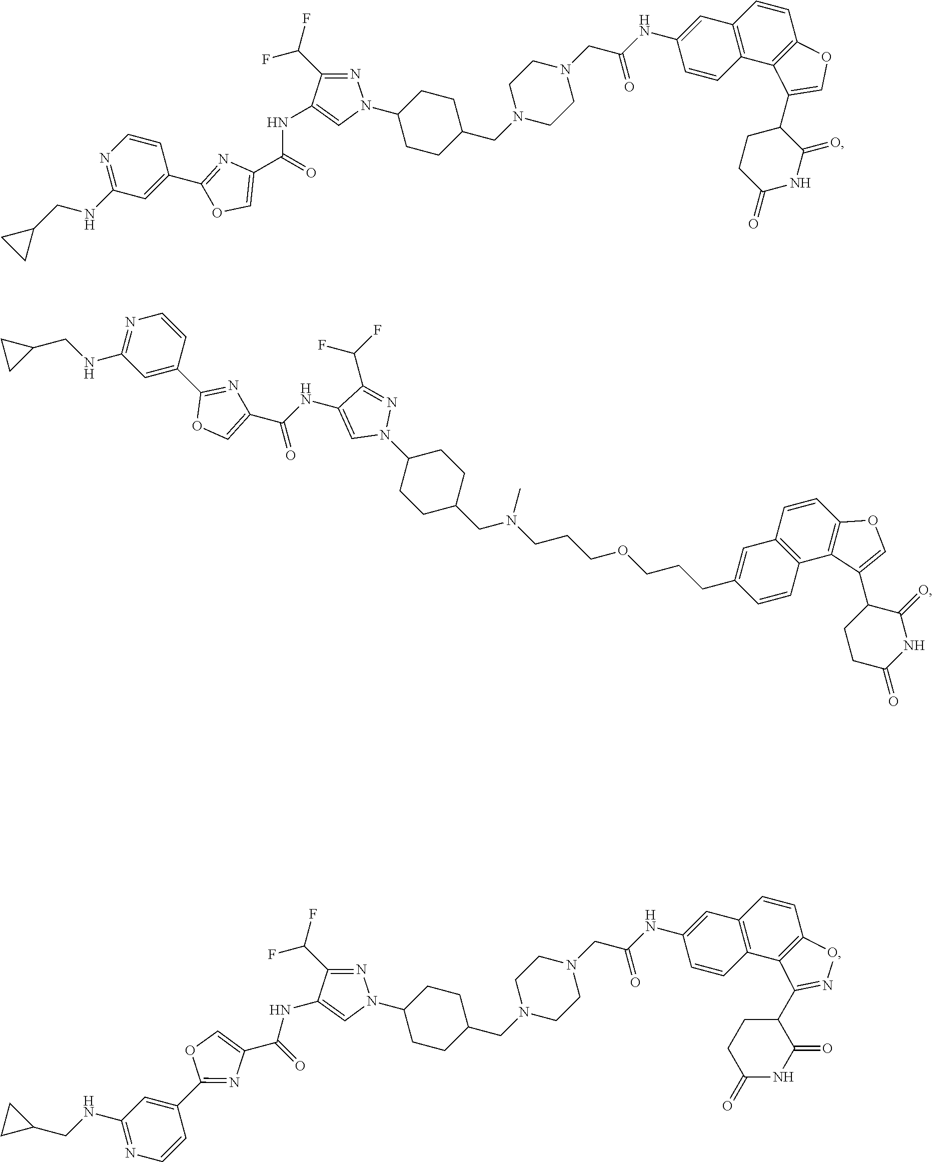 [SMILES: O=C1CCC(c2coc3ccc4cc(NC(=O)CN5CCN(CC6CCC(n7cc(NC(=O)c8coc(-c9ccnc(NCC%10CC%10)c9)n8)c(C(F)F)n7)CC6)CC5)ccc4c23)C(=O)N1 ; CN(CCCOCCCc1ccc2c(ccc3occ(C4CCC(=O)NC4=O)c32)c1)CC1CCC(n2cc(NC(=O)c3coc(-c4ccnc(NCC5CC5)c4)n3)c(C(F)F)n2)CC1 ; O=C1CCC(c2noc3ccc4cc(NC(=O)CN5CCN(CC6CCC(n7cc(NC(=O)c8coc(-c9ccnc(NCC%10CC%10)c9)n8)c(C(F)F)n7)CC6)CC5)ccc4c23)C(=O)N1]

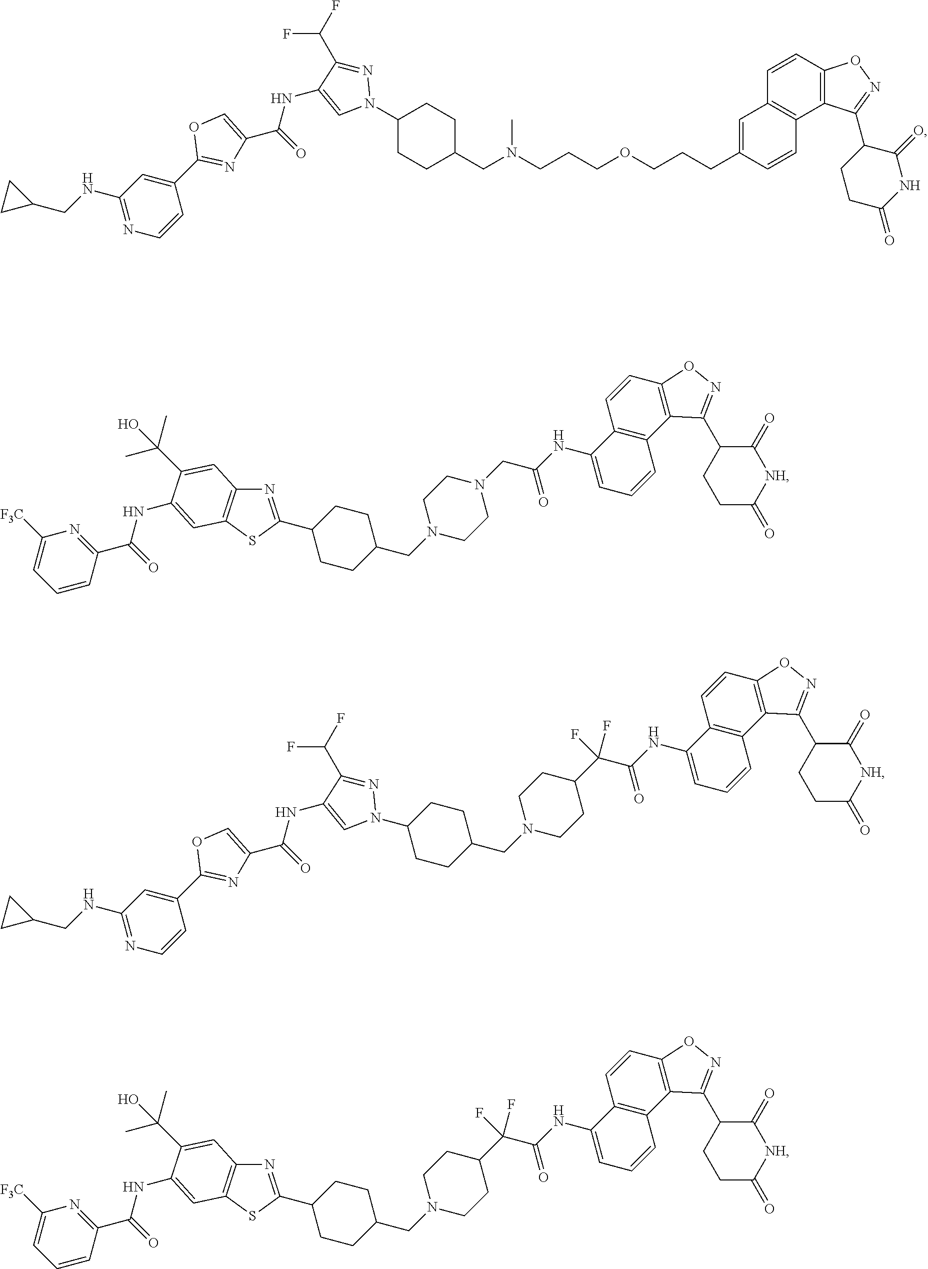 [SMILES: CN(CCCOCCCc1ccc2c(ccc3onc(C4CCC(=O)NC4=O)c32)c1)CC1CCC(n2cc(NC(=O)c3coc(-c4ccnc(NCC5CC5)c4)n3)c(C(F)F)n2)CC1 ; CC(C)(O)c1cc2nc(C3CCC(CN4CCN(CC(=O)Nc5cccc6c5ccc5onc(C7CCC(=O)NC7=O)c56)CC4)CC3)sc2cc1NC(=O)c1cccc(C(F)(F)F)n1 ; O=C1CCC(c2noc3ccc4c(NC(=O)C(F)(F)C5CCN(CC6CCC(n7cc(NC(=O)c8coc(-c9ccnc(NCC%10CC%10)c9)n8)c(C(F)F)n7)CC6)CC5)cccc4c23)C(=O)N1 ; CC(C)(O)c1cc2nc(C3CCC(CN4CCC(C(F)(F)C(=O)Nc5cccc6c5ccc5onc(C7CCC(=O)NC7=O)c56)CC4)CC3)sc2cc1NC(=O)c1cccc(C(F)(F)F)n1]

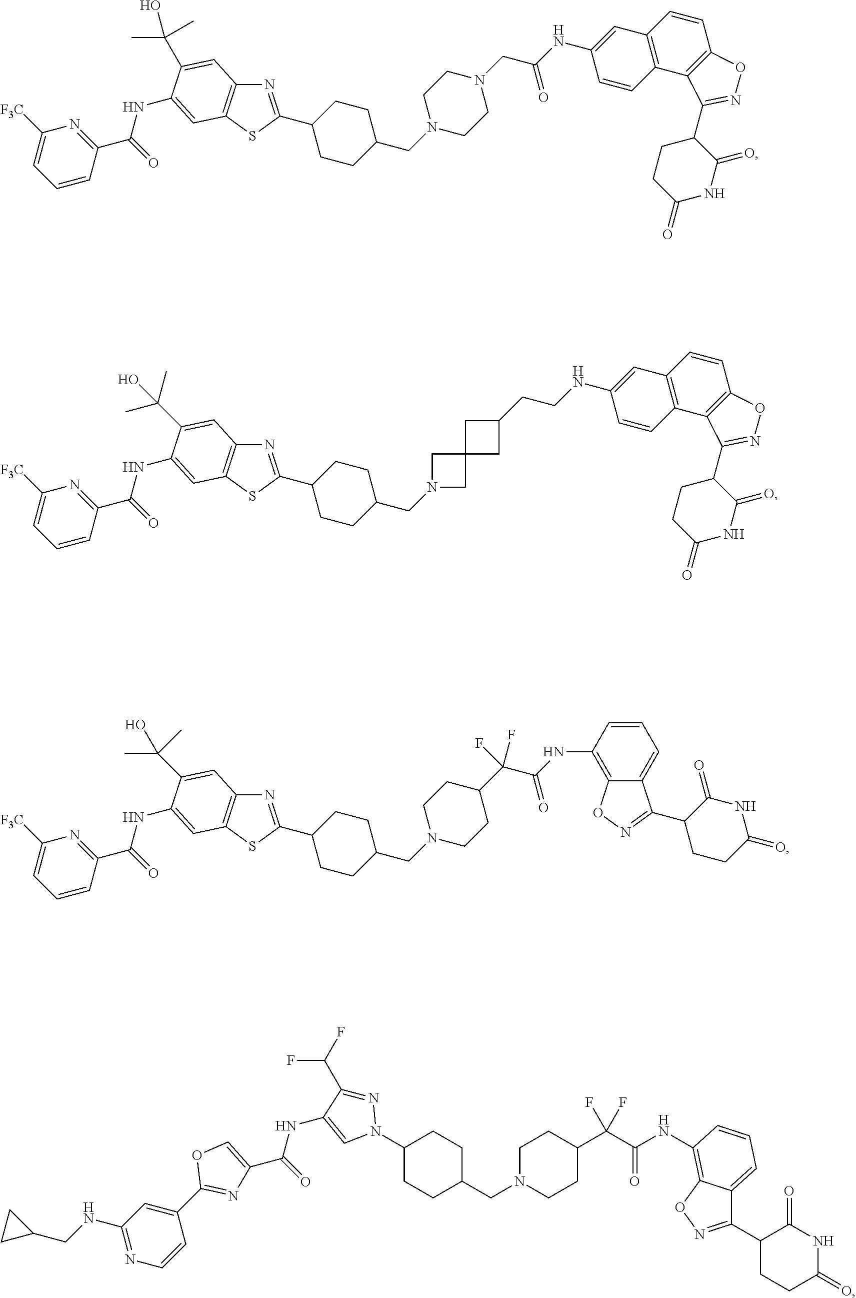 [SMILES: CC(C)(O)c1cc2nc(C3CCC(CN4CCN(CC(=O)Nc5ccc6c(ccc7onc(C8CCC(=O)NC8=O)c76)c5)CC4)CC3)sc2cc1NC(=O)c1cccc(C(F)(F)F)n1 ; CC(C)(O)c1cc2nc(C3CCC(CN4CC5(CC(CCNc6ccc7c(ccc8onc(C9CCC(=O)NC9=O)c87)c6)C5)C4)CC3)sc2cc1NC(=O)c1cccc(C(F)(F)F)n1 ; CC(C)(O)c1cc2nc(C3CCC(CN4CCC(C(F)(F)C(=O)Nc5cccc6c(C7CCC(=O)NC7=O)noc56)CC4)CC3)sc2cc1NC(=O)c1cccc(C(F)(F)F)n1 ; O=C1CCC(c2noc3c(NC(=O)C(F)(F)C4CCN(CC5CCC(n6cc(NC(=O)c7coc(-c8ccnc(NCC9CC9)c8)n7)c(C(F)F)n6)CC5)CC4)cccc23)C(=O)N1]

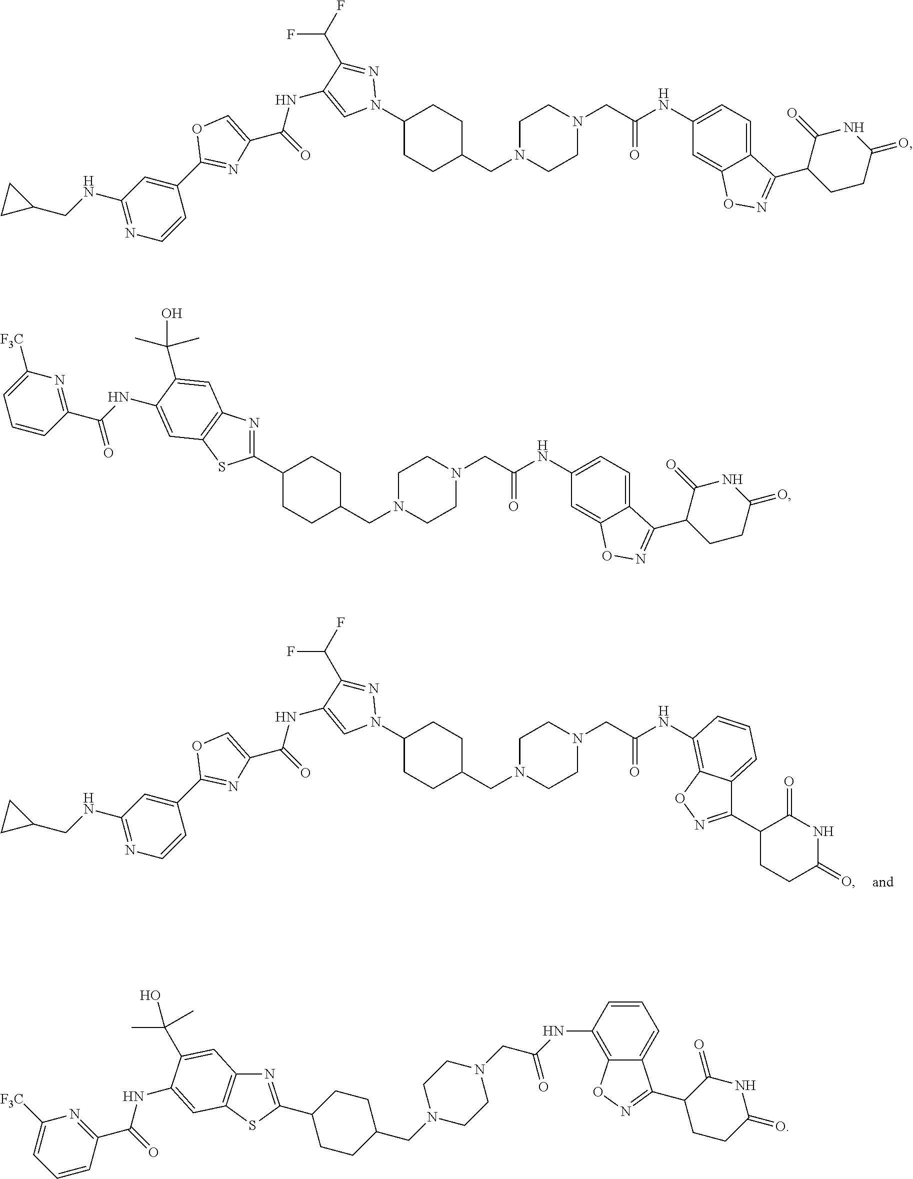 [SMILES: O=C1CCC(c2noc3cc(NC(=O)CN4CCN(CC5CCC(n6cc(NC(=O)c7coc(-c8ccnc(NCC9CC9)c8)n7)c(C(F)F)n6)CC5)CC4)ccc23)C(=O)N1 ; CC(C)(O)c1cc2nc(C3CCC(CN4CCN(CC(=O)Nc5ccc6c(C7CCC(=O)NC7=O)noc6c5)CC4)CC3)sc2cc1NC(=O)c1cccc(C(F)(F)F)n1 ; O=C1CCC(c2noc3c(NC(=O)CN4CCN(CC5CCC(n6cc(NC(=O)c7coc(-c8ccnc(NCC9CC9)c8)n7)c(C(F)F)n6)CC5)CC4)cccc23)C(=O)N1 ; CC(C)(O)c1cc2nc(C3CCC(CN4CCN(CC(=O)Nc5cccc6c(C7CCC(=O)NC7=O)noc56)CC4)CC3)sc2cc1NC(=O)c1cccc(C(F)(F)F)n1]

13. The compound, the stereoisomer thereof, or the pharmaceutically acceptable salt thereof according to claim 12, wherein the compound is selected from

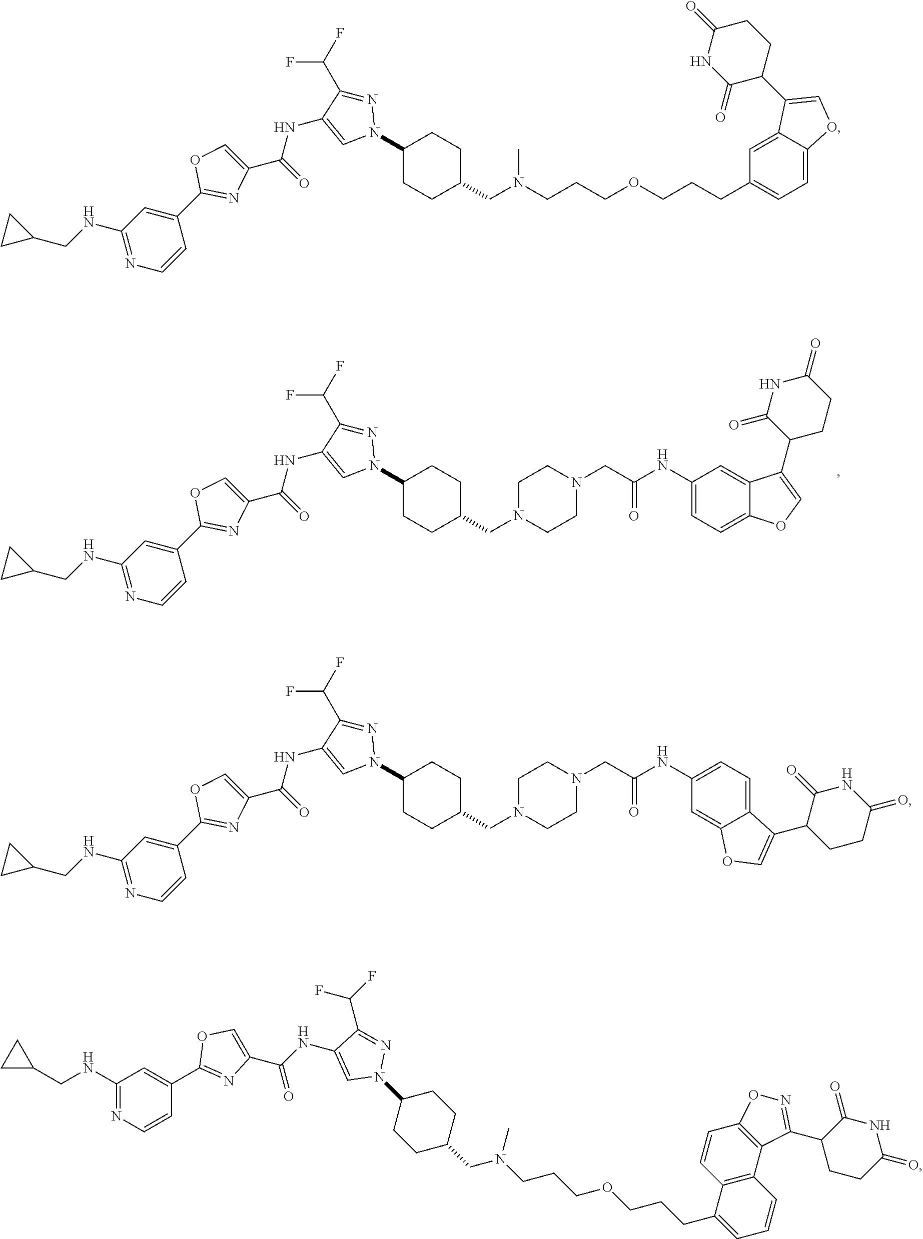

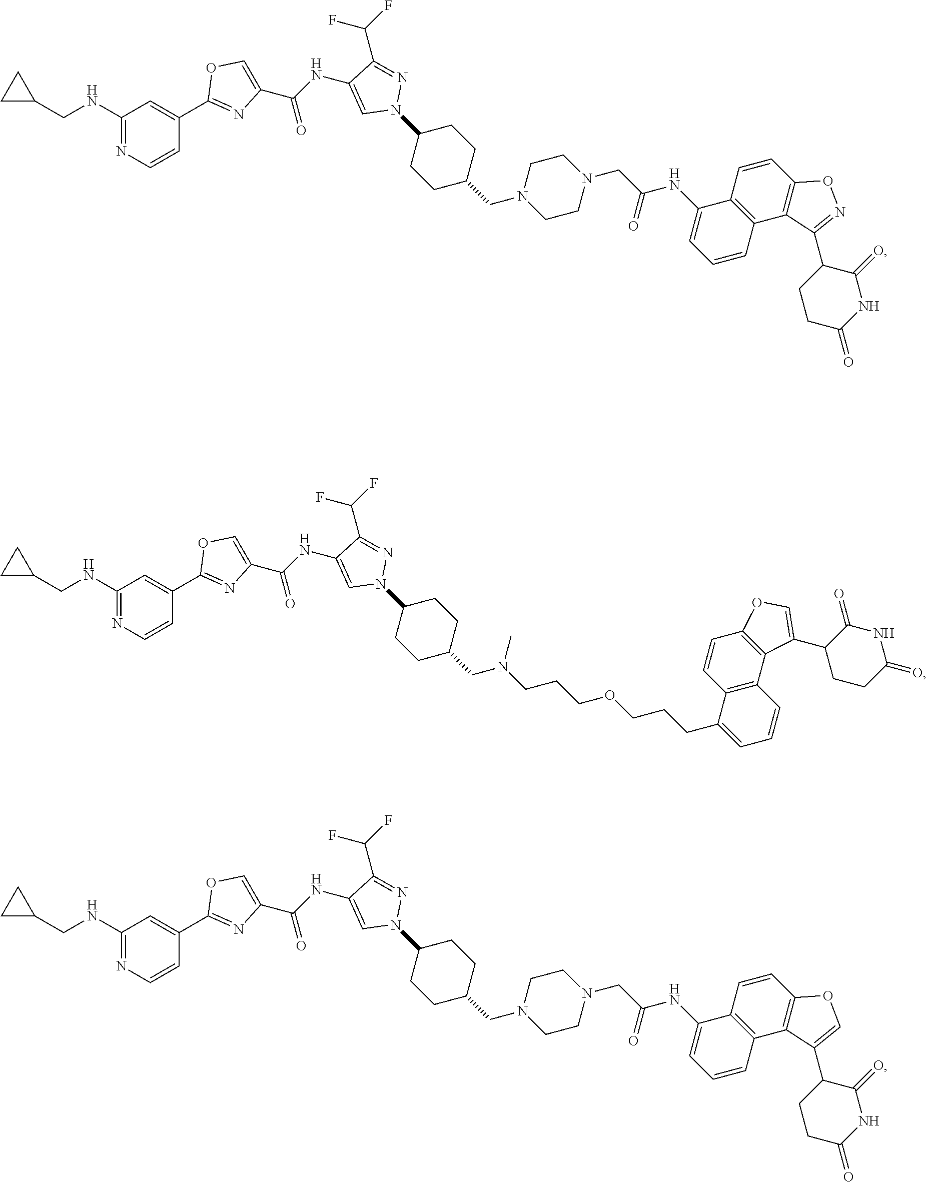

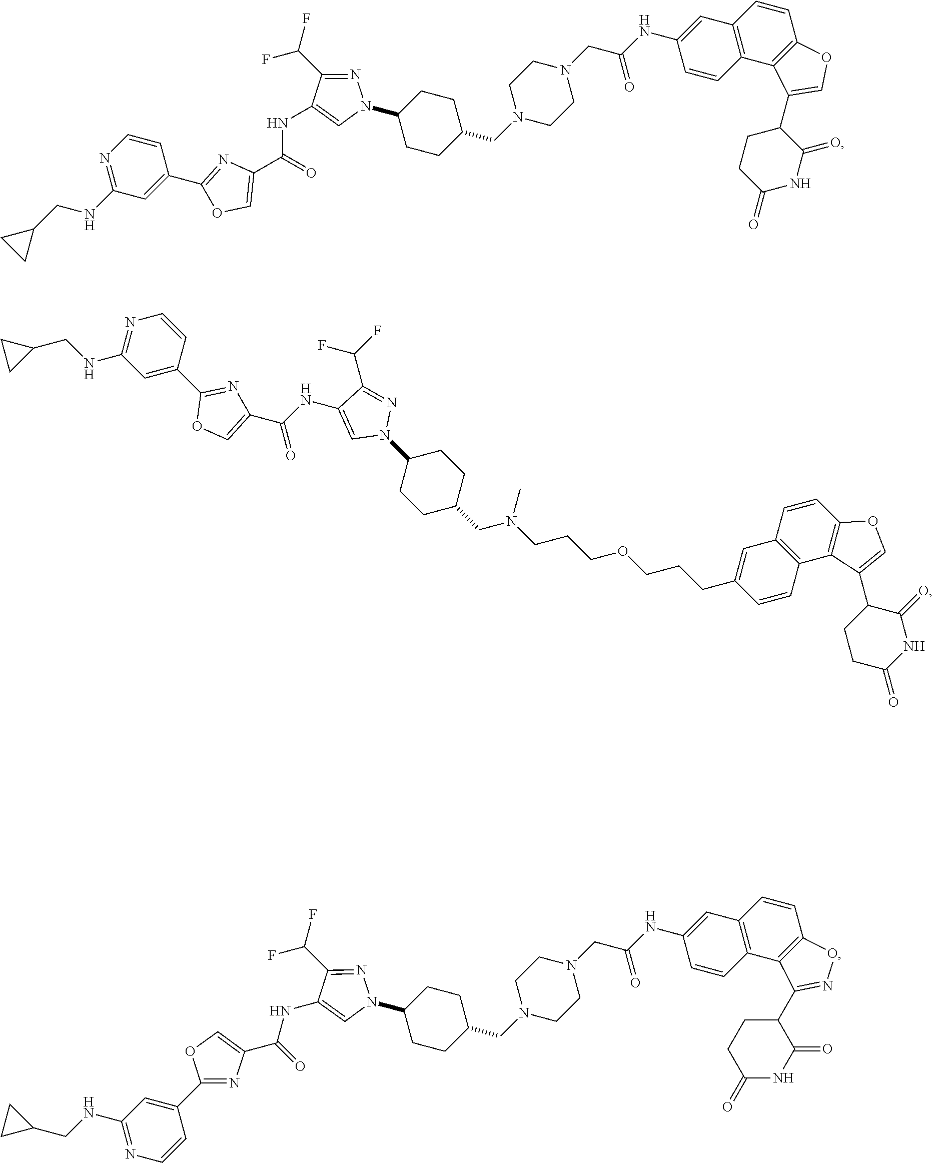

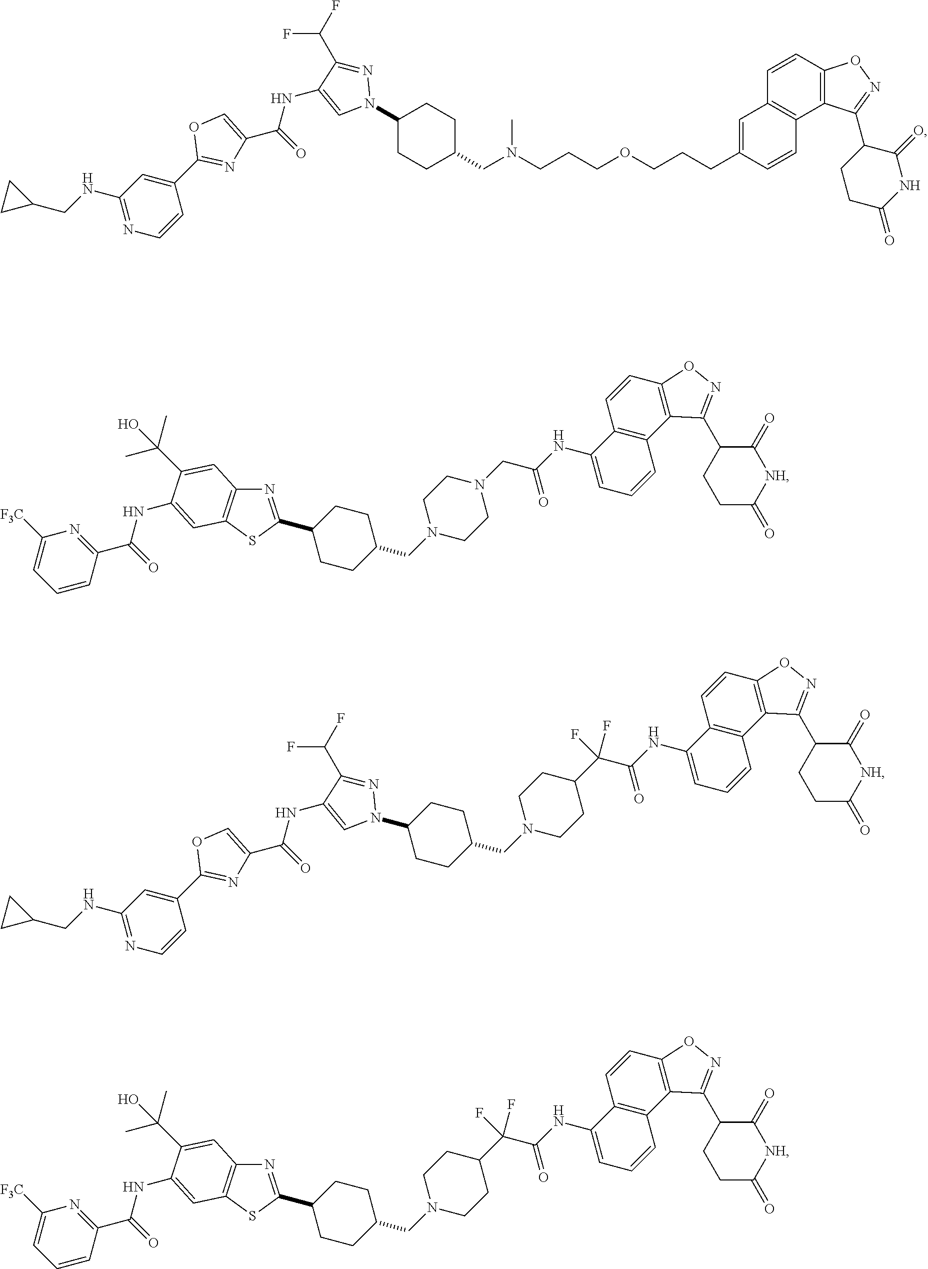

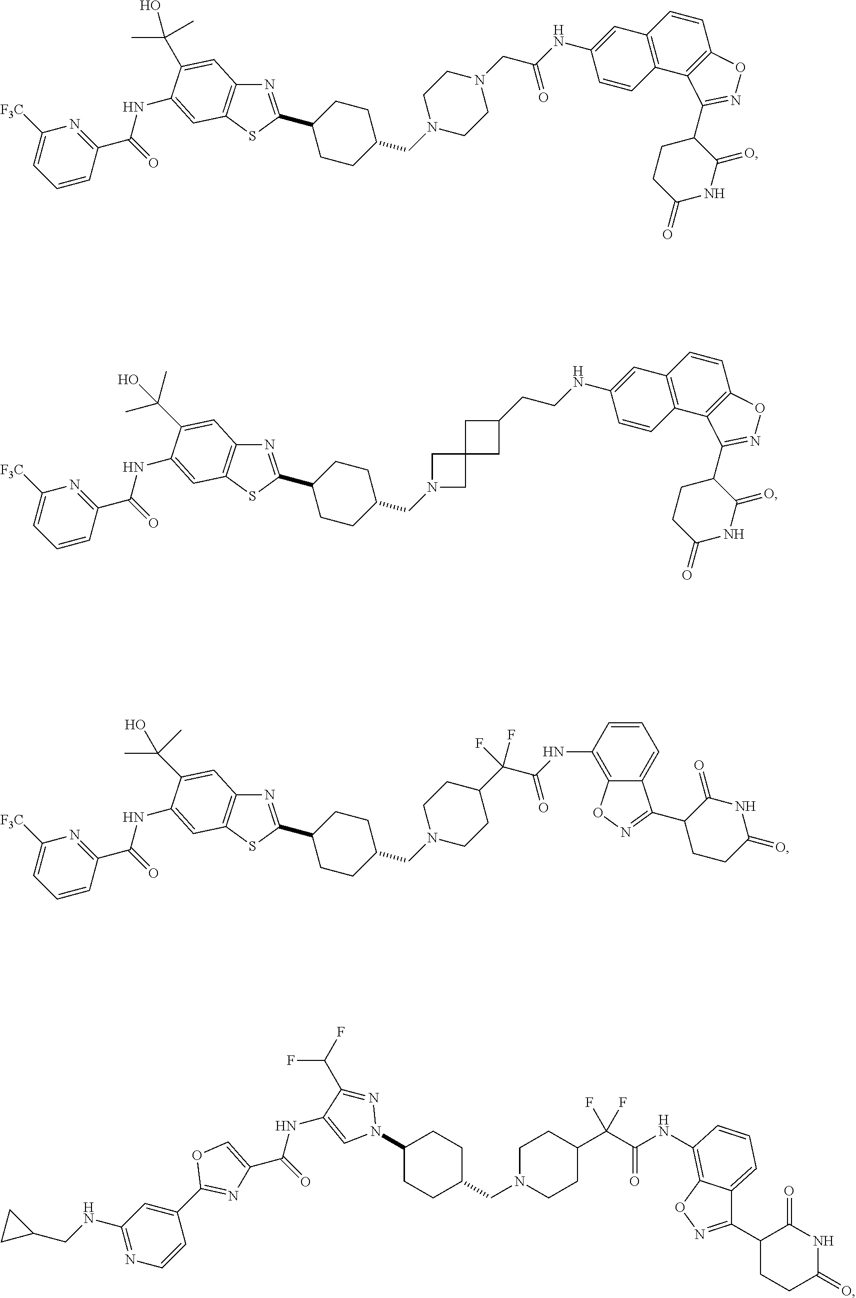

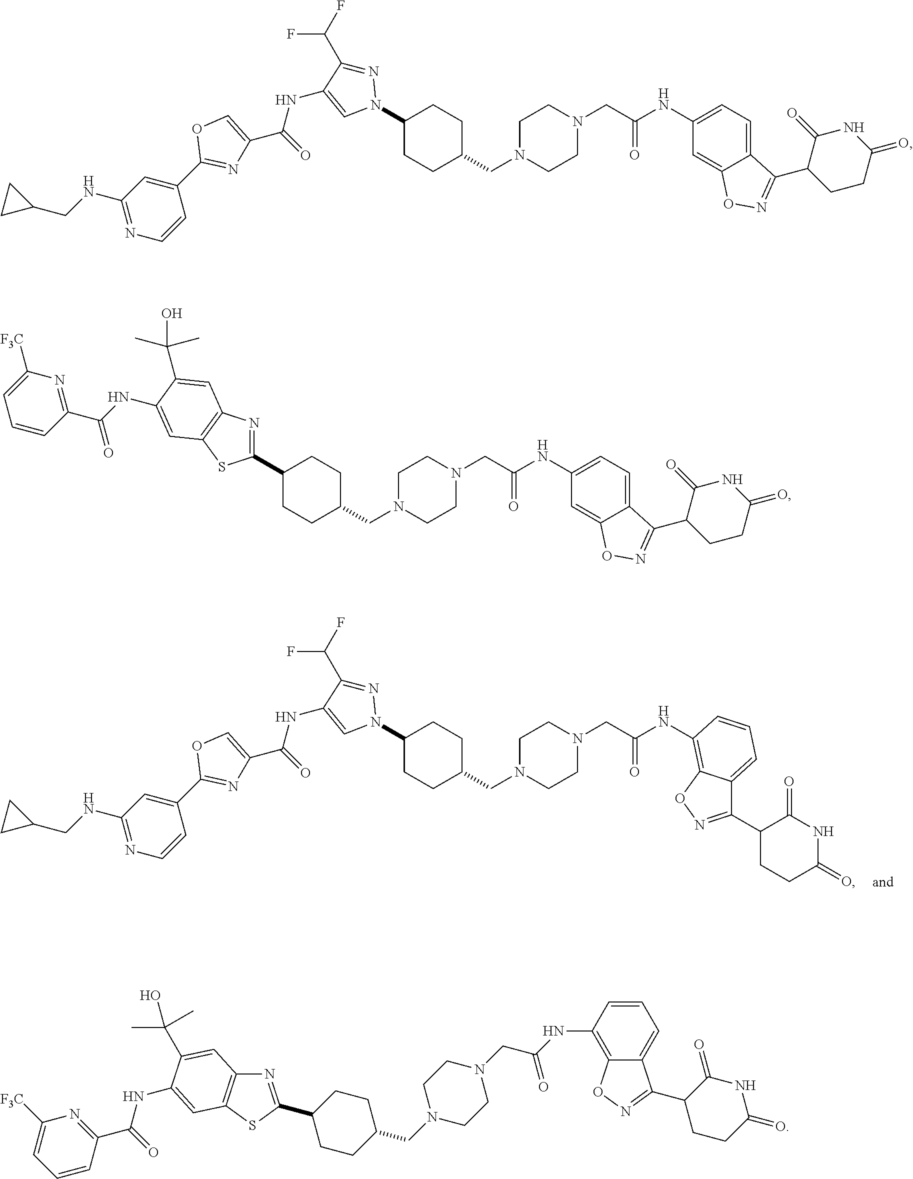

14. The compound, the stereoisomer thereof, or the pharmaceutically acceptable salt thereof according to claim 13, wherein the compound is selected from

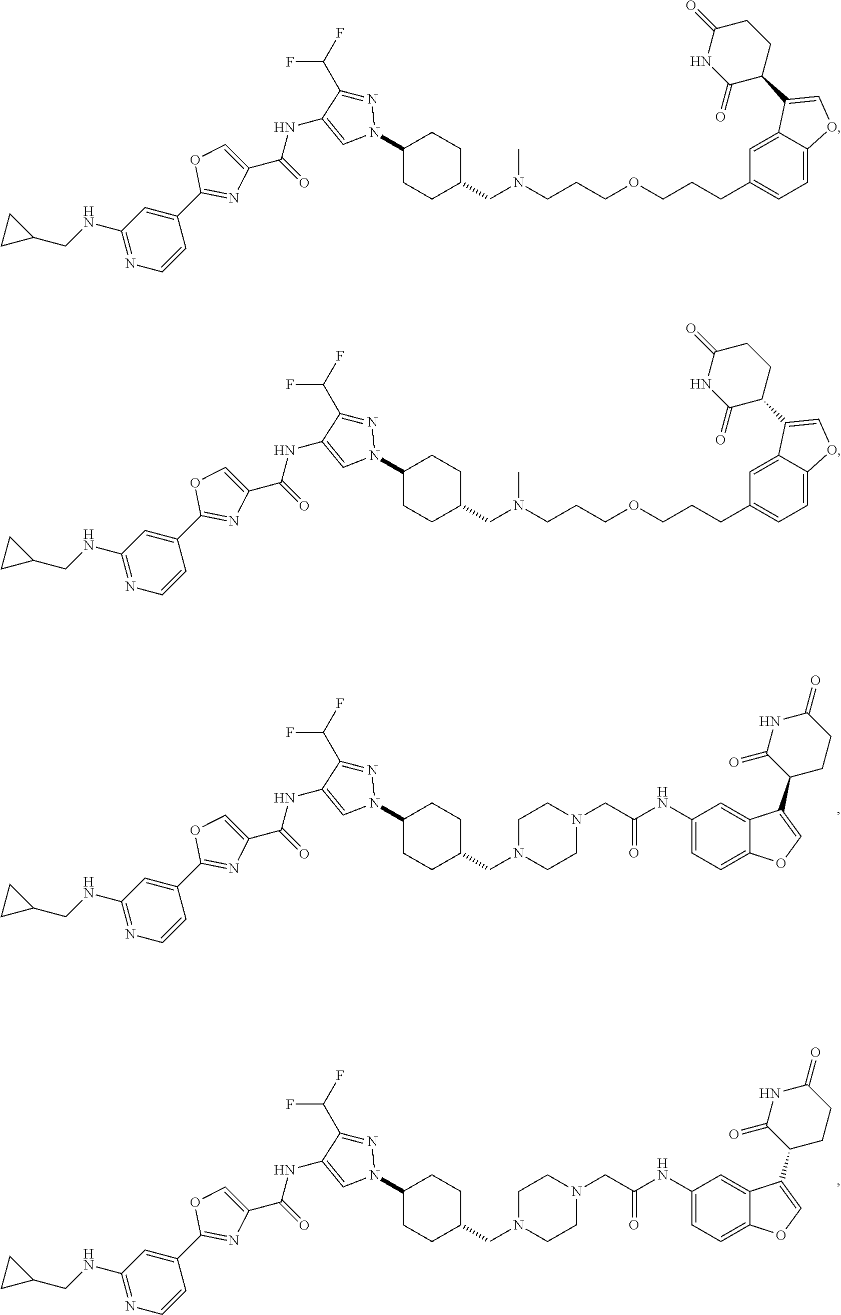

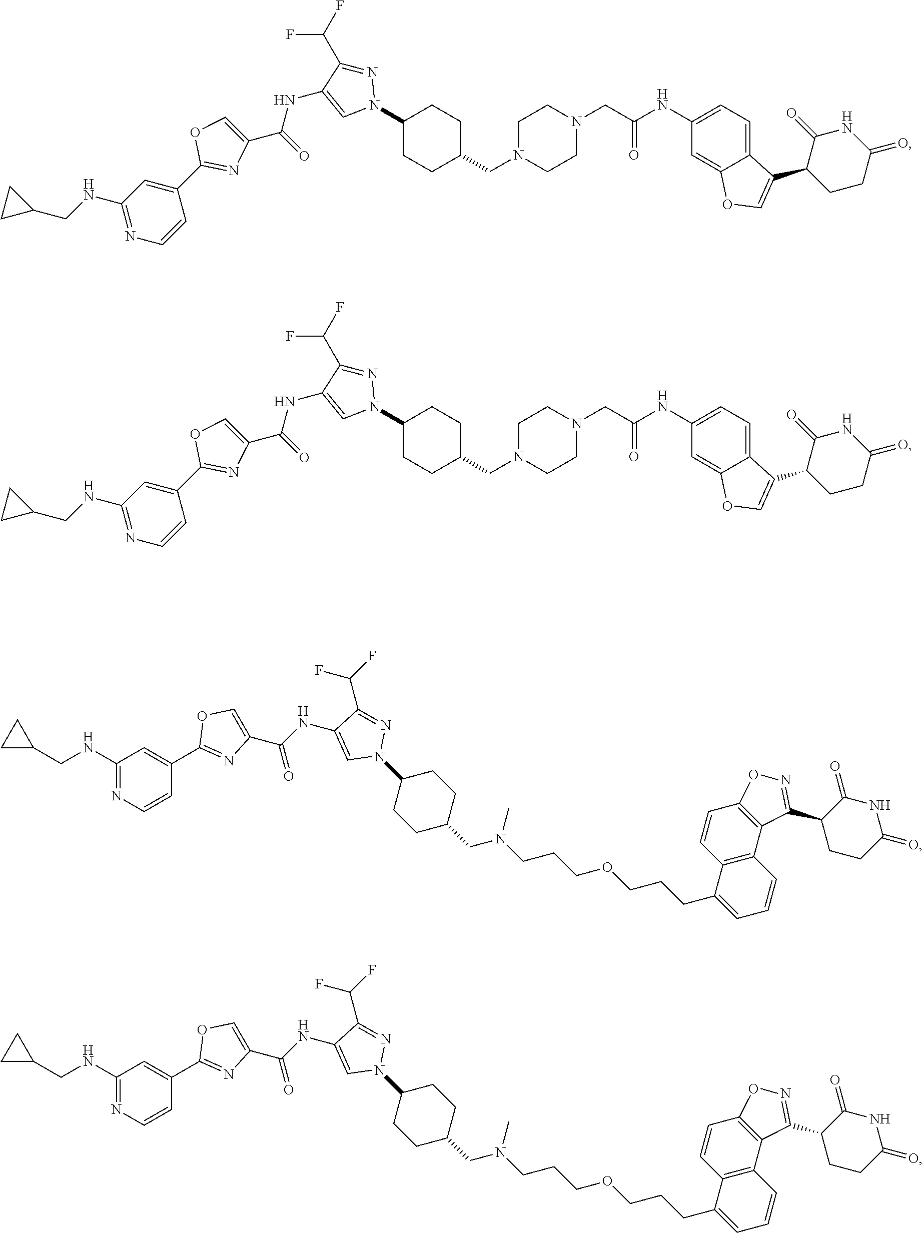

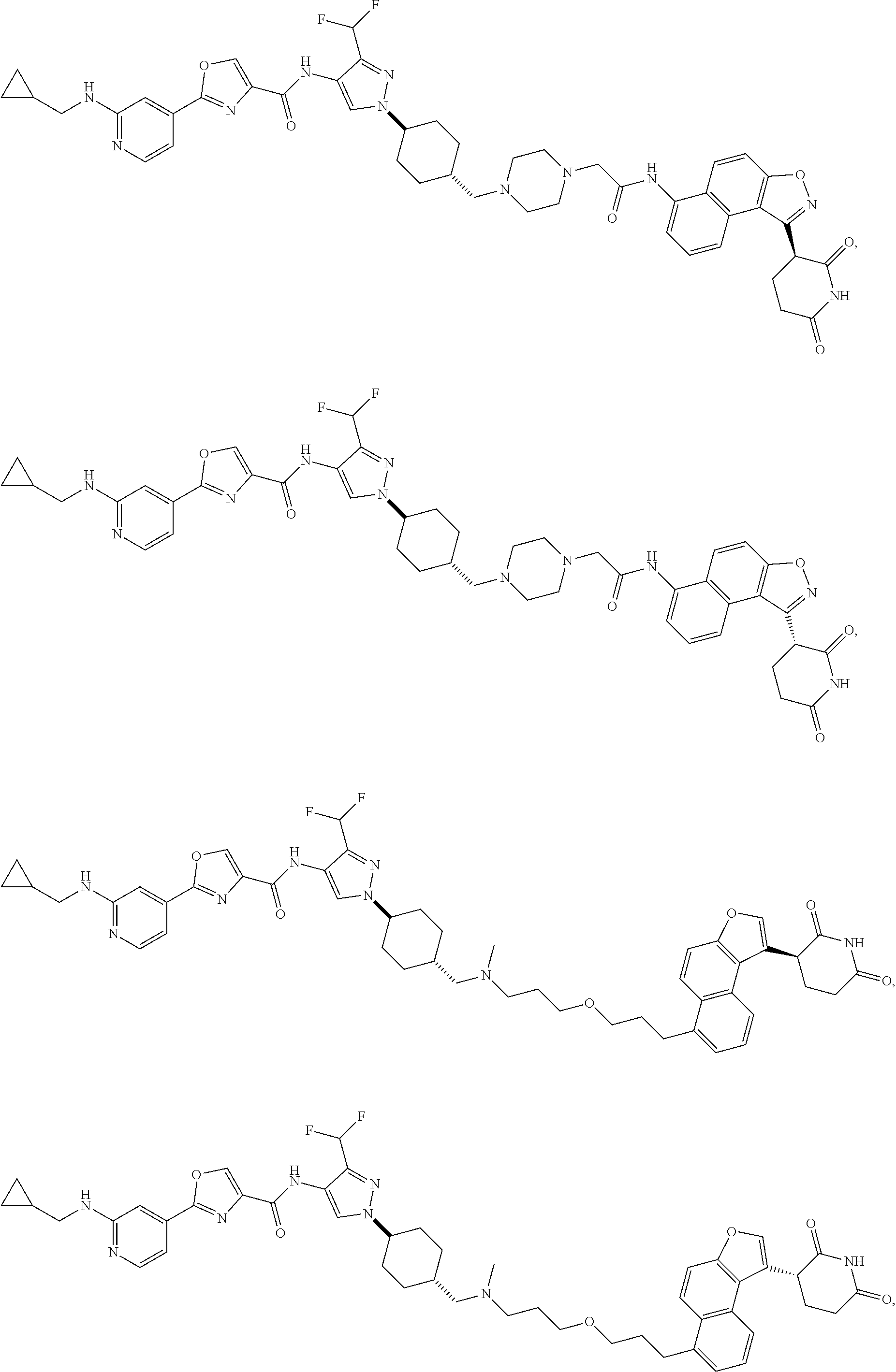

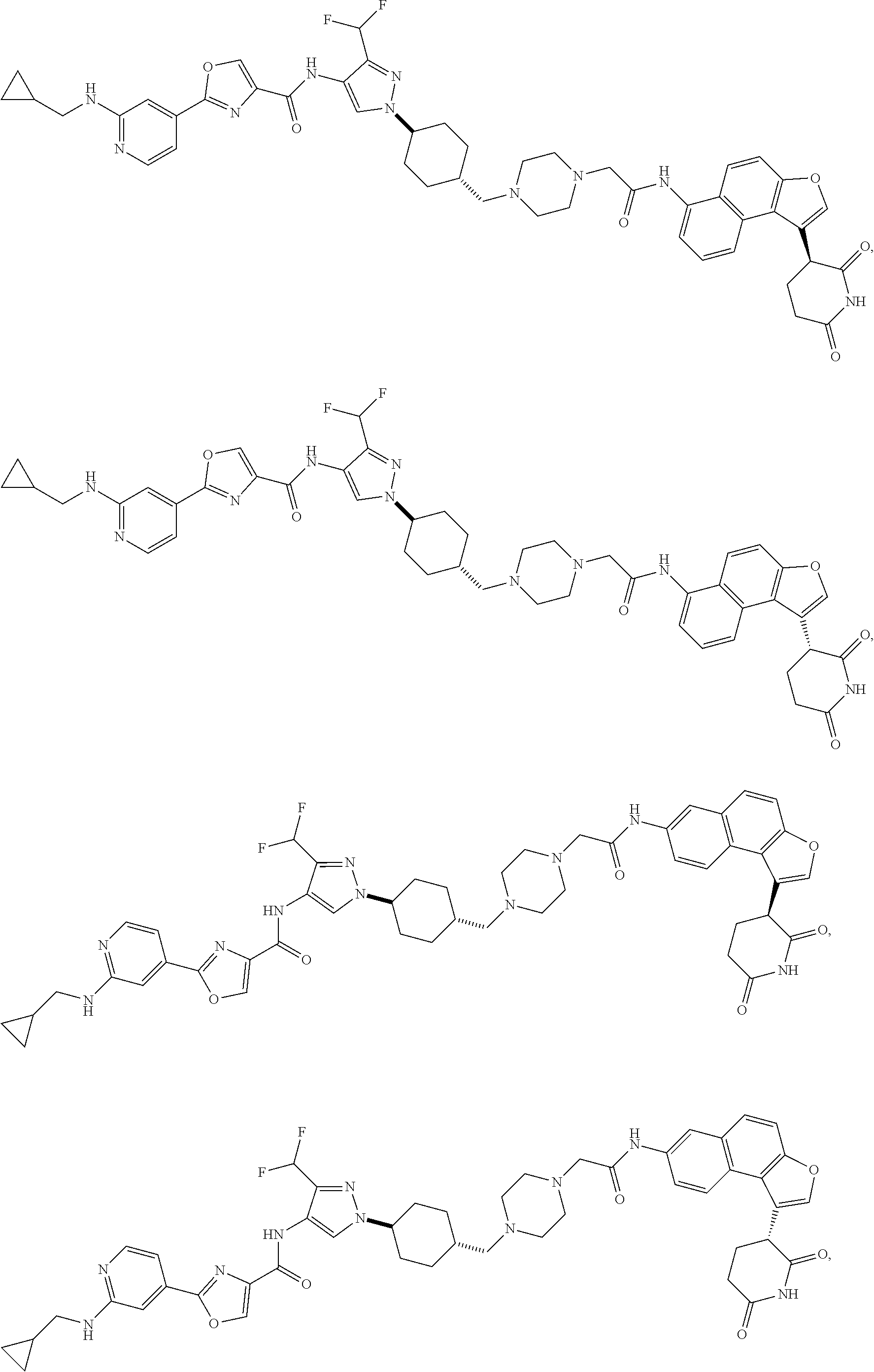

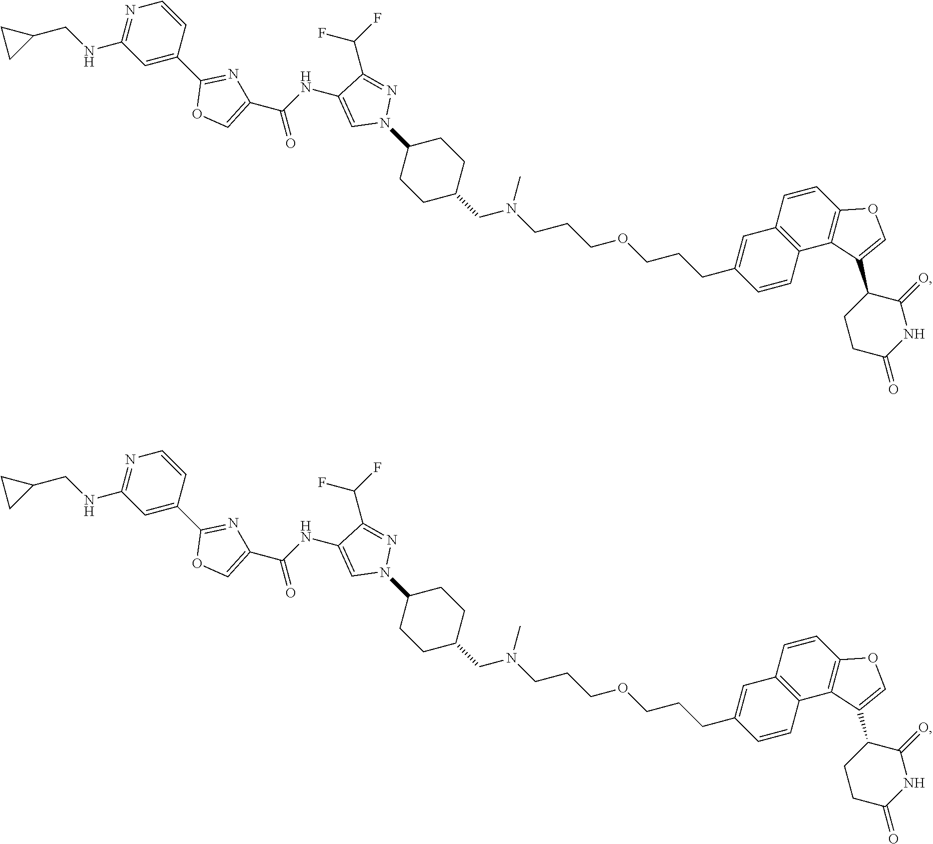

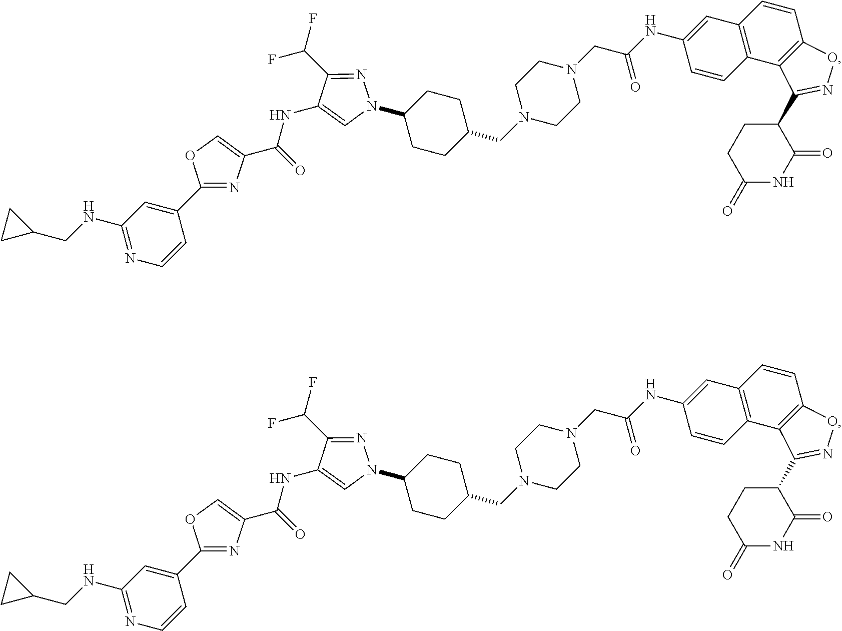

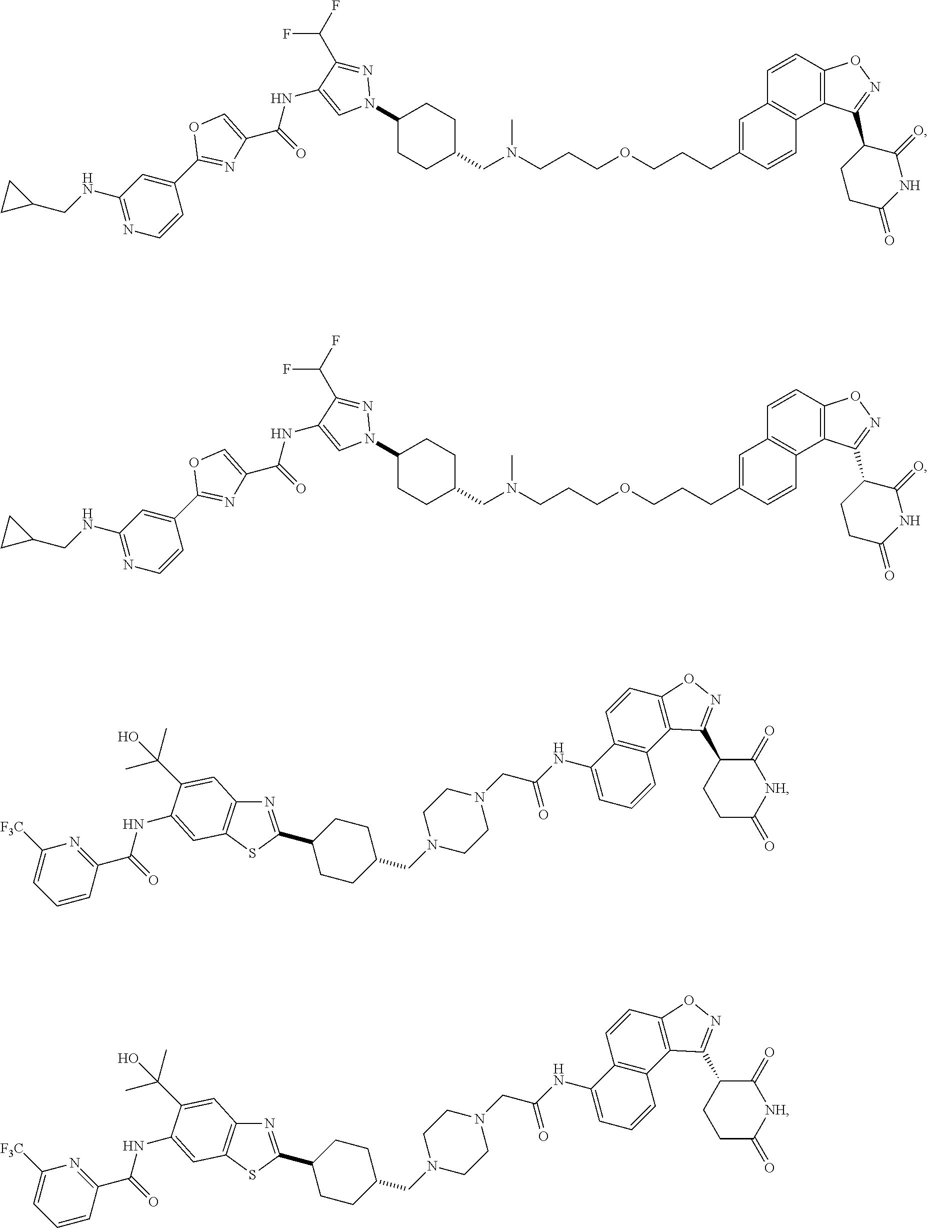

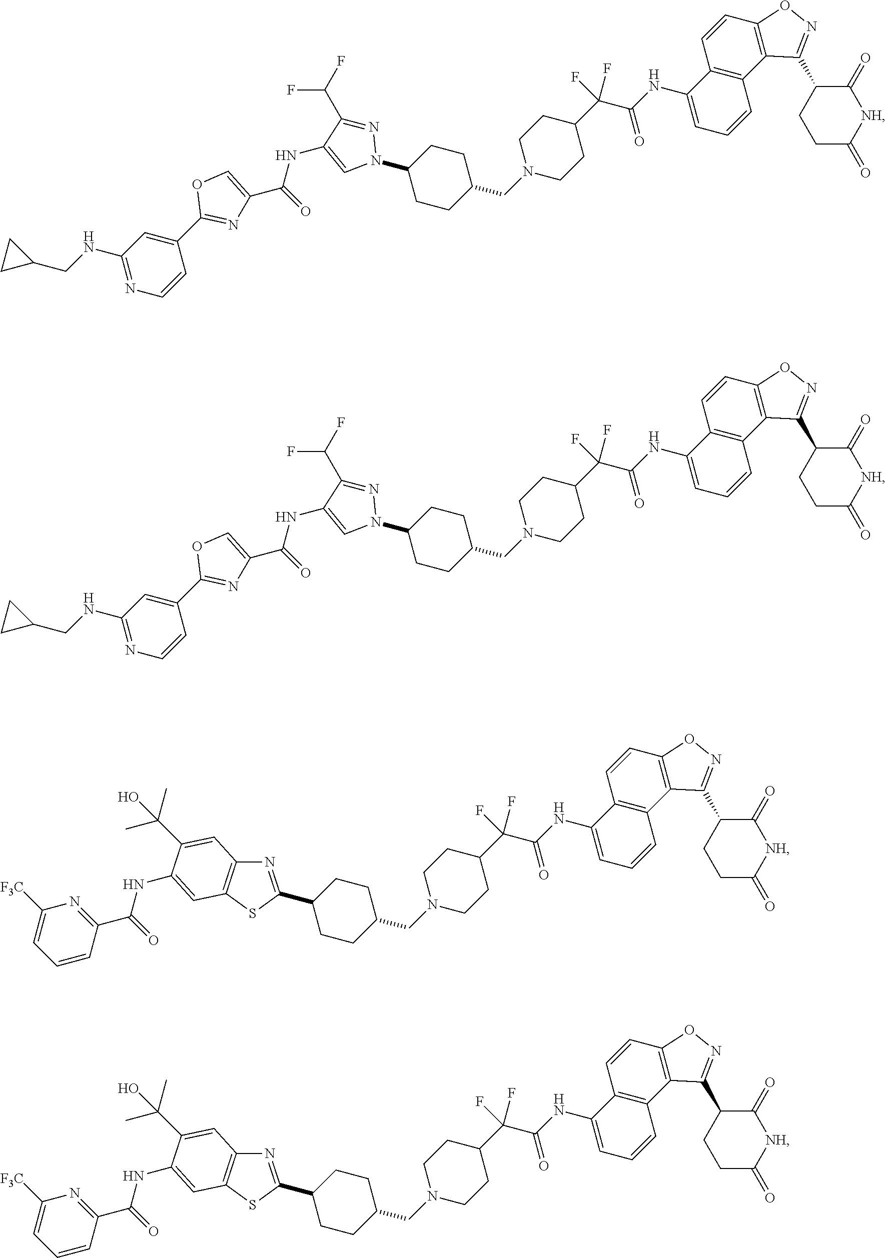

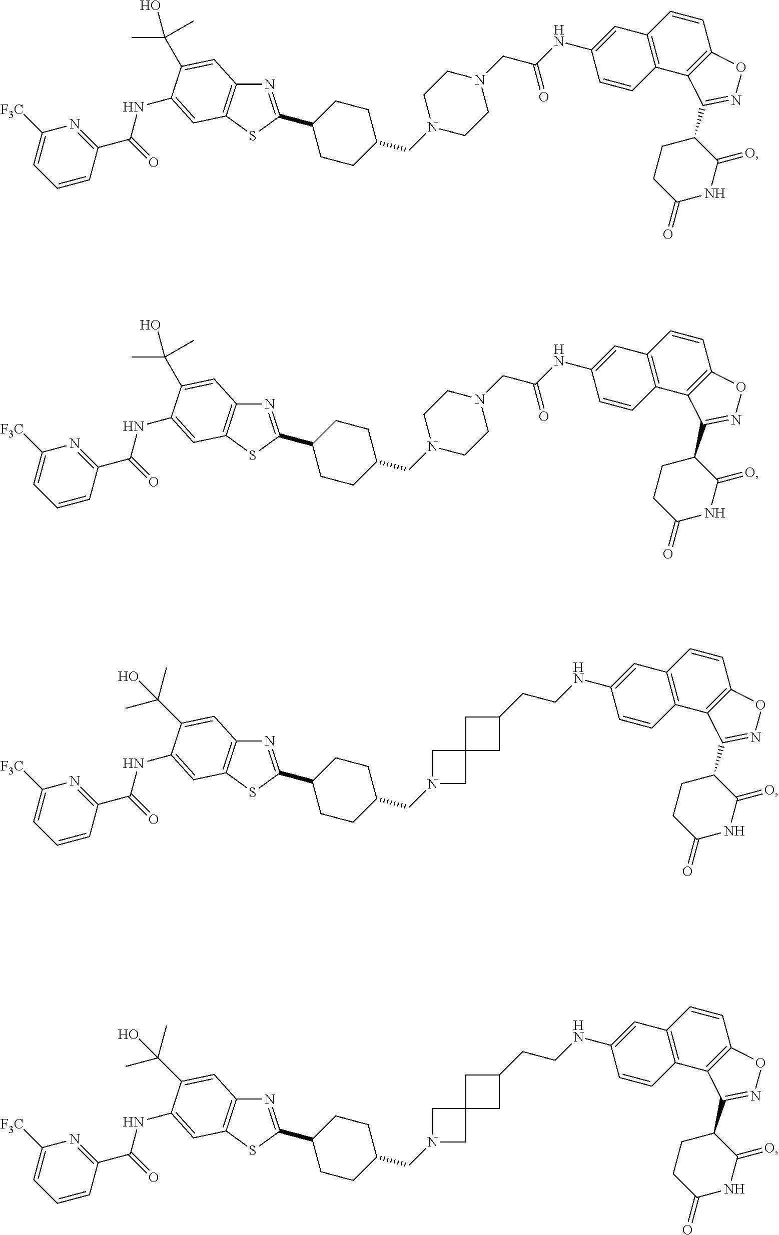

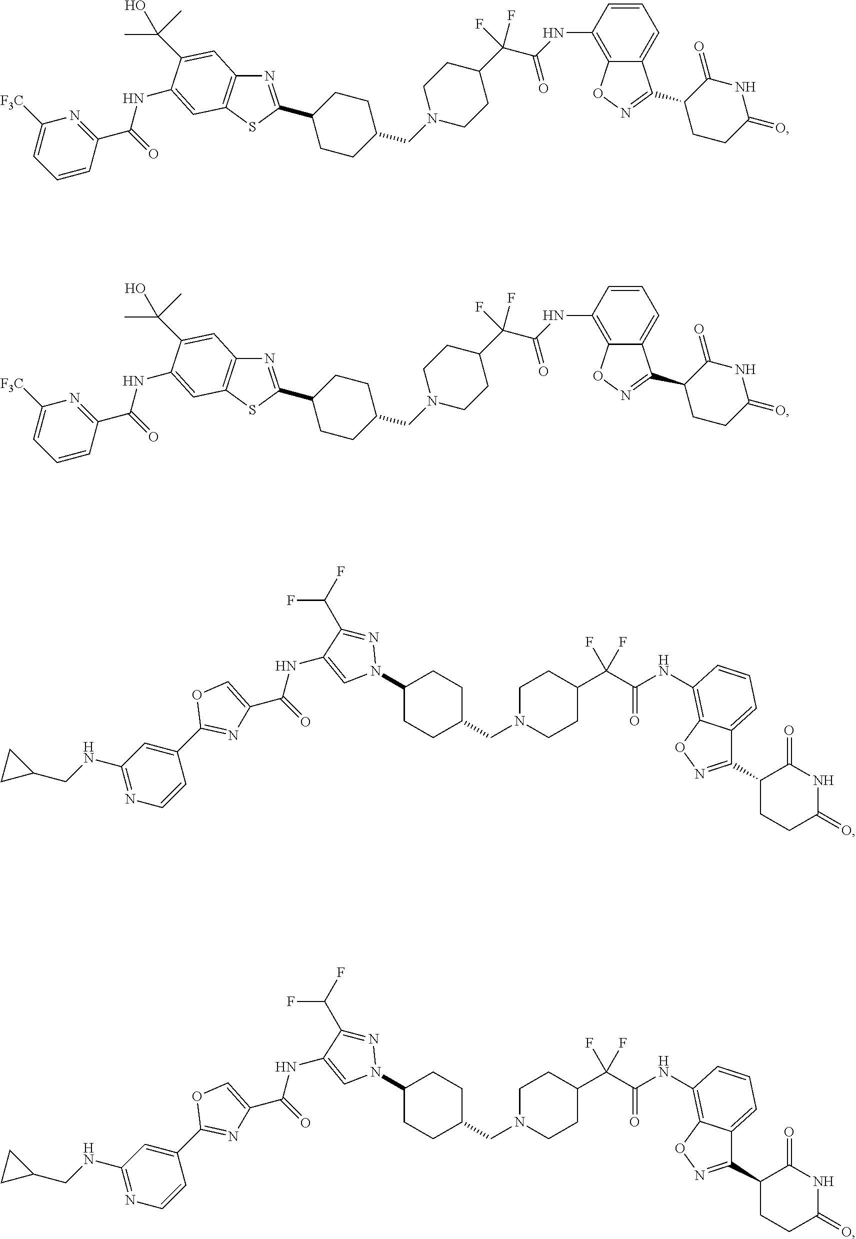

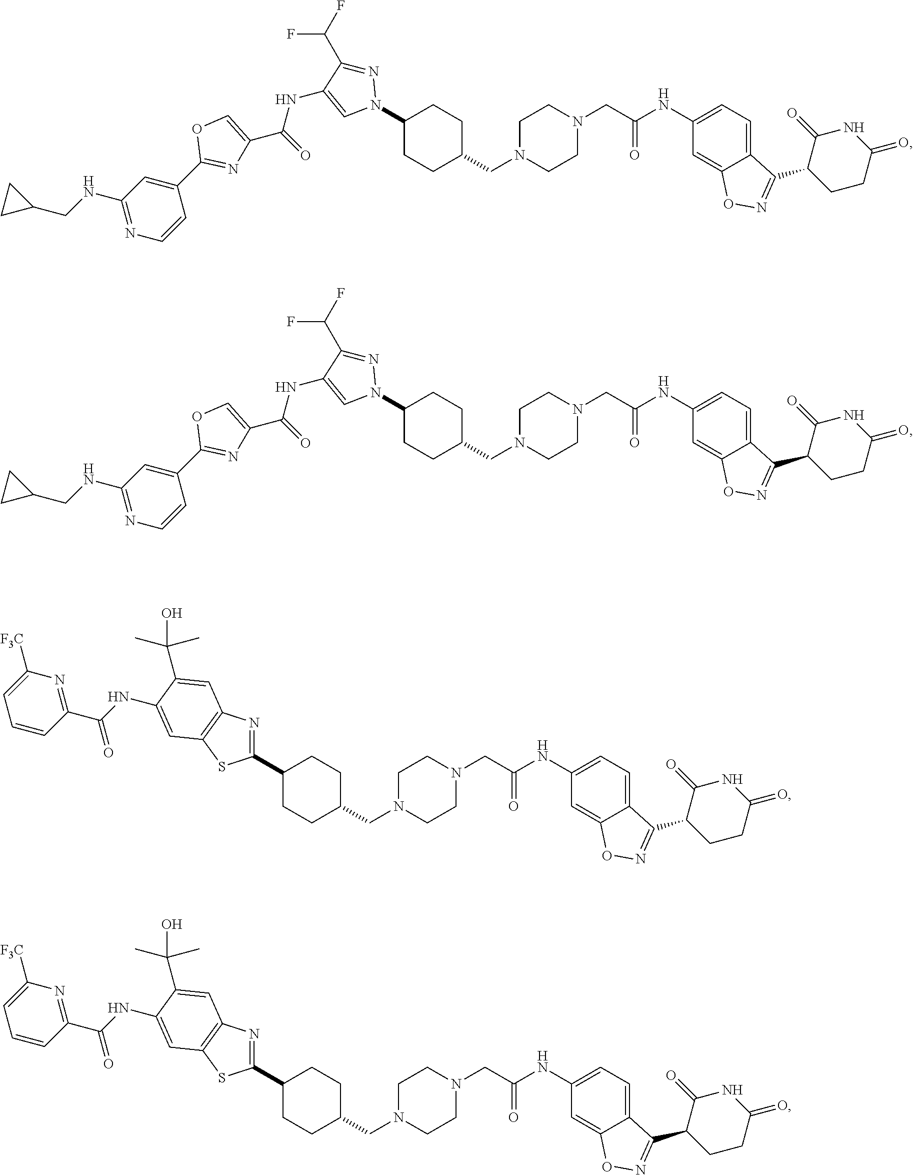

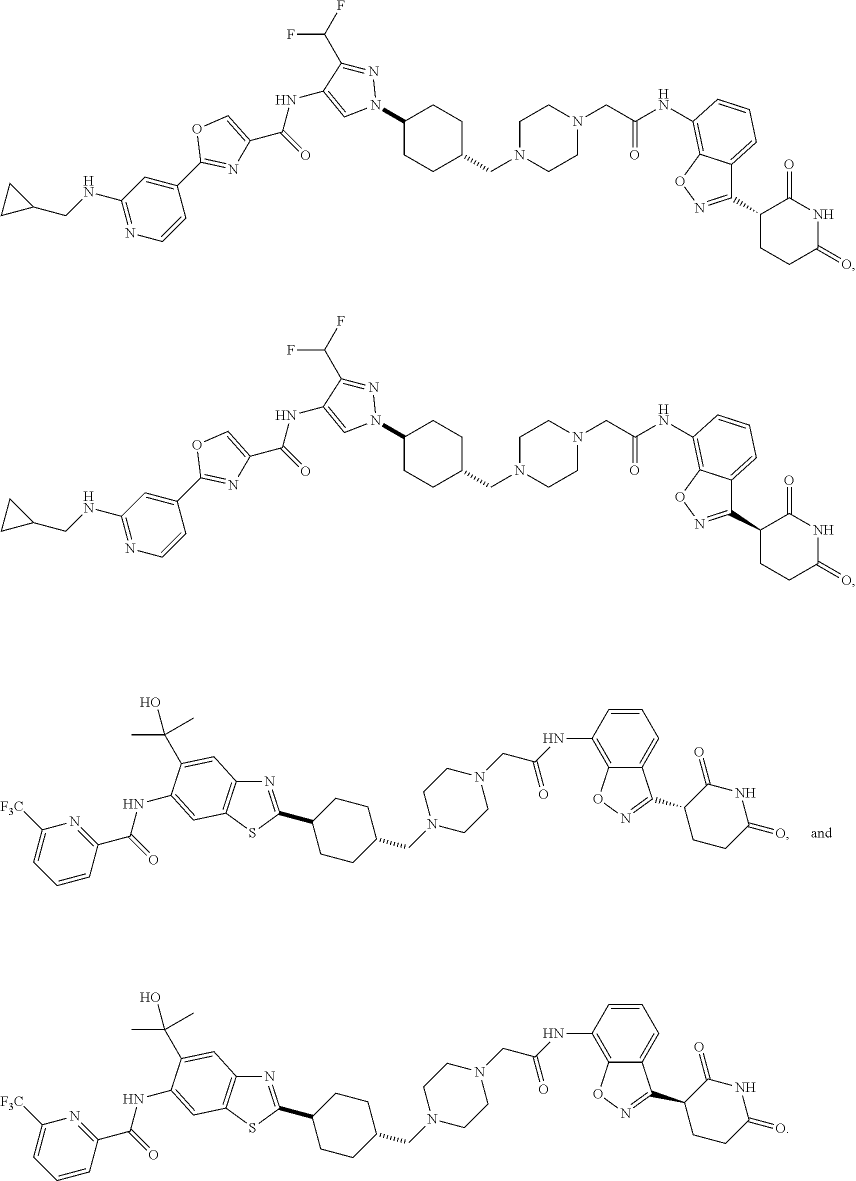

15. A pharmaceutical composition comprising a therapeutically effective amount of the compound, the stereoisomer thereof, or the pharmaceutically acceptable salt thereof according to claim 1.

16. A method for treating tumors associated with proteolysis targeting chimeras of Interleukin-1 Receptor-Associated Kinase 4 in a subject in need thereof, comprising administering the compound, the stereoisomer thereof, or the pharmaceutically acceptable salt thereof according to claim 1.

17. The method according to claim 16, wherein the tumor associated with proteolysis targeting chimeras of Interleukin-1 Receptor-Associated Kinase 4 is B-cell lymphoma.

18. A method for treating tumors associated with proteolysis targeting chimeras of Interleukin-1 Receptor-Associated Kinase 4 in a subject in need thereof, comprising administering the compound, the stereoisomer thereof, or the pharmaceutically acceptable salt thereof according to claim 12.

19. The method according to claim 18, wherein the tumor associated with proteolysis targeting chimeras of Interleukin-1 Receptor-Associated Kinase 4 is B-cell lymphoma.
